(19) United States
(12) Patent Application Publication
(10) Pub. No.: US 2026/0021088 A1
(43) Pub. Date: Jan. 22, 2026

(54) METHODS OF USING ANTI-CD79b IMMUNOCONJUGATES TO TREAT FOLLICULAR LYMPHOMA

(71) Applicant: Genentech, Inc., South San Francisco, CA (US)

(72) Inventors: Lisa MUSICK, Kentfield, CA (US); Jamie Harue HIRATA, San Carlos, CA (US)

(73) Assignee: Genentech, Inc., South San Francisco, CA (US)

(21) Appl. No.: 19/338,441

(22) Filed: Sep. 24, 2025

Related U.S. Application Data
(63) Continuation of application No. 17/525,780, filed on Nov. 12, 2021, now abandoned.
Application No. 17/525,780 is a continuation of application No. PCT/US2020/032745, filed on May 13, 2020.
(60) Provisional application No. 62/847,847, filed on May 14, 2019.
(60) Provisional application No. 62/855,869, filed on May 31, 2019.
(60) Provisional application No. 62/894,602, filed on Aug. 30, 2019.
(60) Provisional application No. 62/931,205, filed on Nov. 5, 2019.
(60) Provisional application No. 62/944,305, filed on Dec. 5, 2019.

(51) Int. Cl. A61P 35/00; A61K 31/454; A61K 39/00; A61K 45/06; A61K 47/68; C07K 16/28

(52) U.S. Cl. CPC A61K 31/454; A61K 45/06; A61K 47/68031; A61K 47/6849; A61K 47/6867; A61P 35/00; C07K 16/2803; C07K 16/2887; A61K 2039/507; A61K 2039/545

(57) ABSTRACT

Provided herein are methods of treating B-cell proliferative disorders (such as Follicular Lymphoma “FL”) using immunoconjugates comprising anti-CD79b antibodies in combination with an immunomodulatory agent (such as lenalidomide) and an anti-CD20 antibody (such as obinutuzumab or rituximab).

### CROSS-REFERENCE TO RELATED APPLICATION

This application is a continuation of U.S. Non-Provisional application Ser. No. 17/525,780, filed Nov. 12, 2021, which is a continuation of International Application No. PCT/US2020/032745, filed May 13, 2020, which claims the benefit of U.S. Provisional Application 62/847,847, filed May 14, 2019, U.S. Provisional Application 62/855,869, filed May 31, 2019, U.S. Provisional Application 62/894,602, filed Aug. 30, 2019, U.S. Provisional Application 62/931,205, filed Nov. 5, 2019, and U.S. Provisional Application 62/944,305, filed Dec. 5, 2019, each of which is hereby incorporated by reference in its entirety.

### REFERENCE TO AN ELECTRONIC SEQUENCE LISTING

The content of the electronic sequence listing (146392048002seglist.xml; Size: 76,504 bytes; and Date of Creation: Sep. 2, 2025) is herein incorporated by reference in its entirety.

### FIELD OF THE INVENTION

The present disclosure relates to methods of treating B-cell proliferative disorders, e.g., follicular lymphoma (FL) by administering an immunoconjugate comprising anti-CD79b antibody in combination with an immunomodulatory agent (e.g., lenalidomide) and an anti-CD20 antibody (e.g., obinutuzumab or rituximab).

### BACKGROUND OF THE INVENTION

Follicular lymphoma (FL) is the most common subtype of indolent B-cell lymphoma, and FL accounts for about 22% of all newly diagnosed cases of B-cell lymphoma (Armitage et al. (1998) “New approach to classifying non-Hodgkin's lymphomas: clinical features of the major histologic subtypes. Non Hodgkin's Lymphoma Classification Project.” J Clin Oncol. 16:2780-95). Approximately 90% of the cases have a t(14:18) translocation, which juxtaposes BCL2 with the IgH locus and results in deregulated expression of Bcl-2. FL remains an incurable disease with the currently available therapies. The addition of rituximab, an anti-CD20 monoclonal antibody, to commonly used induction chemotherapy, including CHOP (cyclophosphamide, doxorubicin, vincristine, and prednisolone or prednisone), CVP (cyclophosphamide, vincristine, and prednisone), fludarabine, or bendamustine (Zelenetz et al. (2014) “Non-Hodgkin's lymphoma, Version 2.2014.” J Natl Compr Canc Netw. 12:916-46; Dreyling et al. (2014). “Newly diagnosed and relapsed follicular lymphoma: ESMO clinical recommendations for diagnosis, treatment and follow-up.” Ann Oncol. 25: iii76-82), followed by rituximab maintenance therapy led to prolonged remission and improved patient outcomes (Salles et al. (2013) “Updated 6 year follow-up of the PRIMA study confirms the benefit of 2-year rituximab maintenance in follicular lymphoma patients responding to frontline immunochemotherapy.” Blood. Abstract 509).

However, despite significant therapeutic progress with the use of chemoimmunotherapy as first-line treatment, most patients will eventually relapse. Relapses are characterized by increasing refractoriness and decreasing duration of response to subsequent lines of therapy. Thus, there is a need in the art for new treatments to provide additional therapeutic options and improve outcomes for such patients.

All references cited herein, including patent applications and publications, are hereby incorporated by reference in their entirety.

### SUMMARY

In one aspect, the present disclosure provides methods for treating follicular lymphoma (FL) in a human in need thereof comprising administering to the human an effective amount of: (a) an immunoconjugate comprising the formula

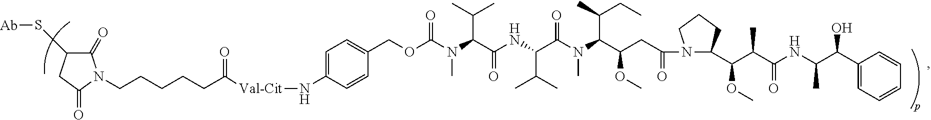

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, (b) an immunomodulatory agent, and (c) an anti-CD20 antibody; and wherein the human achieves at least a complete response (CR) following the treatment. In some embodiments, among a plurality of humans treated, at least 60%, at least 65%, at least 70%, or at least 75% of the humans achieve a complete response. In some embodiments, the anti-CD79b antibody comprises (i) a heavy chain variable domain (VH) comprising the amino acid sequence of SEQ ID NO: 19 and (ii) a light chain variable domain (VL) comprising the amino acid sequence of SEQ ID NO: 20. In some embodiments, the anti-CD79b antibody comprises (i) a heavy chain comprising the amino acid sequence of SEQ ID NO: 36 and (ii) a light chain comprising the amino acid sequence of SEQ ID NO: 35. In some embodiments, the immunoconjugate is polatuzumab vedotin. In some embodiments, the immunomodulatory agent is lenalidomide. In some embodiments, the anti-CD20 antibody is obinutuzumab. In some embodiments, the immunoconjugate is administered at a dose between about 1.4 mg/kg and about 1.8 mg/kg, the lenalidomide is administered at a dose between about 10 mg and about 20 mg, and the obinutuzumab is administered at a dose of about 1000 mg. In some embodiments, the immunoconjugate, the lenalidomide, and the obinutuzumab are administered during an induction phase for at least six 28-day cycles, wherein the immunoconjugate is administered intravenously at a dose between about 1.4 mg/kg and about 1.8 mg/kg on Day 1, the lenalidomide is administered orally at a dose between about 10 mg and about 20 mg, on each of Days 1-21, and the obinutuzumab is administered intravenously at a dose of about 1000 mg on each of Days 1, 8, and 15 of the first 28-day cycle, and wherein the immunoconjugate is administered intravenously at a dose between about 1.4 mg/kg and about 1.8 mg/kg on Day 1, the lenalidomide is administered orally at a dose between about 10 mg and about 20 mg on each of Days 1-21, and the obinutuzumab is administered intravenously at a dose of about 1000 mg on Day 1 of each of the second, third, fourth, fifth, and sixth 28-day cycles. In some embodiments, the immunoconjugate, the immunomodulatory agent, and the anti-CD20 antibody are administered sequentially. In some embodiments, the lenalidomide is administered prior to the obinutuzumab, and wherein the obinutuzumab is administered prior to the immunoconjugate on Day 1 and wherein the lenalidomide is administered prior to the obinutuzumab on each of Days 8 and 15 of the first 28-day cycle, and wherein the lenalidomide is administered prior to the obinutuzumab, and wherein the obinutuzumab is administered prior to the immunoconjugate on Day 1 of each of the second, third, fourth, fifth, and sixth 28-day cycles. In some embodiments, the lenalidomide and the obinutuzumab are further administered during a maintenance phase following the sixth 28-day cycle. In some embodiments, the lenalidomide is administered orally at a dose of about 10 mg on each of Days 1-21 of each month during the maintenance phase following the sixth 28-day cycle, and wherein the obinutuzumab is administered intravenously at a dose of about 1000 mg on Day 1 of every other month during the maintenance phase following the sixth 28-day cycle. In some embodiments, the lenalidomide is administered for a maximum of 12 months during the maintenance phase following the sixth 28-day cycle. In some embodiments, the obinutuzumab is administered for a maximum of 24 months during the maintenance phase following the sixth 28-day cycle. In some embodiments, the lenalidomide and the obinutuzumab are administered sequentially during the maintenance phase following the sixth 28-day cycle. In some embodiments, the lenalidomide is administered prior to the obinutuzumab on Day 1 of each of the first, third, fifth, seventh, ninth, and eleventh months during the maintenance phase following the sixth 28-day cycle.

In another aspect, provided is a method for treating follicular lymphoma (FL) in a human in need thereof comprising administering to the human an effective amount of: (a) an immunoconjugate comprising the formula

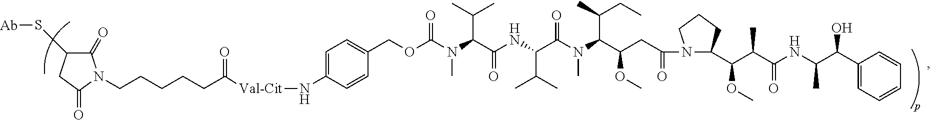

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, (b) an immunomodulatory agent, and (c) an anti-CD20 antibody; and wherein the human does not demonstrate disease progression within at least about 12 months. In some embodiments, the human does not demonstrate disease progression within at least about 12 months after the start of treatment with the immunoconjugate, the immunomodulatory agent, and the anti-CD20 antibody. In some embodiments, among a plurality of humans treated, at least 75%, at least 80%, at least 85%, or at least 90% of the humans do not demonstrate disease progression within at least about 12 months after the start of treatment with the immunoconjugate, the immunomodulatory agent, and the anti-CD20 antibody. In another aspect, provided is a method for treating follicular lymphoma (FL) in a human in need thereof comprising administering to the human an effective amount of:

(a) an immunoconjugate comprising the formula

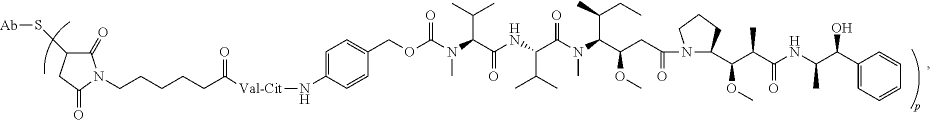

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, (b) an immunomodulatory agent, and (c) an anti-CD20 antibody; and wherein the human demonstrates 12-month progression-free survival. In some embodiments, the human demonstrates 12-month progression-free survival, measured after the start of treatment with the immunoconjugate, the immunomodulatory agent, and the anti-CD20 antibody. In some embodiments, among a plurality of humans treated, the 12-month progression-free survival rate is at least 75%, at least 80%, at least 85%, or at least 90%, measured after the start of treatment with the immunoconjugate, the immunomodulatory agent, and the anti-CD20 antibody. In some embodiments, among a plurality of humans treated, at least 60%, at least 65%, at least 70%, or at least 75% of the humans achieve a complete response. In some embodiments, the anti-CD79b antibody comprises (i) a heavy chain variable domain (VH) comprising the amino acid sequence of SEQ ID NO: 19 and (ii) a light chain variable domain (VL) comprising the amino acid sequence of SEQ ID NO: 20. In some embodiments, the anti-CD79b antibody comprises (i) a heavy chain comprising the amino acid sequence of SEQ ID NO: 36 and (ii) a light chain comprising the amino acid sequence of SEQ ID NO: 35. In some embodiments, the immunoconjugate is polatuzumab vedotin. In some embodiments, the immunomodulatory agent is lenalidomide. In some embodiments, the anti-CD20 antibody is obinutuzumab. In some embodiments, the immunoconjugate is administered at a dose between about 1.4 mg/kg and about 1.8 mg/kg, the lenalidomide is administered at a dose between about 10 mg and about 20 mg, and the obinutuzumab is administered at a dose of about 1000 mg. In some embodiments, the immunoconjugate, the lenalidomide, and the obinutuzumab are administered during an induction phase for at least six 28-day cycles, wherein the immunoconjugate is administered intravenously at a dose between about 1.4 mg/kg and about 1.8 mg/kg on Day 1, the lenalidomide is administered orally at a dose between about 10 mg and about 20 mg, on each of Days 1-21, and the obinutuzumab is administered intravenously at a dose of about 1000 mg on each of Days 1, 8, and 15 of the first 28-day cycle, and wherein the immunoconjugate is administered intravenously at a dose between about 1.4 mg/kg and about 1.8 mg/kg on Day 1, the lenalidomide is administered orally at a dose between about 10 mg and about 20 mg on each of Days 1-21, and the obinutuzumab is administered intravenously at a dose of about 1000 mg on Day 1 of each of the second, third, fourth, fifth, and sixth 28-day cycles. In some embodiments, the immunoconjugate, the immunomodulatory agent, and the anti-CD20 antibody are administered sequentially. In some embodiments, the lenalidomide is administered prior to the obinutuzumab, and wherein the obinutuzumab is administered prior to the immunoconjugate on Day 1 and wherein the lenalidomide is administered prior to the obinutuzumab on each of Days 8 and 15 of the first 28-day cycle, and wherein the lenalidomide is administered prior to the obinutuzumab, and wherein the obinutuzumab is administered prior to the immunoconjugate on Day 1 of each of the second, third, fourth, fifth, and sixth 28-day cycles. In some embodiments, the lenalidomide and the obinutuzumab are further administered during a maintenance phase following the sixth 28-day cycle. In some embodiments, the lenalidomide is administered orally at a dose of about 10 mg on each of Days 1-21 of each month during the maintenance phase following the sixth 28-day cycle, and wherein the obinutuzumab is administered intravenously at a dose of about 1000 mg on Day 1 of every other month during the maintenance phase following the sixth 28-day cycle. In some embodiments, the lenalidomide is administered for a maximum of 12 months during the maintenance phase following the sixth 28-day cycle. In some embodiments, the obinutuzumab is administered for a maximum of 24 months during the maintenance phase following the sixth 28-day cycle. In some embodiments, the lenalidomide and the obinutuzumab are administered sequentially during the maintenance phase following the sixth 28-day cycle. In some embodiments, the lenalidomide is administered prior to the obinutuzumab on Day 1 of each of the first, third, fifth, seventh, ninth, and eleventh months during the maintenance phase following the sixth 28-day cycle.

In another aspect, the present disclosure provides methods of treating follicular lymphoma in a human in need thereof, comprising administering to the human an effective amount of: (a) an immunoconjugate comprising the formula

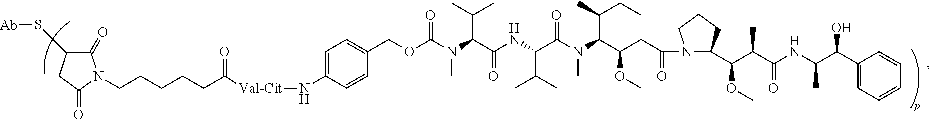

wherein Ab is an anti-CD79b antibody comprising (i) a heavy chain variable domain (VH) comprising the amino acid sequence of SEQ ID NO: 19 and (ii) a light chain variable domain (VL) comprising the amino acid sequence of SEQ ID NO: 20, and wherein p is between 2 and 5, (b) lenalidomide and (c) obinutuzumab, wherein the immunoconjugate is administered at a dose between about 1.4 mg/kg and about 1.8 mg/kg, the lenalidomide is administered at a dose between about 10 mg and about 20 mg, and the obinutuzumab is administered at a dose of about 1000 mg, and wherein the human achieves at least complete response (CR) following the treatment. In some embodiments, among a plurality of humans treated, at least 60%, at least 65%, at least 70%, or at least 75% of the humans achieve a complete response. In some embodiments, p is between 3 and 4. In some embodiments, the antibody comprises (i) a heavy chain comprising the amino acid sequence of SEQ ID NO: 36 and wherein (ii) a light chain comprising the amino acid sequence of SEQ ID NO: 35. In some embodiments, the immunoconjugate is polatuzumab vedotin. In some embodiments, the immunoconjugate, the lenalidomide, and the obinutuzumab are administered during an induction phase for at least six 28-day cycles, wherein the immunoconjugate is administered intravenously at a dose between about 1.4 mg/kg and about 1.8 mg/kg on Day 1, the lenalidomide is administered orally at a dose between about 10 mg and about 20 mg on each of Days 1-21, and the obinutuzumab is administered intravenously at a dose of about 1000 mg on each of Days 1, 8, and 15 of the first 28 day cycle, and wherein the immunoconjugate is administered intravenously at a dose between about 1.4 mg/kg and about 1.8 mg/kg on Day 1, the lenalidomide is administered orally at a dose between about 10 mg and about 20 mg on each of Days 1-21, and the obinutuzumab is administered intravenously at a dose of about 1000 mg on Day 1 of each of the second, third, fourth, fifth, and sixth 28-day cycles. In some embodiments, the lenalidomide is administered orally at a dose of about 10 mg on each of each of Days 1-21 of each month during the maintenance phase following the sixth 28-day cycle, and wherein the obinutuzumab is administered intravenously at a dose of about 1000 mg on Day 1 of every other month during the maintenance phase following the sixth 28-day cycle. In some embodiments, the lenalidomide is administered for a maximum of 12 months during the maintenance phase following the sixth 28-day cycle. In some embodiments, the obinutuzumab is administered for a maximum of 24 months during the maintenance phase following the sixth 28-day cycle. In some embodiments, the lenalidomide and the obinutuzumab are administered sequentially during the maintenance phase following the sixth 28-day cycle. In some embodiments, the lenalidomide is administered prior to the obinutuzumab on Day 1 of each of the first, third, fifth, seventh, ninth, and eleventh months during the maintenance phase following the sixth 28-day cycle. In some embodiments, among a plurality of humans treated, at least 75%, at least 80%, at least 85%, or at least 90% of the humans do not demonstrate disease progression within at least about 12 months after the start of treatment with the immunoconjugate, the lenalidomide, and the obinutuzumab. In some embodiments, among a plurality of humans treated, the 12-month progression-free survival rate is at least 75%, at least 80%, at least 85%, or at least 90%, measured after the start of treatment with the immunoconjugate, the lenalidomide, and the obinutuzumab. In some embodiments, among a plurality of humans treated, at least 75%, at least 80%, at least 85%, or at least 90% of the humans do not demonstrate disease progression within at least about 12 months after Day 1 of the first 28 day cycle during the induction phase. In some embodiments, among a plurality of humans treated, the 12-month progression-free survival rate is at least 75%, at least 80%, at least 85%, or at least 90%, measured after Day 1 of the first 28 day cycle during the induction phase.

In another aspect, the present disclosure provides methods of treating follicular lymphoma (FL) in a human in need thereof, comprising administering to the human, during an induction phase, an effective amount of: (a) polatuzumab vedotin; (b) lenalidomide; and (c) obinutuzumab, wherein, during the induction phase, the polatuzumab vedotin is administered at a dose of about 1.4 mg/kg, the lenalidomide is administered at a dose of about 10 mg, and the obinutuzumab is administered at a dose of about 1000 mg, and wherein, the human achieves a complete response following the induction phase. In another aspect, the present disclosure provides methods of treating follicular lymphoma (FL) in a human in need thereof, comprising administering to the human, during an induction phase, an effective amount of: (a) polatuzumab vedotin; (b) lenalidomide; and (c) obinutuzumab, wherein, during the induction phase, the polatuzumab vedotin is administered at a dose of about 1.4 mg/kg, the lenalidomide is administered at a dose of about 15 mg, and the obinutuzumab is administered at a dose of about 1000 mg, and wherein, the human achieves a complete response following the induction phase. In another aspect, the present disclosure provides methods of treating follicular lymphoma (FL) in a human in need thereof, comprising administering to the human, during an induction phase, an effective amount of: (a) polatuzumab vedotin; (b) lenalidomide; and (c) obinutuzumab, wherein, during the induction phase, the polatuzumab vedotin is administered at a dose of about 1.4 mg/kg, the lenalidomide is administered at a dose of about 20 mg, and the obinutuzumab is administered at a dose of about 1000 mg, and wherein, the human achieves a complete response following the induction phase. In some embodiments, the polatuzumab vedotin, the lenalidomide, and the obinutuzumab are administered during the induction phase for at least six 28-day cycles, wherein the polatuzumab vedotin is administered intravenously at a dose of about 1.4 mg/kg on Day 1, the lenalidomide is administered orally at a dose of about 20 mg on each of Days 1-21, and the obinutuzumab is administered intravenously at a dose of about 1000 mg on each of Days 1, 8, and 15 of the first 28 day cycle, and wherein the polatuzumab vedotin is administered intravenously at a dose of about 1.4 mg/kg on Day 1, the lenalidomide is administered orally at a dose between about 20 mg on each of Days 1-21, and the obinutuzumab is administered intravenously at a dose of about 1000 mg on Day 1 of each of the second, third, fourth, fifth, and sixth 28-day cycles. In some embodiments, the induction phase is followed by a maintenance phase, wherein the lenalidomide is administered at a dose of about 10 mg and the obinutuzumab is administered at a dose of about 1000 mg during the maintenance phase. In some embodiments, the lenalidomide is administered orally at a dose of about 10 mg on each of Days 1-21 of each month during the maintenance phase following the sixth 28-day cycle, and wherein the obinutuzumab is administered intravenously at a dose of about 1000 mg on Day 1 of every other month during the maintenance phase following the sixth 28-day cycle. In some embodiments, the human does not demonstrate disease progression within at least about 12 months after the start of the induction phase. In some embodiments, the human demonstrates 12-month progression-free survival, measured after the start of the induction phase.

In another aspect, the present disclosure provides methods of treating follicular lymphoma (FL) in a plurality of humans in need thereof, comprising administering to the humans, during an induction phase, an effective amount of: (a) polatuzumab vedotin; (b) lenalidomide; and (c) obinutuzumab, wherein, during the induction phase, the polatuzumab vedotin is administered at a dose of about 1.4 mg/kg, the lenalidomide is administered at a dose of about 10 mg, and the obinutuzumab is administered at a dose of about 1000 mg, and wherein, at least 60% of the humans in the plurality achieve a complete response following the induction phase. In another aspect, the present disclosure provides methods of treating follicular lymphoma (FL) in a plurality of humans in need thereof, comprising administering to the humans, during an induction phase, an effective amount of: (a) polatuzumab vedotin; (b) lenalidomide; and (c) obinutuzumab, wherein, during the induction phase, the polatuzumab vedotin is administered at a dose of about 1.4 mg/kg, the lenalidomide is administered at a dose of about 15 mg, and the obinutuzumab is administered at a dose of about 1000 mg, and wherein, at least 60% of the humans in the plurality achieve a complete response following the induction phase. In another aspect, the present disclosure provides methods of treating follicular lymphoma (FL) in a plurality of humans in need thereof, comprising administering to the humans, during an induction phase, an effective amount of: (a) polatuzumab vedotin; (b) lenalidomide; and (c) obinutuzumab, wherein, during the induction phase, the polatuzumab vedotin is administered at a dose of about 1.4 mg/kg, the lenalidomide is administered at a dose of about 20 mg, and the obinutuzumab is administered at a dose of about 1000 mg, and wherein, at least 60% of the humans in the plurality achieve a complete response following the induction phase. In some embodiments, the polatuzumab vedotin, the lenalidomide, and the obinutuzumab are administered during the induction phase for at least six 28-day cycles, wherein the polatuzumab vedotin is administered intravenously at a dose of about 1.4 mg/kg on Day 1, the lenalidomide is administered orally at a dose of about 20 mg on each of Days 1-21, and the obinutuzumab is administered intravenously at a dose of about 1000 mg on each of Days 1, 8, and 15 of the first 28 day cycle, and wherein the polatuzumab vedotin is administered intravenously at a dose of about 1.4 mg/kg on Day 1, the lenalidomide is administered orally at a dose between about 20 mg on each of Days 1-21, and the obinutuzumab is administered intravenously at a dose of about 1000 mg on Day 1 of each of the second, third, fourth, fifth, and sixth 28-day cycles. In some embodiments, the induction phase is followed by a maintenance phase, wherein the lenalidomide is administered at a dose of about 10 mg and the obinutuzumab is administered at a dose of about 1000 mg during the maintenance phase. In some embodiments, the lenalidomide is administered orally at a dose of about 10 mg on each of Days 1-21 of each month during the maintenance phase following the sixth 28-day cycle, and wherein the obinutuzumab is administered intravenously at a dose of about 1000 mg on Day 1 of every other month during the maintenance phase following the sixth 28-day cycle. In some embodiments, among a plurality of humans treated, at least 75%, at least 80%, at least 85%, or at least 90% of the humans do not demonstrate disease progression within at least 12 months, measured after the start of treatment with the immunoconjugate or the polatuzumab vedotin, the immunomodulatory agent or the lenalidomide, and the anti-CD20 antibody or the obinutuzumab. In some embodiments, among a plurality of humans treated, the 12-month progression-free survival rate is at least 75%, at least 80%, at least 85%, or at least 90%, measured after the start of treatment with the immunoconjugate or the polatuzumab vedotin, the immunomodulatory agent or the lenalidomide, and the anti-CD20 antibody or the obinutuzumab.

In another aspect, the present disclosure provides methods of treating follicular lymphoma (FL) in a human in need thereof, comprising administering to the human, during an induction phase, an effective amount of: (a) polatuzumab vedotin; (b) lenalidomide; and (c) obinutuzumab, wherein, during the induction phase, the polatuzumab vedotin is administered at a dose of about 1.8 mg/kg, the lenalidomide is administered at a dose of about 10 mg, and the obinutuzumab is administered at a dose of about 1000 mg, and wherein, the human achieves a complete response following the induction phase. In another aspect, the present disclosure provides methods of treating follicular lymphoma (FL) in a human in need thereof, comprising administering to the human, during an induction phase, an effective amount of: (a) polatuzumab vedotin; (b) lenalidomide; and (c) obinutuzumab, wherein, during the induction phase, the polatuzumab vedotin is administered at a dose of about 1.8 mg/kg, the lenalidomide is administered at a dose of about 15 mg, and the obinutuzumab is administered at a dose of about 1000 mg, and wherein, the human achieves a complete response following the induction phase. In another aspect, the present disclosure provides methods of treating follicular lymphoma (FL) in a human in need thereof, comprising administering to the human, during an induction phase, an effective amount of: (a) polatuzumab vedotin; (b) lenalidomide; and (c) obinutuzumab, wherein, during the induction phase, the polatuzumab vedotin is administered at a dose of about 1.8 mg/kg, the lenalidomide is administered at a dose of about 20 mg, and the obinutuzumab is administered at a dose of about 1000 mg, and wherein, the human achieves a complete response following the induction phase. In some embodiments, the polatuzumab vedotin, the lenalidomide, and the obinutuzumab are administered during the induction phase for at least six 28-day cycles, wherein the polatuzumab vedotin is administered intravenously at a dose of about 1.8 mg/kg on Day 1, the lenalidomide is administered orally at a dose of about 20 mg on each of Days 1-21, and the obinutuzumab is administered intravenously at a dose of about 1000 mg on each of Days 1, 8, and 15 of the first 28 day cycle, and wherein the polatuzumab vedotin is administered intravenously at a dose of about 1.8 mg/kg on Day 1, the lenalidomide is administered orally at a dose between about 20 mg on each of Days 1-21, and the obinutuzumab is administered intravenously at a dose of about 1000 mg on Day 1 of each of the second, third, fourth, fifth, and sixth 28-day cycles. In some embodiments, the induction phase is followed by a maintenance phase, wherein the lenalidomide is administered at a dose of about 10 mg and the obinutuzumab is administered at a dose of about 1000 mg during the maintenance phase. In some embodiments, the lenalidomide is administered orally at a dose of about 10 mg on each of Days 1-21 of each month during the maintenance phase following the sixth 28-day cycle, and wherein the obinutuzumab is administered intravenously at a dose of about 1000 mg on Day 1 of every other month during the maintenance phase following the sixth 28-day cycle. In some embodiments, the human does not demonstrate disease progression within at least 12 months after the start of the induction phase. In some embodiments, the human demonstrates 12-month progression-free survival, measured after the start of the induction phase.

In another aspect, the present disclosure provides methods of treating follicular lymphoma (FL) in a plurality of humans in need thereof, comprising administering to the humans, during an induction phase, an effective amount of: (a) polatuzumab vedotin; (b) lenalidomide; and (c) obinutuzumab, wherein, during the induction phase, the polatuzumab vedotin is administered at a dose of about 1.8 mg/kg, the lenalidomide is administered at a dose of about 10 mg, and the obinutuzumab is administered at a dose of about 1000 mg, and wherein, at least 60% of the humans in the plurality achieve a complete response following the induction phase. In another aspect, the present disclosure provides methods of treating follicular lymphoma (FL) in a plurality of humans in need thereof, comprising administering to the humans, during an induction phase, an effective amount of: (a) polatuzumab vedotin; (b) lenalidomide; and (c) obinutuzumab, wherein, during the induction phase, the polatuzumab vedotin is administered at a dose of about 1.8 mg/kg, the lenalidomide is administered at a dose of about 15 mg, and the obinutuzumab is administered at a dose of about 1000 mg, and wherein, at least 60% of the humans in the plurality achieve a complete response following the induction phase. In another aspect, the present disclosure provides methods of treating follicular lymphoma (FL) in a plurality of humans in need thereof, comprising administering to the humans, during an induction phase, an effective amount of: (a) polatuzumab vedotin; (b) lenalidomide; and (c) obinutuzumab, wherein, during the induction phase, the polatuzumab vedotin is administered at a dose of about 1.8 mg/kg, the lenalidomide is administered at a dose of about 20 mg, and the obinutuzumab is administered at a dose of about 1000 mg, and wherein, at least 60% of the humans in the plurality achieve a complete response following the induction phase. In some embodiments, the polatuzumab vedotin, the lenalidomide, and the obinutuzumab are administered during the induction phase for at least six 28-day cycles, wherein the polatuzumab vedotin is administered intravenously at a dose of about 1.8 mg/kg on Day 1, the lenalidomide is administered orally at a dose of about 20 mg on each of Days 1-21, and the obinutuzumab is administered intravenously at a dose of about 1000 mg on each of Days 1, 8, and 15 of the first 28 day cycle, and wherein the polatuzumab vedotin is administered intravenously at a dose of about 1.8 mg/kg on Day 1, the lenalidomide is administered orally at a dose between about 20 mg on each of Days 1-21, and the obinutuzumab is administered intravenously at a dose of about 1000 mg on Day 1 of each of the second, third, fourth, fifth, and sixth 28-day cycles. In some embodiments, the induction phase is followed by a maintenance phase, wherein the lenalidomide is administered at a dose of about 10 mg and the obinutuzumab is administered at a dose of about 1000 mg during the maintenance phase. In some embodiments the lenalidomide is administered orally at a dose of about 10 mg on each of Days 1-21 of each month during the maintenance phase following the sixth 28-day cycle, and wherein the obinutuzumab is administered intravenously at a dose of about 1000 mg on Day 1 of every other month during the maintenance phase following the sixth 28-day cycle. In some embodiments, among a plurality of humans treated, at least 75%, at least 80%, at least 85%, or at least 90% of the humans do not demonstrate disease progression within at least 12 months after the start of the induction phase. In some embodiments, among a plurality of humans treated, the 12-month progression-free survival rate is at least 75%, at least 80%, at least 85%, or at least 90%, measured after the start of the induction phase.

In some embodiments, the human has received at least one prior therapy for FL. In some embodiments, the at least one prior therapy was a chemoimmunotherapy that included an anti-CD20 antibody. In some embodiments, the FL is CD20-positive FL. In some embodiments, the human has received at least one prior therapy for FL. In some embodiments, the human has received at least two prior therapies for FL. In some embodiments, the human has received at least three prior therapies for FL. In some embodiments, the human has received between one and five prior therapies for FL. In some embodiments, the human has received between one and seven prior therapies for FL. In some embodiments, the human was refractory to their most recent therapy for FL. In some embodiments, the human exhibited progression or relapse of FL within about six months from the end date of their most recent therapy for FL. In some embodiments, the human exhibited no response to their most recent therapy for FL. In some embodiments, the human was refractory to a prior therapy for FL with an anti-CD20 agent. In some embodiments, the human exhibited progression or relapse of FL within about 6 months of a prior therapy for FL with an anti-CD20 agent. In some embodiments, the human exhibited no response to a prior therapy for FL with an anti-CD20 agent. In some embodiments, the human had progression of disease within 24 months of initiation of their first FL treatment with chemoimmunotherapy. In some embodiments, the FL is relapsed/refractory FL. In some embodiments, the FL is a positron emission tomography (PET)-positive lymphoma. In some embodiments, the human does not have central nervous system (CNS) lymphoma or leptomeningeal infiltration. In some embodiments, the human has not received prior allogenic stem cell transplantation (SCT). In some embodiments, the human has an Eastern Cooperative Oncology Group Performance Status score of 0-1. In some embodiments, the human has FL with an Ann Arbor Stage of III or IV. In some embodiments, the human has bulky disease FL (≥7 cm). In some embodiments, the human has 3-5 Follicular Lymphoma International Prognostic Index (FLIPI) risk factors. In some embodiments, the human has 1-2 FLIPI risk factors. In some embodiments, the human has FL with bone marrow involvement. In some embodiments, administration of the immunoconjugate or polatuzumab vedotin, the immunomodulatory agent or lenalidomide, and the anti-CD20 antibody or obinutuzumab does not result in peripheral neuropathy in the human of grade 3 or greater.

In another aspect, the present disclosure provides kits comprising an immunoconjugate comprising the formula

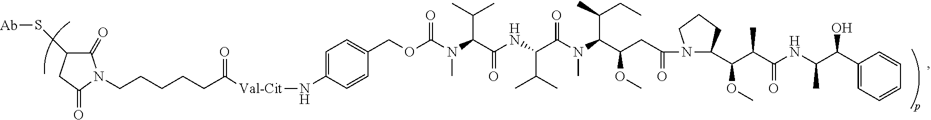

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, for use in combination with an immunomodulatory agent and an anti-CD20 antibody for treating a human in need thereof having follicular lymphoma (FL) according to any method of the present disclosure. In another aspect, the present disclosure provides kits comprising an immunoconjugate comprising the formula

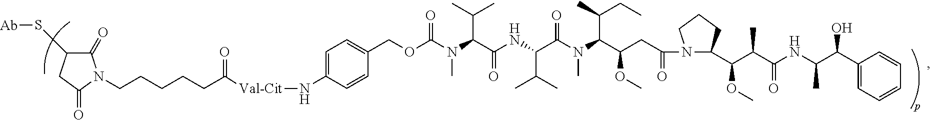

wherein Ab is an anti-CD79b antibody comprising (i) a heavy chain variable domain (VH) comprising the amino acid sequence of SEQ ID NO: 19 and (ii) a light chain variable domain (VL) comprising the amino acid sequence of SEQ ID NO: 20, and wherein p is between 2 and 5, for use in combination with lenalidomide and obinutuzumab for treating a human in need thereof having follicular lymphoma (FL) according to any method of the present disclosure. In some embodiments, p is between 3 and 4. In some embodiments, the antibody comprises (i) a heavy chain comprising the amino acid sequence of SEQ ID NO: 36 and (ii) a light chain comprising the amino acid sequence of SEQ ID NO: 35.

In another aspect, the present disclosure provides kits comprising polatuzumab vedotin for use in combination with lenalidomide and obinutuzumab for treating a human in need thereof having follicular lymphoma (FL) according to any method of the present disclosure. In some embodiments, the FL is relapsed/refractory FL.

In another aspect, the present disclosure provides immunoconjugates comprising the formula

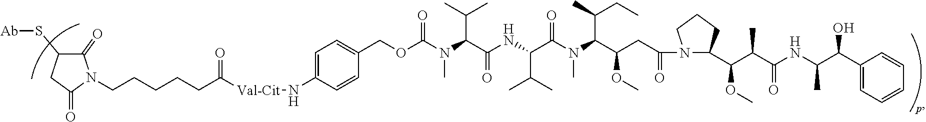

wherein Ab is an anti-CD79b antibody comprising (i) an a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8 for use in any method of treating follicular lymphoma (FL) according to the present disclosure. In some embodiments, the anti-CD79b antibody comprises (i) a heavy chain variable domain (VH) that comprises the amino acid sequence of SEQ ID NO: 19 and (ii) a light chain variable domain (VL) that comprises the amino acid sequence of SEQ ID NO: 20.

In another aspect, the present disclosure provides immunoconjugates comprising the formula

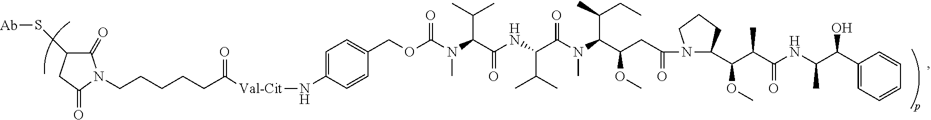

wherein Ab is an anti-CD79b antibody that comprises (i) a heavy chain variable domain (VH) comprising the amino acid sequence of SEQ ID NO: 19 and (ii) a light chain variable domain (VL) comprising the amino acid sequence of SEQ ID NO: 20, and wherein p is between 2 and 5, for use in any method of treating follicular lymphoma (FL) according to the present disclosure. In some embodiments, p is between 3 and 4. In some embodiments, the anti-CD79b antibody comprises (i) a heavy chain comprising the amino acid sequence of SEQ ID NO: 36 and (ii) a light chain comprising the amino acid sequence of SEQ ID NO: 35.

In another aspect, the present disclosure provides polatuzumab vedotin for use in any method of treating follicular lymphoma (FL) according to the present disclosure.

In some embodiments of any of the above aspects, the FL is relapsed/refractory FL. In some embodiments of any of the above aspects, among a plurality of humans treated, at least 89% achieve an overall response.

In another aspect, the present disclosure provides a use of an immunoconjugate comprising the formula

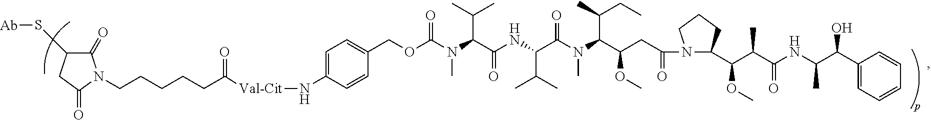

wherein Ab is an anti-CD79b antibody comprising (i) an a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, in the manufacture of a medicament for treating follicular lymphoma (FL) according to the methods provided herein. In some embodiments, p is between 3 and 4. In some embodiments, p is between 2 and 5. In some embodiments, the anti-CD79b antibody comprises (i) a heavy chain variable domain (VH) that comprises the amino acid sequence of SEQ ID NO: 19 and (ii) a light chain variable domain (VL) that comprises the amino acid sequence of SEQ ID NO: 20.

In another aspect, the present disclosure provides a use of an immunoconjugate comprising the formula

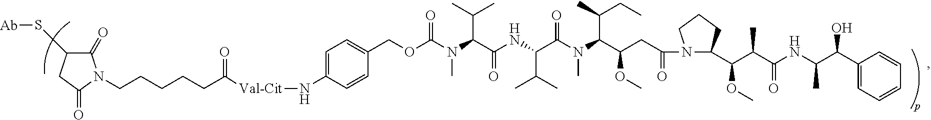

wherein Ab is an anti-CD79b antibody that comprises (i) a heavy chain variable domain (VH) comprising the amino acid sequence of SEQ ID NO: 19 and (ii) a light chain variable domain (VL) comprising the amino acid sequence of SEQ ID NO: 20, and wherein p is between 2 and 5, in the manufacture of a medicament for treating follicular lymphoma (FL) according to the methods provided herein. In some embodiments, p is between 3 and 4. In some embodiments, the anti-CD79b antibody comprises (i) a heavy chain comprising the amino acid sequence of SEQ ID NO: 36 and (ii) a light chain comprising the amino acid sequence of SEQ ID NO: 35.

In another aspect, the present disclosure provides a use of polatuzumab vedotin in the manufacture of a medicament for treating follicular lymphoma (FL) according to the methods of the present disclosure.

In some embodiments of any of the above aspects, the FL is relapsed/refractory FL. In some embodiments of any of the above aspects, among a plurality of humans treated, at least 89% achieve an overall response.

In some embodiments of any of the above aspects, the anti-CD20 antibody is rituximab. In some embodiments, the polatuzumab vedotin is administered at a dose of about 1.8 mg/kg, the lenalidomide is administered at a dose between about 10 mg and about 20 mg, and the rituximab is administered at a dose of about 375 mg/m².

In one aspect, the present disclosure provides methods for treating follicular lymphoma (FL) in a human in need thereof comprising administering to the human an effective amount of: (a) an immunoconjugate comprising the formula

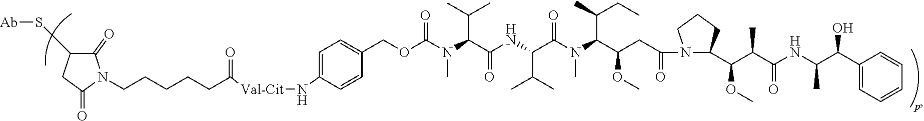

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, (b) an immunomodulatory agent, and (c) an anti-CD20 antibody; and wherein the human achieves at least a complete response (CR) following the treatment. In some embodiments, among a plurality of humans treated, at least 60%, at least 65%, at least 70%, or at least 75% of the humans achieve a complete response. In some embodiments, the anti-CD79b antibody comprises (i) a heavy chain variable domain (VH) comprising the amino acid sequence of SEQ ID NO: 19 and (ii) a light chain variable domain (VL) comprising the amino acid sequence of SEQ ID NO: 20. In some embodiments, the anti-CD79b antibody comprises (i) a heavy chain comprising the amino acid sequence of SEQ ID NO: 36 and (ii) a light chain comprising the amino acid sequence of SEQ ID NO: 35. In some embodiments, the immunoconjugate is polatuzumab vedotin. In some embodiments, the immunomodulatory agent is lenalidomide. In some embodiments, the anti-CD20 antibody is rituximab. In some embodiments, the immunoconjugate is administered at a dose between about 1.4 mg/kg and about 1.8 mg/kg, the lenalidomide is administered at a dose between about 10 mg and about 20 mg, and the rituximab is administered at a dose of about 375 mg/m². In some embodiments, the immunoconjugate, the lenalidomide, and the rituximab are administered during an induction phase for at least six 28-day cycles, wherein the immunoconjugate is administered intravenously at a dose between about 1.4 mg/kg and about 1.8 mg/kg on Day 1, the lenalidomide is administered orally at a dose between about 10 mg and about 20 mg, on each of Days 1-21, and the rituximab is administered intravenously at a dose of about 375 mg/m² on each of Days 1, 8, and 15 of the first 28-day cycle, and wherein the immunoconjugate is administered intravenously at a dose between about 1.4 mg/kg and about 1.8 mg/kg on Day 1, the lenalidomide is administered orally at a dose between about 10 mg and about 20 mg on each of Days 1-21, and the rituximab is administered intravenously at a dose of about 375 mg/m² on Day 1 of each of the second, third, fourth, fifth, and sixth 28-day cycles. In some embodiments, the immunoconjugate, the immunomodulatory agent, and the anti-CD20 antibody are administered sequentially. In some embodiments, the lenalidomide is administered prior to the rituximab, and wherein the rituximab is administered prior to the immunoconjugate on Day 1 and wherein the lenalidomide is administered prior to the rituximab on each of Days 8 and 15 of the first 28-day cycle, and wherein the lenalidomide is administered prior to the rituximab, and wherein the rituximab is administered prior to the immunoconjugate on Day 1 of each of the second, third, fourth, fifth, and sixth 28-day cycles. In some embodiments, the lenalidomide and the rituximab are further administered during a maintenance phase following the sixth 28-day cycle. In some embodiments, the lenalidomide is administered orally at a dose of about 10 mg on each of Days 1-21 of each month during the maintenance phase following the sixth 28-day cycle, and wherein the rituximab is administered intravenously at a dose of about 375 mg/m² on Day 1 of every other month during the maintenance phase following the sixth 28-day cycle. In some embodiments, the lenalidomide is administered for a maximum of 12 months during the maintenance phase following the sixth 28-day cycle. In some embodiments, the rituximab is administered for a maximum of 24 months during the maintenance phase following the sixth 28-day cycle. In some embodiments, the lenalidomide and the rituximab are administered sequentially during the maintenance phase following the sixth 28-day cycle. In some embodiments, the lenalidomide is administered prior to the rituximab on Day 1 of each of the first, third, fifth, seventh, ninth, and eleventh months during the maintenance phase following the sixth 28-day cycle.

In another aspect, the present disclosure provides methods of treating follicular lymphoma in a human in need thereof, comprising administering to the human an effective amount of: (a) an immunoconjugate comprising the formula

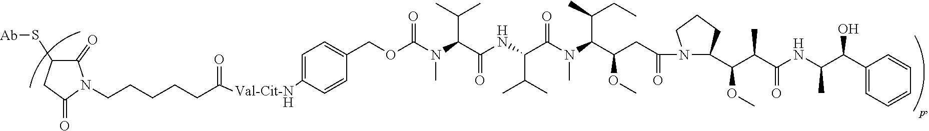

wherein Ab is an anti-CD79b antibody comprising (i) a heavy chain variable domain (VH) comprising the amino acid sequence of SEQ ID NO: 19 and (ii) a light chain variable domain (VL) comprising the amino acid sequence of SEQ ID NO: 20, and wherein p is between 2 and 5, (b) lenalidomide and (c) rituximab, wherein the immunoconjugate is administered at a dose between about 1.4 mg/kg and about 1.8 mg/kg, the lenalidomide is administered at a dose between about 10 mg and about 20 mg, and the rituximab is administered at a dose of about 375 mg/m², and wherein the human achieves at least complete response (CR) following the treatment. In some embodiments, among a plurality of humans treated, at least 60%, at least 65%, at least 70%, or at least 75% of the humans achieve a complete response. In some embodiments, p is between 3 and 4. In some embodiments, the antibody comprises (i) a heavy chain comprising the amino acid sequence of SEQ ID NO: 36 and wherein (ii) a light chain comprising the amino acid sequence of SEQ ID NO: 35. In some embodiments, the immunoconjugate is polatuzumab vedotin. In some embodiments, the immunoconjugate, the lenalidomide, and the rituximab are administered during an induction phase for at least six 28-day cycles, wherein the immunoconjugate is administered intravenously at a dose between about 1.4 mg/kg and about 1.8 mg/kg on Day 1, the lenalidomide is administered orally at a dose between about 10 mg and about 20 mg on each of Days 1-21, and the rituximab is administered intravenously at a dose of about 375 mg/m² on each of Days 1, 8, and 15 of the first 28 day cycle, and wherein the immunoconjugate is administered intravenously at a dose between about 1.4 mg/kg and about 1.8 mg/kg on Day 1, the lenalidomide is administered orally at a dose between about 10 mg and about 20 mg on each of Days 1-21, and the rituximab is administered intravenously at a dose of about 375 mg/m² on Day 1 of each of the second, third, fourth, fifth, and sixth 28-day cycles. In some embodiments, the lenalidomide is administered orally at a dose of about 10 mg on each of each of Days 1-21 of each month during the maintenance phase following the sixth 28-day cycle, and wherein the rituximab is administered intravenously at a dose of about 375 mg/m² on Day 1 of every other month during the maintenance phase following the sixth 28-day cycle. In some embodiments, the lenalidomide is administered for a maximum of 12 months during the maintenance phase following the sixth 28-day cycle. In some embodiments, the rituximab is administered for a maximum of 24 months during the maintenance phase following the sixth 28-day cycle. In some embodiments, the lenalidomide and the rituximab are administered sequentially during the maintenance phase following the sixth 28-day cycle. In some embodiments, the lenalidomide is administered prior to the rituximab on Day 1 of each of the first, third, fifth, seventh, ninth, and eleventh months during the maintenance phase following the sixth 28-day cycle. In some embodiments, among a plurality of humans treated, at least 75%, at least 80%, at least 85%, or at least 90% of the humans do not demonstrate disease progression within at least about 12 months after the start of treatment with the immunoconjugate, the lenalidomide, and the rituximab. In some embodiments, among a plurality of humans treated, the 12-month progression-free survival rate is at least 75%, at least 80%, at least 85%, or at least 90%, measured after the start of treatment with the immunoconjugate, the lenalidomide, and the rituximab. In some embodiments, among a plurality of humans treated, at least 75%, at least 80%, at least 85%, or at least 90% of the humans do not demonstrate disease progression within at least about 12 months after Day 1 of the first 28-day cycle during the induction phase. In some embodiments, among a plurality of humans treated, the 12-month progression-free survival rate is at least 75%, at least 80%, at least 85%, or at least 90%, measured after Day 1 of the first 28-day cycle during the induction phase.

In another aspect, the present disclosure provides methods of treating follicular lymphoma (FL) in a human in need thereof, comprising administering to the human, during an induction phase, an effective amount of: (a) polatuzumab vedotin; (b) lenalidomide; and (c) rituximab, wherein, during the induction phase, the polatuzumab vedotin is administered at a dose of about 1.4 mg/kg, the lenalidomide is administered at a dose of about 10 mg, and the rituximab is administered at a dose of about 375 mg/m², and wherein, the human achieves a complete response following the induction phase. In another aspect, the present disclosure provides methods of treating follicular lymphoma (FL) in a human in need thereof, comprising administering to the human, during an induction phase, an effective amount of: (a) polatuzumab vedotin; (b) lenalidomide; and (c) rituximab, wherein, during the induction phase, the polatuzumab vedotin is administered at a dose of about 1.4 mg/kg, the lenalidomide is administered at a dose of about 15 mg, and the rituximab is administered at a dose of about 375 mg/m², and wherein, the human achieves a complete response following the induction phase. In another aspect, the present disclosure provides methods of treating follicular lymphoma (FL) in a human in need thereof, comprising administering to the human, during an induction phase, an effective amount of: (a) polatuzumab vedotin; (b) lenalidomide; and (c) rituximab, wherein, during the induction phase, the polatuzumab vedotin is administered at a dose of about 1.4 mg/kg, the lenalidomide is administered at a dose of about 20 mg, and the rituximab is administered at a dose of about 375 mg/m², and wherein, the human achieves a complete response following the induction phase. In some embodiments, the polatuzumab vedotin, the lenalidomide, and the rituximab are administered during the induction phase for at least six 28-day cycles, wherein the polatuzumab vedotin is administered intravenously at a dose of about 1.4 mg/kg on Day 1, the lenalidomide is administered orally at a dose of about 20 mg on each of Days 1-21, and the rituximab is administered intravenously at a dose of about 375 mg/m² on each of Days 1, 8, and 15 of the first 28 day cycle, and wherein the polatuzumab vedotin is administered intravenously at a dose of about 1.4 mg/kg on Day 1, the lenalidomide is administered orally at a dose between about 20 mg on each of Days 1-21, and the rituximab is administered intravenously at a dose of about 375 mg/m² on Day 1 of each of the second, third, fourth, fifth, and sixth 28-day cycles. In some embodiments, the induction phase is followed by a maintenance phase, wherein the lenalidomide is administered at a dose of about 10 mg and the rituximab is administered at a dose of about 375 mg/m² during the maintenance phase. In some embodiments, the lenalidomide is administered orally at a dose of about 10 mg on each of Days 1-21 of each month during the maintenance phase following the sixth 28-day cycle, and wherein the rituximab is administered intravenously at a dose of about 375 mg/m² on Day 1 of every other month during the maintenance phase following the sixth 28-day cycle. In some embodiments, the human does not demonstrate disease progression within at least about 12 months after the start of the induction phase. In some embodiments, the human demonstrates 12-month progression-free survival, measured after the start of the induction phase.

In another aspect, the present disclosure provides methods of treating follicular lymphoma (FL) in a plurality of humans in need thereof, comprising administering to the humans, during an induction phase, an effective amount of: (a) polatuzumab vedotin; (b) lenalidomide; and (c) rituximab, wherein, during the induction phase, the polatuzumab vedotin is administered at a dose of about 1.4 mg/kg, the lenalidomide is administered at a dose of about 10 mg, and the rituximab is administered at a dose of about 375 mg/m², and wherein, at least 60% of the humans in the plurality achieve a complete response following the induction phase. In another aspect, the present disclosure provides methods of treating follicular lymphoma (FL) in a plurality of humans in need thereof, comprising administering to the humans, during an induction phase, an effective amount of: (a) polatuzumab vedotin; (b) lenalidomide; and (c) rituximab, wherein, during the induction phase, the polatuzumab vedotin is administered at a dose of about 1.4 mg/kg, the lenalidomide is administered at a dose of about 15 mg, and the rituximab is administered at a dose of about 375 mg/m², and wherein, at least 60% of the humans in the plurality achieve a complete response following the induction phase. In another aspect, the present disclosure provides methods of treating follicular lymphoma (FL) in a plurality of humans in need thereof, comprising administering to the humans, during an induction phase, an effective amount of: (a) polatuzumab vedotin; (b) lenalidomide; and (c) rituximab, wherein, during the induction phase, the polatuzumab vedotin is administered at a dose of about 1.4 mg/kg, the lenalidomide is administered at a dose of about 20 mg, and the rituximab is administered at a dose of about 375 mg/m², and wherein, at least 60% of the humans in the plurality achieve a complete response following the induction phase. In some embodiments, the polatuzumab vedotin, the lenalidomide, and the rituximab are administered during the induction phase for at least six 28-day cycles, wherein the polatuzumab vedotin is administered intravenously at a dose of about 1.4 mg/kg on Day 1, the lenalidomide is administered orally at a dose of about 20 mg on each of Days 1-21, and the rituximab is administered intravenously at a dose of about 375 mg/m² on each of Days 1, 8, and 15 of the first 28 day cycle, and wherein the polatuzumab vedotin is administered intravenously at a dose of about 1.4 mg/kg on Day 1, the lenalidomide is administered orally at a dose between about 20 mg on each of Days 1-21, and the rituximab is administered intravenously at a dose of about 375 mg/m² on Day 1 of each of the second, third, fourth, fifth, and sixth 28-day cycles. In some embodiments, the induction phase is followed by a maintenance phase, wherein the lenalidomide is administered at a dose of about 10 mg and the rituximab is administered at a dose of about 375 mg/m² during the maintenance phase. In some embodiments, the lenalidomide is administered orally at a dose of about 10 mg on each of Days 1-21 of each month during the maintenance phase following the sixth 28-day cycle, and wherein the rituximab is administered intravenously at a dose of about 375 mg/m² on Day 1 of every other month during the maintenance phase following the sixth 28-day cycle. In some embodiments, among a plurality of humans treated, at least 75%, at least 80%, at least 85%, or at least 90% of the humans do not demonstrate disease progression within at least 12 months, measured after the start of treatment with the immunoconjugate or the polatuzumab vedotin, the immunomodulatory agent or the lenalidomide, and the anti-CD20 antibody or the rituximab. In some embodiments, among a plurality of humans treated, the 12-month progression-free survival rate is at least 75%, at least 80%, at least 85%, or at least 90%, measured after the start of treatment with the immunoconjugate or the polatuzumab vedotin, the immunomodulatory agent or the lenalidomide, and the anti-CD20 antibody or the rituximab.

In another aspect, the present disclosure provides methods of treating follicular lymphoma (FL) in a human in need thereof, comprising administering to the human, during an induction phase, an effective amount of: (a) polatuzumab vedotin; (b) lenalidomide; and (c) rituximab, wherein, during the induction phase, the polatuzumab vedotin is administered at a dose of about 1.8 mg/kg, the lenalidomide is administered at a dose of about 10 mg, and the rituximab is administered at a dose of about 375 mg/m², and wherein, the human achieves a complete response following the induction phase. In another aspect, the present disclosure provides methods of treating follicular lymphoma (FL) in a human in need thereof, comprising administering to the human, during an induction phase, an effective amount of: (a) polatuzumab vedotin; (b) lenalidomide; and (c) rituximab, wherein, during the induction phase, the polatuzumab vedotin is administered at a dose of about 1.8 mg/kg, the lenalidomide is administered at a dose of about 15 mg, and the rituximab is administered at a dose of about 375 mg/m², and wherein, the human achieves a complete response following the induction phase. In another aspect, the present disclosure provides methods of treating follicular lymphoma (FL) in a human in need thereof, comprising administering to the human, during an induction phase, an effective amount of: (a) polatuzumab vedotin; (b) lenalidomide; and (c) rituximab, wherein, during the induction phase, the polatuzumab vedotin is administered at a dose of about 1.8 mg/kg, the lenalidomide is administered at a dose of about 20 mg, and the rituximab is administered at a dose of about 375 mg/m², and wherein, the human achieves a complete response following the induction phase. In some embodiments, the polatuzumab vedotin, the lenalidomide, and the rituximab are administered during the induction phase for at least six 28-day cycles, wherein the polatuzumab vedotin is administered intravenously at a dose of about 1.8 mg/kg on Day 1, the lenalidomide is administered orally at a dose of about 20 mg on each of Days 1-21, and the rituximab is administered intravenously at a dose of about 375 mg/m² on each of Days 1, 8, and 15 of the first 28 day cycle, and wherein the polatuzumab vedotin is administered intravenously at a dose of about 1.8 mg/kg on Day 1, the lenalidomide is administered orally at a dose between about 20 mg on each of Days 1-21, and the rituximab is administered intravenously at a dose of about 375 mg/m² on Day 1 of each of the second, third, fourth, fifth, and sixth 28-day cycles. In some embodiments, the induction phase is followed by a maintenance phase, wherein the lenalidomide is administered at a dose of about 10 mg and the rituximab is administered at a dose of about 375 mg/m² during the maintenance phase. In some embodiments, the lenalidomide is administered orally at a dose of about 10 mg on each of Days 1-21 of each month during the maintenance phase following the sixth 28-day cycle, and wherein the rituximab is administered intravenously at a dose of about 375 mg/m² on Day 1 of every other month during the maintenance phase following the sixth 28-day cycle. In some embodiments, the human does not demonstrate disease progression within at least 12 months after the start of the induction phase. In some embodiments, the human demonstrates 12-month progression-free survival, measured after the start of the induction phase.

In another aspect, the present disclosure provides methods of treating follicular lymphoma (FL) in a plurality of humans in need thereof, comprising administering to the humans, during an induction phase, an effective amount of: (a) polatuzumab vedotin; (b) lenalidomide; and (c) rituximab, wherein, during the induction phase, the polatuzumab vedotin is administered at a dose of about 1.8 mg/kg, the lenalidomide is administered at a dose of about 10 mg, and the rituximab is administered at a dose of about 375 mg/m², and wherein, at least 60% of the humans in the plurality achieve a complete response following the induction phase. In another aspect, the present disclosure provides methods of treating follicular lymphoma (FL) in a plurality of humans in need thereof, comprising administering to the humans, during an induction phase, an effective amount of: (a) polatuzumab vedotin; (b) lenalidomide; and (c) rituximab, wherein, during the induction phase, the polatuzumab vedotin is administered at a dose of about 1.8 mg/kg, the lenalidomide is administered at a dose of about 15 mg, and the rituximab is administered at a dose of about 375 mg/m², and wherein, at least 60% of the humans in the plurality achieve a complete response following the induction phase. In another aspect, the present disclosure provides methods of treating follicular lymphoma (FL) in a plurality of humans in need thereof, comprising administering to the humans, during an induction phase, an effective amount of: (a) polatuzumab vedotin; (b) lenalidomide; and (c) rituximab, wherein, during the induction phase, the polatuzumab vedotin is administered at a dose of about 1.8 mg/kg, the lenalidomide is administered at a dose of about 20 mg, and the rituximab is administered at a dose of about 375 mg/m², and wherein, at least 60% of the humans in the plurality achieve a complete response following the induction phase. In some embodiments, the polatuzumab vedotin, the lenalidomide, and the rituximab are administered during the induction phase for at least six 28-day cycles, wherein the polatuzumab vedotin is administered intravenously at a dose of about 1.8 mg/kg on Day 1, the lenalidomide is administered orally at a dose of about 20 mg on each of Days 1-21, and the rituximab is administered intravenously at a dose of about 375 mg/m² on each of Days 1, 8, and 15 of the first 28 day cycle, and wherein the polatuzumab vedotin is administered intravenously at a dose of about 1.8 mg/kg on Day 1, the lenalidomide is administered orally at a dose between about 20 mg on each of Days 1-21, and the rituximab is administered intravenously at a dose of about 375 mg/m² on Day 1 of each of the second, third, fourth, fifth, and sixth 28-day cycles. In some embodiments, the induction phase is followed by a maintenance phase, wherein the lenalidomide is administered at a dose of about 10 mg and the rituximab is administered at a dose of about 375 mg/m² during the maintenance phase. In some embodiments the lenalidomide is administered orally at a dose of about 10 mg on each of Days 1-21 of each month during the maintenance phase following the sixth 28-day cycle, and wherein the rituximab is administered intravenously at a dose of about 375 mg/m² on Day 1 of every other month during the maintenance phase following the sixth 28-day cycle. In some embodiments, among a plurality of humans treated, at least 75%, at least 80%, at least 85%, or at least 90% of the humans do not demonstrate disease progression within at least 12 months after the start of the induction phase. In some embodiments, among a plurality of humans treated, the 12-month progression-free survival rate is at least 75%, at least 80%, at least 85%, or at least 90%, measured after the start of the induction phase.

In some embodiments, the human has received at least one prior therapy for FL. In some embodiments, the at least one prior therapy was a chemoimmunotherapy that included an anti-CD20 antibody. In some embodiments, the FL is CD20-positive FL. In some embodiments, the human has received at least one prior therapy for FL. In some embodiments, the human has received at least two prior therapies for FL. In some embodiments, the human has received at least three prior therapies for FL. In some embodiments, the human has received between one and five prior therapies for FL. In some embodiments, the human has received between one and seven prior therapies for FL. In some embodiments, the human was refractory to their most recent therapy for FL. In some embodiments, the human exhibited progression or relapse of FL within about six months from the end date of their most recent therapy for FL. In some embodiments, the human exhibited no response to their most recent therapy for FL. In some embodiments, the human was refractory to a prior therapy for FL with an anti-CD20 agent. In some embodiments, the human exhibited progression or relapse of FL within about 6 months of a prior therapy for FL with an anti-CD20 agent. In some embodiments, the human exhibited no response to a prior therapy for FL with an anti-CD20 agent. In some embodiments, the human had progression of disease within 24 months of initiation of their first FL treatment with chemoimmunotherapy. In some embodiments, the FL is relapsed/refractory FL. In some embodiments, the FL is a positron emission tomography (PET)-positive lymphoma. In some embodiments, the human does not have central nervous system (CNS) lymphoma or leptomeningeal infiltration. In some embodiments, the human has not received prior allogenic stem cell transplantation (SCT). In some embodiments, the human has an Eastern Cooperative Oncology Group Performance Status score of 0-1. In some embodiments, the human has FL with an Ann Arbor Stage of III or IV. In some embodiments, the human has bulky disease FL (≥7 cm). In some embodiments, the human has 3-5 Follicular Lymphoma International Prognostic Index (FLIPI) risk factors. In some embodiments, the human has 1-2 FLIPI risk factors. In some embodiments, the human has FL with bone marrow involvement. In some embodiments, administration of the immunoconjugate or polatuzumab vedotin, the immunomodulatory agent or lenalidomide, and the anti-CD20 antibody or rituximab does not result in peripheral neuropathy in the human of grade 3 or greater.

In another aspect, the present disclosure provides kits comprising an immunoconjugate comprising the formula

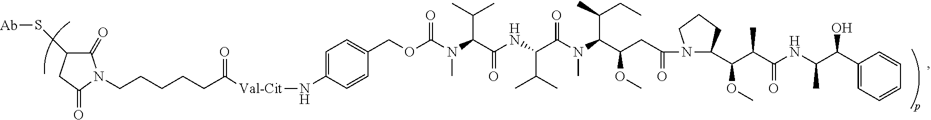

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, for use in combination with an immunomodulatory agent and an anti-CD20 antibody for treating a human in need thereof having follicular lymphoma (FL) according to any method of the present disclosure. In another aspect, the present disclosure provides kits comprising an immunoconjugate comprising the formula

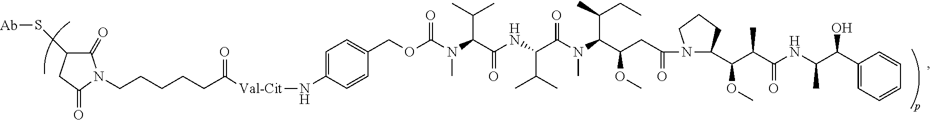

wherein Ab is an anti-CD79b antibody comprising (i) a heavy chain variable domain (VH) comprising the amino acid sequence of SEQ ID NO: 19 and (ii) a light chain variable domain (VL) comprising the amino acid sequence of SEQ ID NO: 20, and wherein p is between 2 and 5, for use in combination with lenalidomide and rituximab for treating a human in need thereof having follicular lymphoma (FL) according to any method of the present disclosure. In some embodiments, p is between 3 and 4. In some embodiments, the antibody comprises (i) a heavy chain comprising the amino acid sequence of SEQ ID NO: 36 and (ii) a light chain comprising the amino acid sequence of SEQ ID NO: 35.

In another aspect, the present disclosure provides kits comprising polatuzumab vedotin for use in combination with lenalidomide and rituximab for treating a human in need thereof having follicular lymphoma (FL) according to any method of the present disclosure. In some embodiments, the FL is relapsed/refractory FL.

In some embodiments of any of the above aspects, the FL is relapsed/refractory FL. In some embodiments of any of the above aspects, among a plurality of humans treated, at least 89% achieve an overall response.

In some embodiments of any of the above aspects, the anti-CD20 antibody is rituximab. In some embodiments, the polatuzumab vedotin is administered at a dose of about 1.8 mg/kg, the lenalidomide is administered at a dose between about 10 mg and about 20 mg, and the rituximab is administered at a dose of about 375 mg/m².

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 provides a schematic of the study design for the Phase Ib/II clinical trial described in Example 1. C=cycle; CR=complete response; D=day; EOI=end of induction; FL=follicular lymphoma; G=obinutuzumab; Len=lenalidomide; PO=by mouth; Pola=polatuzumab vedotin; PR=partial response; QD=every day; Q2M=every 2 months; RP2D=recommended Phase II dose; SD=stable disease. Each cycle is 28 days. A month is defined as 28 days. All patients (i.e., in the dose escalation phase and in the expansion phase) receive 6 cycles of induction with obinutuzumab, polatuzumab vedotin, and lenalidomide. ^{a}FL patients enrolled in the dose-escalation phase who achieve a CR, PR, or SD at EOI received maintenance treatment with G+Len following the maintenance schedule outlined for patients with FL during the expansion phase. ^{b}Maintenance treatment commenced 8 weeks (±1 week) after Day 1 of Cycle 6.

FIGS. 2A-2B provide a schematic of the induction (FIG. 2A) and post-induction (FIG. 2B) study treatments in the Phase Ib/II clinical trial described in Example 1. FL=follicular lymphoma; IV=intravenous; PO=by mouth; RP2D=recommended Phase II dose. During induction, treatments were administered sequentially in the following order: lenalidomide, obinutuzumab, and polatuzumab vedotin. During post-induction, treatment was administered in the following order: lenalidomide followed by obinutuzumab.

FIG. 3 provides a schematic of the dose-escalation plan for patients with FL treated with G+Len+Pola. A standard 3+3 dose escalation schema was used. The obinutuzumab dose remained fixed at 1000 mg. In Cohort 1, the starting doses are 1.4 mg/kg for Pola and 10 mg for Len. In Cohorts 2-6, dose escalation of Pola and Len proceeded in increments. For Pola, there were two possible dose levels: 1.4 mg/kg and 1.8 mg/kg. For Len, there were three possible dose levels: 10 mg, 15 mg, or 20 mg.

FIGS. 4A-4B provide a schematic of the guidelines for obinutuzumab infusions used in the Phase Ib/II clinical trial described in Example 1. FIG. 4A provides the guidelines for the first infusion of obinutuzumab and FIG. 4B provides the guidelines for the second and subsequent infusions of obinutuzumab. IRR=infusion-related reaction; q30=every 30. In FIG. 4A, ^{a}All patients received full premedication with an oral corticosteroid, antihistamine, and oral analgesic/antipyretic prior to the first obinutuzumab infusion; ^{b}Supportive treatment included acetaminophen/paracetamol and an antihistamine such as diphenhydramine, if not administered within the previous 4 hours. For bronchospasm, urticaria, or dyspnea, patients may have required antihistamines, oxygen, corticosteroids (e.g., 100 mg oral prednisone or equivalent), and/or bronchodilators. In FIG. 4B, ^{a}Patients received full premedication with an oral corticosteroid, antihistamine, and oral analgesic/antipyretic if they experienced an IRR ≥Grade 3 during the prior obinutuzumab infusion. In the case of a recurrent Grade 3 IRR, obinutuzumab may be discontinued at the discretion of the investigator, following an individual benefit-risk assessment; ^{b}Patients who experienced wheezing, urticaria, or other symptoms of anaphylaxis (see Example 1) received full premedication prior to all subsequent doses.

FIG. 5 provides a Kaplan-Meier Plot of progression-free survival (PFS) for efficacy evaluable patients (n=18) in the Phase Ib/II clinical trial described in Example 1. The median duration of follow up was 16.6 months (3.2-25.1 months). The median PFS was not reached. The 12-month PFS rate was 90%. Of 17 responders, two patients experienced disease progression to date and the remaining patients have ongoing responses with the longest being >21 months. The 12-month PFS rate was measured starting from initiation of study treatment (Cycle 1, day 1 of the induction phase).

FIG. 6 provides a schematic of the dose-escalation phase for patients with FL treated with G+Len+Pola. A standard 3+3 dose escalation schema was used. The obinutuzumab dose remained fixed at 1000 mg. For Pola, there were two possible dose levels: 1.4 mg/kg and 1.8 mg/kg. For Len, there were three possible dose levels: 10 mg, 15 mg, or 20 mg. Cohort 2 was halted due to dose-limiting toxicities (DLTs). Consequently, Cohorts 4 and 6 were not opened. Cohorts 1 and 3 were opened and cleared, and the dosing regimen for Cohort 5 of 1.4 mg/kg polatuzumab vedotin and 20 mg lenalidomide was determined to be the recommended Phase II dose (RP2D) when combined with a fixed dose of 1000 mg obinutuzumab.

FIGS. 7A-7D show analyses of the complete response (CR) and partial response (PR) rates (based on assessments by the IRC using the Lugano 2014 criteria) in the indicated patient subgroups from the efficacy evaluable population. FIG. 7A provides a comparison of the CR and PR rates between patients with progression of disease within 24 months of initiation of the first anti-lymphoma treatment with chemoimmunotherapy (POD24 on first line treatment) and without POD24 on first line treatment.

FIG. 7B provides a comparison of the CR and PR rates between patients classified as being in the High Risk Group, with 3-5 FLIPI Risk Factors (FLIPI High (3-5)) and patients classified as having 1-2 FLIPI Risk Factors (FLIPI 1-2). FIG. 7C provides a comparison of the CR and PR rates between patients that had disease refractory to the last line of treatment (Refractory) and patients that had disease not refractory to the last line of treatment (Not Refractory). FIG. 7D provides a comparison of the CR and PR rates between patients that had ≥3 prior lines of treatment and patients that had 1-2 prior lines of treatment.

FIG. 8 shows a summary of the follow-up period for each patient in the efficacy-evaluable population. The times of death, study discontinuation, determination of progressive disease (PD), determination of first partial response (PR), and determination of first complete response (CR) are indicated. The times of the last day of lenalidomide treatment and the last day of polatuzumab vedotin treatment are also provided. In addition, patients that remained on treatment are indicated.

FIG. 9 shows a Kaplan-Meier Plot of progression-free survival (PFS) for the efficacy-evaluable population in the Phase Ib/II clinical trial described in Example 2. The 12 month PFS, measured from the initiation of study treatment, was 83.4% (Confidence Interval: 70.85-95.96). The median duration of follow-up was 15.1 months. The median PFS was not reached. ¹PFS was determined by the investigator. CI=confidence interval; NE=not evaluable.

FIGS. 10A-10D show analyses of the complete response (CR), partial response (PR), and overall response (ORR) rates in the indicated patient subgroups from the efficacy evaluable population based on assessments by the IRC using the Lugano criteria. FIG. 10A provides a comparison of the CR, PR, and ORR rates between patients with progression of disease within 24 months of initiation of the first anti-lymphoma treatment with chemoimmunotherapy (POD24 on first line treatment) and patients without POD24 on first line treatment. FIG. 10B provides a comparison of the CR, PR, and ORR rates between patients classified as being in the High Risk Group, with 3-5 FLIPI Risk Factors (FLIPI high (3-5)), and patients classified as being in the Low Risk Group, with 0-2 FLIPI Risk Factors (FLIPI low (0-2)). FIG. 10C provides a comparison of the CR, PR, and ORR rates between patients that had disease refractory to the last line of treatment (Refractory) and patients that did not have disease refractory to the last line of treatment (Not Refractory). Refractory disease was defined as no response, progression, or relapse within 6 months of the last anti-lymphoma therapy end date. FIG. 10D provides a comparison of the CR, PR, and ORR rates between patients that had ≥3 prior lines of treatment and patients that had 1-2 prior lines of treatment.

### DETAILED DESCRIPTION

As used herein, the term “polatuzumab vedotin” refers to an anti-CD79b immunoconjugate having the IUPHAR/BPS Number 8404, the KEGG Number D10761, or the CAS Registry Number 1313206-42-6. Polatuzumab vedotin is also interchangeably referred to as “polatuzumab vedotin-piiq”, “huMA79bv28-MC-vc-PAB-MMAE”, “DCDS4501A”, or “RG7596.”

Provided herein are methods for treating or delaying progression of lymphoma (such as follicular lymphoma (FL), e.g., relapsed/refractory FL) in an individual (e.g., a human) comprising administering to the individual an effective amount of an anti-CD79b immunoconjugate (e.g., huMA79bv28-MC-vc-PAB-MMAE, which is also known as polatuzumab vedotin), an immunomodulatory agent (e.g., lenalidomide) and an anti-CD20 agent (e.g., an anti-CD20 antibody such as obinutuzumab or rituximab), wherein the individual achieves a response of at least stable disease (SD) (e.g., such as least SD, at least a partial response (PR) or a complete remission/complete response (CR)) following treatment.

In some embodiments, the method comprises treating an individual having follicular lymphoma (FL), e.g., relapsed/refractory FL, by administering to the individual (a) an immunoconjugate comprising the formula

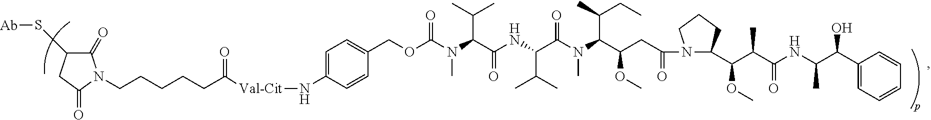

wherein Ab is an anti-CD79b antibody comprising (i) an HVR-H1 that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8 (e.g., between 2 and 5, or between 3 and 4), (b) an immunomodulatory agent (e.g., lenalidomide), and (c) an anti-CD20 agent (e.g., obinutuzumab or rituximab). In some embodiments, the immunoconjugate is administered at a dose between about 1.4 mg/kg and about 1.8 mg/kg, the immunomodulatory agent (e.g., lenalidomide) is administered at a dose between about 10 mg and about 20 mg, and the anti-CD20 agent (e.g., obinutuzumab) is administered at a dose of 1000 mg, and wherein the individual achieves a response of at least stable disease (SD) (e.g., at least SD, at least a partial response (PR), or a complete response or complete remission (CR)). In some embodiments, the immunoconjugate is administered at a dose between about 1.4 mg/kg and about 1.8 mg/kg, the immunomodulatory agent (e.g., lenalidomide) is administered at a dose between about 10 mg and about 20 mg, and the anti-CD20 agent (e.g., rituximab) is administered at a dose of 375 mg/m², and wherein the individual achieves a response of at least stable disease (SD) (e.g., at least SD, at least a partial response (PR), or a complete response or complete remission (CR)).

### I. General Techniques

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001).

### II. Definitions

Before describing the invention in detail, it is to be understood that this invention is not limited to particular compositions or biological systems, which can, of course, vary. It is also to be understood that the terminology used herein is for the purpose of describing particular embodiments only, and is not intended to be limiting.

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

The term “CD79b,” as used herein, refers to any native CD79b from any vertebrate source, including mammals such as primates (e.g., humans, cynomolgous monkey (“cyno”)) and rodents (e.g., mice and rats), unless otherwise indicated. Human CD79b is also referred herein to as “Igβ,” “B29,” “DNA225786” or “PRO36249.” An exemplary CD79b sequence including the signal sequence is shown in SEQ ID NO: 1. An exemplary CD79b sequence without the signal sequence is shown in SEQ ID NO: 2. The term “CD79b” encompasses “full-length,” unprocessed CD79b as well as any form of CD79b that results from processing in the cell. The term also encompasses naturally occurring variants of CD79b, e.g., splice variants, allelic variants and isoforms. The CD79b polypeptides described herein may be isolated from a variety of sources, such as from human tissue types or from another source, or prepared by recombinant or synthetic methods. A “native sequence CD79b polypeptide” comprises a polypeptide having the same amino acid sequence as the corresponding CD79b polypeptide derived from nature. Such native sequence CD79b polypeptides can be isolated from nature or can be produced by recombinant or synthetic means. The term “native sequence CD79b polypeptide” specifically encompasses naturally-occurring truncated or secreted forms of the specific CD79b polypeptide (e.g., an extracellular domain sequence), naturally-occurring variant forms (e.g., alternatively spliced forms) and naturally-occurring allelic variants of the polypeptide.

“CD20” as used herein refers to the human B-lymphocyte antigen CD20 (also known as CD20, B-lymphocyte surface antigen B1, Leu-16, Bp35, BM5, and LF5; the sequence is characterized by the SwissProt database entry P11836) is a hydrophobic transmembrane protein with a molecular weight of approximately 35 kD located on pre-B and mature B lymphocytes. (Valentine, M. A., et al., J. Biol. Chem. 264(19) (1989 11282-11287; Tedder, T. F., et al, Proc. Natl. Acad. Sci. U.S.A. 85 (1988) 208-12; Stamenkovic, I., et al., J. Exp. Med. 167 (1988) 1975-80; Einfeld, D. A. et al., EMBO J. 7 (1988) 711-7; Tedder, T. F., et al., J. Immunol. 142 (1989) 2560-8). The corresponding human gene is Membrane-spanning 4-domains, subfamily A, member 1, also known as MS4A1. This gene encodes a member of the membrane-spanning 4A gene family. Members of this nascent protein family are characterized by common structural features and similar intron/exon splice boundaries and display unique expression patterns among hematopoietic cells and nonlymphoid tissues. This gene encodes the B-lymphocyte surface molecule which plays a role in the development and differentiation of B-cells into plasma cells. This family member is localized to 11q12, among a cluster of family members. Alternative splicing of this gene results in two transcript variants which encode the same protein.

The terms “CD20” and “CD20 antigen” are used interchangeably herein, and include any variants, isoforms and species homologs of human CD20 which are naturally expressed by cells or are expressed on cells transfected with the CD20 gene. Binding of an antibody of the invention to the CD20 antigen mediate the killing of cells expressing CD20 (e.g., a tumor cell) by inactivating CD20. The killing of the cells expressing CD20 may occur by one or more of the following mechanisms: Cell death/apoptosis induction, ADCC and CDC. Synonyms of CD20, as recognized in the art, include B-lymphocyte antigen CD20, B-lymphocyte surface antigen B1, Leu-16, Bp35, BM5, and LF5.

The term “expression of the CD20” antigen is intended to indicate a significant level of expression of the CD20 antigen in a cell, e.g., a T- or B-Cell. In one embodiment, patients to be treated according to the methods of this invention express significant levels of CD20 on a B-cell tumor or cancer. Patients having a “CD20 expressing cancer” can be determined by standard assays known in the art. E.g., CD20 antigen expression is measured using immunohistochemical (IHC) detection, FACS or via PCR-based detection of the corresponding mRNA.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The term “epitope” refers to the particular site on an antigen molecule to which an antibody binds.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, δ, γ, and μ, respectively.

The term “anti-CD79b antibody” or “an antibody that binds to CD79b” refers to an antibody that is capable of binding CD79b with sufficient affinity such that the antibody is useful as a diagnostic and/or therapeutic agent in targeting CD79b. Preferably, the extent of binding of an anti-CD79b antibody to an unrelated, non-CD79b protein is less than about 10% of the binding of the antibody to CD79b as measured, e.g., by a radioimmunoassay (RIA). In certain embodiments, an antibody that binds to CD79b has a dissociation constant (Kd) of ≤1 μM, ≤100 nM, ≤10 nM, ≤1 nM, or ≤0.1 nM. In certain embodiments, anti-CD79b antibody binds to an epitope of CD79b that is conserved among CD79b from different species.

The term “anti-CD20 antibody” according to the invention refers to an antibody that is capable of binding CD20 with sufficient affinity such that the antibody is useful as a diagnostic and/or therapeutic agent in targeting CD20. Preferably, the extent of binding of an anti-CD20 antibody to an unrelated, non-CD20 protein is less than about 10% of the binding of the antibody to CD20 as measured, e.g., by a radioimmunoassay (RIA). In certain embodiments, an antibody that binds to CD20 has a dissociation constant (Kd) of ≤1 μM, ≤100 nM, ≤10 nM, ≤1 nM, or ≤0.1 nM. In certain embodiments, anti-CD20 antibody binds to an epitope of CD20 that is conserved among CD20 from different species.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007). The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

“Isolated nucleic acid encoding an anti-CD79b antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR,” as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987).) Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, MD (1991).) With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro and Fransson, Front. Biosci. 13:1619-1633 (2008).) Unless otherwise indicated, HVR residues and other residues in the variable domain (e.g., FR residues) are numbered herein according to Kabat et al., supra.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B-cell receptor); and B-cell activation.

“CD79b polypeptide variant” means a CD79b polypeptide, preferably an active CD79b polypeptide, as defined herein having at least about 80% amino acid sequence identity with a full-length native sequence CD79b polypeptide sequence as disclosed herein, a CD79b polypeptide sequence lacking the signal peptide as disclosed herein, an extracellular domain of a CD79b polypeptide, with or without the signal peptide, as disclosed herein or any other fragment of a full-length CD79b polypeptide sequence as disclosed herein (such as those encoded by a nucleic acid that represents only a portion of the complete coding sequence for a full-length CD79b polypeptide). Such CD79b polypeptide variants include, for instance, CD79b polypeptides wherein one or more amino acid residues are added, or deleted, at the N- or C-terminus of the full-length native amino acid sequence. Ordinarily, a CD79b polypeptide variant will have at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to a full-length native sequence CD79b polypeptide sequence as disclosed herein, a CD79b polypeptide sequence lacking the signal peptide as disclosed herein, an extracellular domain of a CD79b polypeptide, with or without the signal peptide, as disclosed herein or any other specifically defined fragment of a full-length CD79b polypeptide sequence as disclosed herein. Ordinarily, CD79b variant polypeptides are at least about 10 amino acids in length, alternatively at least about 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600 amino acids in length, or more. Optionally, CD79b variant polypeptides will have no more than one conservative amino acid substitution as compared to the native CD79b polypeptide sequence, alternatively no more than 2, 3, 4, 5, 6, 7, 8, 9, or 10 conservative amino acid substitution as compared to the native CD79b polypeptide sequence.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

An “immunoconjugate” is an antibody conjugated to one or more heterologous molecule(s), including but not limited to a cytotoxic agent.

In the context of the formulas provided herein, “p” refers to the average number of drug moieties per antibody, which can range, e.g., from about 1 to about 20 drug moieties per antibody, and in certain embodiments, from 1 to about 8 drug moieties per antibody. The invention includes a composition comprising a mixture of antibody-drug compounds of Formula I where the average drug loading per antibody is about 2 to about 5, or about 3 to about 4, (e.g., about 3.5).

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

The terms “cancer” and “cancerous” refer to or describe the physiological condition in mammals that is typically characterized by unregulated cell growth. Examples of cancer include but are not limited to, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. More specific examples include, but are not limited to, relapsed or refractory NHL, front line low grade NHL, Stage III/IV NHL, chemotherapy resistant NHL, precursor B lymphoblastic leukemia and/or lymphoma, small lymphocytic lymphoma, B-cell chronic lymphocytic leukemia and/or prolymphocytic leukemia and/or small lymphocytic lymphoma, B-cell prolymphocytic lymphoma, immunocytoma and/or lymphoplasmacytic lymphoma, lymphoplasmacytic lymphoma, marginal zone B-cell lymphoma, splenic marginal zone lymphoma, extranodal marginal zone-MALT lymphoma, nodal marginal zone lymphoma, hairy cell leukemia, plasmacytoma and/or plasma cell myeloma, low grade/follicular lymphoma, intermediate grade/follicular NHL, mantle cell lymphoma, follicle center lymphoma (follicular), follicular lymphoma (e.g., relapsed/refractory follicular lymphoma) intermediate grade diffuse NHL, diffuse large B-cell lymphoma (DLBCL), aggressive NHL (including aggressive front-line NHL and aggressive relapsed NHL), NHL relapsing after or refractory to autologous stem cell transplantation, primary mediastinal large B-cell lymphoma, primary effusion lymphoma, high grade immunoblastic NHL, high grade lymphoblastic NHL, high grade small non-cleaved cell NHL, bulky disease NHL, Burkitt's lymphoma, precursor (peripheral) large granular lymphocytic leukemia, mycosis fungoides and/or Sezary syndrome, skin (cutaneous) lymphomas, anaplastic large cell lymphoma, angiocentric lymphoma.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “effective amount” of an agent, e.g., a pharmaceutical formulation, refers to an amount effective, at dosages and for periods of time necessary, to achieve the desired therapeutic or prophylactic result.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, reduction of free light chain, preventing occurrence or recurrence of disease, alleviation of symptoms, diminishment of any direct or indirect pathological consequences of the disease, decreasing the rate of disease progression, amelioration or palliation of the disease state, and remission or improved prognosis. In some embodiments, the antibodies described herein are used to delay development of a disease or to slow the progression of a disease.

The term “CD79b-positive cancer” refers to a cancer comprising cells that express CD79b on their surface. In some embodiments, expression of CD79b on the cell surface is determined, for example, using antibodies to CD79b in a method such as immunohistochemistry, FACS, etc. Alternatively, CD79b mRNA expression is considered to correlate to CD79b expression on the cell surface and can be determined by a method selected from in situ hybridization and RT-PCR (including quantitative RT-PCR).

As used herein, “in conjunction with” refers to administration of one treatment modality in addition to another treatment modality. As such, “in conjunction with” refers to administration of one treatment modality before, during, or after administration of the other treatment modality to the individual.

A “chemotherapeutic agent” is a chemical compound useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, everolimus, sotrastaurin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin. Additional examples include of chemotherapeutic agents include bendamustine (or bendamustine-HCl) (TREANDA®), ibrutinib, lenalidomide, and/or idelalisib (GS-1101).

Additional examples of chemotherapeutic agents include anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY117018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRH) agonists such as leuprolide acetate (LUPRON® and ELIGARD®), goserelin acetate, buserelin acetate and tripterelin; anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®). In addition, such definition of chemotherapeutic agents includes bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); as well as troxacitabine (a 1,3-dioxolane nucleoside cytosine analog); anti-sense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine.

In some embodiments, the chemotherapeutic agent includes topoisomerase 1 inhibitor (e.g., LURTOTECAN®); an anti-estrogen such as fulvestrant; a Kit inhibitor such as imatinib or EXEL-0862 (a tyrosine kinase inhibitor); EGFR inhibitor such as erlotinib or cetuximab; an anti-VEGF inhibitor such as bevacizumab; arinotecan; rmRH (e.g., ABARELIX®); lapatinib and lapatinib ditosylate (an ErbB-2 and EGFR dual tyrosine kinase small-molecule inhibitor also known as GW572016); 17AAG (geldanamycin derivative that is a heat shock protein (Hsp) 90 poison), and pharmaceutically acceptable salts, acids or derivatives of any of the above.

Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), ublituximab, ofatumumab, ibritumomab tiuxetan, pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG1 λ antibody genetically modified to recognize interleukin-12 p40 protein.

The term “package insert” is used to refer to instructions customarily included in commercial packages of therapeutic products, that contain information about the indications, usage, dosage, administration, combination therapy, contraindications and/or warnings concerning the use of such therapeutic products.

“Alkyl” is C₁-C₁₈ hydrocarbon containing normal, secondary, tertiary or cyclic carbon atoms. Examples are methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃.

The term “C₁-C₈ alkyl,” as used herein refers to a straight chain or branched, saturated or unsaturated hydrocarbon having from 1 to 8 carbon atoms. Representative “C₁-C₈ alkyl” groups include, but are not limited to, -methyl, -ethyl, -n-propyl, -n-butyl, -n-pentyl, -n-hexyl, -n-heptyl, -n-octyl, -n-nonyl and -n-decyl; while branched C₁-C₈ alkyls include, but are not limited to, -isopropyl, -sec-butyl, -isobutyl, -tert-butyl, -isopentyl, 2-methylbutyl, unsaturated C₁-C₈ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl,-acetylenyl, -propynyl, -1-butynyl, -2-butynyl, -1-pentynyl, -2-pentynyl, -3-methyl-1 butynyl. A C₁-C₈ alkyl group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —SO₃R′, —S(O)₂R′, —S(O)R′, —OH, -halogen, —N₃, —NH₂, —NH(R′), —N(R′)₂ and —CN; where each R′ is independently selected from H, —C₁-C₈ alkyl and aryl.

The term “C₁-C₁₂ alkyl,” as used herein refers to a straight chain or branched, saturated or unsaturated hydrocarbon having from 1 to 12 carbon atoms. A C₁-C₁₂ alkyl group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —SO₃R′, —S(O)₂R′, —S(O)R′, —OH, -halogen, —N₃, —NH₂, —NH(R′), —N(R′)₂ and —CN; where each R′ is independently selected from H, —C₁-C₈ alkyl and aryl.

The term “C₁-C₆ alkyl,” as used herein refers to a straight chain or branched, saturated or unsaturated hydrocarbon having from 1 to 6 carbon atoms. Representative “C₁-C₆ alkyl” groups include, but are not limited to, -methyl, -ethyl, -n-propyl, -n-butyl, -n-pentyl, -and n-hexyl; while branched C₁-C₆ alkyls include, but are not limited to, -isopropyl, -sec-butyl, -isobutyl, -tert-butyl, -isopentyl, and 2-methylbutyl; unsaturated C₁-C₆ alkyls include, but are not limited to, -vinyl, -allyl, -1-butenyl, -2-butenyl, and -isobutylenyl, -1-pentenyl, -2-pentenyl, -3-methyl-1-butenyl, -2-methyl-2-butenyl, -2,3-dimethyl-2-butenyl, 1-hexyl, 2-hexyl, and 3-hexyl. A C₁-C₆ alkyl group can be unsubstituted or substituted with one or more groups, as described above for C₁-C₈ alkyl group.

The term “C₁-C₄ alkyl,” as used herein refers to a straight chain or branched, saturated or unsaturated hydrocarbon having from 1 to 4 carbon atoms. Representative “C₁-C₄ alkyl” groups include, but are not limited to, -methyl, -ethyl, -n-propyl, -n-butyl; while branched C₁-C₄ alkyls include, but are not limited to, -isopropyl, -sec-butyl, -isobutyl, -tert-butyl; unsaturated C₁-C₄ alkyls include, but are not limited to, -vinyl, -allyl, -1-butenyl, -2-butenyl, and -isobutylenyl. A C₁-C₄ alkyl group can be unsubstituted or substituted with one or more groups, as described above for C₁-C₈ alkyl group.

“Alkoxy” is an alkyl group singly bonded to an oxygen. Exemplary alkoxy groups include, but are not limited to, methoxy (—OCH₃) and ethoxy (—OCH₂CH₃). A “C₁-C₈ alkoxy” is an alkoxy group with 1 to 5 carbon atoms. Alkoxy groups may can be unsubstituted or substituted with one or more groups, as described above for alkyl groups.

“Alkenyl” is C₂-C₁₈ hydrocarbon containing normal, secondary, tertiary or cyclic carbon atoms with at least one site of unsaturation, i.e. a carbon-carbon, sp² double bond. Examples include, but are not limited to: ethylene or vinyl (—CH═CH₂), allyl (—CH₂CH═CH₂), cyclopentenyl (—C₅H₇), and 5-hexenyl (—CH₂CH₂CH₂CH₂CH═CH₂). A “C₂-C₈ alkenyl” is a hydrocarbon containing 2 to 8 normal, secondary, tertiary or cyclic carbon atoms with at least one site of unsaturation, i.e. a carbon-carbon, sp² double bond.

“Alkynyl” is C₂-C₁₈ hydrocarbon containing normal, secondary, tertiary or cyclic carbon atoms with at least one site of unsaturation, i.e. a carbon-carbon, sp triple bond. Examples include, but are not limited to: acetylenic (—C≡CH) and propargyl (—CH₂C≡CH). A “C₂-C₈ alkynyl” is a hydrocarbon containing 2 to 8 normal, secondary, tertiary or cyclic carbon atoms with at least one site of unsaturation, i.e. a carbon-carbon, sp triple bond.

“Alkylene” refers to a saturated, branched or straight chain or cyclic hydrocarbon radical of 1-18 carbon atoms, and having two monovalent radical centers derived by the removal of two hydrogen atoms from the same or two different carbon atoms of a parent alkane. Typical alkylene radicals include, but are not limited to: methylene (—CH₂—) 1,2-ethyl (—CH₂CH₂—), 1,3-propyl (—CH₂CH₂CH₂—), 1,4-butyl (—CH₂CH₂CH₂CH₂—), and the like.

A “C₁-C₁₀ alkylene” is a straight chain, saturated hydrocarbon group of the formula —(CH₂)₁₋₁₀—. Examples of a C₁-C₁₀ alkylene include methylene, ethylene, propylene, butylene, pentylene, hexylene, heptylene, ocytylene, nonylene and decalene.

“Alkenylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkene. Typical alkenylene radicals include, but are not limited to: 1,2-ethylene (—CH═CH—).

“Alkynylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkyne. Typical alkynylene radicals include, but are not limited to: acetylene (—C≡C—), propargyl (—CH₂C≡C—), and 4-pentynyl (—CH₂CH₂CH₂C≡C—).

“Aryl” refers to a carbocyclic aromatic group. Examples of aryl groups include, but are not limited to, phenyl, naphthyl and anthracenyl. A carbocyclic aromatic group or a heterocyclic aromatic group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; wherein each R′ is independently selected from H, —C₁-C₈ alkyl and aryl.

A “C₈-C₂₀ aryl” is an aryl group with 5 to 20 carbon atoms in the carbocyclic aromatic rings. Examples of C₈-C₂₀ aryl groups include, but are not limited to, phenyl, naphthyl and anthracenyl. A C₈-C₂₀ aryl group can be substituted or unsubstituted as described above for aryl groups. A “C₈-C₁₄ aryl” is an aryl group with 5 to 14 carbon atoms in the carbocyclic aromatic rings. Examples of C₈-C₁₄ aryl groups include, but are not limited to, phenyl, naphthyl and anthracenyl. A C₈-C₁₄ aryl group can be substituted or unsubstituted as described above for aryl groups.

An “arylene” is an aryl group which has two covalent bonds and can be in the ortho, meta, or para configurations as shown in the following structures:

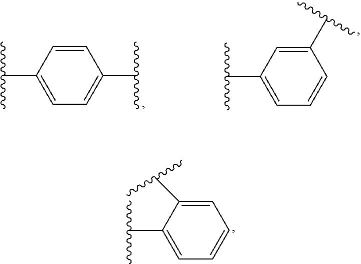 [SMILES: *c1ccc(*)cc1 ; *c1cccc(*)c1 ; *c1ccccc1*]

in which the phenyl group can be unsubstituted or substituted with up to four groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; wherein each R′ is independently selected from H, —C₁-C₈ alkyl and aryl.

“Arylalkyl” refers to an acyclic alkyl radical in which one of the hydrogen atoms bonded to a carbon atom, typically a terminal or sp³ carbon atom, is replaced with an aryl radical. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. The arylalkyl group comprises 6 to 20 carbon atoms, e.g., the alkyl moiety, including alkanyl, alkenyl or alkynyl groups, of the arylalkyl group is 1 to 6 carbon atoms and the aryl moiety is 5 to 14 carbon atoms.

“Heteroarylalkyl” refers to an acyclic alkyl radical in which one of the hydrogen atoms bonded to a carbon atom, typically a terminal or sp³ carbon atom, is replaced with a heteroaryl radical. Typical heteroarylalkyl groups include, but are not limited to, 2-benzimidazolylmethyl, 2-furylethyl, and the like. The heteroarylalkyl group comprises 6 to 20 carbon atoms, e.g., the alkyl moiety, including alkanyl, alkenyl or alkynyl groups, of the heteroarylalkyl group is 1 to 6 carbon atoms and the heteroaryl moiety is 5 to 14 carbon atoms and 1 to 3 heteroatoms selected from N, O, P, and S. The heteroaryl moiety of the heteroarylalkyl group may be a monocycle having 3 to 7 ring members (2 to 6 carbon atoms or a bicycle having 7 to 10 ring members (4 to 9 carbon atoms and 1 to 3 heteroatoms selected from N, O, P, and S), for example: a bicyclo [4,5], [5,5], [5,6], or [6,6] system.

“Substituted alkyl,” “substituted aryl,” and “substituted arylalkyl” mean alkyl, aryl, and arylalkyl respectively, in which one or more hydrogen atoms are each independently replaced with a substituent. Typical substituents include, but are not limited to, —X, —R, —O, —OR, —SR, —S, —NR₂, —NR₃, ═NR, —CX₃, —CN, —OCN, —SCN, —N═C═O, —NCS, —NO, —NO₂, ═N₂, —N₃, NC(═O)R, —C(═O)R, —C(═O)NR₂, —SO₃⁻, —SO₃H, —S(═O)₂R, —OS(═O)₂OR, —S(═O)₂NR, —S(═O)R, —OP(═O)(OR)₂, —P(═O)(OR)₂, —PO⁻₃, —PO₃H₂, —C(═O)R, —C(═O)X, —C(═S)R, —CO₂R, —CO₂, —C(═S)OR, —C(═O)SR, —C(═S)SR, —C(═O)NR₂, —C(═S)NR₂, —C(═NR)NR₂, where each X is independently a halogen: F, Cl, Br, or I; and each R is independently —H, C₂-C₁₈ alkyl, C₆-C₂₀ aryl, C₃-C₁₄ heterocycle, protecting group or prodrug moiety. Alkylene, alkenylene, and alkynylene groups as described above may also be similarly substituted.

“Heteroaryl” and “heterocycle” refer to a ring system in which one or more ring atoms is a heteroatom, e.g., nitrogen, oxygen, and sulfur. The heterocycle radical comprises 3 to 20 carbon atoms and 1 to 3 heteroatoms selected from N, O, P, and S. A heterocycle may be a monocycle having 3 to 7 ring members (2 to 6 carbon atoms and 1 to 3 heteroatoms selected from N, O, P, and S) or a bicycle having 7 to 10 ring members (4 to 9 carbon atoms and 1 to 3 heteroatoms selected from N, O, P, and S), for example: a bicyclo [4,5], [5,5], [5,6], or [6,6] system.

Exemplary heterocycles are described, e.g., in Paquette, Leo A., “Principles of Modern Heterocyclic Chemistry” (W.A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566.

Examples of heterocycles include by way of example and not limitation pyridyl, dihydroypyridyl, tetrahydropyridyl (piperidyl), thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, bis-tetrahydrofuranyl, tetrahydropyranyl, bis-tetrahydropyranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, and isatinoyl.

By way of example and not limitation, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline. Still more typically, nitrogen bonded heterocycles include 1-aziridyl, 1-azetedyl, 1-pyrrolyl, 1-imidazolyl, 1-pyrazolyl, and 1-piperidinyl.

A “C₃-C₈ heterocycle” refers to an aromatic or non-aromatic C₃-C₈ carbocycle in which one to four of the ring carbon atoms are independently replaced with a heteroatom from the group consisting of O, S and N. Representative examples of a C₃-C₈ heterocycle include, but are not limited to, benzofuranyl, benzothiophene, indolyl, benzopyrazolyl, coumarinyl, isoquinolinyl, pyrrolyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl and tetrazolyl. A C₃-C₈ heterocycle can be unsubstituted or substituted with up to seven groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; wherein each R′ is independently selected from H, —C₁-C₈ alkyl and aryl.

“C₃-C₈ heterocyclo” refers to a C₃-C₈ heterocycle group defined above wherein one of the heterocycle group's hydrogen atoms is replaced with a bond. A C₃-C₈ heterocyclo can be unsubstituted or substituted with up to six groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; wherein each R′ is independently selected from H, —C₁-C₈ alkyl and aryl.

A “C₃-C₂₀ heterocycle” refers to an aromatic or non-aromatic C₃-C₈ carbocycle in which one to four of the ring carbon atoms are independently replaced with a heteroatom from the group consisting of O, S and N. A C₃-C₂₀ heterocycle can be unsubstituted or substituted with up to seven groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; wherein each R′ is independently selected from H, —C₁-C₈ alkyl and aryl.

“C₃-C₂₀ heterocyclo” refers to a C₃-C₂₀ heterocycle group defined above wherein one of the heterocycle group's hydrogen atoms is replaced with a bond.

“Carbocycle” means a saturated or unsaturated ring having 3 to 7 carbon atoms as a monocycle or 7 to 12 carbon atoms as a bicycle. Monocyclic carbocycles have 3 to 6 ring atoms, still more typically 5 or 6 ring atoms. Bicyclic carbocycles have 7 to 12 ring atoms, e.g., arranged as a bicyclo [4,5], [5,5], [5,6] or [6,6] system, or 9 or 10 ring atoms arranged as a bicyclo [5,6] or [6,6] system. Examples of monocyclic carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cycloheptyl, and cyclooctyl.

A “C₃-C₈ carbocycle” is a 3-, 4-, 5-, 6-, 7- or 8-membered saturated or unsaturated non-aromatic carbocyclic ring. Representative C₃-C₈ carbocycles include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. A C₃-C₈ carbocycle group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; where each R′ is independently selected from H, —C₁-C₈ alkyl and aryl.

A “C₃-C₈ carbocyclo” refers to a C₃-C₈ carbocycle group defined above wherein one of the carbocycle groups' hydrogen atoms is replaced with a bond.

“Linker” refers to a chemical moiety comprising a covalent bond or a chain of atoms that covalently attaches an antibody to a drug moiety. In various embodiments, linkers include a divalent radical such as an alkyldiyl, an aryldiyl, a heteroaryldiyl, moieties such as: —(CR₂)ₙO(CR₂)ₙ—, repeating units of alkyloxy (e.g., polyethylenoxy, PEG, polymethyleneoxy) and alkylamino (e.g., polyethyleneamino, Jeffamine™); and diacid ester and amides including succinate, succinamide, diglycolate, malonate, and caproamide. In various embodiments, linkers can comprise one or more amino acid residues, such as valine, phenylalanine, lysine, and homolysine.

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

“Leaving group” refers to a functional group that can be substituted by another functional group. Certain leaving groups are well known in the art, and examples include, but are not limited to, a halide (e.g., chloride, bromide, iodide), methanesulfonyl (mesyl), p-toluenesulfonyl (tosyl), trifluoromethylsulfonyl (triflate), and trifluoromethylsulfonate.

The term “protecting group” refers to a substituent that is commonly employed to block or protect a particular functionality while reacting other functional groups on the compound. For example, an “amino-protecting group” is a substituent attached to an amino group that blocks or protects the amino functionality in the compound. Suitable amino-protecting groups include, but are not limited to, acetyl, trifluoroacetyl, t-butoxycarbonyl (BOC), benzyloxycarbonyl (CBZ) and 9-fluorenylmethylenoxycarbonyl (Fmoc). For a general description of protecting groups and their use, see T. W. Greene, Protective Groups in Organic Synthesis, John Wiley & Sons, New York, 1991, or a later edition.

### III. Methods

Provided herein are methods of treating a B-cell proliferative disorder (such as follicular lymphoma (FL), e.g., relapsed/refractory FL) in an individual (a human individual) in need thereof comprising administering to the individual an effective amount of (a) an immunoconjugate comprising an antibody which binds CD79b linked to a cytotoxic agent and (b) at least one additional therapeutic agent, wherein the individual achieves a response of at least stable disease (SD) (such as at least SD, at least partial response (PR), or a complete response/complete remission (CR)) following treatment (e.g., treatment regimen) (Additional details regarding SD, PR, and CR are provided herein below.) In some embodiments, the at least one additional therapeutic agent is a chemotherapeutic agent. In some embodiments, the at least one additional therapeutic agent is cytotoxic agent.

Provided herein are methods for treating a B-cell proliferative disorder (such as follicular lymphoma (FL), e.g., relapsed/refractory FL) in an individual (a human individual) in need thereof comprising administering to the individual an effective amount of (a) an immunoconjugate comprising an anti-CD79b antibody linked to a cytotoxic agent (i.e., anti-CD79b immunoconjugate and (b) an immunomodulatory agent, and (c) and anti-CD20 agent (such as an anti-CD20 antibody), wherein the individual achieves a response of at least stable disease (SD) (such as at least SD, at least partial response (PR), or a complete response/complete remission (CR)) following treatment. In some embodiments, the anti-CD79b immunoconjugate is huMA79bv28-MC-vc-PAB-MMAE. In some embodiments, the immunoconjugate is polatuzumab vedotin (CAS Registry Number 1313206-42-6). In some embodiments, the anti-CD79b immunoconjugate is huMA79bv28-MC-vc-PAB-MMAE. In some embodiments, the immunoconjugate is polatuzumab vedotin (CAS Registry Number 1313206-42-6). In some embodiments, the immunomodulatory agent is lenalidomide. In some embodiments, the anti-CD20 agent is an anti-CD20 antibody. In some embodiments, the anti-CD20 antibody is a humanized B-Ly1 antibody. In some embodiments, the humanized B-Ly1 antibody is obinutuzumab. In some embodiments, the anti-CD20 antibody is rituximab. In some embodiments, the anti-CD20 antibody is ofatumumab, ublituximab, and/or ibritumomab tiuxetan.

The terms “co-administration” or “co-administering” refer to the administration of the anti-CD79b immunoconjugate and the at least one additional therapeutic agent (e.g., an immunomodulatory agent and an anti-CD20 agent) as two (or more) separate formulations (or as one single formulation comprising the antiCD79b immunoconjugate and the at least one addition agent). Where separate formulations are used, the co-administration can be simultaneous or sequential in either order, wherein preferably there is a time period while all active agents simultaneously exert their biological activities. The anti-CD79b immunoconjugate and the at least additional therapeutic agent (e.g., an immunomodulatory agent and an anti-CD20 agent) are co-administered either simultaneously or sequentially. In some embodiments, when all therapeutic agents are co-administered sequentially, the dose is administered either on the same day in two separate administrations, or one of the agents is administered on day 1, the other agent(s) are co-administered between day 2 to day 7, such as between day 2 to 4. In some embodiments, the term “sequentially” means within 7 days after the dose of the first component, e.g., within 4 days after the dose of the first component; and the term “simultaneously” means at the same time. The term “co-administration” with respect to the maintenance doses of the anti-CD79b immunoconjugate and the at least one additional therapeutic agent (e.g., an immunomodulatory agent and an anti-CD20 agent) means that the maintenance doses can be either co-administered simultaneously, if the treatment cycle is appropriate for all drugs, e.g., every week. Alternatively, the anti-CD79b immunoconjugate is e.g., administered e.g., every first to third day and the at least one additional therapeutic agent (e.g., an immunomodulatory agent and an anti-CD20 agent) is administered every week. Alternatively, the maintenance doses are co-administered sequentially, either within one or within several days.

Anti-CD79b immunoconjugates and additional therapeutic agents (e.g., an immunomodulatory agent and an anti-CD20 agent) provided herein for use in any of the therapeutic methods described herein would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The immunoconjugate need not be, but is optionally formulated with one or more agents currently used to prevent or treat the disorder in question.

The amount of co-administration of the anti-CD79b immunoconjugate and the additional therapeutic agent and the timing of co-administration will depend on the type (species, gender, age, weight, etc.) and condition of the patient being treated and the severity of the disease or condition being treated. The anti-CD79b immunoconjugate and the at least one additional therapeutic agent (e.g., an immunomodulatory agent and an anti-CD20 agent) are suitably co-administered to the patient at one time or over a series of treatments e.g., on the same day or on the day after.

In some embodiments, the dosage of anti-CD79b immunoconjugate (such as huMA79bv28-MC-vc-PAB-MMAE or polatuzumab vedotin) is between about any of 1.4-5 mg/kg, 1.4-4 mg/kg, 1.4-3.2 mg/kg, 1.4-2.4 mg/kg, or 1.4-1.8 mg/kg. In some embodiments of any of the methods, the dosage of anti-CD79 immunoconjugate is about any of 1.4, 1.5. 1.6. 1.7, 1.8, 1.9 2.0, 2.2, 2.4, 2.6, 2.8, 3.0. 3.2, 3.4, 3.6, 3.8, 4.0, 4.2, 4.4, 4.6, and/or 4.8 mg/kg. In some embodiments, the dosage of anti-CD79b immunoconjugate is about 1.4 mg/kg. In some embodiments, the dosage of anti-CD79b immunoconjugate is about 1.8 mg/kg. In some embodiments, the dosage of anti-CD79b immunoconjugate is about 2.4 mg/kg. In some embodiments, the dosage of anti-CD79b immunoconjugate is about 3.2 mg/kg. In some embodiments, the dosage of anti-CD79b immunoconjugate is about 3.6 mg/kg. In some embodiments of any of the methods, the anti-CD79b immunoconjugate is administered q3wk. In some embodiments, the anti-CD79b immunoconjugate is administered via intravenous infusion. In some embodiments, the dosage administered via infusion is in the range of about 1 mg to about 1,500 mg per dose, generally one dose per week for a total of one, two, three or four doses. Alternatively, the dosage range is of about 1 mg to about 1,500 mg, about 1 mg to about 1,000 mg, about 400 mg to about 1200 mg, about 600 mg to about 1000 mg, about 10 mg to about 500 mg, about 10 mg to about 300 mg, about 10 mg to about 200 mg, and about 1 mg to about 200 mg. In some embodiments, the dosage administered via infusion is in the range of about 1 μg/m² to about 10,000 μg/m² per dose, generally one dose per week for a total of one, two, three or four doses. Alternatively, the dosage range is of about 1 μg/m² to about 1000 μg/m², about 1 μg/m² to about 800 μg/m², about 1 μg/m² to about 600 μg/m², about 1 μg/m² to about 400 μg/m², about 10 μg/m² to about 500 μg/m², about 10 μg/m² to about 300 μg/m², about 10 μg/m² to about 200 μg/m², and about 1 μg/m² to about 200 μg/m². The dose may be administered once per day, once per week, multiple times per week, but less than once per day, multiple times per month but less than once per day, multiple times per month but less than once per week, once per month or intermittently to relieve or alleviate symptoms of the disease. Administration may continue at any of the disclosed intervals until remission of the tumor or symptoms of the B-cell proliferative disorder being treated. Administration may continue after remission or relief of symptoms is achieved where such remission or relief is prolonged by such continued administration.

In some embodiments, the dosage of the anti-CD20 agent (e.g., anti-CD20 antibody) is between about 300-1600 mg/m² and/or 300-2000 mg. In some embodiments, the dosage of the anti-CD20 antibody is about any of 300, 375, 600, 1000, or 1250 mg/m² and/or 300, 1000, or 2000 mg. In some embodiments, the anti-CD20 antibody is rituximab and the dosage administered is 375 mg/m². In some embodiments, the anti-CD20 antibody is obinutuzumab and the dosage administered is 1000 mg. In some embodiments, the anti-CD20 antibody is administered q3w (i.e., every 3 weeks). In some embodiments, the dosage of said afucosylated anti-CD20 antibody (preferably the afucosylated humanized B-Ly1 antibody) may be 800 to 1600 mg (in one embodiment 800 to 1200 mg, such as 1000 mg) on day 1, 8, 15 of a 3- to 6-week dosage cycle and then in a dosage of 400 to 1200 (in one embodiment 800 to 1200 mg on day 1 of up to nine 3- to 4-week dosage cycles. In some embodiments, the dose is a flat dose 1000 mg in a three-weeks-dosage schedule, with the possibility of an additional cycle of a flat dose of 1000 mg in the second week.

Exemplary dosing regimens for the combination therapy of anti-CD79b immunoconjugates (such as huMA79bv28-MC-vc-PAB-MMAE or polatuzumab vedotin) and other agents include, but are not limited to, anti-CD79 immunoconjugate (such as huMA79bv28-MC-vc-PAB-MMAE) administered at about 1.4-5 mg/kg q4w, plus 375 mg/m² q4w rituximab, and 10-20 mg of lenalidomide on Days 1-21 of a 28-day cycle (e.g., each of days 1-21 q4w). In some embodiments, the anti-CD79 immunoconjugate is administered at about any of 1.4 mg/kg 1.8 mg/kg, 2.0 mg/kg, 2.2 mg/kg, 2.4 mg/kg, 3.2 mg/kg, or 4.0 mg/kg. In some embodiments, the anti-CD79b immunoconjugate is administered at about 1.4 mg/kg. In some embodiments, the anti-CD79b immunoconjugate is administered at about 1.8 mg/kg. In some embodiments, the anti-CD79b immunoconjugate is administered at about 2.4 mg/kg. In some embodiments, immunomodulatory agent (e.g., lenalidomide) is administered at about 10 mg. In some embodiments, immunomodulatory agent (e.g., lenalidomide) is administered at about 15 mg. In some embodiments, immunomodulatory agent (e.g., lenalidomide) is administered at about 20 mg.

Another exemplary dosage regimen for the combination therapy of anti-CD79b immunoconjugates (such as huMA79bv28-MC-vc-PAB-MMAE or polatuzumab vedotin) and other agents include, but are not limited to, anti-CD79 immunoconjugate (such as huMA79bv28-MC-vc-PAB-MMAE or polatuzumab vedotin) administered at about 1.4-5 mg/kg q4w, plus 1000 mg q4w obinutuzumab, and 10-20 mg/m² lenalidomide administered on Days 1-21 of a 28-day cycle (e.g., each of days 1-21 q4w). In some embodiments, the anti-CD79 immunoconjugate is administered at about any of 1.4 mg/kg, 1.8 mg/kg, 2.0 mg/kg, 2.2 mg/kg, 2.4 mg/kg, 3.2 mg/kg, or 4.0 mg/kg. In some embodiments, the anti-CD79b immunoconjugate is administered at about 1.8 mg/kg. In some embodiments, the anti-CD79b immunoconjugate is administered at about 1.8 mg/kg. In some embodiments, the anti-CD79b immunoconjugate is administered at about 2.4 mg/kg. In some embodiments, immunomodulatory agent (e.g., lenalidomide) is administered at about 10 mg. In some embodiments, immunomodulatory agent (e.g., lenalidomide) is administered at about 15 mg. In some embodiments, immunomodulatory agent (e.g., lenalidomide) is administered at about 20 mg.

An immunoconjugate provided herein (and any additional therapeutic agents, e.g., an immunomodulatory agent and an anti-CD20 agent) for use in any of the therapeutic methods described herein can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Provided herein are methods of treating follicular lymphoma (FL, e.g., relapsed/refractory FL) in an individual (a human individual) in need thereof comprising administering to the individual an effective amount of: (a) an immunoconjugate comprising the formula

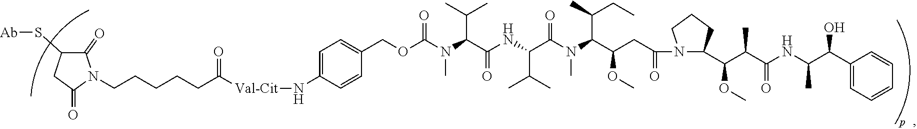

wherein Ab is an anti-CD79b antibody comprising (i) an HVR-H1 that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8; (b) an immunomodulatory agent, and (c) an anti-CD20 antibody, wherein the individual achieves a response of at least SD (e.g., at least SD, at least partial response (PR), or complete response or complete remission (CR)) following treatment (e.g., the treatment regimen) with the immunoconjugate, the immunomodulatory agent, and the anti-CD20 antibody. In some embodiments, the immunoconjugate comprises an anti-CD79 antibody that comprises a heavy chain variable domain (VH) comprising the amino acid sequence of SEQ ID NO: 19 and a light chain variable domain (VL) comprising the amino acid sequence of SEQ ID NO: 20. In some embodiments, the immunoconjugate comprises an anti-CD79 antibody that comprises a heavy chain comprising the amino acid sequence of SEQ ID NO: 37 and a light chain comprising the amino acid sequence of SEQ ID NO: 35. In some embodiments, the immunoconjugate comprises an anti-CD79 antibody that comprises a heavy chain comprising the amino acid sequence of SEQ ID NO: 36 and a light chain comprising the amino acid sequence of SEQ ID NO: 38. In some embodiments, the immunoconjugate comprises an anti-CD79 antibody that comprises a heavy chain comprising the amino acid sequence of SEQ ID NO: 36 and a light chain comprising the amino acid sequence of SEQ ID NO: 35. In some embodiments, p is between 2 and 7, between 2 and 6, between 2 and 5, between 3 and 5, or between 3 and 4. In some embodiments, p is 3.4. In some embodiments, the anti-CD79b immunoconjugate is huMA79bv28-MC-vc-PAB-MMAE. In some embodiments, the immunoconjugate is polatuzumab vedotin (CAS Registry Number 1313206-42-6). In some embodiment, the immunomodulatory agent is lenalidomide. In some embodiments, the anti-CD20 antibody is rituximab, a humanized B-Ly1 antibody, obinutuzumab, ofatumumab, ublituximab, or ibritumomab tiuxetan.

The anti-CD79b immunoconjugate (e.g., huMA79bv28-MC-vc-PAB-MMAE or polatuzumab vedotin), the immunomodulatory agent (such as lenalidomide) and the anti-CD20 antibody (such as obinutuzumab or rituximab) may be administered by the same route of administration or by different routes of administration. In some embodiments, the anti-CD79b immunoconjugate is administered intravenously, intramuscularly, subcutaneously, topically, orally, transdermally, intraperitoneally, intraorbitally, by implantation, by inhalation, intrathecally, intraventricularly, or intranasally. In some embodiments, the immunomodulatory agent (such as lenalidomide) is administered intravenously, intramuscularly, subcutaneously, topically, orally, transdermally, intraperitoneally, intraorbitally, by implantation, by inhalation, intrathecally, intraventricularly, or intranasally. In some embodiments, the anti-CD20 antibody (such as obinutuzumab or rituximab) is administered intravenously, intramuscularly, subcutaneously, topically, orally, transdermally, intraperitoneally, intraorbitally, by implantation, by inhalation, intrathecally, intraventricularly, or intranasally. In some embodiments, the anti-CD79b immunoconjugate and the anti-CD20 antibody (such as obinutuzumab or rituximab) are each administered via intravenous infusion, and the immunomodulatory agent (such as lenalidomide) is administered orally. An effective amount of the anti-CD79b immunoconjugate, the immunomodulatory agent (such as lenalidomide) and the anti-CD20 antibody (such as rituximab) may be administered for prevention or treatment of disease.

In some embodiments, the anti-CD79b immunoconjugate (e.g., huMA79bv28-MC-vc-PAB-MMAE or polatuzumab vedotin) is administered at a dose between about 1.4 mg/kg to about 1.8 mg/kg. In some embodiments, the anti-CD79b immunoconjugate (e.g., huMA79bv28-MC-vc-PAB-MMAE or polatuzumab vedotin) is administered at a dose of 1.4 mg/kg. In some embodiments, the anti-CD79b immunoconjugate (e.g., huMA79bv28-MC-vc-PAB-MMAE or polatuzumab vedotin) is administered at a dose of 1.8 mg/kg. Alternatively or additionally, in some embodiments, the immunomodulatory agent (e.g., lenalidomide) is administered at a dose between about 10 mg and about 20 mg. In some embodiments, the immunomodulatory agent (e.g., lenalidomide) is administered at a dose of 10 mg. In some embodiments, the immunomodulatory agent (e.g., lenalidomide) is administered at a dose of 15 mg. In some embodiments, the immunomodulatory agent (e.g., lenalidomide) is administered at a dose of 20 mg. Alternatively or additionally, in some embodiments, the anti-CD20 antibody is obinutuzumab. In some embodiments, the obinutuzumab is administered at a dose of about 1000 mg. In some embodiments, the anti-CD20 antibody is rituximab. In some embodiments, the rituximab is administered at a dose of about 375 mg/m².

In some embodiments, the anti-CD79b immunoconjugate, the immunomodulatory agent, and the anti-CD20 antibody are administered during an induction phase. An “induction phase” refers to a phase of treatment wherein the anti-CD79b immunoconjugate is administered to a human. In some embodiments, the induction phase comprises less than one complete 28-day cycle. In some embodiments, the induction phase comprises between one and six (e.g., any of 1, 2, 3, 4, 5, or 6) 28-day cycles. In some embodiments, the induction phase comprises at least six 28-day cycles.

In some embodiments, during the induction phase, the immunoconjugate is administered intravenously at a dose of 1.4 mg/kg on Day 1, the immunomodulatory agent is administered intravenously at a dose of 10 mg on each of Days 1-21, and the anti-CD20 antibody is obinutuzumab, and the obinutuzumab is administered intravenously at a dose of 1000 mg on each of Days 1, 8, and 15 of the first 28 day cycle, and the immunoconjugate is administered intravenously at a dose of 1.4 mg/kg on Day 1, the immunomodulatory agent is administered orally at a dose of 10 mg on each of Days 1-21, and the obinutuzumab is administered intravenously at a dose of 1000 mg on Day 1 of each of the second, third, fourth, fifth, and sixth 28-day cycles.

In some embodiments, during the induction phase, the immunoconjugate is administered intravenously at a dose of 1.4 mg/kg on Day 1, the immunomodulatory agent is administered intravenously at a dose of 15 mg on each of Days 1-21, and the anti-CD20 antibody is obinutuzumab, and the obinutuzumab is administered intravenously at a dose of 1000 mg on each of Days 1, 8, and 15 of the first 28 day cycle, and the immunoconjugate is administered intravenously at a dose of 1.4 mg/kg on Day 1, the immunomodulatory agent is administered orally at a dose of 15 mg on each of Days 1-21, and the obinutuzumab is administered intravenously at a dose of 1000 mg on Day 1 of each of the second, third, fourth, fifth, and sixth 28-day cycles.

In some embodiments, during the induction phase, the immunoconjugate is administered intravenously at a dose of 1.4 mg/kg on Day 1, the immunomodulatory agent is administered intravenously at a dose of 20 mg on each of Days 1-21, and the anti-CD20 antibody is obinutuzumab, and the obinutuzumab is administered intravenously at a dose of 1000 mg on each of Days 1, 8, and 15 of the first 28 day cycle, and the immunoconjugate is administered intravenously at a dose of 1.4 mg/kg on Day 1, the immunomodulatory agent is administered orally at a dose of 20 mg on each of Days 1-21, and the obinutuzumab is administered intravenously at a dose of 1000 mg on Day 1 of each of the second, third, fourth, fifth, and sixth 28-day cycles.

In some embodiments, during the induction phase, the immunoconjugate is administered intravenously at a dose of 1.8 mg/kg on Day 1, the immunomodulatory agent is administered intravenously at a dose of 10 mg on each of Days 1-21, and the anti-CD20 antibody is obinutuzumab, and the obinutuzumab is administered intravenously at a dose of 1000 mg on each of Days 1, 8, and 15 of the first 28 day cycle, and the immunoconjugate is administered intravenously at a dose of 1.8 mg/kg on Day 1, the immunomodulatory agent is administered orally at a dose of 10 mg on each of Days 1-21, and the obinutuzumab is administered intravenously at a dose of 1000 mg on Day 1 of each of the second, third, fourth, fifth, and sixth 28-day cycles.

In some embodiments, during the induction phase, the immunoconjugate is administered intravenously at a dose of 1.8 mg/kg on Day 1, the immunomodulatory agent is administered intravenously at a dose of 15 mg on each of Days 1-21, and the anti-CD20 antibody is obinutuzumab, and the obinutuzumab is administered intravenously at a dose of 1000 mg on each of Days 1, 8, and 15 of the first 28 day cycle, and the immunoconjugate is administered intravenously at a dose of 1.8 mg/kg on Day 1, the immunomodulatory agent is administered orally at a dose of 15 mg on each of Days 1-21, and the obinutuzumab is administered intravenously at a dose of 1000 mg on Day 1 of each of the second, third, fourth, fifth, and sixth 28-day cycles.

In some embodiments, during the induction phase, the immunoconjugate is administered intravenously at a dose of 1.8 mg/kg on Day 1, the immunomodulatory agent is administered intravenously at a dose of 20 mg on each of Days 1-21, and the anti-CD20 antibody is obinutuzumab, and the obinutuzumab is administered intravenously at a dose of 1000 mg on each of Days 1, 8, and 15 of the first 28 day cycle, and the immunoconjugate is administered intravenously at a dose of 1.8 mg/kg on Day 1, the immunomodulatory agent is administered orally at a dose of 20 mg on each of Days 1-21, and the obinutuzumab is administered intravenously at a dose of 1000 mg on Day 1 of each of the second, third, fourth, fifth, and sixth 28-day cycles.

In some embodiments, during the induction phase, the immunoconjugate is administered intravenously at a dose of 1.4 mg/kg on Day 1, the immunomodulatory agent is administered intravenously at a dose of 10 mg on each of Days 1-21, and the anti-CD20 antibody is rituximab, and the rituximab is administered intravenously at a dose of 375 mg/m² (such as on each of Days 1, 8, and 15 of the first 28 day cycle), and the immunoconjugate is administered intravenously at a dose of 1.4 mg/kg on Day 1, the immunomodulatory agent is administered orally at a dose of 10 mg on each of Days 1-21, and the a rituximab is administered intravenously at a dose of 375 mg/m² (such as on Day 1) of each of the second, third, fourth, fifth, and sixth 28-day cycles.

In some embodiments, during the induction phase, the immunoconjugate is administered intravenously at a dose of 1.4 mg/kg on Day 1, the immunomodulatory agent is administered intravenously at a dose of 15 mg on each of Days 1-21, and the anti-CD20 antibody is rituximab, and the rituximab is administered intravenously at a dose of 375 mg/m² (such as on each of Days 1, 8, and 15 of the first 28 day cycle), and the immunoconjugate is administered intravenously at a dose of 1.4 mg/kg on Day 1, the immunomodulatory agent is administered orally at a dose of 15 mg on each of Days 1-21, and the a rituximab is administered intravenously at a dose of 375 mg/m² (such as on Day 1) of each of the second, third, fourth, fifth, and sixth 28-day cycles.

In some embodiments, during the induction phase, the immunoconjugate is administered intravenously at a dose of 1.4 mg/kg on Day 1, the immunomodulatory agent is administered intravenously at a dose of 20 mg on each of Days 1-21, and the anti-CD20 antibody is rituximab, and the rituximab is administered intravenously at a dose of 375 mg/m² (such as on each of Days 1, 8, and 15 of the first 28 day cycle), and the immunoconjugate is administered intravenously at a dose of 1.4 mg/kg on Day 1, the immunomodulatory agent is administered orally at a dose of 20 mg on each of Days 1-21, and the a rituximab is administered intravenously at a dose of 375 mg/m² (such as on Day 1) of each of the second, third, fourth, fifth, and sixth 28-day cycles.

In some embodiments, during the induction phase, the immunoconjugate is administered intravenously at a dose of 1.8 mg/kg on Day 1, the immunomodulatory agent is administered intravenously at a dose of 10 mg on each of Days 1-21, and the anti-CD20 antibody is rituximab, and the rituximab is administered intravenously at a dose of 375 mg/m² (such as on each of Days 1, 8, and 15 of the first 28 day cycle), and the immunoconjugate is administered intravenously at a dose of 1.8 mg/kg on Day 1, the immunomodulatory agent is administered orally at a dose of 10 mg on each of Days 1-21, and the a rituximab is administered intravenously at a dose of 375 mg/m² (such as on Day 1) of each of the second, third, fourth, fifth, and sixth 28-day cycles.

In some embodiments, during the induction phase, the immunoconjugate is administered intravenously at a dose of 1.8 mg/kg on Day 1, the immunomodulatory agent is administered intravenously at a dose of 15 mg on each of Days 1-21, and the anti-CD20 antibody is rituximab, and the rituximab is administered intravenously at a dose of 375 mg/m² (such as on each of Days 1, 8, and 15 of the first 28 day cycle), and the immunoconjugate is administered intravenously at a dose of 1.8 mg/kg on Day 1, the immunomodulatory agent is administered orally at a dose of 15 mg on each of Days 1-21, and the a rituximab is administered intravenously at a dose of 375 mg/m² (such as on Day 1) of each of the second, third, fourth, fifth, and sixth 28-day cycles.

In some embodiments, during the induction phase, the immunoconjugate is administered intravenously at a dose of 1.8 mg/kg on Day 1, the immunomodulatory agent is administered intravenously at a dose of 20 mg on each of Days 1-21, and the anti-CD20 antibody is rituximab, and the rituximab is administered intravenously at a dose of 375 mg/m² (such as on each of Days 1, 8, and 15 of the first 28 day cycle), and the immunoconjugate is administered intravenously at a dose of 1.8 mg/kg on Day 1, the immunomodulatory agent is administered orally at a dose of 20 mg on each of Days 1-21, and the a rituximab is administered intravenously at a dose of 375 mg/m² (such as on Day 1) of each of the second, third, fourth, fifth, and sixth 28-day cycles.

The dosing and administration schedules for exemplary induction phases are provided in Tables A-L below:

TABLES A-L
Dosing and Administration Schedules for Exemplary Induction Phases

| | | |
|---|---|---|
| | Cycle 1 | Cycles 2-6 |
| Drugs | (28 days) | (28 days each) |

TABLE A

| | | |
|---|---|---|
| Anti-CD79b | 1.4 mg/kg on Day 1 | 1.4 mg/kg on Day 1 |
| immunoconjugate | | |
| (polatuzumab vedotin) | | |
| Immunomodulatory | 10 mg on each of Days 1-21 | 10 mg on each of Days 1-21 |
| Agent | | |
| (lenalidomide) | | |
| Anti-CD20 Antibody | 1000 mg on each of Days 1, 8, and 15 | 1000 mg on Day 1 |
| (obinutuzumab) | | |

TABLE B

| | | |
|---|---|---|
| Anti-CD79b | 1.4 mg/kg on Day 1 | 1.4 mg/kg on Day 1 |
| immunoconjugate | | |
| (polatuzumab vedotin) | | |
| Immunomodulatory | 15 mg on each of Days 1-21 | 15 mg on each of Days 1-21 |
| Agent | | |
| (lenalidomide) | | |
| Anti-CD20 Antibody | 1000 mg on each of Days 1, 8, and 15 | 1000 mg on Day 1 |
| (obinutuzumab) | | |

TABLE C

| | | |
|---|---|---|
| Anti-CD79b | 1.4 mg/kg on Day 1 | 1.4 mg/kg on Day 1 |
| immunoconjugate | | |
| (polatuzumab vedotin) | | |
| Immunomodulatory | 20 mg on each of Days 1-21 | 20 mg on each of Days 1-21 |
| Agent | | |
| (lenalidomide) | | |
| Anti-CD20 Antibody | 1000 mg on each of Days 1, 8, and 15 | 1000 mg on Day 1 |
| (obinutuzumab) | | |

TABLE D

| | | |
|---|---|---|
| Anti-CD79b | 1.8 mg/kg on Day 1 | 1.8 mg/kg on Day 1 |
| immunoconjugate | | |
| (polatuzumab vedotin) | | |
| Immunomodulatory | 10 mg on each of Days 1-21 | 10 mg on each of Days 1-21 |
| Agent | | |
| (lenalidomide) | | |
| Anti-CD20 Antibody | 1000 mg on each of Days 1, 8, and 15 | 1000 mg on Day 1 |
| (obinutuzumab) | | |

TABLE E

| | | |
|---|---|---|
| Anti-CD79b | 1.8 mg/kg on Day 1 | 1.8 mg/kg on Day 1 |
| immunoconjugate | | |
| (polatuzumab vedotin) | | |
| Immunomodulatory | 15 mg on each of Days 1-21 | 15 mg on each of Days 1-21 |
| Agent | | |
| (lenalidomide) | | |
| Anti-CD20 Antibody | 1000 mg on each of Days 1, 8, and 15 | 1000 mg on Day 1 |
| (obinutuzumab) | | |

TABLE F

| | | |
|---|---|---|
| Anti-CD79b | 1.8 mg/kg on Day 1 | 1.8 mg/kg on Day 1 |
| immunoconjugate | | |
| (polatuzumab vedotin) | | |
| Immunomodulatory | 20 mg on each of Days 1-21 | 20 mg on each of Days 1-21 |
| Agent | | |
| (lenalidomide) | | |
| Anti-CD20 Antibody | 1000 mg on each of Days 1, 8, and 15 | 1000 mg on Day 1 |
| (obinutuzumab) | | |

TABLE G

| | | |
|---|---|---|
| Anti-CD79b | 1.4 mg/kg on Day 1 | 1.4 mg/kg on Day 1 |
| immunoconjugate | | |
| (polatuzumab vedotin) | | |
| Immunomodulatory | 10 mg on each of Days 1-21 | 10 mg on each of Days 1-21 |
| Agent | | |
| (lenalidomide) | | |
| Anti-CD20 Antibody | 375 mg/m² | 375 mg/m² |
| (rituximab) | (e.g., on each of Days 1, 8, and 15) | (e.g., on Day 1) |

TABLE H

| | | |
|---|---|---|
| Anti-CD79b | 1.4 mg/kg on Day 1 | 1.4 mg/kg on Day 1 |
| immunoconjugate | | |
| (polatuzumab vedotin) | | |
| Immunomodulatory | 15 mg on each of Days 1-21 | 15 mg on each of Days 1-21 |
| Agent | | |
| (lenalidomide) | | |
| Anti-CD20 Antibody | 375 mg/m² | 375 mg/m² |
| (rituximab) | (e.g., on each of Days 1, 8, and 15) | (e.g., on Day 1) |

TABLE I

| | | |
|---|---|---|
| Anti-CD79b | 1.4 mg/kg on Day 1 | 1.4 mg/kg on Day 1 |
| immunoconjugate | | |
| (polatuzumab vedotin) | | |
| Immunomodulatory | 20 mg on each of Days 1-21 | 20 mg on each of Days 1-21 |
| Agent | | |
| (lenalidomide) | | |
| Anti-CD20 Antibody | 375 mg/m² | 375 mg/m² |
| (rituximab) | (e.g., on each of Days 1, 8, and 15) | (e.g., on Day 1) |

TABLE J

| | | |
|---|---|---|
| Anti-CD79b | 1.8 mg/kg on Day 1 | 1.8 mg/kg on Day 1 |
| immunoconjugate | | |
| (polatuzumab vedotin) | | |
| Immunomodulatory | 10 mg on each of Days 1-21 | 10 mg on each of Days 1-21 |
| Agent | | |
| (lenalidomide) | | |
| Anti-CD20 Antibody | 375 mg/m² | 375 mg/m² |
| (rituximab) | (e.g., on each of Days 1, 8, and 15) | (e.g., on Day 1) |

TABLE K

| | | |
|---|---|---|
| Anti-CD79b | 1.8 mg/kg on Day 1 | 1.8 mg/kg on Day 1 |
| immunoconjugate | | |
| (polatuzumab vedotin) | | |
| Immunomodulatory | 15 mg on each of Days 1-21 | 15 mg on each of Days 1-21 |
| Agent | | |
| (lenalidomide) | | |
| Anti-CD20 Antibody | 375 mg/m² | 375 mg/m² |
| (rituximab) | (e.g., on each of Days 1, 8, and 15) | (e.g., on Day 1) |

TABLE L

| | | |
|---|---|---|
| Anti-CD79b | 1.8 mg/kg on Day 1 | 1.8 mg/kg on Day 1 |
| immunoconjugate | | |
| (polatuzumab vedotin) | | |
| Immunomodulatory | 20 mg on each of Days 1-21 | 20 mg on each of Days 1-21 |
| Agent | | |
| (lenalidomide) | | |
| Anti-CD20 Antibody | 375 mg/m² | 375 mg/m² |
| (rituximab) | (e.g., on each of Days 1, 8, and 15) | (e.g., on Day 1) |

In some embodiments, the anti-CD79b immunoconjugate (e.g., huMA79bv28-MC-vc-PAB-MMAE or polatuzumab vedotin), the immunomodulatory agent (e.g., lenalidomide), and the anti-CD20 antibody (e.g., obinutuzumab or rituximab) are administered sequentially during the induction phase in the first, second, third, fourth, fifth, and sixth 28-day cycles. In some embodiments, the immunomodulatory agent (e.g., lenalidomide) is administered prior to the anti-CD20 antibody (e.g., obinutuzumab or rituximab), and the anti-CD20 antibody (e.g., obinutuzumab or rituximab) is administered prior to the immunoconjugate (e.g., huMA79bv28-MC-vc-PAB-MMAE or polatuzumab vedotin) on Day 1, and the immunomodulatory agent (e.g., lenalidomide) is administered prior to the anti-CD20 antibody (e.g., obinutuzumab or rituximab) on Days 8 and 15 of the first 28-day cycle. Additionally or alternatively, in some embodiments, the immunomodulatory agent (e.g., lenalidomide) is administered prior to the anti-CD20 antibody (e.g., obinutuzumab or rituximab), and the anti-CD20 antibody (e.g., obinutuzumab or rituximab) is administered prior to the immunoconjugate (e.g., huMA79bv28-MC-vc-PAB-MMAE or polatuzumab vedotin) on Day 1 of each of the second, third, fourth, fifth, and sixth 28-day cycles, i.e., during the induction phase.

In some embodiments, the individual achieves a therapeutic response during or following the during the induction phase, i.e., during or following the first 6 cycles of the treatment comprising the immunoconjugate (e.g., huMA79bv28-MC-vc-PAB-MMAE or polatuzumab vedotin), the immunomodulatory agent (e.g., lenalidomide) and the anti-CD20 antibody (e.g., obinutuzumab or rituximab). In some embodiments, the therapeutic response is at least stable disease (SD) (e.g., at least SD, at least partial response (PR), or a complete response or complete remission (CR). In some embodiments, the therapeutic response is assessed according to Cheson et al. (2014) “Recommendations for Initial Evaluation, Staging and Response Assessment of Hodgkin and Non-Hodgkin Lymphoma: The Lugano Classification.” J. Clin Oncol. 32: 3059-3067.

In some embodiments, the individual achieves at least stable disease (“SD”) during or following the induction phase, e.g., during or following treatment with the immunoconjugate, the immunomodulatory agent (e.g., lenalidomide), and the anti-CD20 antibody (e.g., obinutuzumab or rituximab). In some embodiments the individual achieves at least stable disease (SD) during or following the induction phase (e.g., during or following treatment with the immunoconjugate, the immunomodulatory agent (e.g., lenalidomide), and the anti-CD20 antibody (e.g., obinutuzumab or rituximab) if the “PET-CT SD” criteria are met. The positron emission tomography-computed tomography (PET-CT) SD criteria are met if: (i) the uptake of ¹⁸F-fluorodeoxyglucose (FDG) at the target nodes/nodal masses and extranodal lesions is moderately or markedly higher than liver, but with there is no significant change in FDG uptake compared to baseline at interim or end of treatment; (ii) no new lesions; and (iii) no change in FDG uptake in bone marrow compared to baseline at interim or end of treatment. In some embodiments, the individual who meets the preceding criteria achieves at least “PET-CT SD” or “no metabolic response.” In some embodiments the individual achieves at least SD during or following the induction phase (e.g., during or following treatment with the immunoconjugate, the immunomodulatory agent (e.g., lenalidomide), and the anti-CD20 antibody (e.g., obinutuzumab or rituximab)) if the “CT SD” criteria are met. The computed tomography (CT) SD criteria are met if: (i) there is a <50% decrease from baseline in the sum of the product of the perpendicular diameters (SPD) of up to 6 dominant, measurable target nodes/nodal masses and extranodal sites and no criteria for progressive disease are met (as described in Cheson et al., supra); (ii) no increase in non-measured lesions consistent with progression; (iii) no increase in organ enlargement consistent with progressive disease; and (iv) no new lesions. In some embodiments, the individual who meets the preceding criteria has achieved at least “CT SD.” In some embodiments, among a plurality of individuals treated during an induction phase according to a method described herein, at least about any one of 70%, 75%, 80%, 85%, 90%, 91%, 92%, 93%, 94%, 95%, 96%, 97%, 98%, 99% or 100% of the individuals in the plurality achieve at least SD during or following treatment. Individuals who achieve “at least SD” are those who achieve SD, PR and CR during or following the induction phase (e.g., during or following treatment with the immunoconjugate, the immunomodulatory agent (e.g., lenalidomide), and the anti-CD20 antibody (e.g., obinutuzumab or rituximab)).

In some embodiments the individual has achieved at least partial response or partial remission (PR) during or following the induction phase (e.g., during or following treatment with the immunoconjugate, the immunomodulatory agent (e.g., lenalidomide), and the anti-CD20 antibody (e.g., obinutuzumab or rituximab)). In some embodiments, the individual achieves at least PR during or following the induction phase if the “PET-CT PR” criteria are met. The positron emission tomography-computed tomography (PET-CT) PR criteria are met if: (i) the uptake of ¹⁸F-fluorodeoxyglucose (FDG) at the lymph nodes and extralymphatic sites is moderately or markedly higher than liver, but with there is reduced in FDG uptake compared to baseline and residual mass(es) of any size, wherein at interim, these findings suggest responding disease, and wherein at or following end of treatment, these findings indicate residual disease; (ii) no new lesions; and (iii) there is residual uptake of FDG in the bone marrow that is higher than update in normal bone marrow, but the residual uptake is reduced compared with baseline (diffuse uptake compatible with reactive changes from chemotherapy is allowed). In some embodiments, if there are persistent focal changes in the marrow in the context of a nodal response, a further evaluation with MRI or biopsy or an interval scan is performed. In some embodiments, the individual who has met the preceding criteria has achieved at least “partial metabolic response” or “PET-CT PR.” In some embodiments the individual has achieved at least PR during or following the induction phase (e.g., during or following treatment with the immunoconjugate, the immunomodulatory agent (e.g., lenalidomide), and the anti-CD20 antibody (e.g., obinutuzumab or rituximab)) if the “CT PR” criteria are met. The computed tomography (CT) PR criteria are met if: (i) there is a ≥50% decrease in SPD of up to 6 measurable target nodes/nodal masses and extranodal sites; (ii) non-measured lesions are absent/normal, but have not increased; (iii) no new lesions; and (iii) spleen has regressed by >50% in length beyond normal. In some embodiments, the individual who has met the preceding criteria has achieved at least “CT PR.” In some embodiments, among a plurality of humans treated during an induction phase according to a method described herein, at least about any one of 70%, 75%, 80%, 81%, 82%, 83%, 84%, 85%, 86%, 87%, 88%, 89% 90%, 91%, 92%, 93%, 94%, 95%, 96%, 97%, 98%, 99% or 100% of the humans in the plurality achieve at least PR. Individuals who achieve “at least PR” are those who achieve PR and CR during or following the induction phase (e.g., during or following treatment with the immunoconjugate, the immunomodulatory agent (e.g., lenalidomide), and the anti-CD20 antibody (e.g., obinutuzumab or rituximab)).

In some embodiments the individual has achieved a complete response or complete remission (CR) during or following the induction phase (e.g., during or following treatment with the immunoconjugate, the immunomodulatory agent (e.g., lenalidomide), and the anti-CD20 antibody (e.g., obinutuzumab or rituximab)). In some embodiments the individual has achieved a complete response or complete remission (CR) during or following the induction phase (e.g., during or following treatment with the immunoconjugate, the immunomodulatory agent (e.g., lenalidomide), and the anti-CD20 antibody (e.g., obinutuzumab or rituximab) if the “PET-CT CR” criteria are met. The positron emission tomography-computed tomography (PET-CT) CR criteria are met if: (i) there is no uptake of ¹⁸F-fluorodeoxyglucose (FDG) at the lymph nodes and extralymphatic sites, with or without a residual mass or the uptake is less than that of the mediastinum with or without a residual mass or the uptake is greater than that of the mediastinum but less than or the same as the uptake by the liver is moderately or markedly higher than liver, with or without a residual mass; (iii) no new lesions; and (iv) no evidence of FDG-avid disease in the bone marrow. In some embodiments, if there are persistent focal changes in the marrow in the context of a nodal response, a further evaluation with MRI or biopsy or an interval scan is performed. In some embodiments, the individual who has met the preceding criteria has achieved a “complete metabolic response” or “PET-CT CR.” In some embodiments, a complete metabolic response (PET-CT CR) is achieved if the FDG update at the sites of initial involvement is no greater than surrounding normal tissue, even if the tissue has high physiological FDG uptake. In some embodiments the individual has achieved at least PR during or following the induction phase (e.g., during or following treatment with the immunoconjugate, the immunomodulatory agent (e.g., lenalidomide), and the anti-CD20 antibody (e.g., obinutuzumab or rituximab)) if the “CT CR” criteria are met. The computed tomography (CT) CR criteria are met if: (i) target nodes/nodal masses have regressed to ≤1.5 cm in the longest diameter; (ii) there are no extralymphatic sites of disease; (iii) no non-measured lesions; (iv) no new lesions; (v) size of enlarged organs has regressed to normal; and (vi) bone marrow is normal by morphology and/or or immunohistochemistry. In some embodiments, the individual who has met the preceding criteria has achieved at least “CT CR.” In some embodiments, among a plurality of human treated according to a method described herein, at least about 50%, 55%, 60%, 65%, 70%, 75%, or 80% of the humans in the plurality achieve at least CR, including any range in between these values (e.g., such as between about 61% and about 67%, or about 78%) during or following the induction phase (e.g., during or following treatment with the immunoconjugate, the immunomodulatory agent (e.g., lenalidomide), and the anti-CD20 antibody (e.g., obinutuzumab or rituximab)).

In some embodiments, among a plurality of individuals treated during an induction phase according to a method described herein, at least about any one of 85%, 90%, 91%, 92%, 93%, 94%, 95%, 96%, 97%, 98%, 99% or 100% of the individuals in the plurality achieve an overall response (OR) during or following treatment. In some embodiments, 89% of the individuals in the plurality achieve OR during or following treatment. Individuals who achieve an overall response are those who achieve PR or CR during or following the induction phase (e.g., during or following treatment with the immunoconjugate, the immunomodulatory agent (e.g., lenalidomide), and the anti-CD20 antibody (e.g., obinutuzumab or rituximab)).

In some embodiments, the humans treated during an induction phase according to a method described herein (e.g., treatment with the triple combination of the immunoconjugate, the immunomodulatory agent (e.g., lenalidomide), and the anti-CD20 antibody (e.g., obinutuzumab or rituximab)) achieve an improved response compared to humans treated with the double combination of the immunomodulatory agent (e.g., lenalidomide) and the anti-CD20 antibody (e.g., obinutuzumab or rituximab).

Further details regarding clinical staging of and response criteria for lymphomas such as FL are provided in, e.g., Van Heertum et al. (2017) Drug Des. Devel. Ther. 11: 1719-1728; Cheson et al. (2016) Blood. 128: 2489-2496; Cheson et al. (2014) J. Clin. Oncol. 32(27): 3059-3067; Barrington et al. (2017) J. Clin. Oncol. 32(27): 3048-3058; Gallamini et al. (2014) Haematologica. 99(6): 1107-1113; Barrinton et al. (2010) Eur. J. Nucl. Med. Mol. Imaging. 37(10): 1824-33; Moskwitz (2012) Hematology Am Soc. Hematol. Educ. Program 2012: 397-401; and Follows et al. (2014) Br. J. Haematology 166: 34-49. The progress of any one of the methods of treatment provided herein can be monitored by techniques known in the art.

Provided is a method for treating follicular lymphoma (FL) in a human in need thereof comprising administering to the human an effective amount of (a) an immunoconjugate comprising the formula

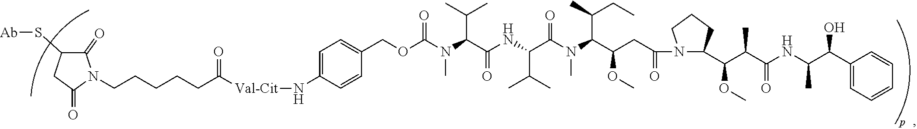

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, (b) an immunomodulatory agent, and (c) an anti-CD20 antibody; and wherein the human does not demonstrate disease progression within at least about 12 months after the start of treatment with the immunoconjugate, the immunomodulatory agent, and the anti-CD20 antibody. In some embodiments, among a plurality of humans treated, at least 75%, at least 80%, at least 85%, or at least 90% of the humans do not demonstrate disease progression within at least about 12 months after the start of treatment with the immunoconjugate, the immunomodulatory agent, and the anti-CD20 antibody. Disease progression is determined according to the Revised/Modified Lugano 2014 criteria (Cheson et al. (2014) J. Clin. Oncol. 32(27): 3059-3068).

In some embodiments, disease progression is measured from initiation of treatment according to the methods provided herein (e.g., from Cycle 1, Day 1 of an induction phase provided herein) to the time of the first occurrence of disease progression or relapse. Thus, if a human does not demonstrate disease progression within at least about 12 months after the start of treatment according to the methods provided herein, the human does not have an occurrence of disease progression or relapse within at least about 12 months after the start of treatment according to the methods provided herein. Alternatively or additionally, if among a plurality of humans treated, at least 75%, at least 80%, at least 85%, or at least 90% of the humans do not demonstrate disease progression within at least about 12 months after the start of treatment according to the methods provided herein, at least 75%, at least 80%, at least 85%, or at least 90% of the humans do not have an occurrence of disease progression or relapse within at least about 12 months after the start of treatment according to the methods provided herein.

In some embodiments, progression-free survival is measured from the start of treatment according to the methods provided herein (e.g., from Cycle 1, Day 1 of an induction phase provided herein) to the time of the first occurrence of disease progression or relapse. Thus, if a human demonstrates 12-month progression-free survival, the human does not have an occurrence of disease progression or relapse within at least about 12 months after the start of treatment according to the methods provided herein. Alternatively or additionally, if among a plurality of humans treated according to the methods provided herein at least 75%, at least 80%, at least 85%, or at least 90% of the humans demonstrate 12-month progression-free survival, at least 75%, at least 80%, at least 85%, or at least 90% of the humans do not have an occurrence of disease progression or relapse within at least about 12 months after the start of treatment according to the methods provided herein.

In some embodiments, disease progression is determined according to the Revised/Modified Lugano 2014 criteria (Cheson et al. (2014) J. Clin. Oncol. 32(27): 3059-3068). In some embodiments, disease progression is determined on the basis of CT-scans alone or death from any cause.

In some embodiments, the immunomodulatory agent (e.g., lenalidomide) and the anti-CD20 antibody (e.g., obinutuzumab or rituximab) are further administered during a maintenance phase following the sixth 28-day cycle. The “maintenance phase” refers to a treatment phase following an induction phase. In some embodiments, the maintenance phase begins immediately after the end of the induction phase. In some embodiment, the induction phase and the maintenance phase are separated by an interval of time. In some embodiments, the maintenance phase begins at least about 1, 2, 3, 4, 5, 6, 7, 8, 9, or 10 weeks after the end of the induction phase. In some embodiments, the immunomodulatory agent (e.g., lenalidomide) is administered orally at a dose between about 10 mg and about 20 mg on each of Days 1-21 of each month during the maintenance phase following the sixth 28-day cycle, the anti-CD20 antibody is obinutuzumab, and the obinutuzumab is administered intravenously at a dose of 1000 mg on Day 1 of every other month during the maintenance phase following the sixth 28-day cycle. In some embodiments, the immunomodulatory agent (e.g., lenalidomide) is administered orally at a dose between about 10 mg and about 20 mg on each of Days 1-21 of each month during the maintenance phase following the sixth 28-day cycle, the anti-CD20 antibody is rituximab, and the rituximab is administered intravenously at a dose of 375 mg/m² (such as on Day 1) of every other month during the maintenance phase following the sixth 28-day cycle. In some embodiments, the immunomodulatory agent (e.g., lenalidomide) is administered orally at a dose of 10 mg on each of Days 1-21 of each month during the maintenance phase following the sixth 28-day cycle, the anti-CD20 antibody is obinutuzumab, and the obinutuzumab is administered intravenously at a dose of 1000 mg on Day 1 of every other month during the maintenance phase following the sixth 28-day cycle. In some embodiments, the immunomodulatory agent (e.g., lenalidomide) is administered orally at a dose of 10 mg on each of Days 1-21 of each month during the maintenance phase following the sixth 28-day cycle, the anti-CD20 antibody is rituximab, and the rituximab is administered intravenously at a dose of 375 mg/m² (such as on Day 1) of every other month during the maintenance phase following the sixth 28-day cycle. In some embodiments, the immunomodulatory agent (e.g., lenalidomide) is administered orally at a dose of 15 mg on each of Days 1-21 of each month during the maintenance phase following the sixth 28-day cycle, the anti-CD20 antibody is obinutuzumab, and the obinutuzumab is administered intravenously at a dose of 1000 mg on Day 1 of every other month during the maintenance phase following the sixth 28-day cycle. In some embodiments, the immunomodulatory agent (e.g., lenalidomide) is administered orally at a dose of 15 mg on each of Days 1-21 of each month during the maintenance phase following the sixth 28-day cycle, the anti-CD20 antibody is rituximab, and the rituximab is administered intravenously at a dose of 375 mg/m² (such as on Day 1) of every other month during the maintenance phase following the sixth 28-day cycle. In some embodiments, the immunomodulatory agent (e.g., lenalidomide) is administered orally at a dose of 20 mg on each of Days 1-21 of each month during the maintenance phase following the sixth 28-day cycle, the anti-CD20 antibody is obinutuzumab, and the obinutuzumab is administered intravenously at a dose of 1000 mg on Day 1 of every other month during the maintenance phase following the sixth 28-day cycle. In some embodiments, the immunomodulatory agent (e.g., lenalidomide) is administered orally at a dose of 20 mg on each of Days 1-21 of each month during the maintenance phase following the sixth 28-day cycle, the anti-CD20 antibody is rituximab, and the rituximab is administered intravenously at a dose of 375 mg/m² (such as on Day 1) of every other month during the maintenance phase following the sixth 28-day cycle. In some embodiments, the immunomodulatory agent (e.g., lenalidomide) is administered for a maximum of 12 months during the maintenance phase following the sixth 28-day cycle. In some embodiments, the anti-CD20 antibody (e.g., obinutuzumab or rituximab) is administered for a maximum of 24 months during the maintenance phase following the sixth 28-day cycle. In some embodiments, the immunomodulatory agent (e.g., lenalidomide) and the anti-CD20 antibody (e.g., obinutuzumab or rituximab) are administered sequentially during the maintenance phase following the sixth 28-day cycle. In some embodiments, the immunomodulatory agent (e.g., lenalidomide) is administered prior to the anti-CD20 antibody (e.g., obinutuzumab or rituximab) on Day 1 of each of the first, third, fifth, seventh, ninth, and eleventh months during the maintenance phase following the sixth 28-day cycle.

The dosing and administration schedules for exemplary maintenance phases are provided in Tables M-Q below:

TABLES M-Q
Dosing and Administration Schedules
for Exemplary Maintenance Phases

| | |
|---|---|
| | Dose and Frequency |
| Drugs | of Administration |

TABLE M

| | |
|---|---|
| Immunomodulatory | 10 mg on each of Days 1-21 every month |
| Agent | |
| (lenalidomide) | |
| Anti-CD20 Antibody | 1000 mg on Day 1 of every other month |
| (obinutuzumab) | |

TABLE N

| | |
|---|---|
| Immunomodulatory | 15 mg on each of Days 1-21 every month |
| Agent | |
| (lenalidomide) | |
| Anti-CD20 Antibody | 1000 mg on Day 1 of every other month |
| (obinutuzumab) | |

TABLE O

| | |
|---|---|
| Immunomodulatory | 20 mg on each of Days 1-21 every month |
| Agent | |
| (lenalidomide) | |
| Anti-CD20 Antibody | 1000 mg on Day 1 of every other month |
| (obinutuzumab) | |

TABLE P

| | |
|---|---|
| Immunomodulatory | 10 mg on each of Days 1-21 every month |
| Agent | |
| (lenalidomide) | |
| Anti-CD20 Antibody | 375 mg/m² (e.g., Day 1 of every other month) |
| (rituximab) | |

TABLE Q

| | |
|---|---|
| Immunomodulatory | 15 mg on each of Days 1-21 every month |
| Agent | |
| (lenalidomide) | |
| Anti-CD20 Antibody | 375 mg/m² (e.g., Day 1 of every other month) |
| (rituximab) | |

TABLE R

| | |
|---|---|
| Immunomodulatory | 20 mg on each of Days 1-21 every month |
| Agent | |
| (lenalidomide) | |
| Anti-CD20 Antibody | 375 mg/m² (e.g., Day 1 of every other month) |
| (rituximab) | |

Any one of the exemplary induction phases shown in Tables A-L may be followed by any one of the exemplary maintenance cycles shown in Tables M-Q.

In some embodiments, the method of treating follicular lymphoma (FL) in a human in need thereof comprises administering to the human, during an induction phase, an effective amount of: (a) polatuzumab vedotin, (b) lenalidomide, and (c) obinutuzumab, wherein, during the induction phase, the polatuzumab vedotin is administered at a dose of about 1.4 mg/kg, the lenalidomide is administered at a dose of about 20 mg, and the obinutuzumab is administered at a dose of about 1000 mg, and wherein, the human achieves a complete response following the induction phase. In some embodiments, the induction phase comprises less than one complete 28-day cycle. In some embodiments, the induction phase comprises between one and six (e.g., any of 1, 2, 3, 4, 5, or 6) 28-day cycles. In some embodiments, the induction phase comprises at least six 28-day cycles. In some embodiments, the immunoconjugate, the lenalidomide, and the obinutuzumab are administered during the induction phase for at least six 28-day cycles. In some embodiments, during the induction phase, the immunoconjugate is administered intravenously at a dose of about 1.4 mg/kg on Day 1, the lenalidomide is administered orally at a dose of about 20 mg on each of Days 1-21, and the obinutuzumab is administered intravenously at a dose of about 1000 mg on each of Days 1, 8, and 15 of the first 28 day cycle, and the immunoconjugate is administered intravenously at a dose of about 1.4 mg/kg on Day 1, the lenalidomide is administered orally at a dose between about 20 mg on each of Days 1-21, and the obinutuzumab is administered intravenously at a dose of about 1000 mg on Day 1 of each of the second, third, fourth, fifth, and sixth 28-day cycles. In some embodiments, the induction phase is followed by a maintenance phase, wherein the lenalidomide is administered at a dose of about 10 mg and the obinutuzumab is administered at a dose of about 1000 mg during the maintenance phase. In some embodiments, during the maintenance phase, the lenalidomide is administered orally at a dose of about 10 mg on each of Days 1-21 of each month during the maintenance phase following the sixth 28-day cycle, and wherein the obinutuzumab is administered intravenously at a dose of about 1000 mg on Day 1 of every other month during the maintenance phase following the sixth 28-day cycle.

Provided is a method of treating follicular lymphoma (FL) in a plurality of humans in need thereof, comprising administering to the humans, during an induction phase, an effective amount of: (a) polatuzumab vedotin, (b) lenalidomide, and (c) obinutuzumab, wherein, during the induction phase, the polatuzumab vedotin is administered at a dose of about 1.4 mg/kg, the lenalidomide is administered at a dose of about 20 mg, and the obinutuzumab is administered at a dose of about 1000 mg, and wherein, at least about 50%, 55%, 60%, 65%, 70%, 75%, or 80% of the humans in the plurality achieve at least CR, including any range in between these values (e.g., such as between about 61% and about 67%, or about 78%) following the induction phase (e.g., by the end of the induction phase). In some embodiments, the induction phase comprises less than one complete 28-day cycle. In some embodiments, the induction phase comprises between one and six (e.g., any of 1, 2, 3, 4, 5, or 6) 28-day cycles. In some embodiments, the induction phase comprises at least six 28-day cycles. In some embodiments, the immunoconjugate, the lenalidomide, and the obinutuzumab are administered during the induction phase for at least six 28-day cycles. In some embodiments, during the induction phase, the immunoconjugate is administered intravenously at a dose of about 1.4 mg/kg on Day 1, the lenalidomide is administered orally at a dose of about 20 mg on each of Days 1-21, and the obinutuzumab is administered intravenously at a dose of about 1000 mg on each of Days 1, 8, and 15 of the first 28 day cycle, and the immunoconjugate is administered intravenously at a dose of about 1.4 mg/kg on Day 1, the lenalidomide is administered orally at a dose between about 20 mg on each of Days 1-21, and the obinutuzumab is administered intravenously at a dose of about 1000 mg on Day 1 of each of the second, third, fourth, fifth, and sixth 28-day cycles. In some embodiments, the induction phase is followed by a maintenance phase, wherein the lenalidomide is administered at a dose of about 10 mg and the obinutuzumab is administered at a dose of about 1000 mg during the maintenance phase. In some embodiments, the lenalidomide is administered orally at a dose of about 10 mg on each of Days 1-21 of each month during the maintenance phase following the sixth 28-day cycle, and wherein the obinutuzumab is administered intravenously at a dose of about 1000 mg on Day 1 of every other month during the maintenance phase following the sixth 28-day cycle. In some embodiments, at least about 60%, 65%, 70%, 75%, 80%, 85%, 86%, 87%, 88%, 89%, 90%, 91%, 92%, 93%, 94%, or 95% of the humans in the plurality achieve progression free survival (PFS) (e.g., do not demonstrate progressive disease according to criteria described elsewhere herein) for at least about 12 months following the start of treatment (e.g., following the start of the induction phase), including any range in between these values.

In some embodiments, a method of treating follicular lymphoma (FL) in a human in need thereof, comprises administering to the human, during an induction phase, an effective amount of: (a) polatuzumab vedotin, (b) lenalidomide, and (c) obinutuzumab, wherein, during the induction phase, the polatuzumab vedotin is administered at a dose of about 1.8 mg/kg, the lenalidomide is administered at a dose of about 20 mg, and the obinutuzumab is administered at a dose of about 1000 mg, and wherein, the human achieves a complete response following the induction phase. In some embodiments, the induction phase comprises less than one complete 28-day cycle. In some embodiments, the induction phase comprises between one and six (e.g., any of 1, 2, 3, 4, 5, or 6) 28-day cycles. In some embodiments, the induction phase comprises at least six 28-day cycles. In some embodiments, the immunoconjugate, the lenalidomide, and the obinutuzumab are administered during the induction phase for at least six 28-day cycles, wherein the immunoconjugate is administered intravenously at a dose of about 1.8 mg/kg on Day 1, the lenalidomide is administered orally at a dose of about 20 mg on each of Days 1-21, and the obinutuzumab is administered intravenously at a dose of about 1000 mg on each of Days 1, 8, and 15 of the first 28 day cycle, and wherein the immunoconjugate is administered intravenously at a dose of about 1.8 mg/kg on Day 1, the lenalidomide is administered orally at a dose between about 20 mg on each of Days 1-21, and the obinutuzumab is administered intravenously at a dose of about 1000 mg on Day 1 of each of the second, third, fourth, fifth, and sixth 28-day cycles. In some embodiments, the induction phase is followed by a maintenance phase, wherein the lenalidomide is administered at a dose of about 10 mg and the obinutuzumab is administered at a dose of about 1000 mg during the maintenance phase. In some embodiments, the lenalidomide is administered orally at a dose of about 10 mg on each of Days 1-21 of each month during the maintenance phase following the sixth 28-day cycle, and wherein the obinutuzumab is administered intravenously at a dose of about 1000 mg on Day 1 of every other month during the maintenance phase following the sixth 28-day cycle.

Provided herein is a method of treating follicular lymphoma (FL) in a plurality of humans in need thereof, comprising administering to the humans, during an induction phase, an effective amount of: (a) polatuzumab vedotin, (b) lenalidomide, and (c) obinutuzumab, wherein, during the induction phase, the polatuzumab vedotin is administered at a dose of about 1.8 mg/kg, the lenalidomide is administered at a dose of about 20 mg, and the obinutuzumab is administered at a dose of about 1000 mg, and wherein, at least about 50%, 55%, 60%, 65%, 70%, 75%, or 80% of the patients in the plurality achieve at least CR, including any range in between these values (e.g., such as between about 61% and about 67%, or about 78%) following the induction phase (e.g., by the end of the induction phase). In some embodiments, the induction phase comprises less than one complete 28-day cycle. In some embodiments, the induction phase comprises between one and six (e.g., any of 1, 2, 3, 4, 5, or 6) 28-day cycles. In some embodiments, the induction phase comprises six 28-day cycles. In some embodiments, during the induction phase, the immunoconjugate is administered intravenously at a dose of about 1.8 mg/kg on Day 1, the lenalidomide is administered orally at a dose of about 20 mg on each of Days 1-21, and the obinutuzumab is administered intravenously at a dose of about 1000 mg on each of Days 1, 8, and 15 of the first 28 day cycle, and wherein the immunoconjugate is administered intravenously at a dose of about 1.8 mg/kg on Day 1, the lenalidomide is administered orally at a dose between about 20 mg on each of Days 1-21, and the obinutuzumab is administered intravenously at a dose of about 1000 mg on Day 1 of each of the second, third, fourth, fifth, and sixth 28-day cycles. In some embodiments, the induction phase is followed by a maintenance phase, wherein the lenalidomide is administered at a dose of about 10 mg and the obinutuzumab is administered at a dose of about 1000 mg during the maintenance phase. In some embodiments, the lenalidomide is administered orally at a dose of about 10 mg on each of Days 1-21 of each month during the maintenance phase following the sixth 28-day cycle, and wherein the obinutuzumab is administered intravenously at a dose of about 1000 mg on Day 1 of every other month during the maintenance phase following the sixth 28-day cycle. In some embodiments, at least about 60%, 65%, 70%, 75%, 80%, 85%, 86%, 87%, 88%, 89%, 90%, 91%, 92%, 93%, 94%, or 95% of the humans in the plurality achieve progression free survival (PFS) (e.g., do not demonstrate progressive disease according to criteria described elsewhere herein) for at least about 12 months following the start of treatment (e.g., following the start of the induction phase), including any range in between these values.

In some embodiments, the individual is an adult. In some embodiments, the individual has received at least one (e.g., any of 1, 2, 3, 4, 5, 6, 7, 8, 9, 10, or more) prior treatment for FL. In some embodiments, the individual relapsed after at least one prior treatment for FL. In some embodiments, the individual was refractory to at least one prior treatment for FL. In some embodiments, the individual exhibited progression or relapse of FL within about six months from the end date of their most recent therapy for FL. In some embodiments, the individual exhibited no response to their most recent therapy for FL. In some embodiments, the at least one prior treatment for FL was a chemoimmunotherapy regimen that included an anti-CD20 monoclonal antibody. In some embodiments, the individual was refractory to a prior therapy for FL with an anti-CD20 agent (e.g., an anti-CD20 antibody). In some embodiments, the individual exhibited progression or relapse of FL within about 6 months of a prior therapy for FL with an anti-CD20 agent (e.g., an anti-CD20 antibody). In some embodiments, the individual exhibited no response to a prior therapy for FL with an anti-CD20 agent (e.g., an anti-CD20 antibody). In some embodiments, the individual had progression of disease within 24 months of initiation of their first FL treatment with chemoimmunotherapy.

In some embodiments, the individual has histologically documented CD20-positive B-cell lymphoma. In some embodiments, the individual has ¹⁸fluorodeoxyglucose-avid (i.e., FDG-avid) lymphoma (i.e., PET-positive or PET-CT-positive lymphoma). In some embodiments, the individual has at least one bi-dimensionally measurable lesion (>1.5 cm in its largest dimension by computed tomography (CT) scan or magnetic resonance imaging (MRI)). In some embodiments, the individual has an Eastern Cooperative Oncology Group (ECOG) performance score (PS) of 0-2. In some embodiments, the individual has an ECOG score of 0-1. In some embodiments, the individual has FL with an Ann Arbor Stage of III or IV. In some embodiments, the individual has bulky disease FL (≥7 cm). In some embodiments, the individual has 3-5 Follicular Lymphoma International Prognostic Index (FLIPI) risk factors. In some embodiments, the individual has 1-2 FLIPI risk factors. In some embodiments, the individual has FL with bone marrow involvement.

In some embodiments, the FL is not CD20-negative at relapse or progression. In some embodiments, the individual does not have central nervous system lymphoma or leptomeningeal infiltration. In some embodiments, the individual does not have Grade 3b FL. In some embodiments, the individual has not undergone prior allogeneic stem-cell transplantation (SCT). In some embodiments, the individual has not undergone or completed autologous SCT within 100 days prior to the start of treatment with the immunoconjugate, the immunomodulatory agent, and the anti-CD20 antibody. In some embodiment, the individual is not refractory to lenalidomide. In some embodiments, the individual does not have a history of resistance to lenalidomide or response duration of <1 year, i.e., if the patient demonstrated a response to a prior lenalidomide-containing regimen. In some embodiments, the individual has not received lenalidomide, fludarabine, or alemtuzumab within 12 months prior to the start of treatment with the immunoconjugate, the immunomodulatory agent, and the anti-CD20 antibody. In some embodiments, the individual has not received radioimmunoconjugate within 12 weeks (e.g., 3 months) prior to the start of treatment with the immunoconjugate, the immunomodulatory agent, and the anti-CD20 antibody. In some embodiments, the individual has not received monoclonal antibody or antibody-drug conjugate therapy within about 4 weeks prior to the start of treatment with the immunoconjugate, the immunomodulatory agent, and the anti-CD20 antibody. In some embodiments, the individual has not received radiotherapy, chemotherapy, hormonal therapy, or targeted small-molecule therapy within 2 weeks prior the start of treatment with the immunoconjugate, the immunomodulatory agent, and the anti-CD20 antibody. In some embodiments, the individual has not received treatment with systemic immunosuppressive medications (including, but not limited to, e.g., prednisone, azathioprine, methotrexate, thalidomide, and anti-tumor necrosis factor agents) within 2 weeks prior to the start of treatment with the immunoconjugate, the immunomodulatory agent, and the anti-CD20 antibody. In some embodiments, treatment with inhaled corticosteroids and mineralocorticoids is not considered a systemic immunosuppressive therapy if the inhaled corticosteroids and mineralocorticoids treatment is required for lymphoma symptom control prior to the start of treatment with the immunoconjugate, the immunomodulatory agent, and the anti-CD20 antibody. In some embodiments the individual does not have inadequate hematologic function, unless due to underlying lymphoma. In some embodiments, the individual does not have Grade >1 peripheral neuropathy. In some embodiments, inadequate hematologic function is characterized by one or more of: Hemoglobin <9 g/dL; absolute neutrophil count (ANC)<1.5×10⁹/L; and platelet count <75×10⁹/L. In some embodiments the individual does not have: (i) calculated creatinine clearance <50 mL/min (using the Cockcroft-Gault formula); (ii) aspartate aminotransferase (AST) or alanine aminotransferase (ALT) >2.5× upper limit of normal (ULN); (iii) serum total bilirubin >1.5×ULN (or >3×ULN for patients with Gilbert syndrome); (iv) international normalized ratio (INR) or prothrombin time (PT) >1.5×ULN in the absence of therapeutic anticoagulation; and (v) partial thromboplastin time (PTT) or activated partial thromboplastin time (aPTT) >1.5×ULN in the absence of a lupus anticoagulant, unless the one or more of (i)-(v) are due to underlying lymphoma.

Provided is an immunoconjugate comprising the formula

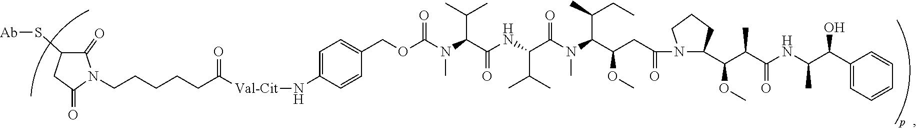

wherein Ab is an anti-CD79b antibody comprising (i) an HVR-H1 that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8 for use in a method of treating follicular lymphoma (FL), e.g., relapsed/refractory FL, in an individual (a human individual) in need thereof, the method comprising administering to the individual an effective amount of the immunoconjugate, an immunomodulatory agent, and an anti-CD20 antibody (e.g., obinutuzumab or rituximab), wherein the individual achieves at least stable disease (SD) (e.g., at least SD, at least partial response (PR) or a complete response (CR)) during or following treatment with the immunoconjugate, the immunomodulatory drug (e.g., lenalidomide), and the anti-CD20 antibody (e.g., obinutuzumab or rituximab). In some embodiments, the immunoconjugate is for use in a method described herein. In some embodiments, the immunoconjugate comprises an anti-CD79b antibody comprising (i) a VH comprising the amino acid sequence of SEQ ID NO: 19 and (ii) a VL comprising the amino acid sequence of SEQ ID NO: 20. In some embodiments, the immunoconjugate is polatuzumab vedotin.

Also provided is the use of an immunoconjugate comprising the formula

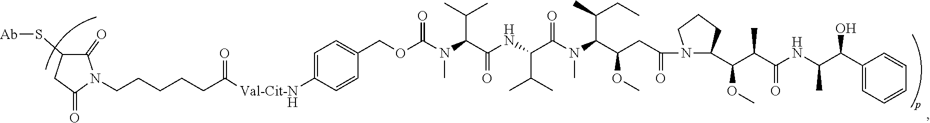

wherein Ab is an anti-CD79b antibody comprising (i) an HVR-H1 that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8 in the manufacture of a medicament for treating follicular lymphoma (FL), e.g., relapsed/refractory FL, in an individual (a human individual) in need thereof, wherein the medicament is for (e.g., formulated for) administration in combination with an immunomodulatory agent (e.g., lenalidomide), and an anti-CD20 antibody (e.g., obinutuzumab or rituximab), wherein the individual achieves at least stable disease (SD) (e.g., at least SD, at least partial response (PR) or a complete response (CR)) during or following treatment with the medicament, the immunomodulatory drug (e.g., lenalidomide), and the anti-CD20 antibody (e.g., obinutuzumab or rituximab). In some embodiments, the medicament (i.e., the medicament comprising the immunoconjugate) is for use in a method described herein. In some embodiments, the immunoconjugate comprises an anti-CD79b antibody comprising (i) a VH comprising the amino acid sequence of SEQ ID NO: 19 and (ii) a VL comprising the amino acid sequence of SEQ ID NO: 20. In some embodiments, the immunoconjugate is polatuzumab vedotin.

Provided is an immunoconjugate comprising the formula

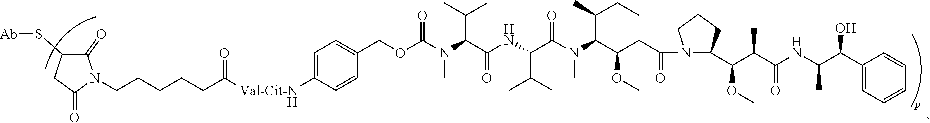

wherein Ab is an anti-CD79b antibody that comprises (i) a VH comprising the amino acid sequence of SEQ ID NO: 19 and (ii) a VL comprising the amino acid sequence of SEQ ID NO: 20, and wherein p is between 2 and 5, for use in a method of treating follicular lymphoma (FL), e.g., relapsed/refractory FL, in an individual (a human individual) in need thereof, the method comprising administering to the individual an effective amount of (a) the immunoconjugate, (b) lenalidomide, and (c) obinutuzumab, wherein the immunoconjugate is administered at a dose between about 1.4 and about 1.8 mg/kg, the lenalidomide is administered at a dose between about 10 mg and 20 mg, and the obinutuzumab is administered at a dose 1000 mg, and wherein the individual achieves at least stable disease (SD) (e.g., at least SD, at least partial response (PR) or a complete response (CR)) during or following treatment with the immunoconjugate, the immunomodulatory drug (e.g., lenalidomide), and the anti-CD20 antibody (e.g., obinutuzumab). In some embodiments, the immunoconjugate is for use according to a method described herein. In some embodiments, p is between 3 and 4. In some embodiments, p is 3.5. In some embodiments, the immunoconjugate comprises an anti-CD79b antibody comprising a heavy chain comprises the amino acid sequence of SEQ ID NO: 36, and wherein the light chain comprises the amino acid sequence of SEQ ID NO: 35. In some embodiments, the immunoconjugate comprises an anti-CD79 antibody that comprises a heavy chain comprising the amino acid sequence of SEQ ID NO: 37 and a light chain comprising the amino acid sequence of SEQ ID NO: 35. In some embodiments, the immunoconjugate comprises an anti-CD79 antibody that comprises a heavy chain comprising the amino acid sequence of SEQ ID NO: 36 and a light chain comprising the amino acid sequence of SEQ ID NO: 38. In some embodiments, the immunoconjugate is polatuzumab vedotin.

Also provided is an immunoconjugate comprising the formula

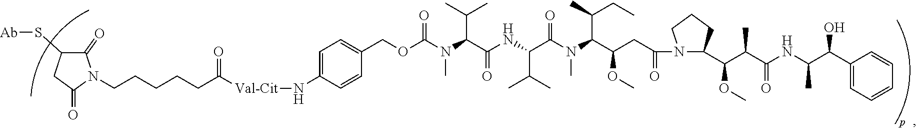

wherein Ab is an anti-CD79b antibody that comprises (i) a VH comprising the amino acid sequence of SEQ ID NO: 19 and (ii) a VL comprising the amino acid sequence of SEQ ID NO: 20, and wherein p is between 2 and 5, for use in the manufacture of a medicament for treating follicular lymphoma (FL), e.g., relapsed/refractory FL, in an individual (a human individual) in need thereof, wherein the medicament is for (e.g., formulated for) administration in combination with lenalidomide, and obinutuzumab, wherein the medicament is formulated for administration of the immunoconjugate at a dose between about 1.4 and about 1.8 mg/kg, the lenalidomide is for administration at a dose between about 10 mg and 20 mg, and the obinutuzumab is for administration at a dose 1000 mg, and wherein the individual achieves at least stable disease (SD) (e.g., at least SD, at least partial response (PR) or a complete response (CR)) during or following the treatment with the medicament, the immunomodulatory drug (e.g., lenalidomide), and the anti-CD20 antibody (e.g., obinutuzumab). In some embodiments, the medicament (i.e., the medicament comprising the immunoconjugate) is for use according to a method described herein. In some embodiments, p is between 3 and 4. In some embodiments, p is 3.5. In some embodiments, the immunoconjugate comprises an anti-CD79b antibody comprising a heavy chain comprises the amino acid sequence of SEQ ID NO: 36, and wherein the light chain comprises the amino acid sequence of SEQ ID NO: 35. In some embodiments, the immunoconjugate comprises an anti-CD79 antibody that comprises a heavy chain comprising the amino acid sequence of SEQ ID NO: 37 and a light chain comprising the amino acid sequence of SEQ ID NO: 35. In some embodiments, the immunoconjugate comprises an anti-CD79 antibody that comprises a heavy chain comprising the amino acid sequence of SEQ ID NO: 36 and a light chain comprising the amino acid sequence of SEQ ID NO: 38. In some embodiments, the immunoconjugate is polatuzumab vedotin.

### IV. Immunoconjugates Comprising an Anti-CD79b Antibody and a Drug/Cytotoxic Agent (“Anti-CD79b Immunoconjugates”)

In some embodiments, the anti-CD79b immunoconjugate comprises an anti-CD79b antibody (Ab) which targets a cancer cell (such as a follicular lymphoma (FL) cell), a drug moiety (D), and a linker moiety (L) that attaches Ab to D. In some embodiments, the anti-CD79b antibody is attached to the linker moiety (L) through one or more amino acid residues, such as lysine and/or cysteine. In some formula Ab-(L-D)p, wherein: (a) Ab is the anti-CD79b antibody which binds CD79b on the surface of a cancer cell (e.g., an FL cell); (b) L is a linker; (c) D is a cytotoxic agent; and (d) p ranges from 1-8.

An exemplary anti-CD79b immunoconjugate comprises Formula I:

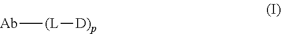

wherein p is 1 to about 20 (e.g., 1 to 15, 1 to 10, 1 to 8, 2 to 5, or 3 to 4). In some embodiments, the number of drug moieties that can be conjugated to the anti-CD79b antibody is limited by the number of free cysteine residues. In some embodiments, free cysteine residues are introduced into the antibody amino acid sequence by the methods described elsewhere herein. Exemplary anti-CD79b immunoconjugates of Formula I comprise, but are not limited to, anti-CD79b antibodies that comprise 1, 2, 3, or 4 engineered cysteine amino acids (Lyon, R. et al (2012) Methods in Enzym. 502:123-138). In some embodiments, one or more free cysteine residues are already present in the anti-CD79b antibody, without the use of engineering, in which case the existing free cysteine residues may be used to conjugate the anti-CD79b antibody to the drug/cytotoxic agent. In some embodiments, the anti-CD79b antibody is exposed to reducing conditions prior to conjugation of the antibody to the drug/cytotoxic agent in order to generate one or more free cysteine residues.

### A. Exemplary Linkers

A “linker” (L) is a bifunctional or multifunctional moiety that can be used to link one or more drug moieties (D) to the anti-CD79b antibody (Ab) to form an anti-CD79b immunoconjugate of Formula I. In some embodiments, anti-CD79b immunoconjugate can be prepared using a linker having reactive functionalities for covalently attaching to the drug and to the anti-CD79b antibody. For example, in some embodiments, a cysteine thiol of the anti-CD79b antibody (Ab) can form a bond with a reactive functional group of a linker or a drug-linker intermediate to make the anti-CD79b immunoconjugate.

In one aspect, a linker has a functionality that is capable of reacting with a free cysteine present on the anti-CD79b antibody to form a covalent bond. Exemplary reactive functionalities include, without limitation, e.g., maleimide, haloacetamides, α-haloacetyl, activated esters such as succinimide esters, 4-nitrophenyl esters, pentafluorophenyl esters, tetrafluorophenyl esters, anhydrides, acid chlorides, sulfonyl chlorides, isocyanates, and isothiocyanates. See, e.g., the conjugation method at page 766 of Klussman, et al (2004), Bioconjugate Chemistry 15(4):765-773, and the Examples herein.

In some embodiments, a linker has a functionality that is capable of reacting with an electrophilic group present on the anti-CD79b antibody. Exemplary electrophilic groups include, without limitation, e.g., aldehyde and ketone carbonyl groups. In some embodiments, a heteroatom of the reactive functionality of the linker can react with an electrophilic group on an antibody and form a covalent bond to an antibody unit. Exemplary reactive functionalities include, but are not limited to, e.g., hydrazide, oxime, amino, hydrazine, thiosemicarbazone, hydrazine carboxylate, and arylhydrazide.

In some embodiments, the linker comprises one or more linker components. Exemplary linker components include, e.g., 6-maleimidocaproyl (“MC”), maleimidopropanoyl (“MP”), valine-citrulline (“val-cit” or “vc”), alanine-phenylalanine (“ala-phe”), p-aminobenzyloxycarbonyl (a “PAB”), N-Succinimidyl 4-(2-pyridylthio) pentanoate (“SPP”), and 4-(N-maleimidomethyl) cyclohexane-1 carboxylate (“MCC”). Various linker components are known in the art, some of which are described below.

In some embodiments, the linker is a “cleavable linker,” facilitating release of a drug. Nonlimiting exemplary cleavable linkers include acid-labile linkers (e.g., comprising hydrazone), protease-sensitive (e.g., peptidase-sensitive) linkers, photolabile linkers, or disulfide-containing linkers (Chari et al., Cancer Research 52:127-131 (1992); U.S. Pat. No. 5,208,020).

In certain embodiments, a linker (L) has the following Formula II:

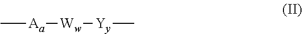

wherein A is a “stretcher unit,” and a is an integer from 0 to 1; W is an “amino acid unit,” and w is an integer from 0 to 12; Y is a “spacer unit,” and y is 0, 1, or 2; and Ab, D, and p are defined as above for Formula I. Exemplary embodiments of such linkers are described in U.S. Pat. No. 7,498,298, which is expressly incorporated herein by reference.

In some embodiments, a linker component comprises a “stretcher unit” that links an antibody to another linker component or to a drug moiety. Nonlimiting exemplary stretcher units are shown below (wherein the wavy line indicates sites of covalent attachment to an antibody, drug, or additional linker components):

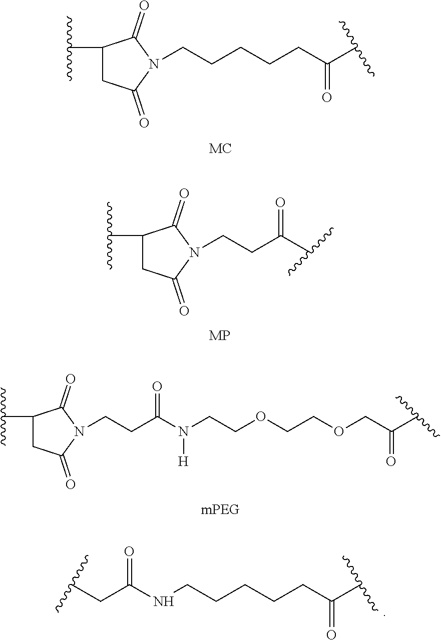 [SMILES: *C(=O)CCCCCN1C(=O)CC(*)C1=O ; *C(=O)CCN1C(=O)CC(*)C1=O ; *C(=O)COCCOCCNC(=O)CCN1C(=O)CC(*)C1=O ; *CC(=O)NCCCCCC(*)=O]

In some embodiments, a linker component comprises an “amino acid unit.” In some such embodiments, the amino acid unit allows for cleavage of the linker by a protease, thereby facilitating release of the drug/cytotoxic agent from the anti-CD79b immunoconjugate upon exposure to intracellular proteases, such as lysosomal enzymes (Doronina et al. (2003) Nat. Biotechnol. 21:778-784). Exemplary amino acid units include, but are not limited to, dipeptides, tripeptides, tetrapeptides, and pentapeptides. Exemplary dipeptides include, but are not limited to, valine-citrulline (vc or val-cit), alanine-phenylalanine (af or ala-phe); phenylalanine-lysine (fk or phe-lys); phenylalanine-homolysine (phe-homolys); and N-methyl-valine-citrulline (Me-val-cit). Exemplary tripeptides include, but are not limited to, glycine-valine-citrulline (gly-val-cit) and glycine-glycine-glycine (gly-gly-gly). An amino acid unit may comprise amino acid residues that occur naturally and/or minor amino acids and/or non-naturally occurring amino acid analogs, such as citrulline. Amino acid units can be designed and optimized for enzymatic cleavage by a particular enzyme, for example, a tumor-associated protease, cathepsin B, C and D, or a plasmin protease.

In some embodiments, a linker component comprises a “spacer” unit that links the antibody to a drug moiety, either directly or through a stretcher unit and/or an amino acid unit. A spacer unit may be “self-immolative” or a “non-self-immolative.” A “non-self-immolative” spacer unit is one in which part or all of the spacer unit remains bound to the drug moiety upon cleavage of the ADC. Examples of non-self-immolative spacer units include, but are not limited to, a glycine spacer unit and a glycine-glycine spacer unit. In some embodiments, enzymatic cleavage of an ADC containing a glycine-glycine spacer unit by a tumor-cell associated protease results in release of a glycine-glycine-drug moiety from the remainder of the ADC. In some such embodiments, the glycine-glycine-drug moiety is subjected to a hydrolysis step in the tumor cell, thus cleaving the glycine-glycine spacer unit from the drug moiety.

A “self-immolative” spacer unit allows for release of the drug moiety. In certain embodiments, a spacer unit of a linker comprises a p-aminobenzyl unit. In some such embodiments, a p-aminobenzyl alcohol is attached to an amino acid unit via an amide bond, and a carbamate, methylcarbamate, or carbonate is made between the benzyl alcohol and the drug (Hamann et al. (2005) Expert Opin. Ther. Patents (2005) 15:1087-1103). In some embodiments, the spacer unit is p-aminobenzyloxycarbonyl (PAB). In some embodiments, an anti-CD79b immunoconjugate comprises a self-immolative linker that comprises the structure:

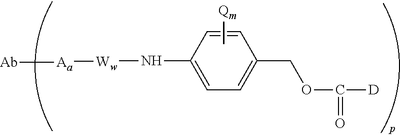

wherein Q is —C₁-C₈ alkyl, —O—(C₁-C₈ alkyl), -halogen, -nitro, or -cyno; m is an integer ranging from 0 to 4; and p ranges from 1 to about 20. In some embodiments, p ranges from 1 to 10, 1 to 7, 1 to 5, or 1 to 4.

Other examples of self-immolative spacers include, but are not limited to, aromatic compounds that are electronically similar to the PAB group, such as 2-aminoimidazol-5-methanol derivatives (U.S. Pat. No. 7,375,078; Hay et al. (1999) Bioorg. Med. Chem. Lett. 9:2237) and ortho- or para-aminobenzylacetals. In some embodiments, spacers can be used that undergo cyclization upon amide bond hydrolysis, such as substituted and unsubstituted 4-aminobutyric acid amides (Rodrigues et al (1995) Chemistry Biology 2:223), appropriately substituted bicyclo [2.2.1] and bicyclo [2.2.2] ring systems (Storm et al (1972) J. Amer. Chem. Soc. 94:5815) and 2-aminophenylpropionic acid amides (Amsberry, et al (1990) J. Org. Chem. 55:5867). Linkage of a drug to the α-carbon of a glycine residue is another example of a self-immolative spacer that may be useful in ADC (Kingsbury et al (1984) J. Med. Chem. 27:1447).

In some embodiments, linker L may be a dendritic type linker for covalent attachment of more than one drug moiety to an antibody through a branching, multifunctional linker moiety (Sun et al (2002) Bioorganic & Medicinal Chemistry Letters 12:2213-2215; Sun et al (2003) Bioorganic & Medicinal Chemistry 11:1761-1768). Dendritic linkers can increase the molar ratio of drug to antibody, i.e. loading, which is related to the potency of the ADC. Thus, where an antibody bears only one reactive cysteine thiol group, a multitude of drug moieties may be attached through a dendritic linker.

Nonlimiting exemplary linkers are shown below in the context of an anti-CD79 immunoconjugates of Formulas III, IV, V:

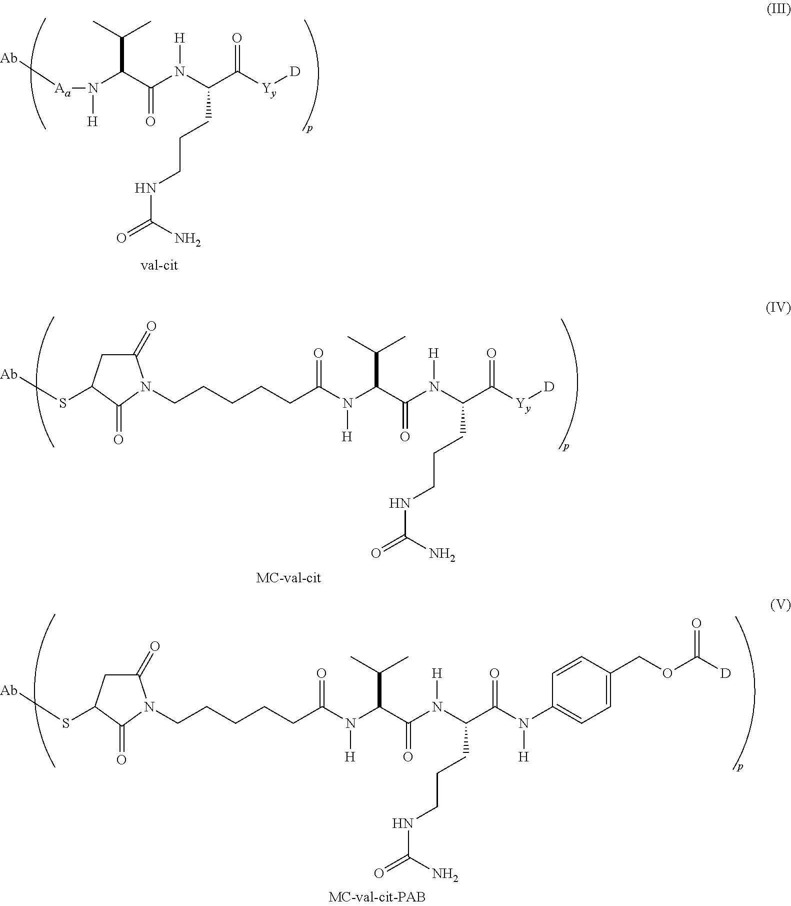

Wherein (Ab) is an anti-CD79b antibody, (D) is a drug/cytotoxic agent, “Val-Cit” is a valine-citrulline dipeptide, MC is 6-maleimidocaproyl, PAB is p-aminobenzyloxycarbonyl, and p is 1 to about 20 (e.g., 1 to 15, 1 to 10, 1 to 8, 2 to 5, or 3 to 4).

In some embodiments, the anti-CD79b immunoconjugate comprises a structure of any one of formulas VI-V below:

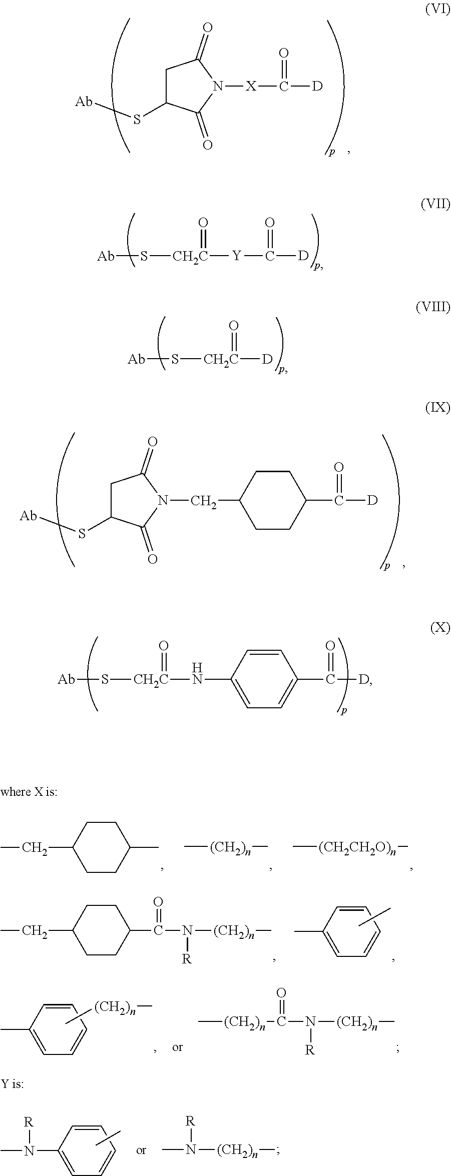

each R is independently H or C₁-C₆ alkyl; and n is 1 to 12.

Typically, peptide-type linkers can be prepared by forming a peptide bond between two or more amino acids and/or peptide fragments. Such peptide bonds can be prepared, for example, according to a liquid phase synthesis method (e.g., E. Schröder and K. Lübke (1965) “The Peptides”, volume 1, pp 76-136, Academic Press).

In some embodiments, a linker is substituted with groups that modulate solubility and/or reactivity. As a nonlimiting example, a charged substituent such as sulfonate (—SO₃⁻) or ammonium may increase water solubility of the linker reagent and facilitate the coupling reaction of the linker reagent with the antibody and/or the drug moiety, or facilitate the coupling reaction of Ab-L (anti-CD79b antibody-linker intermediate) with D, or D-L (drug/cytotoxic agent-linker intermediate) with Ab, depending on the synthetic route employed to prepare the anti-CD79b immunoconjugate. In some embodiments, a portion of the linker is coupled to the antibody and a portion of the linker is coupled to the drug, and then the anti-CD79 Ab-(linker portion)^{a} is coupled to drug/cytotoxic agent-(linker portion)^{b} to form the anti-CD79b immunoconjugate of Formula I. In some such embodiments, the anti-CD79b antibody comprises more than one (linker portion)^{a} substituents, such that more than one drug/cytotoxic agent is coupled to the anti-CD79b antibody in the anti-CD79b immunoconjugate of Formula I.

The anti-CD79b immunoconjugates provided herein expressly contemplate, but are not limited to, anti-CD79b immunoconjugates prepared with the following linker reagents: bis-maleimido-trioxyethylene glycol (BMPEO), N-(β-maleimidopropyloxy)-N-hydroxy succinimide ester (BMPS), N-(ε-maleimidocaproyloxy) succinimide ester (EMCS), N-[γ-maleimidobutyryloxy]succinimide ester (GMBS), 1,6-hexane-bis-vinylsulfone (HBVS), succinimidyl 4-(N-maleimidomethyl)cyclohexane-1-carboxy-(6-amidocaproate) (LC-SMCC), m-maleimidobenzoyl-N-hydroxysuccinimide ester (MBS), 4-(4-N-Maleimidophenyl)butyric acid hydrazide (MPBH), succinimidyl 3-(bromoacetamido)propionate (SBAP), succinimidyl iodoacetate (SIA), succinimidyl (4-iodoacetyl)aminobenzoate (SIAB), N-succinimidyl-3-(2-pyridyldithio) propionate (SPDP), N-succinimidyl-4-(2-pyridylthio)pentanoate (SPP), succinimidyl 4-(N-maleimidomethyl)cyclohexane-1-carboxylate (SMCC), succinimidyl 4-(p-maleimidophenyl)butyrate (SMPB), succinimidyl 6-[(beta-maleimidopropionamido)hexanoate](SMPH), iminothiolane (IT), sulfo-EMCS, sulfo-GMBS, sulfo-KMUS, sulfo-MBS, sulfo-SIAB, sulfo-SMCC, and sulfo-SMPB, and succinimidyl-(4-vinylsulfone)benzoate (SVSB), and including bis-maleimide reagents: dithiobismaleimidoethane (DTME), 1,4-Bismaleimidobutane (BMB), 1,4 Bismaleimidyl-2,3-dihydroxybutane (BMDB), bismaleimidohexane (BMH), bismaleimidoethane (BMOE), BM(PEG)₂ (shown below), and BM(PEG)₃ (shown below); bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). In some embodiments, bis-maleimide reagents allow the attachment of the thiol group of a cysteine in the antibody to a thiol-containing drug moiety, linker, or linker-drug intermediate. Other functional groups that are reactive with thiol groups include, but are not limited to, iodoacetamide, bromoacetamide, vinyl pyridine, disulfide, pyridyl disulfide, isocyanate, and isothiocyanate.

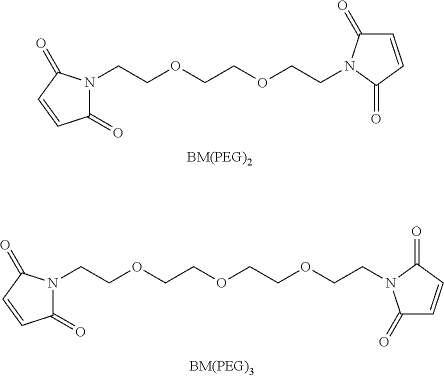 [SMILES: O=C1C=CC(=O)N1CCOCCOCCN1C(=O)C=CC1=O ; O=C1C=CC(=O)N1CCOCCOCCOCCN1C(=O)C=CC1=O]

Certain useful linker reagents can be obtained from various commercial sources, such as Pierce Biotechnology, Inc. (Rockford, IL), Molecular Biosciences Inc. (Boulder, CO), or synthesized in accordance with procedures described in the art; for example, in Toki et al (2002) J. Org. Chem. 67:1866-1872; Dubowchik, et al. (1997) Tetrahedron Letters, 38:5257-60; Walker, M. A. (1995) J. Org. Chem. 60:5352-5355; Frisch et al (1996) Bioconjugate Chem. 7:180-186; U.S. Pat. No. 6,214,345; WO 02/088172; US 2003130189; US2003096743; WO 03/026577; WO 03/043583; and WO 04/032828.

Carbon-14-labeled 1-isothiocyanatobenzyl-3-methyldiethylene triaminepentaacetic acid (MX-DTPA) is an exemplary chelating agent for conjugation of radionucleotide to the antibody. See, e.g., WO94/11026.

### B. Anti-CD79b Antibodies

In some embodiments, the immunoconjugate (e.g., anti-CD79b immunoconjugate) comprises an anti-CD79b antibody that comprises at least one, two, three, four, five, or six HVRs selected from (a) HVR-H1 comprising the amino acid sequence of SEQ ID NO: 21; (b) HVR-H2 comprising the amino acid sequence of SEQ ID NO: 22; (c) HVR-H3 comprising the amino acid sequence of SEQ ID NO: 23; (d) HVR-L1 comprising an amino acid sequence of SEQ ID NO: 24; (e) HVR-L2 comprising the amino acid sequence of SEQ ID NO: 25; and (f) HVR-L3 comprising the amino acid sequence of SEQ ID NO: 26. In some such embodiments, the immunoconjugate comprises an anti-CD79 antibody comprising at least one of: (i) HVR-H3 comprising the amino acid sequence of SEQ ID NO: 23, and/or (ii) HVR-L1 comprising an amino acid sequence of SEQ ID NO: 24. In some embodiments, the immunoconjugate comprises an anti-CD79 antibody comprising at least one of: (i) HVR-H3 comprising the amino acid sequence of SEQ ID NO: 23, and/or (ii) HVR-L1 comprising the amino acid sequence of SEQ ID NO: 24. In some embodiments, the immunoconjugate comprises an anti-CD79b antibody comprising at least one, at least two, or all three VH HVR sequences selected from (a) HVR-H1 comprising the amino acid sequence of SEQ ID NO: 21; (b) HVR-H2 comprising the amino acid sequence of SEQ ID NO: 22; and (c) HVR-H3 comprising the amino acid sequence of SEQ ID NO: 23. In some embodiments, the immunoconjugate comprises an anti-CD79b antibody that comprises an HVR-H3 comprising the amino acid sequence of SEQ ID NO: 23. In some embodiments, the immunoconjugate comprises an anti-CD79b antibody that comprises an HVR-H3 comprising the amino acid sequence of SEQ ID NO: 23 and an HVR-L3 comprising the amino acid sequence of SEQ ID NO: 26. In some embodiments, the immunoconjugate comprises an anti-CD79b antibody that comprises an HVR-H3 comprising the amino acid sequence of SEQ ID NO: 23, an HVR-L3 comprising the amino acid sequence of SEQ ID NO: 26, and an HVR-H2 comprising the amino acid sequence of SEQ ID NO: 22. In some embodiments, the immunoconjugate comprises an anti-CD79b antibody that comprises (a) HVR-H1 comprising the amino acid sequence of SEQ ID NO: 21; (b) HVR-H2 comprising the amino acid sequence of SEQ ID NO: 22; and (c) HVR-H3 comprising the amino acid sequence of SEQ ID NO: 23.

In some embodiments, the immunoconjugate comprises an anti-CD79b antibody comprising at least one, at least two, or all three VL HVR sequences selected from (a) HVR-L1 comprising an amino acid sequence of SEQ ID NO: 24; (b) HVR-L2 comprising the amino acid sequence of SEQ ID NO: 25; and (c) HVR-L3 comprising the amino acid sequence of SEQ ID NO: 26. In some embodiments, the immunoconjugate comprises an anti-CD79b antibody that comprises at least one, at least two, or all three VL HVR sequences selected from (a) HVR-L1 comprising the amino acid sequence of SEQ ID NO: 24; (b) HVR-L2 comprising the amino acid sequence of SEQ ID NO: 25; and (c) HVR-L3 comprising the amino acid sequence of SEQ ID NO: 26. In some embodiments, the immunoconjugate comprises (a) HVR-L1 comprising an amino acid sequence of SEQ ID NO: 24; (b) HVR-L2 comprising the amino acid sequence of SEQ ID NO: 25; and (c) HVR-L3 comprising the amino acid sequence of SEQ ID NO: 26. In some embodiments, the immunoconjugate comprises an anti-CD79b antibody that comprises an HVR-L1 comprising the amino acid sequence of SEQ ID NO: 24 In some embodiments, the immunoconjugate comprises an anti-CD79b antibody that comprises (a) HVR-L1 comprising the amino acid sequence of SEQ ID NO: 24; (b) HVR-L2 comprising the amino acid sequence of SEQ ID NO: 25; and (c) HVR-L3 comprising the amino acid sequence of SEQ ID NO: 26.

In some embodiments, the immunoconjugate comprises an anti-CD79b antibody comprising (a) a VH domain comprising at least one, at least two, or all three VH HVR sequences selected from (i) HVR-H1 comprising the amino acid sequence of SEQ ID NO: 21, (ii) HVR-H2 comprising the amino acid sequence of SEQ ID NO: 22, and (iii) HVR-H3 comprising an amino acid sequence selected from SEQ ID NO:23; and (b) a VL domain comprising at least one, at least two, or all three VL HVR sequences selected from (i) HVR-L1 comprising an amino acid sequence of SEQ ID NO: 24, (ii) HVR-L2 comprising the amino acid sequence of SEQ ID NO: 25, and (iii) HVR-L3 comprising the amino acid sequence of SEQ ID NO: 26. In some embodiments, the immunoconjugate comprises an anti-CD79b antibody that comprises at least one of: (i) HVR-H3 comprising the amino acid sequence of SEQ ID NO: 23, and/or (ii) HVR-L1 comprising the amino acid sequence of SEQ ID NO: 24.

In some embodiments, the immunoconjugate comprises an anti-CD79b antibody that comprises (a) HVR-H1 comprising the amino acid sequence of SEQ ID NO: 21; (b) HVR-H2 comprising the amino acid sequence of SEQ ID NO: 22; (c) HVR-H3 comprising the amino acid sequence of SEQ ID NO: 23; (d) HVR-L1 comprising an amino acid sequence of SEQ ID NO: 24; (e) HVR-L2 comprising the amino acid sequence of SEQ ID NO: 25; and (f) HVR-L3 comprising the amino acid sequence of SEQ ID NO: 26. In some embodiments, the immunoconjugate comprises at least one of: HVR-H3 comprising the amino acid sequence of SEQ ID NO: 23 and/or HVR-L1 comprising an amino acid sequence of SEQ ID NO: 24. In some embodiments, the immunoconjugate comprises an anti-CD79b antibody that comprises (a) HVR-H1 comprising the amino acid sequence of SEQ ID NO: 21; (b) HVR-H2 comprising the amino acid sequence of SEQ ID NO: 22; (c) HVR-H3 comprising the amino acid sequence of SEQ ID NO: 23; (d) HVR-L1 comprising the amino acid sequence of SEQ ID NO: 24; (e) HVR-L2 comprising the amino acid sequence of SEQ ID NO: 25; and (f) HVR-L3 comprising the amino acid sequence of SEQ ID NO: 26.

In some embodiments, the anti-CD79b immunoconjugates comprises a humanized anti-CD79b antibody. In some embodiments, an anti-CD79b antibody comprises HVRs as in any of the embodiments provided herein, and further comprises a human acceptor framework, e.g., a human immunoglobulin framework or a human consensus framework. In some embodiments, the human acceptor framework is the human VL kappa 1 (VL_{KI}) framework and/or the VH framework VH_{III}. In some embodiments, a humanized anti-CD79b antibody comprises (a) HVR-H1 comprising the amino acid sequence of SEQ ID NO: 21; (b) HVR-H2 comprising the amino acid sequence of SEQ ID NO: 22; (c) HVR-H3 comprising the amino acid sequence of SEQ ID NO: 23; (d) HVR-L1 comprising an amino acid sequence of SEQ ID NO: 24; (e) HVR-L2 comprising the amino acid sequence of SEQ ID NO: 25; and (f) HVR-L3 comprising the amino acid sequence of SEQ ID NO: 26. In some embodiments, a humanized anti-CD79b antibody comprises (a) HVR-H1 comprising the amino acid sequence of SEQ ID NO: 21; (b) HVR-H2 comprising the amino acid sequence of SEQ ID NO: 22; (c) HVR-H3 comprising the amino acid sequence of SEQ ID NO: 23; (d) HVR-L1 comprising the amino acid sequence of SEQ ID NO: 24; (e) HVR-L2 comprising the amino acid sequence of SEQ ID NO: 25; and (f) HVR-L3 comprising the amino acid sequence of SEQ ID NO: 26.

In some embodiments, the immunoconjugate (e.g., the anti-CD79b immunoconjugate) comprises an anti-CD79 antibody comprising a heavy chain variable domain (VH) sequence having at least 90%, 91%, 92%, 93%, 94%, 95%, 96%, 97%, 98%, 99%, or 100% sequence identity to the amino acid sequence of SEQ ID NO: 19. In some embodiments, a VH sequence having at least 90%, 91%, 92%, 93%, 94%, 95%, 96%, 97%, 98%, or 99% identity to the amino acid sequence of SEQ ID NO: 19 contains substitutions (e.g., conservative substitutions), insertions, or deletions relative to the reference sequence, but an anti-CD79b immunoconjugate comprising that sequence retains the ability to bind to CD79b. In some embodiments, a total of 1 to 10 amino acids have been substituted, inserted and/or deleted in SEQ ID NO: 19. In some embodiments, a total of 1 to 5 amino acids have been substituted, inserted and/or deleted in SEQ ID NO: 19. In some embodiments, substitutions, insertions, or deletions occur in regions outside the HVRs (i.e., in the FRs). In some embodiments, the immunoconjugate (e.g., the anti-CD79b immunoconjugate) comprises the VH sequence of SEQ ID NO: 19, including post-translational modifications of that sequence. In some embodiments, the VH comprises one, two or three HVRs selected from: (a) HVR-H1 comprising the amino acid sequence of SEQ ID NO: 21, (b) HVR-H2 comprising the amino acid sequence of SEQ ID NO: 22, and (c) HVR-H3 comprising the amino acid sequence of SEQ ID NO: 17 or SEQ ID NO: 23.

In some embodiments, the immunoconjugate (e.g., the anti-CD79b immunoconjugate) comprises an anti-CD79b antibody that comprises a light chain variable domain (VL) having at least 90%, 91%, 92%, 93%, 94%, 95%, 96%, 97%, 98%, 99%, or 100% sequence identity to the amino acid sequence of SEQ ID NO: 20. In certain embodiments, a VL sequence having at least 90%, 91%, 92%, 93%, 94%, 95%, 96%, 97%, 98%, or 99% identity to the amino acid sequence of SEQ ID NO: 20 contains substitutions (e.g., conservative substitutions), insertions, or deletions relative to the reference sequence, but an anti-CD79b immunoconjugate comprising that sequence retains the ability to bind to CD79b. In certain embodiments, a total of 1 to 10 amino acids have been substituted, inserted and/or deleted in SEQ ID NO: 20. In certain embodiments, a total of 1 to 5 amino acids have been substituted, inserted and/or deleted in SEQ ID NO: 20. In certain embodiments, the substitutions, insertions, or deletions occur in regions outside the HVRs (i.e., in the FRs). In some embodiments, the anti-CD79b immunoconjugate comprises an anti-CD79b antibody that comprises the VL sequence of SEQ ID NO: 20, including post-translational modifications of that sequence. In some embodiments, the VL comprises one, two or three HVRs selected from (a) HVR-L1 comprising an amino acid sequence of SEQ ID NO: 24; (b) HVR-L2 comprising the amino acid sequence of SEQ ID NO: 25; and (c) HVR-L3 comprising the amino acid sequence of SEQ ID NO: 26. In some embodiments, the VL comprises one, two or three HVRs selected from (a) HVR-L1 comprising the amino acid sequence of SEQ ID NO: 24; (b) HVR-L2 comprising the amino acid sequence of SEQ ID NO: 25; and (c) HVR-L3 comprising the amino acid sequence of SEQ ID NO: 26.

In some embodiments, the immunoconjugate (e.g., the anti-CD79b immunoconjugate) comprises an anti-CD79b antibody that comprises VH as in any of the embodiments provided herein, and a VL as in any of the embodiments provided herein. In some embodiments, the immunoconjugate comprises an anti-CD79b antibody that comprises the VH and VL sequences in SEQ ID NO: 19 and SEQ ID NO: 20, respectively, including post-translational modifications of those sequences.

In some embodiments, the immunoconjugate (e.g., anti-CD79b immunoconjugate) comprises an anti-CD79b antibody that binds to the same epitope as an anti-CD79b antibody described herein. For example, in some embodiments, the immunoconjugate (e.g., anti-CD79b immunoconjugate) comprises an anti-CD79b antibody that binds to the same epitope as an anti-CD79b antibody comprising a VH sequence of SEQ ID NO: 19 and a VL sequence of SEQ ID NO: 20.

In some embodiments, the immunoconjugate comprises an anti-CD79b antibody that is a monoclonal antibody, a chimeric antibody, humanized antibody, or human antibody. In some embodiments, immunoconjugate comprises an antigen-binding fragment of an anti-CD79b antibody described herein, e.g., a Fv, Fab, Fab′, scFv, diabody, or F(ab′)₂ fragment. In some embodiments, the immunoconjugate comprises a substantially full length anti-CD79b antibody, e.g., an IgG1 antibody or other antibody class or isotype as described elsewhere herein.

In some embodiments, the immunoconjugate comprises an anti-CD79b antibody comprising a heavy chain comprises the amino acid sequence of SEQ ID NO: 36, and wherein the light chain comprises the amino acid sequence of SEQ ID NO: 35. In some embodiments, the immunoconjugate comprises an anti-CD79 antibody that comprises a heavy chain comprising the amino acid sequence of SEQ ID NO: 37 and a light chain comprising the amino acid sequence of SEQ ID NO: 35. In some embodiments, the immunoconjugate comprises an anti-CD79 antibody that comprises a heavy chain comprising the amino acid sequence of SEQ ID NO: 36 and a light chain comprising the amino acid sequence of SEQ ID NO: 38.

In some embodiments, the immunoconjugate is polatuzumab vedotin, as described in WHO Drug Information, Vol. 26, No. 4, 2012 (Proposed INN: List 108), which is expressly incorporated by reference herein in its entirety. As shown in WHO Drug Information, Vol. 26, No. 4, 2012, polatuzumab vedotin has the following structure: immunoglobulin G1-kappa auristatin E conjugate, anti-[Homo sapiens CD79B (immunoglobulin-associated CD79 beta)], humanized monoclonal antibody conjugated to auristatin E; gamma1 heavy chain (1-447) [humanized VH (Homo sapiens IGHV3-66*01 (79.60%)-(IGHD)-IGHJ4*01) [8.8.13](1-120)-Homo sapiens IGHG1*03 (CH1 R120>K (214) (121-218), hinge (219-233), CH2 (234-343), CH3 (344-448), CHS (449-450)) (121-450)], (220-218′)-disulfide (if not conjugated) with kappa light chain (1′-218′) [humanized V-KAPPA (Homo sapiens IGKV1-39*01 (80.00%)-IGKJ1*01) [11.3.9](1′-112′)-Homo sapiens IGKC*01 (113′-218′)]; dimer (226-226″:229-229″)-bisdisulfide; conjugated, on an average of 3 to 4 cysteinyl, to monomethylauristatin E (MMAE), via a cleavable maleimidecaproyl-valyl-citrullinyl-p-aminobenzylcarbamate (mc-val-cit-PABC) linker;

| the heavy chain of polatuzumab has the following sequence: | |
|---|---|
| (SEQ ID NO: 56) | |
| EVQLVESGGG LVQPGGSLRL SCAASGYTFS SYWIEWVRQA PGKGLEWIGE | 50 |
| ILPGGGDTNY NEIFKGRATF SADTSKNTAY LQMNSLRAED TAVYYCTRRV | 100 |
| PIRLDYWGQG TLVTVSSAST KGPSVFPLAP SSKSTSGGTA ALGCLVKDYF | 150 |
| PEPVTVSWNS GALTSGVHTF PAVLQSSGLY SLSSVVTVPS SSLGTQTYIC | 200 |
| NVNHKPSNTK VDKKVEPKSC DKTHTCPPCP APELLGGPSV FLFPPKPKDT | 250 |
| LMISRTPEVT CVVVDVSHED PEVKFNWYVD GVEVHNAKTK PREEQYNSTY | 300 |
| RVVSVLTVLH QDWLNGKEYK CKVSNKALPA PIEKTISKAK GQPREPQVYT | 350 |
| LPPSREEMTK NQVSLTCLVK GFYPSDIAVE WESNGQPENN YKTTPPVLDS | 400 |
| DGSFFLYSKL TVDKSRWQQG NVFSCSVMHE ALHNHYTQKS LSLSPGK; | 447 |
| the light chain of polatuzumab has the following sequence: | |
| (SEQ ID NO: 35) | |
| DIQLTQSPSS LSASVGDRVT ITCKASQSVD YEGDSFLNWY QQKPGKAPKL | 50 |
| LIYAASNLES GVPSRFSGSG SGTDFTLTIS SLQPEDFATY YCQQSNEDPL | 100 |
| TFGQGTKVEI KRTVAAPSVF IFPPSDEQLK SGTASVVCLL NNFYPREAKV | 150 |
| QWKVDNALQS GNSQESVTEQ DSKDSTYSLS STLTLSKADY EKHKVYACEV | 200 |
| THQGLSSPVT KSFNRGEC; | 218 |

the disulfide bridge locations are:
Intra-H 22-96 144-200 261-321 367-425
   22″-96″ 147-203″ 261″-321″ 367-425″
Intra-L 23′-92′ 138′-198′
   23′″-92′″138′″-198′″
Inter-H-L* 220-218′ 220″-218′″
Inter-H—H* 226-226″ 229-229″
*Two or three of the inter-chain disulfide bridges are not present, the antibody being conjugated to an average of 3 to 4 drug linkers each via a thioether bond;
the N-glycosylation sites are H CH2 N84.4: 297, 297″ but lacking carbohydrate;
and other post-translational modifications are: lacking H chain C-terminal lysine.

### C. Drugs/Cytotoxic Agents

Anti-CD79 immunoconjugates comprise an anti-CD79b antibody (e.g., an anti-CD79b antibody described herein) conjugated to one or more drugs/cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes (i.e., a radioconjugate). Such immunoconjugates are targeted chemotherapeutic molecules which combine properties of both antibodies and cytotoxic drugs by targeting potent cytotoxic drugs to antigen-expressing cancer cells (such as tumor cells) (Teicher, B. A. (2009) Current Cancer Drug Targets 9:982-1004), thereby enhancing the therapeutic index by maximizing efficacy and minimizing off-target toxicity (Carter, P. J. and Senter P. D. (2008) The Cancer Jour. 14(3):154-169; Chari, R. V. (2008) Acc. Chem. Res. 41:98-107. That is, the anti-CD79 immunoconjugates selectively deliver an effective dose of a drug to cancerous cells/tissues whereby greater selectivity, i.e. a lower efficacious dose, may be achieved while increasing the therapeutic index (“therapeutic window”) (Polakis P. (2005) Current Opinion in Pharmacology 5:382-387).

Anti-CD79 immunoconjugates used in the methods provided herein include those with anticancer activity. In some embodiments, the anti-CD79 immunoconjugate comprises an anti-CD79b antibody conjugated, i.e. covalently attached, to the drug moiety. In some embodiments, the anti-CD79b antibody is covalently attached to the drug moiety through a linker. The drug moiety (D) of t the anti-CD79 immunoconjugate may include any compound, moiety or group that has a cytotoxic or cytostatic effect. Drug moieties may impart their cytotoxic and cytostatic effects by mechanisms including but not limited to tubulin binding, DNA binding or intercalation, and inhibition of RNA polymerase, protein synthesis, and/or topoisomerase. Exemplary drug moieties include, but are not limited to, a maytansinoid, dolastatin, auristatin, calicheamicin, anthracycline, duocarmycin, vinca alkaloid, taxane, trichothecene, CC1065, camptothecin, elinafide, and stereoisomers, isosteres, analogs, and derivatives thereof that have cytotoxic activity.

### (i) Maytansine and Maytansinoids

In some embodiments, an anti-CD79b immunoconjugate comprises an anti-CD79b antibody conjugated to one or more maytansinoid molecules. Maytansinoids are derivatives of maytansine, and are mitototic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinoids are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533.

Maytansinoid drug moieties are attractive drug moieties in antibody-drug conjugates because they are: (i) relatively accessible to prepare by fermentation or chemical modification or derivatization of fermentation products, (ii) amenable to derivatization with functional groups suitable for conjugation through non-disulfide linkers to antibodies, (iii) stable in plasma, and (iv) effective against a variety of tumor cell lines.

Certain maytansinoids suitable for use as maytansinoid drug moieties are known in the art and can be isolated from natural sources according to known methods or produced using genetic engineering techniques (see, e.g., Yu et al (2002) PNAS 99:7968-7973). Maytansinoids may also be prepared synthetically according to known methods.

Exemplary maytansinoid drug moieties include, but are not limited to, those having a modified aromatic ring, such as: C-19-dechloro (U.S. Pat. No. 4,256,746) (prepared, for example, by lithium aluminum hydride reduction of ansamytocin P2); C-20-hydroxy (or C-20-demethyl)+/−C-19-dechloro (U.S. Pat. Nos. 4,361,650 and 4,307,016) (prepared, for example, by demethylation using Streptomyces or Actinomyces or dechlorination using LAH); and C-20-demethoxy, C-20-acyloxy (—OCOR), +/−dechloro (U.S. Pat. No. 4,294,757) (prepared, for example, by acylation using acyl chlorides), and those having modifications at other positions of the aromatic ring.

Exemplary maytansinoid drug moieties also include those having modifications such as: C-9-SH (U.S. Pat. No. 4,424,219) (prepared, for example, by the reaction of maytansinol with H₂S or P₂S₅); C-14-alkoxymethyl(demethoxy/CH₂OR)(U.S. Pat. No. 4,331,598); C-14-hydroxymethyl or acyloxymethyl (CH₂OH or CH₂OAc) (U.S. Pat. No. 4,450,254) (prepared, for example, from Nocardia); C-15-hydroxy/acyloxy (U.S. Pat. No. 4,364,866) (prepared, for example, by the conversion of maytansinol by Streptomyces); C-15-methoxy (U.S. Pat. Nos. 4,313,946 and 4,315,929) (for example, isolated from Trewia nudlflora); C-18-N-demethyl (U.S. Pat. Nos. 4,362,663 and 4,322,348) (prepared, for example, by the demethylation of maytansinol by Streptomyces); and 4,5-deoxy (U.S. Pat. No. 4,371,533) (prepared, for example, by the titanium trichloride/LAH reduction of maytansinol).

Many positions on maytansinoid compounds are useful as the linkage position. For example, an ester linkage may be formed by reaction with a hydroxyl group using conventional coupling techniques. In some embodiments, the reaction may occur at the C-3 position having a hydroxyl group, the C-14 position modified with hydroxymethyl, the C-15 position modified with a hydroxyl group, and the C-20 position having a hydroxyl group. In some embodiments, the linkage is formed at the C-3 position of maytansinol or a maytansinol analogue.

Maytansinoid drug moieties include those having the structure:

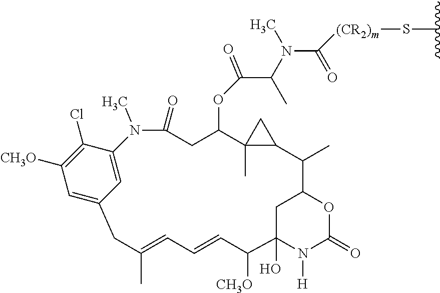

where the wavy line indicates the covalent attachment of the sulfur atom of the maytansinoid drug moiety to a linker of an anti-CD79b immunoconjugate. Each R may independently be H or a C₁-C₆ alkyl. The alkylene chain attaching the amide group to the sulfur atom may be methanyl, ethanyl, or propyl, i.e., m is 1, 2, or 3 (U.S. Pat. Nos. 633,410; 5,208,020; Chari et al (1992) Cancer Res. 52:127-131; Liu et al (1996) Proc. Natl. Acad. Sci USA 93:8618-8623).

All stereoisomers of the maytansinoid drug moiety are contemplated for the anti-CD79b immunoconjugate used in a method provided herein, i.e. any combination of R and S configurations at the chiral carbons (U.S. Pat. Nos. 7,276,497; 6,913,748; 6,441,163; 633,410 (RE39151); U.S. Pat. No. 5,208,020; Widdison et al (2006) J. Med. Chem. 49:4392-4408, which are incorporated by reference in their entirety). In some embodiments, the maytansinoid drug moiety has the following stereochemistry:

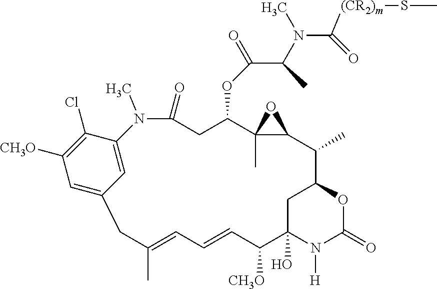

Exemplary embodiments of maytansinoid drug moieties include, but are not limited to, DM1; DM3; and DM4, having the structures:

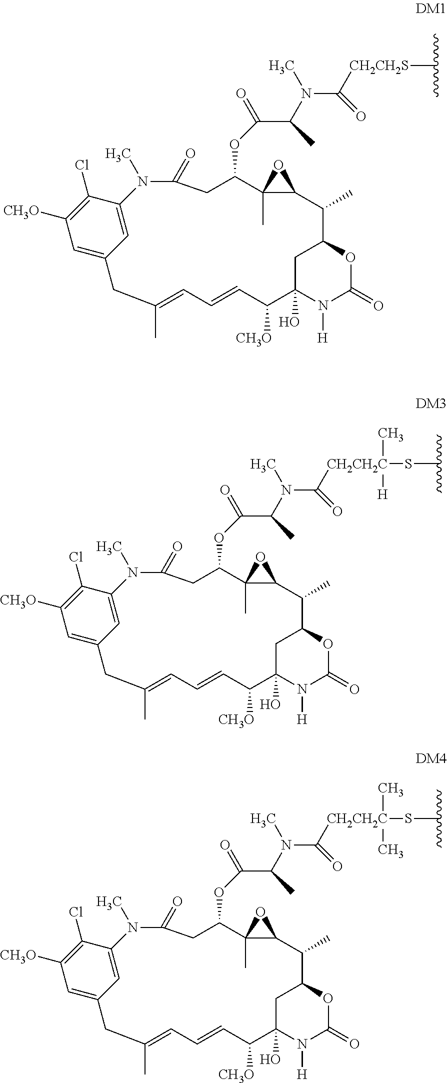

wherein the wavy line indicates the covalent attachment of the sulfur atom of the drug to a linker (L) of an anti-CD79b immunoconjugate.

Other exemplary maytansinoid anti-CD79b immunoconjugates have the following structures and abbreviations (wherein Ab is an anti-CD79b antibody and p is 1 to about 20. In some embodiments, p is 1 to 10, p is 1 to 7, p is 1 to 5, or p is 1 to 4):

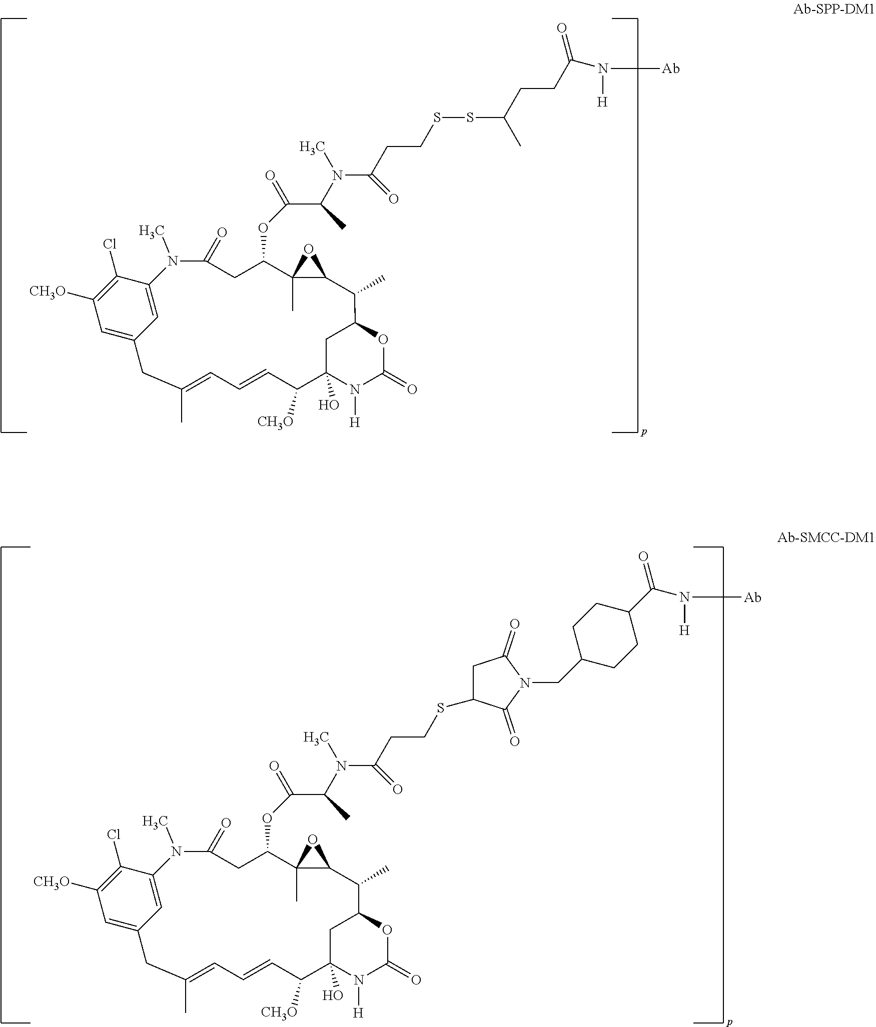

Exemplary antibody-drug conjugates where DM1 is linked through a BMPEO linker to a thiol group of the antibody have the structure and abbreviation:

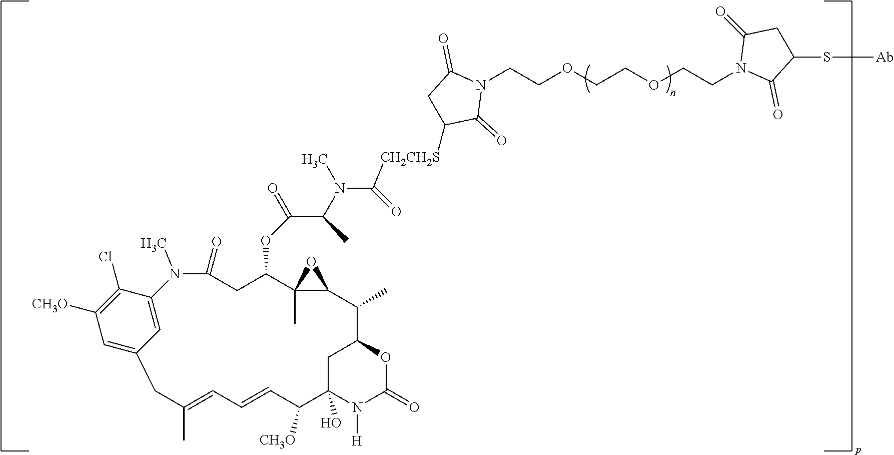

where Ab is an anti-CD79b antibody; n is 0, 1, or 2; and p is 1 to about 20. In some embodiments, p is 1 to 10, p is 1 to 7, p is 1 to 5, or p is 1 to 4.

Immunoconjugates containing maytansinoids, methods of making the same, and their therapeutic use are disclosed, for example, in U.S. Pat. Nos. 5,208,020 and 5,416,064; US 2005/0276812 A1; and European Patent EP 0 425 235 B1, the disclosures of which are hereby expressly incorporated by reference. See also Liu et al. Proc. Natl. Acad. Sci. USA 93:8618-8623 (1996); and Chari et al. Cancer Research 52:127-131 (1992).

In some embodiments, anti-CD79b antibody-maytansinoid conjugates may be prepared by chemically linking an anti-CD79b antibody to a maytansinoid molecule without significantly diminishing the biological activity of either the antibody or the maytansinoid molecule. See, e.g., U.S. Pat. No. 5,208,020 (the disclosure of which is hereby expressly incorporated by reference). In some embodiments, an anti-CD79b immunoconjugate with an average of 3-4 maytansinoid molecules conjugated per antibody molecule has shown efficacy in enhancing cytotoxicity of target cells without negatively affecting the function or solubility of the antibody. In some instances, even one molecule of toxin/antibody is expected to enhance cytotoxicity over the use of naked anti-CD79b antibody.

Exemplary linking groups for making antibody-maytansinoid conjugates include, for example, those described herein and those disclosed in U.S. Pat. No. 5,208,020; EP Patent 0 425 235 B1; Chari et al. Cancer Research 52:127-131 (1992); US 2005/0276812 A1; and US 2005/016993 A1, the disclosures of which are hereby expressly incorporated by reference.

### (2) Auristatins and Dolastatins

Drug moieties include dolastatins, auristatins, and analogs and derivatives thereof (U.S. Pat. Nos. 5,635,483; 5,780,588; 5,767,237; 6,124,431). Auristatins are derivatives of the marine mollusk compound dolastatin-10. While not intending to be bound by any particular theory, dolastatins and auristatins have been shown to interfere with microtubule dynamics, GTP hydrolysis, and nuclear and cellular division (Woyke et al (2001) Antimicrob. Agents and Chemother. 45(12):3580-3584) and have anticancer (U.S. Pat. No. 5,663,149) and antifungal activity (Pettit et al (1998) Antimicrob. Agents Chemother. 42:2961-2965). The dolastatin/auristatin drug moiety may be attached to the antibody through the N (amino) terminus or the C (carboxyl) terminus of the peptidic drug moiety (WO 02/088172; Doronina et al (2003) Nature Biotechnology 21(7):778-784; Francisco et al (2003) Blood 102(4):1458-1465).

Exemplary auristatin embodiments include the N-terminus linked monomethylauristatin drug moieties D_{E} and D_{F}, disclosed in U.S. Pat. Nos. 7,498,298 and 7,659,241, the disclosures of which are expressly incorporated by reference in their entirety:

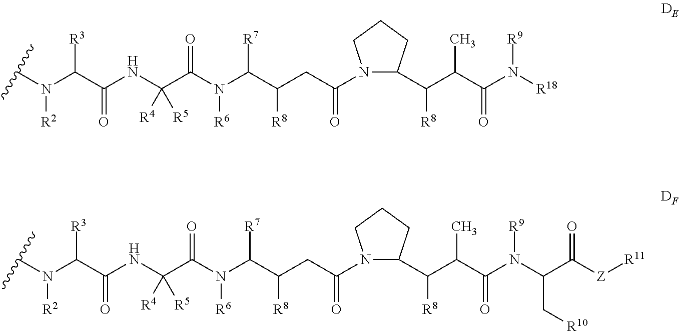

wherein the wavy line of D_{E} and D_{F} indicates the covalent attachment site to an antibody or antibody-linker component, and independently at each location:
R² is selected from H and C₁-C₈ alkyl;
R³ is selected from H, C₁-C₈ alkyl, C₃-C₈ carbocycle, aryl, C₁-C₈ alkyl-aryl, C₁-C₈ alkyl-(C₃-C₈ carbocycle), C₃-C₈ heterocycle and C₁-C₈ alkyl-(C₃-C₈ heterocycle);
R⁴ is selected from H, C₁-C₈ alkyl, C₃-C₈ carbocycle, aryl, C₁-C₈ alkyl-aryl, C₁-C₈ alkyl-(C₃-C₈ carbocycle), C₃-C₈ heterocycle and C₁-C₈ alkyl-(C₃-C₈ heterocycle);
R⁵ is selected from H and methyl;
or R⁴ and R⁵ jointly form a carbocyclic ring and have the formula —(CR^{a}R^{b})ₙ— wherein R^{a} and R^{b} are independently selected from H, C₁-C₈ alkyl and C₃-C₈ carbocycle and n is selected from 2, 3, 4, 5 and 6;
R⁶ is selected from H and C₁-C₈ alkyl;
R⁷ is selected from H, C₁-C₈ alkyl, C₃-C₈ carbocycle, aryl, C₁-C₈ alkyl-aryl, C₁-C₈ alkyl-(C₃-C₈ carbocycle), C₃-C₈ heterocycle and C₁-C₈ alkyl-(C₃-C₈ heterocycle);
each R⁸ is independently selected from H, OH, C₁-C₈ alkyl, C₃-C₈ carbocycle and O—(C₁-C₈ alkyl);
R⁹ is selected from H and C₁-C₈ alkyl;
R¹⁰ is selected from aryl or C₃-C₈ heterocycle;
Z is O, S, NH, or NR¹², wherein R¹² is C₁-C₈ alkyl;
R¹¹ is selected from H, C₁-C₂₀ alkyl, aryl, C₃-C₈ heterocycle, —(R¹³O)ₘ—R¹⁴, or —(R¹³O)ₘ—CH(R¹⁵)₂;
m is an integer ranging from 1-1000;
R¹³ is C₂-C₈ alkyl;
R¹⁴ is H or C₁-C₈ alkyl;
each occurrence of R¹⁵ is independently H, COOH, —(CH₂)ₙ—N(R¹⁶)₂, —(CH₂)ₙ—SO₃H, or —(CH₂)ₙ—SO₃—C₁-C₈ alkyl;
each occurrence of R¹⁶ is independently H, C₁-C₈ alkyl, or —(CH₂)ₙ—COOH;
R¹⁸ is selected from —C(R⁸)₂—C(R⁸)₂-aryl, —C(R⁸)₂—C(R⁸)₂—(C₃-C₈ heterocycle), and —C(R⁸)₂—C(R⁸)₂—(C₃-C₈ carbocycle); and n is an integer ranging from 0 to 6.

In one embodiment, R³, R⁴ and R⁷ are independently isopropyl or sec-butyl and R⁵ is —H or methyl. In an exemplary embodiment, R³ and R⁴ are each isopropyl, R⁵ is —H, and R⁷ is sec-butyl.

In yet another embodiment, R² and R⁶ are each methyl, and R⁹ is —H.

In still another embodiment, each occurrence of R⁸ is —OCH₃.

In an exemplary embodiment, R³ and R⁴ are each isopropyl, R² and R⁶ are each methyl, R⁵ is —H, R⁷ is sec-butyl, each occurrence of R⁸ is —OCH₃, and R⁹ is —H.

In one embodiment, Z is —O— or —NH—.

In one embodiment, R¹⁰ is aryl.

In an exemplary embodiment, R¹⁰ is -phenyl.

In an exemplary embodiment, when Z is —O—, R¹¹ is —H, methyl or t-butyl.

In one embodiment, when Z is —NH, R¹ is —CH(R¹⁵)₂, wherein R¹⁵ is —(CH₂)ₙ—N(R¹⁶)₂, and R¹⁶ is —C₁-C₈ alkyl or —(CH₂)ₙ—COOH.

In another embodiment, when Z is —NH, R¹¹ is —CH(R¹⁵)₂, wherein R¹⁵ is —(CH₂)ₙ—SO₃H.

An exemplary auristatin embodiment of formula D_{E} is MMAE, wherein the wavy line indicates the covalent attachment to a linker (L) of an anti-CD79b immunoconjugate:

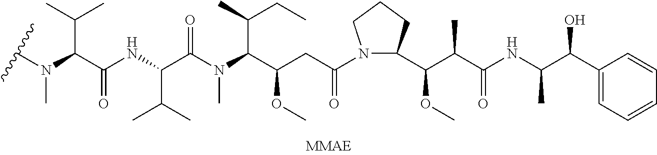 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

An exemplary auristatin embodiment of formula D_{F} is MMAF, wherein the wavy line indicates the covalent attachment to a linker (L) of an anti-CD79b immunoconjugate:

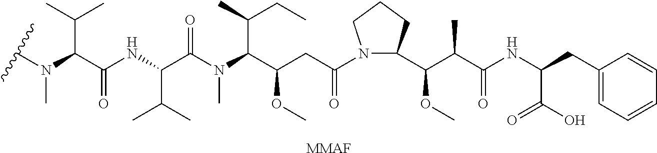

Other exemplary embodiments include monomethylvaline compounds having phenylalanine carboxy modifications at the C-terminus of the pentapeptide auristatin drug moiety (WO 2007/008848) and monomethylvaline compounds having phenylalanine sidechain modifications at the C-terminus of the pentapeptide auristatin drug moiety (WO 2007/008603).

Nonlimiting exemplary embodiments of an anti-CD79b immunoconjugate of Formula I comprising MMAE or MMAF and various linker components have the following structures and abbreviations (wherein “Ab” is an anti-CD79b antibody; p is 1 to about 8, “Val-Cit” is a valine-citrulline dipeptide; and “S” is a sulfur atom:

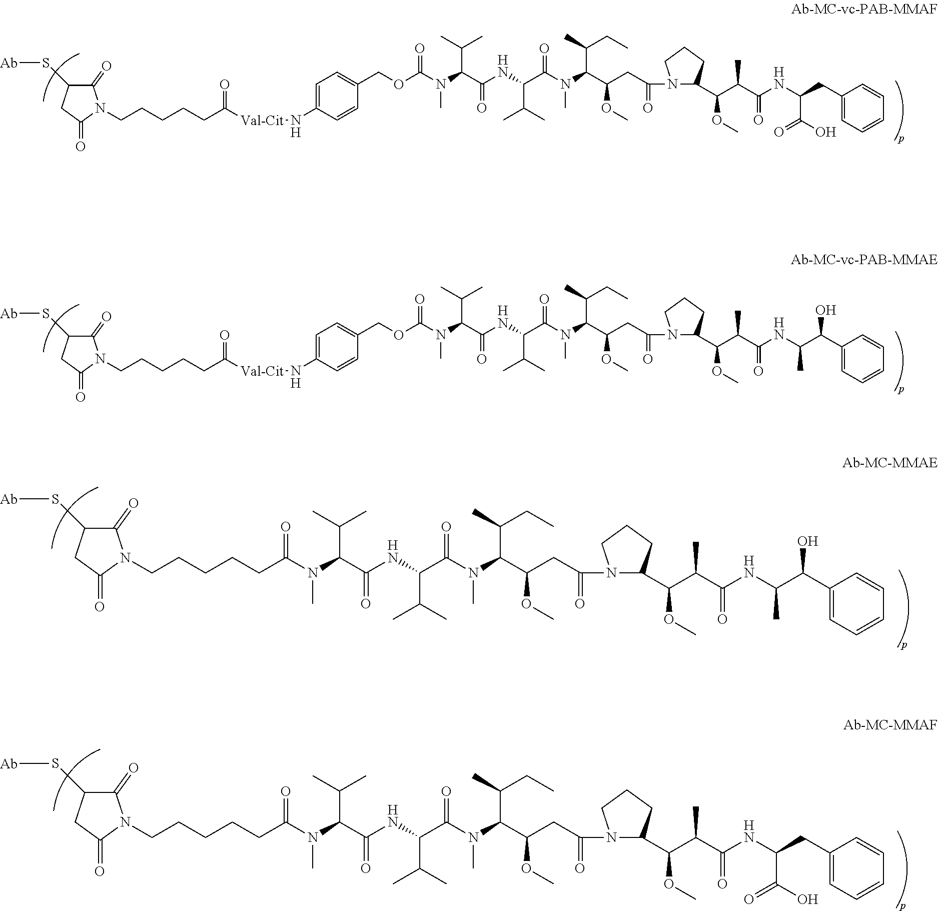

In certain embodiments, the anti-CD79b immunoconjugate comprises the structure of Ab-MC-vc-PAB-MMAE, wherein p is, e.g., about 1 to about 8; about 2 to about 7; about 3 to about 5; about 3 to about 4; or about 3.5. In some embodiments, the anti-CD79b immunoconjugate is huMA79bv28-MC-vc-PAB-MMAE, e.g., an anti-CD79b immunoconjugate comprising the structure of MC-vc-PAB-MMAE, wherein p is, e.g., about 1 to about 8; about 2 to about 7; about 3 to about 5; about 3 to about 4; or about 3.5, wherein the anti-CD79 antibody comprises a heavy chain comprising the amino acid sequence of SEQ ID NO: 36, and wherein the light chain comprises the amino acid sequence of SEQ ID NO: 35. In some embodiments, the anti-CD79b immunoconjugate is polatuzumab vedotin (CAS Number 1313206-42-6). Polatuzumab vedotin has the IUPHAR/BPS Number 8404, the KEGG Number D10761, the INN number 9714, and can also be referred to as “DCDS4501A,” or “RG7596.”

Nonlimiting exemplary embodiments of anti-CD79b immunoconjugates of Formula I comprising MMAF and various linker components further include Ab-MC-PAB-MMAF and Ab-PAB-MMAF. Immunoconjugates comprising MMAF attached to an antibody by a linker that is not proteolytically cleavable have been shown to possess activity comparable to immunoconjugates comprising MMAF attached to an antibody by a proteolytically cleavable linker (Doronina et al. (2006) Bioconjugate Chem. 17:114-124). In some such embodiments, drug release is believed to be effected by antibody degradation in the cell.

Typically, peptide-based drug moieties can be prepared by forming a peptide bond between two or more amino acids and/or peptide fragments. Such peptide bonds can be prepared, for example, according to a liquid phase synthesis method (see, e.g., E. Schröder and K. Lübke, “The Peptides”, volume 1, pp 76-136, 1965, Academic Press). Auristatin/dolastatin drug moieties may, in some embodiments, be prepared according to the methods of: U.S. Pat. Nos. 7,498,298; 5,635,483; 5,780,588; Pettit et al (1989) J. Am. Chem. Soc. 111:5463-5465; Pettit et al (1998) Anti-Cancer Drug Design 13:243-277; Pettit, G. R., et al. Synthesis, 1996, 719-725; Pettit et al (1996) J. Chem. Soc. Perkin Trans. 1 5:859-863; and Doronina (2003) Nat. Biotechnol. 21(7):778-784.

In some embodiments, auristatin/dolastatin drug moieties of formulas D_{E} such as MMAE, and D_{F}, such as MMAF, and drug-linker intermediates and derivatives thereof, such as MC-MMAF, MC-MMAE, MC-vc-PAB-MMAF, and MC-vc-PAB-MMAE, may be prepared using methods described in U.S. Pat. No. 7,498,298; Doronina et al. (2006) Bioconjugate Chem. 17:114-124; and Doronina et al. (2003) Nat. Biotech. 21:778-784 and then conjugated to an antibody of interest.

### (3) Calicheamicin

In some embodiments, the anti-CD79b immunoconjugate comprises an anti-CD79b antibody conjugated to one or more calicheamicin molecules. The calicheamicin family of antibiotics, and analogues thereof, are capable of producing double-stranded DNA breaks at sub-picomolar concentrations (Hinman et al., (1993) Cancer Research 53:3336-3342; Lode et al., (1998) Cancer Research 58:2925-2928). Calicheamicin has intracellular sites of action but, in certain instances, does not readily cross the plasma membrane. Therefore, cellular uptake of these agents through antibody-mediated internalization may, in some embodiments, greatly enhance their cytotoxic effects. Nonlimiting exemplary methods of preparing anti-CD79b antibody immunoconjugates with a calicheamicin drug moiety are described, for example, in U.S. Pat. Nos. 5,712,374; 5,714,586; 5,739,116; and 5,767,285.

### (4) Other Drug Moieties

In some embodiments, an anti-CD79b immunoconjugate comprises geldanamycin (Mandler et al (2000) J. Nat. Cancer Inst. 92(19):1573-1581; Mandler et al (2000) Bioorganic & Med. Chem. Letters 10:1025-1028; Mandler et al (2002) Bioconjugate Chem. 13:786-791); and/or enzymatically active toxins and fragments thereof, including, but not limited to, diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes. See, e.g., WO 93/21232.

Drug moieties also include compounds with nucleolytic activity (e.g., a ribonuclease or a DNA endonuclease).

In certain embodiments, an anti-CD79b immunoconjugate comprises a highly radioactive atom. A variety of radioactive isotopes are available for the production of radioconjugated antibodies. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. In some embodiments, when an anti-CD79b immunoconjugate is used for detection, it may comprise a radioactive atom for scintigraphic studies, for example Tc⁹⁹ or I¹²³, or a spin label for nuclear magnetic resonance (NMR) imaging (also known as magnetic resonance imaging, MRI), such as zirconium-89, iodine-123, iodine-131, indium-111, fluorine-19, carbon-13, nitrogen-15, oxygen-17, gadolinium, manganese or iron. Zirconium-89 may be complexed to various metal chelating agents and conjugated to antibodies, e.g., for PET imaging (WO 2011/056983).

The radio- or other labels may be incorporated in the anti-CD79b immunoconjugate in known ways. For example, a peptide may be biosynthesized or chemically synthesized using suitable amino acid precursors comprising, for example, one or more fluorine-19 atoms in place of one or more hydrogens. In some embodiments, labels such as Tc⁹⁹, I¹²³, Re¹⁸⁶, Re¹⁸⁸ and In¹¹¹ can be attached via a cysteine residue in the anti-CD79b antibody. In some embodiments, yttrium-90 can be attached via a lysine residue of the anti-CD79b antibody. In some embodiments, the IODOGEN method (Fraker et al (1978) Biochem. Biophys. Res. Commun. 80: 49-57 can be used to incorporate iodine-123. “Monoclonal Antibodies in Immunoscintigraphy” (Chatal, CRC Press 1989) describes certain other methods.

In certain embodiments, an anti-CD79b immunoconjugate may comprise an anti-CD79b antibody conjugated to a prodrug-activating enzyme. In some such embodiments, a prodrug-activating enzyme converts a prodrug (e.g., a peptidyl chemotherapeutic agent, see WO 81/01145) to an active drug, such as an anti-cancer drug. Such immunoconjugates are useful, in some embodiments, in antibody-dependent enzyme-mediated prodrug therapy (“ADEPT”). Enzymes that may be conjugated to an anti-CD79b antibody include, but are not limited to, alkaline phosphatases, which are useful for converting phosphate-containing prodrugs into free drugs; arylsulfatases, which are useful for converting sulfate-containing prodrugs into free drugs; cytosine deaminase, which is useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), which are useful for converting peptide-containing prodrugs into free drugs; D-alanylcarboxypeptidases, which are useful for converting prodrugs that contain D-amino acid substituents; carbohydrate-cleaving enzymes such as β-galactosidase and neuraminidase, which are useful for converting glycosylated prodrugs into free drugs; β-lactamase, which is useful for converting drugs derivatized with β-lactams into free drugs; and penicillin amidases, such as penicillin V amidase and penicillin G amidase, which are useful for converting drugs derivatized at their amine nitrogens with phenoxyacetyl or phenylacetyl groups, respectively, into free drugs. In some embodiments, enzymes may be covalently bound to antibodies by recombinant DNA techniques well known in the art. See, e.g., Neuberger et al., Nature 312:604-608 (1984).

### D. Drug Loading

Drug loading is represented by p, the average number of drug moieties per anti-CD79b antibody in a molecule of Formula I. Drug loading may range from 1 to 20 drug moieties (D) per antibody. Anti-CD79b immunoconjugates of Formula I include collections of anti-CD79b antibodies conjugated with a range of drug moieties, from 1 to 20. The average number of drug moieties per anti-CD79b antibody in preparations of anti-CD79b immunoconjugates from conjugation reactions may be characterized by conventional means such as mass spectroscopy, ELISA assay, and HPLC. The quantitative distribution of anti-CD79b immunoconjugates in terms of p may also be determined. In some instances, separation, purification, and characterization of homogeneous anti-CD79b immunoconjugates where p is a certain value from anti-CD79b immunoconjugates with other drug loadings may be achieved by means such as reverse phase HPLC or electrophoresis.

For some anti-CD79b immunoconjugates, p may be limited by the number of attachment sites on the anti-CD79b antibody. For example, where the attachment is a cysteine thiol, as in certain exemplary embodiments above, an anti-CD79b antibody may have only one or several cysteine thiol groups, or may have only one or several sufficiently reactive thiol groups through which a linker may be attached. In certain embodiments, higher drug loading, e.g., p >5, may cause aggregation, insolubility, toxicity, or loss of cellular permeability of certain anti-CD79b immunoconjugates. In certain embodiments, the average drug loading for an anti-CD79b immunoconjugates ranges from 1 to about 8; from about 2 to about 6; from about 3 to about 5; or from about 3 to about 4. Indeed, it has been shown that for certain antibody-drug conjugates, the optimal ratio of drug moieties per antibody may be less than 8, and may be about 2 to about 5 (U.S. Pat. No. 7,498,298). In certain embodiments, the optimal ratio of drug moieties per antibody is about 3 to about 4. In certain embodiments, the optimal ratio of drug moieties per antibody is about 3.5.

In certain embodiments, fewer than the theoretical maximum of drug moieties are conjugated to the anti-CD79b antibody during a conjugation reaction. An antibody may contain, for example, lysine residues that do not react with the drug-linker intermediate or linker reagent, as discussed below. Generally, antibodies do not contain many free and reactive cysteine thiol groups which may be linked to a drug moiety; indeed most cysteine thiol residues in antibodies exist as disulfide bridges. In certain embodiments, an anti-CD79b antibody may be reduced with a reducing agent such as dithiothreitol (DTT) or tricarbonylethylphosphine (TCEP), under partial or total reducing conditions, to generate reactive cysteine thiol groups. In certain embodiments, an anti-CD79b antibody is subjected to denaturing conditions to reveal reactive nucleophilic groups such as lysine or cysteine.

The loading (drug/antibody ratio) of an anti-CD79b immunoconjugate may be controlled in different ways, and for example, by: (i) limiting the molar excess of drug-linker intermediate or linker reagent relative to antibody, (ii) limiting the conjugation reaction time or temperature, and (iii) partial or limiting reductive conditions for cysteine thiol modification.

It is to be understood that where more than one nucleophilic group reacts with a drug-linker intermediate or linker reagent, then the resulting product is a mixture of anti-CD79b immunoconjugate compounds with a distribution of one or more drug moieties attached to an anti-CD79b antibody. The average number of drugs per antibody may be calculated from the mixture by a dual ELISA antibody assay, which is specific for antibody and specific for the drug. Individual anti-CD79b immunoconjugate molecules may be identified in the mixture by mass spectroscopy and separated by HPLC, e.g., hydrophobic interaction chromatography (see, e.g., McDonagh et al (2006) Prot. Engr. Design & Selection 19(7):299-307; Hamblett et al (2004) Clin. Cancer Res. 10:7063-7070; Hamblett, K. J., et al. “Effect of drug loading on the pharmacology, pharmacokinetics, and toxicity of an anti-CD30 antibody-drug conjugate,” Abstract No. 624, American Association for Cancer Research, 2004 Annual Meeting, Mar. 27-31, 2004, Proceedings of the AACR, Volume 45, March 2004; Alley, S. C., et al. “Controlling the location of drug attachment in antibody-drug conjugates,” Abstract No. 627, American Association for Cancer Research, 2004 Annual Meeting, Mar. 27-31, 2004, Proceedings of the AACR, Volume 45, March 2004). In certain embodiments, a homogeneous anti-CD79b immunoconjugate with a single loading value may be isolated from the conjugation mixture by electrophoresis or chromatography.

### E. Methods of Preparing Anti-CD79b Immunoconjugates

An anti-CD79b immunoconjugate of Formula I may be prepared by several routes employing organic chemistry reactions, conditions, and reagents known to those skilled in the art, including, but not limited to, e.g., (1) reaction of a nucleophilic group of an anti-CD79b antibody with a bivalent linker reagent to form Ab-L via a covalent bond, followed by reaction with a drug moiety D; and (2) reaction of a nucleophilic group of a drug moiety with a bivalent linker reagent, to form D-L, via a covalent bond, followed by reaction with a nucleophilic group of an anti-CD79b antibody. Exemplary methods for preparing an anti-CD79b immunoconjugate of Formula I via the latter route are described in U.S. Pat. No. 7,498,298, which is expressly incorporated herein by reference.

Nucleophilic groups on antibodies include, but are not limited to: (i) N-terminal amine groups, (ii) side chain amine groups, e.g., lysine, (iii) side chain thiol groups, e.g., cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; and (iii) aldehydes, ketones, carboxyl, and maleimide groups. Certain antibodies have reducible interchain disulfides, i.e. cysteine bridges. Anti-CD79b antibodies may be made reactive for conjugation with linker reagents by treatment with a reducing agent such as DTT (dithiothreitol) or tricarbonylethylphosphine (TCEP), such that the anti-CD79b antibody is fully or partially reduced. Each cysteine bridge will thus form, theoretically, two reactive thiol nucleophiles. Additional nucleophilic groups can be introduced into anti-CD79b antibodies through modification of lysine residues, e.g., by reacting lysine residues with 2-iminothiolane (Traut's reagent), resulting in conversion of an amine into a thiol. Reactive thiol groups may also be introduced into an anti-CD79b antibody by introducing one, two, three, four, or more cysteine residues (e.g., by preparing variant antibodies comprising one or more non-native cysteine amino acid residues).

Anti-CD79b immunoconjugates described herein may also be produced by reaction between an electrophilic group on an anti-CD79b antibody, such as an aldehyde or ketone carbonyl group, with a nucleophilic group on a linker reagent or drug. Useful nucleophilic groups on a linker reagent include, but are not limited to, hydrazide, oxime, amino, hydrazine, thiosemicarbazone, hydrazine carboxylate, and arylhydrazide. In one embodiment, an anti-CD79b antibody is modified to introduce electrophilic moieties that are capable of reacting with nucleophilic substituents on the linker reagent or drug. In another embodiment, the sugars of glycosylated anti-CD79b antibodies may be oxidized, e.g., with periodate oxidizing reagents, to form aldehyde or ketone groups which may react with the amine group of linker reagents or drug moieties. The resulting imine Schiff base groups may form a stable linkage, or may be reduced, e.g., by borohydride reagents to form stable amine linkages. In one embodiment, reaction of the carbohydrate portion of a glycosylated anti-CD79b antibody with either galactose oxidase or sodium meta-periodate may yield carbonyl (aldehyde and ketone) groups in the anti-CD79b antibody that can react with appropriate groups on the drug (Hermanson, Bioconjugate Techniques). In another embodiment, anti-CD79b antibodies containing N-terminal serine or threonine residues can react with sodium meta-periodate, resulting in production of an aldehyde in place of the first amino acid (Geoghegan & Stroh, (1992) Bioconjugate Chem. 3:138-146; U.S. Pat. No. 5,362,852). Such an aldehyde can be reacted with a drug moiety or linker nucleophile.

Exemplary nucleophilic groups on a drug moiety include, but are not limited to: amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups.

Nonlimiting exemplary cross-linker reagents that may be used to prepare anti-CD79b immunoconjugates are described herein in the section titled “Exemplary Linkers.” Methods of using such cross-linker reagents to link two moieties, including a proteinaceous moiety and a chemical moiety, are known in the art. In some embodiments, a fusion protein comprising an anti-CD79b antibody and a cytotoxic agent may be made, e.g., by recombinant techniques or peptide synthesis. A recombinant DNA molecule may comprise regions encoding the antibody and cytotoxic portions of the conjugate either adjacent to one another or separated by a region encoding a linker peptide which does not destroy the desired properties of the conjugate. In yet another embodiment, an anti-CD79b antibody may be conjugated to a “receptor” (such as streptavidin) for utilization in tumor pre-targeting wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a drug or radionucleotide). Additional details regarding anti-CD79b immunoconjugates are provided in U.S. Pat. No. 8,545,850 and WO/2016/049214, the contents of which are expressly incorporated by reference herein in their entirety.

### V. Immunomodulatory Agents

Immunomodulatory agents (e.g., thalidomide, lenalidomide, and pomalidomide, which are also known as “IMiDs®”) are a class of orally available antineoplastic or anticancer drugs that exhibit pleiotropic properties. For example, immunomodulatory agents stimulate NK-cell and T-cell activity and exhibit anti-angiogenic, anti-inflammatory, pro-apoptotic, and anti-proliferative effects, as well. The mechanisms of action by which immunomodulatory drugs exert their effects have not yet been fully characterized.

Lenalidomide is an exemplary immunomodulatory agent used in the methods described herein. The chemical name for lenalidomide is 3-(4-amino-1-oxo-2,3-dihydro-1H-isoindol-2-yl)piperidine-2,6-dione, and lenalidomide has the following chemical structure:

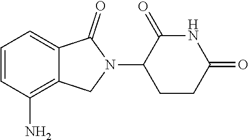 [SMILES: Nc1cccc2c1CN(C1CCC(=O)NC1=O)C2=O]

Lenalidomide (CAS Registry #191732-72-6) has the molecular formula of C₁₃H₁₃N₃O₃ and a molecular weight of 259.261 g/mol. Lenalidomide is also known as CC-5103, IMiD3 cdp. It is commercially available for therapeutic use under the trade name REVLIMID®, and is provided as 2.5 mg, 5 mg, 10 mg, 15 mg, 20 mg, and 25 mg capsules. Lenalidomide may be provided in a dose of, for example, 2.5 mg, 5 mg, 10 mg, 15 mg, 20 mg, or 25 mg.

### VI. Anti-CD20 Agents

Depending on binding properties and biological activities of anti-CD20 antibodies to the CD20 antigen, two types of anti-CD20 antibodies (type I and type II anti-CD20 antibodies) can be distinguished according to Cragg, M. S., et al., Blood 103 (2004) 2738-2743; and Cragg, M. S., et al., Blood 101 (2003) 1045-1052, see Table C.

TABLE C
Properties of type I and type II anti-CD20 antibodies

| Type I | Type II |
|---|---|
| anti-CD20 antibodies | anti-CD20 antibodies |
| type I CD20 epitope | type II CD20 epitope |
| Localize CD20 to lipid rafts | Do not localize CD20 to lipid rafts |
| Increased CDC (if IgG1 isotype) | Decreased CDC (if IgG1 isotype) |
| ADCC activity (if IgG1 isotype) | ADCC activity (if IgG1 isotype) |
| Full binding capacity | Reduced binding capacity |
| Homotypic aggregation | Stronger homotypic aggregation |
| Apoptosis induction upon cross-linking | Strong cell death induction without |
| | cross-linking |

Examples of type I anti-CD20 antibodies include e.g., rituximab, H147 IgG3 (ECACC, hybridoma), 2C6 IgG1 (as disclosed in WO 2005/103081), 2F2 IgG1 (as disclosed and WO 2004/035607 and WO 2005/103081) and 2H7 IgG1 (as disclosed in WO 2004/056312).

In some embodiments, the anti-CD20 antibody used a method of treatment provided herein is rituximab. In some embodiments, the rituximab (reference antibody; example of a type I anti-CD20 antibody) is a genetically engineered chimeric human gamma 1 murine constant domain containing monoclonal antibody directed against the human CD20 antigen. However this antibody is not glycoengineered and not afucosylated and thus has an amount of fucose of at least 85%. This chimeric antibody comprises human gamma 1 constant domains and is identified by the name “C2B8” in U.S. Pat. No. 5,736,137 (Andersen, et. al.) issued on Apr. 17, 1998, assigned to IDEC Pharmaceuticals Corporation. Rituximab is approved for the treatment of patients with relapsed or refracting low-grade or follicular, CD20 positive, B-cell non-Hodgkin's lymphoma. In vitro mechanism of action studies have shown that rituximab exhibits human complement-dependent cytotoxicity (CDC) (Reff, M. E., et. al, Blood 83(2) (1994) 435-445). Additionally, it exhibits activity in assays that measure antibody-dependent cellular cytotoxicity (ADCC).

In some embodiments, the anti-CD20 antibody used in a method of treatment provided herein is an afucosylated anti-CD20 antibody.

Examples of type II anti-CD20 antibodies include e.g., humanized B-Ly1 antibody IgG1 (a chimeric humanized IgG1 antibody as disclosed in WO 2005/044859), 11B8 IgG1 (as disclosed in WO 2004/035607), and AT80 IgG1. Typically type II anti-CD20 antibodies of the IgG1 isotype show characteristic CDC properties. Type II anti-CD20 antibodies have a decreased CDC (if IgG1 isotype) compared to type I antibodies of the IgG1 isotype. In some embodiments the type II anti-CD20 antibody, e.g., a GA101 antibody, has increased antibody dependent cellular cytotoxicity (ADCC). In some embodiments, the type II anti-CD20 antibodies, more preferably an afucosylated humanized B-Ly1 antibody as described in WO 2005/044859 and WO 2007/031875.

In some embodiments, the anti-CD20 antibody used in a method of treatment provided herein is GA101 antibody. In some embodiments, the GA101 antibody as used herein refers to any one of the following antibodies that bind human CD20: (1) an antibody comprising an HVR-H1 comprising the amino acid sequence of SEQ ID NO:5, an HVR-H2 comprising the amino acid sequence of SEQ ID NO:6, an HVR-H3 comprising the amino acid sequence of SEQ ID NO:7, an HVR-L1 comprising the amino acid sequence of SEQ ID NO:8, an HVR-L2 comprising the amino acid sequence of SEQ ID NO:9, and an HVR-L3 comprising the amino acid sequence of SEQ ID NO:10; (2) an antibody comprising a VH domain comprising the amino acid sequence of SEQ ID NO:11 and a VL domain comprising the amino acid sequence of SEQ ID NO:12, (3) an antibody comprising an amino acid sequence of SEQ ID NO:13 and an amino acid sequence of SEQ ID NO: 14; (4) an antibody known as obinutuzumab, or (5) an antibody that comprises an amino acid sequence that has at least 95%, 96%, 97%, 98% or 99% sequence identity with amino acid sequence of SEQ ID NO:13 and that comprises an amino acid sequence that has at least 95%, 96%, 97%, 98% or 99% sequence identity with an amino acid sequence of SEQ ID NO: 14. In one embodiment, the GA101 antibody is an IgG1 isotype antibody.

In some embodiments, the anti-CD20 antibody used in a method of treatment provided herein is a humanized B-Ly1 antibody. In some embodiments, the humanized B-Ly1 antibody refers to humanized B-Ly1 antibody as disclosed in WO 2005/044859 and WO 2007/031875, which were obtained from the murine monoclonal anti-CD20 antibody B-Ly1 (variable region of the murine heavy chain (VH): SEQ ID NO: 3; variable region of the murine light chain (VL): SEQ ID NO: 4—see Poppema, S. and Visser, L., Biotest Bulletin 3 (1987) 131-139) by chimerization with a human constant domain from IgG1 and following humanization (see WO 2005/044859 and WO 2007/031875). The humanized B-Ly1 antibodies are disclosed in detail in WO 2005/044859 and WO 2007/031875.

In some embodiments, the humanized B-Ly1 antibody has variable region of the heavy chain (VH) selected from group of SEQ ID NO:15-16 and 40-55 (corresponding to B—HH2 to B—HH9 and B-HL8 to B-HL17 of WO 2005/044859 and WO 2007/031875). In some embodiments, the variable domain is selected from the group consisting of SEQ ID NO: 15, 16, 42, 44, 46, 48 and 50 (corresponding to B—HH2, BHH-3, B—HH6, B—HH8, B-HL8, B-HL11 and B-HL13 of WO 2005/044859 and WO 2007/031875). In some embodiments, the humanized B-Ly1 antibody has variable region of the light chain (VL) of SEQ ID NO:55 (corresponding to B—KV1 of WO 2005/044859 and WO 2007/031875). In some embodiments, the humanized B-Ly1 antibody has a variable region of the heavy chain (VH) of SEQ ID NO:42 (corresponding to B—HH6 of WO 2005/044859 and WO 2007/031875) and a variable region of the light chain (VL) of SEQ ID NO:55 (corresponding to B—KV1 of WO 2005/044859 and WO 2007/031875). In some embodiments, the humanized B-Ly1 antibody is an IgG1 antibody. Such afucosylated humanized B-Ly1 antibodies are glycoengineered (GE) in the Fc region according to the procedures described in WO 2005/044859, WO 2004/065540, WO 2007/031875, Umana, P. et al., Nature Biotechnol. 17 (1999) 176-180 and WO 99/154342. In some embodiments, the afucosylated glyco-engineered humanized B-Ly1 is B-HH6-B-KV1 GE. In some embodiments, the anti-CD20 antibody is obinutuzumab (recommended INN, WHO Drug Information, Vol. 26, No. 4, 2012, p. 453). As used herein, obinutuzumab is synonymous for GA101 or R05072759. It is commercially available for therapeutic use under the trade name GAZYVA®, and is provided as a 1000 mg/40 mL (25 mg/mL) single-dose vial. This replaces all previous versions (e.g., Vol. 25, No. 1, 2011, p. 75-76), and is formerly known as afutuzumab (recommended INN, WHO Drug Information, Vol. 23, No. 2, 2009, p. 176; Vol. 22, No. 2, 2008, p. 124). In some embodiments, the humanized B-Ly1 antibody is an antibody comprising a heavy chain comprising the amino acid sequence of SEQ ID NO:17 and a light chain comprising the amino acid sequence of SEQ ID NO:18, or an antigen-binding fragment thereof such antibody. In some embodiments, the humanized B-Ly1 antibody comprises a heavy chain variable region comprising the three heavy chain CDRs of SEQ ID NO:17 and a light chain variable region comprising the three light chain CDRs of SEQ ID NO:18.

In some embodiments, the humanized B-Ly1 antibody is an afucosylated glyco-engineered humanized B-Ly1. Such glycoengineered humanized B-Ly1 antibodies have an altered pattern of glycosylation in the Fc region, preferably having a reduced level of fucose residues. In some embodiments, the amount of fucose is about 60% or less of the total amount of oligosaccharides at Asn297 (in one embodiment the amount of fucose is between about 40% and about 60%, in another embodiment the amount of fucose is about 50% or less, and in still another embodiment the amount of fucose is about 30% or less). In some embodiments, the oligosaccharides of the Fc region are bisected. These glycoengineered humanized B-Ly1 antibodies have an increased ADCC.

The “ratio of the binding capacities to CD20 on Raji cells (ATCC-No. CCL-86) of an anti-CD20 antibodies compared to rituximab” is determined by direct immunofluorescence measurement (the mean fluorescence intensities (MFI) is measured) using said anti-CD20 antibody conjugated with Cy5 and rituximab conjugated with Cy5 in a FACSArray (Becton Dickinson) with Raji cells (ATCC-No. CCL-86), as described in Example No. 2, and calculated as follows:

$$Ratio of the binding capacities to CD 20 on Raji cells ( ATCC - No . CCL - 86 ) = \frac{MFI ( Cy 5 - anti - CD 20 antibody )}{MFI ( Cy 5 - rituximab )} \times \frac{Cy 5 - labeling ratio ( Cy 5 - rituximab )}{Cy 5 - labeling ratio ( Cy 5 - anti - CD 20 antibody )}$$

MFI is the mean fluorescent intensity. The “Cy5-labeling ratio” as used herein means the number of Cy5-label molecules per molecule antibody.

Typically said type II anti-CD20 antibody has a ratio of the binding capacities to CD20 on Raji cells (ATCC-No. CCL-86) of said second anti-CD20 antibody compared to rituximab of 0.3 to 0.6, and in one embodiment, 0.35 to 0.55, and in yet another embodiment, 0.4 to 0.5.

By “antibody having increased antibody dependent cellular cytotoxicity (ADCC)”, it is meant an antibody, as that term is defined herein, having increased ADCC as determined by any suitable method known to those of ordinary skill in the art.

An exemplary accepted in vitro ADCC assay is described below:
1) the assay uses target cells that are known to express the target antigen recognized by the antigen-binding region of the antibody;
2) the assay uses human peripheral blood mononuclear cells (PBMCs), isolated from blood of a randomly chosen healthy donor, as effector cells;
3) the assay is carried out according to following protocol:
   i) the PBMCs are isolated using standard density centrifugation procedures and are suspended at 5×10⁶ cells/ml in RPMI cell culture medium;
   ii) the target cells are grown by standard tissue culture methods, harvested from the exponential growth phase with a viability higher than 90%, washed in RPMI cell culture medium, labeled with 100 micro-Curies of ⁵¹Cr, washed twice with cell culture medium, and resuspended in cell culture medium at a density of 10′ cells/ml;
   iii) 100 microliters of the final target cell suspension above are transferred to each well of a 96-well microtiter plate;
   iv) the antibody is serially-diluted from 4000 ng/ml to 0.04 ng/ml in cell culture medium and 50 microliters of the resulting antibody solutions are added to the target cells in the 96-well microtiter plate, testing in triplicate various antibody concentrations covering the whole concentration range above;
   v) for the maximum release (MR) controls, 3 additional wells in the plate containing the labeled target cells, receive 50 microliters of a 2% (VN) aqueous solution of non-ionic detergent (Nonidet, Sigma, St. Louis), instead of the antibody solution (point iv above);
   vi) for the spontaneous release (SR) controls, 3 additional wells in the plate containing the labeled target cells, receive 50 microliters of RPMI cell culture medium instead of the antibody solution (point iv above);
   vii) the 96-well microtiter plate is then centrifuged at 50×g for 1 minute and incubated for 1 hour at 4° C.;
   viii) 50 microliters of the PBMC suspension (point i above) are added to each well to yield an effector:target cell ratio of 25:1 and the plates are placed in an incubator under 5% CO2 atmosphere at 37° C. for 4 hours;
   ix) the cell-free supernatant from each well is harvested and the experimentally released radioactivity (ER) is quantified using a gamma counter;
   x) the percentage of specific lysis is calculated for each antibody concentration according to the formula (ER-MR)/(MR-SR)×100, where ER is the average radioactivity quantified (see point ix above) for that antibody concentration, MR is the average radioactivity quantified (see point ix above) for the MR controls (see point V above), and SR is the average radioactivity quantified (see point ix above) for the SR controls (see point vi above);
4) “increased ADCC” is defined as either an increase in the maximum percentage of specific lysis observed within the antibody concentration range tested above, and/or a reduction in the concentration of antibody required to achieve one half of the maximum percentage of specific lysis observed within the antibody concentration range tested above. In one embodiment, the increase in ADCC is relative to the ADCC, measured with the above assay, mediated by the same antibody, produced by the same type of host cells, using the same standard production, purification, formulation and storage methods, which are known to those skilled in the art, except that the comparator antibody (lacking increased ADCC) has not been produced by host cells engineered to overexpress GnTIII and/or engineered to have reduced expression from the fucosyltransferase 8 (FUT8) gene (e.g., including, engineered for FUT8 knock out).

In some embodiments, the “increased ADCC” can be obtained by, for example, mutating and/or glycoengineering of said antibodies. In some embodiments, the anti-CD20 antibody is glycoengineered to have a biantennary oligosaccharide attached to the Fc region of the antibody that is bisected by GlcNAc. In some embodiments, the anti-CD20 antibody is glycoengineered to lack fucose on the carbohydrate attached to the Fc region by expressing the antibody in a host cell that is deficient in protein fucosylation (e.g., Lec13 CHO cells or cells having an alpha-1,6-fucosyltransferase gene (FUT8) deleted or the FUT gene expression knocked down). In some embodiments, the anti-CD20 antibody sequence has been engineered in its Fc region to enhance ADCC. In some embodiments, such engineered anti-CD20 antibody variant comprises an Fc region with one or more amino acid substitutions at positions 298, 333, and/or 334 of the Fc region (EU numbering of residues)).

In some embodiments, the term “complement-dependent cytotoxicity (CDC)” refers to lysis of human cancer target cells by the antibody according to the invention in the presence of complement. CDC can be measured by the treatment of a preparation of CD20 expressing cells with an anti-CD20 antibody according to the invention in the presence of complement. CDC is found if the antibody induces at a concentration of 100 nM the lysis (cell death) of 20% or more of the tumor cells after 4 hours. In some embodiments, the assay is performed with ⁵¹Cr or Eu labeled tumor cells and measurement of released ⁵¹Cr or Eu. Controls include the incubation of the tumor target cells with complement but without the antibody.

In some embodiments, the anti-CD20 antibody is a monoclonal antibody, e.g., a human antibody. In some embodiments, the anti-CD20 antibody is an antibody fragment, e.g., a Fv, Fab, Fab′, scFv, diabody, or F(ab′)₂ fragment. In some embodiments, the anti-CD20 antibody is a substantially full length antibody, e.g., an IgG1 antibody, IgG2a antibody or other antibody class or isotype as defined herein.

### VII. Antibodies

In some embodiments, an antibody (e.g., an anti-CD79b antibody or an anti-CD20 antibody) used in a method of treatment provided herein may incorporate any of the features, singly or in combination, as described in below.

### A. Antibody Affinity

In certain embodiments, an antibody (e.g., an anti-CD79b antibody or an anti-CD20 antibody) used in a method of treatment provided herein has a dissociation constant (Kd) of ≤1 μM, ≤100 nM, ≤50 nM, ≤10 nM, ≤5 nM, ≤1 nM, ≤0.1 nM, ≤0.01 nM, or ≤0.001 nM, and optionally is ≥10⁻¹³ M. (e.g., 10⁻⁸ M or less, e.g., from 10⁻⁸ M to 10⁻¹³ M, e.g., from 10⁻⁹ M to 10⁻¹³ M).

In one embodiment, Kd is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 g/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 l/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using surface plasmon resonance assays using a BIACORE®-2000 or a BIACORE®-3000 (BIAcore, Inc., Piscataway, NJ) at 25° C. with immobilized antigen CM5 chips at ˜10 response units (RU). Briefly, carboxymethylated dextran biosensor chips (CM5, BIACORE, Inc.) are activated with N-ethyl-N′—(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 g/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 l/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ See, e.g., Chen et al., J. Mol. Biol. 293:865-881 (1999). If the on-rate exceeds 10⁶M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

### B. Antibody Fragments

In certain embodiments, an antibody (e.g., an anti-CD79b antibody or an anti-CD20 antibody) used in a method of treatment provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as production by recombinant host cells (e.g., E. coli or phage), as described herein.

### C. Chimeric and Humanized Antibodies

In certain embodiments, an antibody a (e.g., an anti-CD79b antibody or an anti-CD20 antibody) used in a method of treatment provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### D. Human Antibodies

In certain embodiments, an antibody (e.g., an anti-CD79b antibody or an anti-CD20 antibody) used in a method of treatment provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### E. Library-Derived Antibodies

In some embodiments, an antibody (e.g., an anti-CD79b antibody or an anti-CD20 antibody) used in a method of treatment provided herein may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, NJ, 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, NJ, 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

### F. Multispecific Antibodies

In certain embodiments, an antibody (e.g., an anti-CD79b antibody or an anti-CD20 antibody) used in a method of treatment provided herein is a multispecific antibody, e.g., a bispecific antibody. Multispecific antibodies are monoclonal antibodies that have binding specificities for at least two different sites. In certain embodiments, one of the binding specificities is for one antigen (e.g., CD79b or CD20) and the other is for any other antigen. In certain embodiments, one of the binding specificities is for one antigen (e.g., CD79b or CD20) and the other is for CD3. See, e.g., U.S. Pat. No. 5,821,337. In certain embodiments, bispecific antibodies may bind to two different epitopes of an single antigen (e.g., CD79b or CD20). Bispecific antibodies may also be used to localize cytotoxic agents to cells which express the antigen (e.g., CD79b or CD20). Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g., US 2006/0025576A1).

The antibody or fragment herein also includes a “Dual Acting FAb” or “DAF” comprising an antigen binding site that binds to CD79b as well as another, different antigen (see, US 2008/0069820, for example).

### G. Antibody Variants

In certain embodiments, amino acid sequence variants of an antibody (e.g., an anti-CD79b antibody or an anti-CD20 antibody) used in a method of treatment provided herein are contemplated. For example, it may be desirable to improve the binding affinity and/or other biological properties of the anti-CD79b antibody or anti-CD20 antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### (i) Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table M under the heading of “preferred substitutions.” More substantial changes are provided in Table M under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE M

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp;Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp(W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Len, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g., binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex is used to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

### (ii) Glycosylation Variants

In certain embodiments, an antibody (e.g., an anti-CD79b antibody or an anti-CD20 antibody) used in a method of treatment provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

(iii) Fc Variants

In certain embodiments, one or more amino acid modifications may be introduced into the Fc region of an antibody (e.g., an anti-CD79b antibody or an anti-CD20 antibody) used in a method of treatment provided herein, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions.

In certain embodiments, the invention contemplates an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express Fc(RIII only, whereas monocytes express Fc(RI, Fc(RII and Fc(RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

### (iv) Cysteine Engineered Antibody Variants

In certain embodiments, it may be desirable to create cysteine engineered antibodies, e.g., “thioMAbs,” in which one or more residues of an anti-CD79b antibody or an anti-CD20 antibody used in a method of treatment provided herein are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

### (v) Antibody Derivatives

In certain embodiments, an antibody (e.g., an anti-CD79b antibody or an anti-CD20 antibody) used in a method of treatment provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### H. Recombinant Methods and Compositions

Antibodies may be produced using recombinant methods and compositions, e.g., as described in U.S. Pat. No. 4,816,567. In one embodiment, isolated nucleic acid encoding an antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g., a Chinese Hamster Ovary (CHO) cell or lymphoid cell (e.g., Y0, NS0, Sp20 cell). In one embodiment, a method of making an antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B.K.C. Lo, ed., Humana Press, Totowa, NJ, 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B.K.C. Lo, ed., Humana Press, Totowa, NJ), pp. 255-268 (2003).

### I. Assays

An antibody (e.g., an anti-CD79b antibody or an anti-CD20 antibody) used in a method of treatment provided herein may be identified, screened for, or characterized for their physical/chemical properties and/or biological activities by various assays known in the art.

In one aspect, an antibody (e.g., an anti-CD79b antibody or an anti-CD20 antibody) used in a method of treatment provided herein is tested for its antigen binding activity, e.g., by known methods such as ELISA, BIACore®, FACS, or Western blot.

In another aspect, competition assays may be used to identify an antibody that competes with any of the antibodies described herein for binding to the target antigen. In certain embodiments, such a competing antibody binds to the same epitope (e.g., a linear or a conformational epitope) that is bound by an antibody described herein. Detailed exemplary methods for mapping an epitope to which an antibody binds are provided in Morris (1996) “Epitope Mapping Protocols,” in Methods in Molecular Biology vol. 66 (Humana Press, Totowa, NJ).

In an exemplary competition assay, immobilized antigen is incubated in a solution comprising a first labeled antibody that binds to antigen (e.g., any of the antibodies described herein) and a second unlabeled antibody that is being tested for its ability to compete with the first antibody for binding to antigen. The second antibody may be present in a hybridoma supernatant. As a control, immobilized antigen is incubated in a solution comprising the first labeled antibody but not the second unlabeled antibody. After incubation under conditions permissive for binding of the first antibody to antigen, excess unbound antibody is removed, and the amount of label associated with immobilized antigen is measured. If the amount of label associated with immobilized antigen is substantially reduced in the test sample relative to the control sample, then that indicates that the second antibody is competing with the first antibody for binding to antigen. See Harlow and Lane (1988) Antibodies: A Laboratory Manual ch. 14 (Cold Spring Harbor Laboratory, Cold Spring Harbor, NY).

### VIII. Pharmaceutical Formulations

Pharmaceutical formulations of any of the agents described herein (e.g., anti-CD79b immunoconjugates, anti-CD20 agents, and immunomodulatory agents) for use in any of the methods as described herein are prepared by mixing such agent(s) having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody or immunoconjugate formulations are described in U.S. Pat. No. 6,267,958. Aqueous antibody or immunoconjugate formulations include those described in U.S. Pat. No. 6,171,586 and WO2006/044908, the latter formulations including a histidine-acetate buffer.

The formulation herein may also contain more than one active ingredient as necessary for the particular indication being treated, preferably those with complementary activities that do not adversely affect each other.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody or immunoconjugate, which matrices are in the form of shaped articles, e.g., films, or microcapsules.

The formulations to be used for in vivo administration are generally sterile. Sterility may be readily accomplished, e.g., by filtration through sterile filtration membranes.

Additional details regarding pharmaceutical formulations comprising an anti-CD79 immunoconjugate are provided in WO 2009/099728 the contents of which are expressly incorporated by reference herein in their entirety.

### IX. Kits and Articles of Manufacture

In another embodiment, an article of manufacture or a kit is provided comprising an anti-CD79b immunoconjugate (such as described herein) and at least one additional agent. In some embodiments the at least one additional agent is an immunomodulatory agent (such as lenalidomide) and an anti-CD20 antibody (such as obinutuzumab or rituximab). In some embodiments, the article of manufacture or kit further comprises package insert comprising instructions for using the anti-CD79b immunoconjugate in conjunction at least one additional agent, such as an immunomodulatory agent (e.g., lenalidomide) and an anti-CD20 antibody (e.g., obinutuzumab or rituximab) to treat or delay progression of a B-cell proliferative disorder (e.g., FL, such as relapsed/refractory FL) in an individual. Any of the anti-CD79b immunoconjugates and anti-cancer agents known in the art may be included in the article of manufacture or kits. In some embodiments, the kit comprises an immunoconjugate comprising the formula

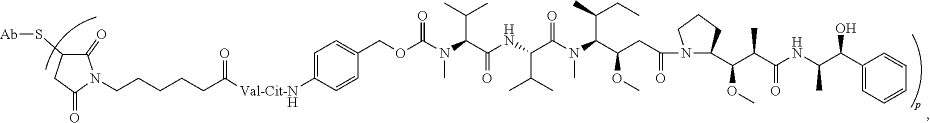

wherein Ab is an anti-CD79b antibody comprising (i) an HVR-H1 that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8. In some embodiments, the kit comprises an immunoconjugate comprising the formula

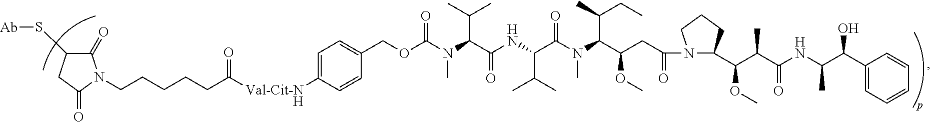

wherein Ab is an anti-CD79b antibody that comprises (i) a heavy chain comprising a VH that comprises the amino acid sequence of SEQ ID NO: 19 and (ii) a light chain comprising a VL that comprises the amino acid sequence of SEQ ID NO: 20, and wherein p is between 2 and 5. In some embodiments, p is between 3 and 4, e.g., 3.5. In some embodiments, the immunoconjugate comprises anti-CD79 antibody comprising a heavy chain comprising the amino acid sequence of SEQ ID NO: 36, and wherein the light chain comprises the amino acid sequence of SEQ ID NO: 35. In certain embodiments, the anti-CD79b immunoconjugate comprises the structure of Ab-MC-vc-PAB-MMAE. In some embodiments, the anti-CD79b immunoconjugate is polatuzumab vedotin (CAS Number 1313206-42-6). In some embodiments, the at least one additional agent is an immunomodulatory agent (such as lenalidomide) and an anti-CD20 antibody (such as obinutuzumab or rituximab).

In some embodiments, the kit is for use in the treatment of FL in an individual (e.g., an individual having one or more characteristics described herein) according to a method provided herein.

In some embodiments, the anti-CD79 immunoconjugate, the immunomodulatory agent (e.g., lenalidomide) and the anti-CD20 antibody (such as obinutuzumab or rituximab) are in the same container or separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

TABLE X
Amino Acid Sequences

| NAME | SEQUENCE | SEQ ID NO |
|---|---|---|
| Human CD79b | RFIARKRGFT VKMHCYMNSA SGNVSWLWKQ EMDENPQQLK | 1 |
| precursor; Acc. No. | LEKGRMEESQ NESLATLTIQ GIRFEDNGIY FCQQKCNNTS | |
| NP_000617.1; signal | EVYQGCGTEL RVMGFSTLAQ LKQRNTLKDG IIMIQTLLII | |
| sequence = amino | LFIIVPIFLL LDKDDSKAGM EEDHTYEGLD IDQTATYEDI | |
| acids 1 to 28 | VTLRTGEVKW SVGEHPGQE | |
| Human mature CD79b, | AR SEDRYRNPKG SACSRIWQSP RFIARKRGFT VKMHCYMNSA | 2 |
| without signal | SGNVSWLWKQ EMDENPQQLK LEKGRMEESQ NESLATLTIQ | |
| sequence; amino | GIRFEDNGIY FCQQKCNNTS EVYQGCGTEL RVMGFSTLAQ | |
| acids 29 to 229 | LKQRNTLKDG IIMIQTLLII LFIIVPIFLL LDKDDSKAGM | |
| | EEDHTYEGLD IDQTATYEDI VTLRTGEVKW SVGEHPGQE | |
| VH of mMAb anti- | Gly Pro Glu Leu Val Lys Pro Gly Ala Ser Val | 3 |
| CD20 antibody B-Ly1 | Lys Ile Ser Cys Lys Ala Ser Gly Tyr Ala Phe | |
| | Ser Tyr Ser Trp Met Asn Trp Val Lys Leu Arg | |
| | Pro Gly Gln Gly Leu Glu Trp Ile Gly Arg Ile | |
| | Phe Pro Gly Asp Gly Asp Thr Asp Tyr Asn Gly | |
| | Lys Phe Lys Gly Lys Ala Thr Leu Thr Ala Asp | |
| | Lys Ser Ser Asn Thr Ala Tyr Met Gln Leu Thr | |
| | Ser Leu Thr Ser Val Asp Ser Ala Val Tyr Leu | |
| | Cys Ala Arg Asn Val Phe Asp Gly Tyr Trp Leu | |
| | Val Tyr Trp Gly Gln Gly Thr Leu Val Thr Val | |
| | Ser Ala | |
| VL of mMAb anti- | Asn Pro Val Thr Leu Gly Thr Ser Ala Ser Ile | 4 |
| CD20 antibody B-Ly1 | Ser Cys Arg Ser Ser Lys Ser Leu Leu His Ser | |
| | Asn Gly Ile Thr Tyr Leu Tyr Trp Tyr Leu Gln | |
| | Lys Pro Gly Gln Ser Pro Gln Leu Leu Ile Tyr | |
| | Gln Met Ser Asn Leu Val Ser Gly Val Pro Asp | |
| | Arg Phe Ser Ser Ser Gly Ser Gly Thr Asp Phe | |
| | Thr Leu Arg Ile Ser Arg Val Glu Ala Glu Asp | |
| | Val Gly Val Tyr Tyr Cys Ala Gln Asn Leu Glu | |
| | Leu Pro Tyr Thr Phe Gly Gly Gly Thr Lys Leu | |
| | Glu Ile Lys Arg | |
| GA101 HVR-H1 | Gly Tyr Ala Phe Ser Tyr | 5 |
| GA101 HVR-H2 | Phe Pro Gly Asp Gly Asp Thr Asp | 6 |
| GA101 HVR-H3 | Asn Val Phe Asp Gly Tyr Trp Leu Val Tyr | 7 |
| GA101 HVR-L1 | Arg Ser Ser Lys Ser Leu Leu His Ser Asn Gly | |
| | Ile Thr Tyr Leu Tyr | 8 |
| GA101 HVR-L2 | Gln Met Ser Asn Leu Val Ser | 9 |
| GA101 HVR-L3 | Ala Gln Asn Leu Glu Leu Pro Tyr Thr | 10 |
| GA101 VH | Gln Val Gln Leu Val Gln Ser Gly Ala Glu Val | 11 |
| | Lys Lys Pro Gly Ser Ser Val Lys Val Ser Cys | |
| | Lys Ala Ser Gly Tyr Ala Phe Ser Tyr Ser Trp | |
| | Ile Asn Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| GA101 VL | Asp Ile Val Met Thr Gln Thr Pro Leu Ser Leu | 12 |
| | Pro Val Thr Pro Gly Glu Pro Ala Ser Ile Ser | |
| | Cys Arg Ser Ser Lys Ser Leu Leu His Ser Asn | |
| | Gly Ile Thr Tyr Leu Tyr Trp Tyr Leu Gln Lys | |
| | Pro Gly Gln Ser Pro Gln Leu Leu Ile Tyr Gln | |
| | Met Ser Asn Leu Val Ser Gly Val Pro Asp Arg | |
| | Phe Ser Gly Ser Gly Ser Gly Thr Asp Phe Thr | |
| | Leu Lys Ile Ser Arg Val Glu Ala Glu Asp Val | |
| | Gly Val Tyr Tyr Cys Ala Gln Asn Leu Glu Leu | |
| | Pro Tyr Thr Phe Gly Gly Gly Thr Lys Val Glu | |
| | Ile Lys Arg Thr Val | |
| GA101 Heavy Chain | Gln Val Gln Leu Val Gln Ser Gly Ala Glu Val | 13 |
| | Lys Lys Pro Gly Ser Ser Val Lys Val Ser Cys | |
| | Lys Ala Ser Gly Tyr Ala Phe Ser Tyr Ser Trp | |
| | Ile Asn Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser Ala Ser | |
| | Thr Lys Gly Pro Ser Val Phe Pro Leu Ala Pro | |
| | Ser Ser Lys Ser Thr Ser Gly Gly Thr Ala Ala | |
| | Leu Gly Cys Leu Val Lys Asp Tyr Phe Pro Glu | |
| | Pro Val Thr Val Ser Trp Asn Ser Gly Ala Leu | |
| | Thr Ser Gly Val His Thr Phe Pro Ala Val Leu | |
| | Gln Ser Ser Gly Leu Tyr Ser Leu Ser Ser Val | |
| | Val Thr Val Pro Ser Ser Ser Leu Gly Thr Gln | |
| | Thr Tyr Ile Cys Asn Val Asn His Lys Pro Ser | |
| | Asn Thr Lys Val Asp Lys Lys Val Glu Pro Lys | |
| | Ser Cys Asp Lys Thr His Thr Cys Pro Pro Cys | |
| | Pro Ala Pro Glu Leu Leu Gly Gly Pro Ser Val | |
| | Phe Leu Phe Pro Pro Lys Pro Lys Asp Thr Leu | |
| | Met Ile Ser Arg Thr Pro Glu Val Thr Cys Val | |
| | Val Val Asp Val Ser His Glu Asp Pro Glu Val | |
| | Lys Phe Asn Trp Tyr Val Asp Gly Val Glu Val | |
| | His Asn Ala Lys Thr Lys Pro Arg Glu Glu Gln | |
| | Tyr Asn Ser Thr Tyr Arg Val Val Ser Val Leu | |
| | Thr Val Leu His Gln Asp Trp Leu Asn Gly Lys | |
| | Glu Tyr Lys Cys Lys Val Ser Asn Lys Ala Leu | |
| | Pro Ala Pro Ile Glu Lys Thr Ile Ser Lys Ala | |
| | Lys Gly Gln Pro Arg Glu Pro Gln Val Tyr Thr | |
| | Leu Pro Pro Ser Arg Asp Glu Leu Thr Lys Asn | |
| | Gln Val Ser Leu Thr Cys Leu Val Lys Gly Phe | |
| | Tyr Pro Ser Asp Ile Ala Val Glu Trp Glu Ser | |
| | Asn Gly Gln Pro Glu Asn Asn Tyr Lys Thr Thr | |
| | Pro Pro Val Leu Asp Ser Asp Gly Ser Phe Phe | |
| | Leu Tyr Ser Lys Leu Thr Val Asp Lys Ser Arg | |
| | Trp Gln Gln Gly Asn Val Phe Ser Cys Ser Val | |
| | Met His Glu Ala Leu His Asn His Tyr Thr Gln | |
| | Lys Ser Leu Ser Leu Ser Pro Gly | |
| GA101 Light Chain | Asp Ile Val Met Thr Gln Thr Pro Leu Ser Leu | 14 |
| | Pro Val Thr Pro Gly Glu Pro Ala Ser Ile Ser | |
| | Cys Arg Ser Ser Lys Ser Leu Leu His Ser Asn | |
| | Gly Ile Thr Tyr Leu Tyr Trp Tyr Leu Gln Lys | |
| | Pro Gly Gln Ser Pro Gln Leu Leu Ile Tyr Gln | |
| | Met Ser Asn Leu Val Ser Gly Val Pro Asp Arg | |
| | Phe Ser Gly Ser Gly Ser Gly Thr Asp Phe Thr | |
| | Leu Lys Ile Ser Arg Val Glu Ala Glu Asp Val | |
| | Gly Val Tyr Tyr Cys Ala Gln Asn Leu Glu Leu | |
| | Pro Tyr Thr Phe Gly Gly Gly Thr Lys Val Glu | |
| | Ile Lys Arg Thr Val Ala Ala Pro Ser Val Phe | |
| | Ile Phe Pro Pro Ser Asp Glu Gln Leu Lys Ser | |
| | Gly Thr Ala Ser Val Val Cys Leu Leu Asn Asn | |
| | Phe Tyr Pro Arg Glu Ala Lys Val Gln Trp Lys | |
| | Val Asp Asn Ala Leu Gln Ser Gly Asn Ser Gln | |
| | Glu Ser Val Thr Glu Gln Asp Ser Lys Asp Ser | |
| | Thr Tyr Ser Leu Ser Ser Thr Leu Thr Leu Ser | |
| | Lys Ala Asp Tyr Glu Lys His Lys Val Tyr Ala | |
| | Cys Glu Val Thr His Gln Gly Leu Ser Ser Pro | |
| | Val Thr Lys Ser Phe Asn Arg Gly Glu Cys | |
| VH of humanized B- | Gln Val Gln Leu Val Gln Ser Gly Ala Glu Val | 15 |
| Ly1 antibody (B- | Lys Lys Pro Gly Ser Ser Val Lys Val Ser Cys | |
| HH2) | Lys Ala Ser Gly Tyr Ala Phe Ser Tyr Ser Trp | |
| | Met Asn Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Gln Val Gln Leu Val Gln Ser Gly Ala Glu Val | 16 |
| Ly1 antibody (B- | Lys Lys Pro Gly Ser Ser Val Lys Val Ser Cys | |
| HH3) | Lys Ala Ser Gly Tyr Ala Phe Ser Tyr Ser Trp | |
| | Met Asn Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Leu Cys Ala Arg Asn | |
| | Val Phe Asp Gly Tyr Trp Leu Val Tyr Trp Gly | |
| | Gln Gly Thr Leu Val Thr Val Ser Ser | |
| humanized B-Ly1 | QVQLVQSGAE VKKPGSSVKV SCKASGYAFS YSWINWVRQA | 17 |
| Heavy Chain | PGQGLEWMGR IFPGDGDTDY NGKFKGRVTI TADKSTSTAY | |
| | MELSSLRSED TAVYYCARNV FDGYWLVYWG QGTLVTVSSA | |
| | STKGPSVFPL APSSKSTSGG TAALGCLVKD YFPEPVTVSW | |
| | NSGALTSGVH TFPAVLQSSG LYSLSSVVTV PSSSLGTQTY | |
| | ICNVNHKPSN TKVDKKVEPK SCDKTHTCPP CPAPELLGGP | |
| | SVFLFPPKPK DTLMISRTPE VTCVVVDVSH EDPEVKFNWY | |
| | VDGVEVHNAK TKPREEQYNS TYRVVSVLTV LHQDWLNGKE | |
| | YKCKVSNKAL PAPIEKTISK AKGQPREPQV YTLPPSRDEL | |
| | TKNQVSLTCL VKGFYPSDIA VEWESNGQPE NNYKTTPPVL | |
| | DSDGSFFLYS KLTVDKSRWQ QGNVFSCSVM HEALHNHYTQ | |
| | KSLSLSPG | |
| humanized B-Ly1 | DIVMTQTPLS LPVTPGEPAS ISCRSSKSLL HSNGITYLYW | 18 |
| Light Chain | YLQKPGQSPQ LLIYQMSNLV SGVPDRFSGS GSGTDFTLKI | |
| | SRVEAEDVGV YYCAQNLELP YTFGGGTKVE IKRTVAAPSV | |
| | FIFPPSDEQL KSGTASVVCL LNNFYPREAK VQWKVDNALQ | |
| | SGNSQESVTE QDSKDSTYSL SSTLTLSKAD YEKHKVYACE | |
| | VTHQGLSSPV TKSFNRGEC | |
| huMA79bv28 heavy | EVQLVESGGG LVQPGGSLRL SCAASGYTFS SYWIEWVRQA | 19 |
| chain variable | PGKGLEWIGE ILPGGGDTNY NEIFKGRATF SADTSKNTAY | |
| region | LQMNSLRAED TAVYYCTRRV PIRLDYWGQG TLVTVSS | |
| huMA79bv28 light | DIQLTQSPSS LSASVGDRVT ITCKASQSVD YEGDSFLNWY | 20 |
| chain variable | QQKPGKAPKL LIYAASNLES GVPSRFSGSG SGTDFTLTIS | |
| region | SLQPEDFATY YCQQSNEDPL TFGQGTKVEI KR | |
| huMA79bv28 HVR H1 | GYTFSSYWIE | 21 |
| huMA79bv28 HVR H2 | GEILPGGGDTNYNEIFKG | 22 |
| huMA79bv28 HVR H3 | TRRVPIRLDY | 23 |
| huMA79bv28 HVR L1 | KASQSVDYEGDSFLN | 24 |
| huMA79bv28 HVR L2 | AASNLES | 25 |
| huMA79bv28 HVR L3 | QQSNEDPLT | 26 |
| huMA79bv28 heavy | EVQLVESGGGLVQPGGSLRLSCAAS | 27 |
| chain (HC) | | |
| framework region | | |
| (FR) 1 | | |
| huMA79bv28 HC FR2 | WVRQAPGKGLEWI | 28 |
| huMA79bv28 HC FR3 | RATFSADTSKNTAYLQMNSLRAEDTAVYYC | 29 |
| huMA79bv28 HC FR4 | WGQGTLVTVSS | 30 |
| huMA79bv28 light | DIQLTQSPSSLSASVGDRVTITC | 31 |
| chain (LC) FR1 | | |
| huMA79bv28 LC FR2 | WYQQKPGKAPKLLIY | 32 |
| huMA79bv28 LC FR3 | GVPSRFSGSGSGTDFTLTISSLQPEDFATYYC | 33 |
| huMA79bv28 LC FR4 | FGQGTKVEIKR | 34 |
| huMA79bv28 light | DIQLTQSPSS LSASVGDRVT ITCKASQSVD YEGDSFLNWY | 35 |
| chain (Igκ) | QQKPGKAPKL LIYAASNLES GVPSRFSGSG SGTDFTLTIS | |
| | SLQPEDFATY YCQQSNEDPL TFGQGTKVEI KRTVAAPSVF | |
| | IFPPSDEQLK SGTASVVCLL NNFYPREAKV QWKVDNALQS | |
| | GNSQESVTEQ DSKDSTYSLS STLTLSKADY EKHKVYACEV | |
| | THQGLSSPVT KSFNRGEC | |
| huMA79bv28 heavy | EVQLVESGGG LVQPGGSLRL SCAASGYTFS SYWIEWVRQA | 36 |
| chain (IgG1) | PGKGLEWIGE ILPGGGDTNY NEIFKGRATF SADTSKNTAY | |
| | LQMNSLRAED TAVYYCTRRV PIRLDYWGQG TLVTVSSAST | |
| | KGPSVFPLAP SSKSTSGGTA ALGCLVKDYF PEPVTVSWNS | |
| | GALTSGVHTF PAVLQSSGLY SLSSVVTVPS SSLGTQTYIC | |
| | NVNHKPSNTK VDKKVEPKSC DKTHTCPPCP APELLGGPSV | |
| | FLFPPKPKDT LMISRTPEVT CVVVDVSHED PEVKFNWYVD | |
| | GVEVHNAKTK PREEQYNSTY RVVSVLTVLH QDWLNGKEYK | |
| | CKVSNKALPA PIEKTISKAK GQPREPQVYT LPPSREEMTK | |
| | NQVSLTCLVK GFYPSDIAVE WESNGQPENN YKTTPPVLDS | |
| | DGSFFLYSKL TVDKSRWQQG NVFSCSVMHE ALHNHYTQKS | |
| | LSLSPG | |
| huMA79bv28 A118C | EVQLVESGGG LVQPGGSLRL SCAASGYTFS SYWIEWVRQA | 37 |
| cysteine engineered | PGKGLEWIGE ILPGGGDTNY NEIFKGRATF SADTSKNTAY | |
| heavy chain (IgG1) | LQMNSLRAED TAVYYCTRRV PIRLDYWGQG TLVTVSSCST | |
| | KGPSVFPLAP SSKSTSGGIA ALGCLVKDYF PEPVTVSWNS | |
| | GALTSGVHTF PAVLQSSGLY SLSSVVTVPS SSLGTQTYIC | |
| | NVNHKPSNTK VDKKVEPKSC DKTHTCPPCP APELLGGPSV | |
| | FLFPPKPKDT LMISRTPEVT CVVVDVSHED PEVKFNWYVD | |
| | GVEVHNAKTK PREEQYNSTY RVVSVLTVLH QDWLNGKEYK | |
| | CKVSNKALPA PIEKTISKAK GQPREPQVYT LPPSREEMTK | |
| | NQVSLTCLVK GFYPSDIAVE WESNGQPENN YKTTPPVLDS | |
| | DGSFFLYSKL TVDKSRWQQG NVFSCSVMHE ALHNHYTQKS | |
| | LSLSPG | |
| huMA79bv28 V205C | DIQLTQSPSS LSASVGDRVT ITCKASQSVD YEGDSFLNWY | 38 |
| cysteine engineered | QQKPGKAPKL LIYAASNLES GVPSRFSGSG SGTDFTLTIS | |
| light chain (Igκ) | SLQPEDFATY YCQQSNEDPL TFGQGTKVEI KRTVAAPSVE | |
| | IFPPSDEQLK SGTASVVCLL NNFYPREAKV QWKVDNALQS | |
| | GNSQESVTEQ DSKDSTYSLS STLTLSKADY EKHKVYACEV | |
| | THQGLSSPCT KSFNRGEC | |
| huMA79bv28 S400C | EVQLVESGGG LVQPGGSLRL SCAASGYTFS SYWIEWVRQA | 39 |
| cysteine engineered | PGKGLEWIGE ILPGGGDTNY NEIFKGRATF SADTSKNTAY | |
| heavy chain (IgG1) | LQMNSLRAED TAVYYCTRRV PIRLDYWGQG TLVTVSSAST | |
| | KGPSVFPLAP SSKSTSGGTA ALGCLVKDYF PEPVTVSWNS | |
| | GALTSGVHTF PAVLQSSGLY SLSSVVTVPS SSLGTQTYIC | |
| | NVNHKPSNTK VDKKVEPKSC DKTHTCPPCP APELLGGPSV | |
| | FLFPPKPKDT LMISRTPEVT CVVVDVSHED PEVKFNWYVD | |
| | GVEVHNAKTK PREEQYNSTY RVVSVLTVLH QDWLNGKEYK | |
| | CKVSNKALPA PIEKTISKAK GQPREPQVYT LPPSREEMTK | |
| | NQVSLTCLVK GFYPSDIAVE WESNGQPENN YKTTPPVLDC | |
| | DGSFFLYSKL TVDKSRWQQG NVFSCSVMHE ALHNHYTQKS | |
| | LSLSPGK | |
| VH of humanized B- | Gln Val Gln Leu Val Gln Ser Gly Ala Glu Val | 40 |
| Ly1 antibody (B- | Lys Lys Pro Gly Ala Ser Val Lys Val Ser Cys | |
| HH4) | Lys Val Ser Gly Tyr Ala Phe Ser Tyr Ser Trp | |
| | Met Asn Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Gln Val Gln Leu Val Gln Ser Gly Ala Glu Val | 41 |
| Ly1 antibody (B- | Lys Lys Pro Gly Ser Ser Val Lys Val Ser Cys | |
| HH5) | Lys Ala Ser Gly Tyr Ala Phe Ser Tyr Ser Trp | |
| | Met Ser Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Gln Val Gln Leu Val Gln Ser Gly Ala Glu Val | 42 |
| Ly1 antibody (B- | Lys Lys Pro Gly Ser Ser Val Lys Val Ser Cys | |
| HH6) | Lys Ala Ser Gly Tyr Ala Phe Ser Tyr Ser Trp | |
| | Ile Asn Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Gln Val Gln Leu Val Gln Ser Gly Ala Glu Val | 43 |
| Ly1 antibody (B- | Lys Lys Pro Gly Ser Ser Val Lys Val Ser Cys | |
| HH7) | Lys Ala Ser Gly Tyr Ala Phe Ser Tyr Ser Trp | |
| | Ile Ser Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Gln Val Gln Leu Val Gln Ser Gly Ala Glu Val | 44 |
| Ly1 antibody (B- | Lys Lys Pro Gly Ala Ser Val Lys Val Ser Cys | |
| HH8) | Lys Ala Ser Gly Tyr Thr Phe Thr Tyr Ser Trp | |
| | Met Asn Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Gln Val Gln Leu Val Gln Ser Gly Ala Glu Val | 45 |
| Ly1 antibody (B- | Lys Lys Pro Gly Ala Ser Val Lys Val Ser Cys | |
| HH9) | Lys Ala Ser Gly Tyr Thr Phe Ser Tyr Ser Trp | |
| | Met Asn Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Glu Val Gln Leu Val Glu Ser Gly Gly Gly Leu | 46 |
| Ly1 antibody (B- | Val Lys Pro Gly Gly Ser Leu Arg Leu Ser Cys | |
| HL8) | Ala Ala Ser Gly Phe Thr Phe Ser Tyr Ser Trp | |
| | Met Asn Trp Val Arg Gln Ala Pro Gly Lys Gly | |
| | Leu Glu Trp Val Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Glu Val Gln Leu Val Glu Ser Gly Gly Gly Leu | 47 |
| Ly1 antibody (B- | Val Lys Pro Gly Gly Ser Leu Arg Leu Ser Cys | |
| HL10) | Ala Ala Ser Gly Phe Ala Phe Ser Tyr Ser Trp | |
| | Met Asn Trp Val Arg Gln Ala Pro Gly Lys Gly | |
| | Leu Glu Trp Val Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Gln Val Gln Leu Val Glu Ser Gly Gly Gly Leu | 48 |
| Ly1 antibody (B- | Val Lys Pro Gly Gly Ser Leu Arg Leu Ser Cys | |
| HL11) | Ala Ala Ser Gly Phe Thr Phe Ser Tyr Ser Trp | |
| | Met Asn Trp Val Arg Gln Ala Pro Gly Lys Gly | |
| | Leu Glu Trp Val Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Glu Val Gln Leu Val Glu Ser Gly Ala Gly Leu | 49 |
| Ly1 antibody (B- | Val Lys Pro Gly Gly Ser Leu Arg Leu Ser Cys | |
| HL12) | Ala Ala Ser Gly Phe Thr Phe Ser Tyr Ser Trp | |
| | Met Asn Trp Val Arg Gln Ala Pro Gly Lys Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Glu Val Gln Leu Val Glu Ser Gly Gly Gly Val | 50 |
| Ly1 antibody (B- | Val Lys Pro Gly Gly Ser Leu Arg Leu Ser Cys | |
| HL13) | Ala Ala Ser Gly Phe Thr Phe Ser Tyr Ser Trp | |
| | Met Asn Trp Val Arg Gln Ala Pro Gly Lys Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Glu Val Gln Leu Val Glu Ser Gly Gly Gly Leu | 51 |
| Ly1 antibody (B- | Lys Lys Pro Gly Gly Ser Leu Arg Leu Ser Cys | |
| HL14) | Ala Ala Ser Gly Phe Thr Phe Ser Tyr Ser Trp | |
| | Met Asn Trp Val Arg Gln Ala Pro Gly Lys Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr s Ala Arg Asn | |
| | Val Phe Asp Gly Tyr Trp Leu Tyr Trp Gly | |
| | Gln Gly Thr Leu Val Thr Val Ser Ser | |
| VH of humanized B- | Glu Val Gln Leu Val Glu Ser Gly Gly Gly Leu | |
| Ly1 antibody (B- | Val Lys Pro Gly Ser Ser Leu Arg Leu Ser Cys | |
| HL15) | Ala Ala Ser Gly Phe Thr Phe Ser Tyr Ser Trp | |
| | Met Asn Trp Val Arg Gln Ala Pro Gly Lys Gly | |
| | Leu Glu Trp Met Gly Arg Ile Phe Pro Gly Asp | |
| | Gly Asp Thr Asp Tyr Asn Gly Lys Phe Lys Gly | 52 |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Glu Val Gln Leu Val Glu Ser Gly Gly Gly Leu | |
| Ly1 antibody (B- | Val Lys Pro Gly Gly Ser Leu Arg Val Ser Cys | |
| HL16) | Ala Ala Ser Gly Phe Thr Phe Ser Tyr Ser Trp | |
| | Met Asn Trp Val Arg Gln Ala Pro Gly Lys Gly | |
| | Leu Glu Trp Met Gly Arg Ile Phe Pro Gly Asp | 53 |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Glu Val Gln Leu Val Glu Ser Gly Gly Gly Leu | |
| Ly1 antibody (B- | Val Lys Pro Gly Gly Ser Leu Arg Leu Ser Cys | |
| HL17) | Ala Ala Ser Gly Phe Thr Phe Ser Tyr Ser Trp | |
| | Met Asn Trp Val Arg Gln Ala Pro Gly Lys Gly | |
| | Leu Glu Trp Met Gly Arg Ile Phe Pro Gly Asp | |
| | Gly Asp Thr Asp Tyr Asn Gly Lys Phe Lys Gly | 54 |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VL of humanized B- | Asp Ile Val Met Thr Gln Thr Pro Leu Ser Leu | |
| Ly1 antibody (B- | Pro Val Thr Pro Gly Glu Pro Ala Ser Ile Ser | |
| KVI) | Cys Arg Ser Ser Lys Ser Leu Leu His Ser Asn | |
| | Gly Ile Thr Tyr Leu Tyr Trp Tyr Leu Gln Lys | |
| | Pro Gly Gln Ser Pro Gln Leu Leu Ile Tyr Gln | |
| | Met Ser Asn Leu Val Ser Gly Val Pro Asp Arg | 55 |
| | Phe Ser Gly Ser Gly Ser Gly Thr Asp Phe Thr | |
| | Leu Lys Ile Ser Arg Val Glu Ala Glu Asp Val | |
| | Gly Val Tyr Tyr Cys Ala Gln Asn Leu Glu Leu | |
| | Pro Tyr Thr Phe Gly Gly Gly Thr Lys Val Glu | |
| | Ile Lys Arg Thr Val | |

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes.

### EXAMPLES

The following are examples of methods and compositions of the disclosure. It is understood that various other embodiments may be practiced, given the general description provided above.

### Example 1: An Anti-CD79b Immunoconjugate (Polatuzumab Vedotin) in Combination with Anti-CD20 Antibody (Obinutuzumab) and Lenalidomide in Relapsed or Refractory Follicular Lymphoma (FL)

Progress has been made in the treatment of follicular lymphoma (FL); however, a significant number of patients will relapse or die of progression or treatment-related toxicity. Patients who relapse after receiving several prior treatments may not be able to tolerate more bone marrow toxicity, thereby limiting their treatment options. There is a need for the continued development of safe and effective therapies for patients with disease that relapses and for patients who develop refractory disease during or after first-line therapy.

This Phase Ib/II, open label, multicenter, non-randomized, dose-escalation study evaluated the safety, efficacy, and pharmacokinetics of induction treatment consisting of obinutuzumab (GA101 or G) in combination with polatuzumab vedotin (anti-CD79b(huMA79b.v28)-MC-vc-PAB-MMAE ADC (DCDS4501A) or Pola) and lenalidomide (Len) (G+Pola+Len) in patients with relapsed or refractory (R/R) FL, followed by post induction treatment with obinutuzumab in combination with lenalidomide. The study included an initial dose-escalation phase, followed by an expansion phase during which polatuzumab vedotin and lenalidomide were given at their recommended Phase II doses (RP2Ds).

Responses were determined by an Independent Review Committee (IRC) and the investigator using Revised/Modified Lugano 2014 criteria (Cheson et al. (2014) J. Clin. Oncol. 32(27): 3059-3068). The primary efficacy endpoint was based on IRC assessment of response. Patients were monitored closely for adverse events throughout the study and for at least 90 days after the last dose of study treatment. To characterize the pharmacokinetic (PK) properties of obinutuzumab, polatuzumab vedotin, and lenalidomide, blood samples were obtained at various timepoints before and during study treatment administration

### Study Objectives

### Primary Efficacy Objective

The primary efficacy objective for this study was to evaluate the efficacy of induction treatment with G+Pola+Len on the basis of the following endpoint:
Percentage of Participants with complete response (CR) at the end of induction (EOI), determined by an IRC on the basis of Positron Emission Tomography (PET) and Computed Tomography (CT) Scans (PET-CT) using the Revised/Modified Lugano Response Criteria for Malignant Lymphoma (Cheson et al. 2014), hereinafter referred to as the Revised/Modified Lugano 2014 criteria or Modified Lugano 2014 Criteria.

The Revised/Modified Lugano 2014 criteria require normal bone marrow for patients with bone marrow involvement at screening (if indeterminate by morphology, immunohistochemistry should be negative). Additionally, designation of PET-CT-based partial response (PR) requires that CT-based response criteria for a CR or PR be met in addition to the PET-CT-based response criteria for a PR.

### Secondary Efficacy Objectives

The secondary efficacy objectives for this study were to evaluate the efficacy of induction treatment with G+Pola+Len and maintenance treatment with G+Len on the basis of the following endpoints:
CR at EOI, as determined by the investigator on the basis of PET-CT scans.
CR at EOI, as determined by the IRC and the investigator on the basis of CT scans alone.
Objective response (defined as a CR or PR) at EOI, as determined by the IRC and by the investigator on the basis of PET-CT scans.
Objective response (defined as a CR or PR) at EOI, as determined by the IRC and by the investigator on the basis of CT scans alone.
Best response of CR or PR during the study, as determined by the investigator on the basis of CT scans alone.

### Exploratory Efficacy Objectives

The exploratory efficacy objectives for this study were to evaluate the long-term efficacy of G+Pola+Len on the basis of the following endpoints:
For patients who had positive PET scans at EOI: CR at 12 months, as determined by the IRC and by the investigator on the basis of PET-CT scans.
PFS, defined as the time from initiation of study treatment (Cycle 1, day 1 of the induction phase) to first occurrence of disease progression or relapse, as determined by investigator on the basis of CT scans alone, or death from any cause.
Event-Free Survival (EFS), defined as the time from initiation of study treatment to any treatment failure, including disease progression or relapse, as determined by investigator on the basis of CT scans alone, initiation of new anti-lymphoma therapy, or death from any cause, whichever occurred first.
Disease-free survival (DFS), defined, among patients achieving a CR, as the time from the first occurrence of a documented CR to relapse, as determined by the investigator on the basis of CT scans alone, or death from any cause, whichever occurred first.
Overall survival (OS), defined as the time from initiation of study treatment to death from any cause.

### Safety Objectives

The safety objectives for this study were as follows:
To determine the recommended Phase II dose (RP2D) for polatuzumab vedotin and lenalidomide when given in combination with a fixed dose of obinutuzumab on the basis of the following endpoint:
   Incidence of DLTs during the first cycle of study treatment.
To evaluate the safety and tolerability of G+Pola+Len on the basis of the following endpoints:
   Nature, frequency, severity, and timing of adverse events, including DLTs.
   Changes in vital signs, ECGs, and clinical laboratory results during and following study treatment administration.

### Pharmacokinetic Objectives

The pharmacokinetic (PK) objective for this study was to characterize the PK profiles of obinutuzumab, polatuzumab vedotin, and lenalidomide when given in combination on the basis of the following endpoints:
Observed serum obinutuzumab concentration at specified timepoints.
Observed serum and plasma concentrations of polatuzumab vedotin and relevant analytes (total antibody, antibody-conjugated mono-methyl auristatin E, and unconjugated mono-methyl auristatin E) at specified timepoints.
Observed plasma lenalidomide concentration at specified timepoints.

### Immunogenicity Objectives

The immunogenicity objective for this study is to evaluate the immune response to obinutuzumab, and polatuzumab vedotin on the basis of the following endpoints:
Incidence of human anti-human antibodies (HAHAs) to obinutuzumab during the study relative to the prevalence of HAHAs at baseline.
Incidence of anti-therapeutic antibodies (ATAs) to polatuzumab vedotin during the study relative to the prevalence of ATAs at baseline.

The exploratory immunogenicity objective for this study was to evaluate potential relationships between HAHAs, and ATAs on the basis of the following endpoint: Correlation between HAHA, and ATA status and efficacy, safety, or PK endpoints.

### Biomarker Objectives

The exploratory biomarker objective for this study was to identify non-inherited biomarkers that are predictive of response to study treatment (i.e., predictive biomarkers), are associated with progression to a more severe disease state (i.e., prognostic biomarkers), are associated with acquired resistance to study treatment, are associated with susceptibility to developing adverse events, can provide evidence of study treatment activity, can increase the knowledge and understanding of lymphoma biology or study treatment mechanism of action, or can contribute to improvement of diagnostic assays on the basis of the following endpoint: Association between non-inherited biomarkers and efficacy, safety, pharmacokinetics, or immunogenicity endpoints.

### Study Design

### Patients

### Inclusion Criteria

For study entry, all patients met the following criteria:
18 years of age or older.
Eastern Cooperative Group (ECOG) Performance Status (PS) of 0-2.
Relapsed or refractory (R/R) FL (Grade 1, 2, 3a) after treatment with at least one prior chemoimmunotherapy regimen that included an anti-CD20 monoclonal antibody and for which no other more appropriate treatment option existed as determined by the investigator.
Histologically documented CD20-positive B-cell lymphoma as determined by the local laboratory.
Fluorodeoxyglucose-avid lymphoma (i.e., PET-positive lymphoma).
At least one bi-dimensionally measurable lesion (≥1.5 cm in its largest dimension by CT scan or magnetic resonance imaging).
Availability of a representative tumor specimen and the corresponding pathology report for retrospective central confirmation of the diagnosis of FL. If the archival tissue was unavailable or unacceptable, a pretreatment core-needle, excisional or incisional tumor biopsy was required. Cytological or fine-needle aspiration samples were not acceptable. If the patient received anti-lymphoma treatment between the time of the most recent available biopsy and initiation of study treatment, a repeat core-needle biopsy was strongly recommended.

### Exclusion Criteria

Patients who met any of the following criteria were excluded from study entry:
Grade 3b follicular lymphoma.
Known CD20-negative status at relapse or progression.
Central nervous system lymphoma or leptomeningeal infiltration.
Prior allogeneic stem-cell transplantation (SCT).
Completion of autologous SCT within 100 days prior to Day 1 of Cycle 1.
History of resistance to lenalidomide or response duration of <1 year (for patients who had a response to a prior lenalidomide-containing regimen).
Prior standard or investigational anti-cancer therapy as specified below:
   Lenalidomide, fludarabine, or alemtuzumab within 12 months prior to Day 1 of Cycle 1; radioimmunoconjugate within 12 weeks prior to Day 1 of Cycle 1.
   Monoclonal antibody or antibody-drug conjugate therapy within 5 half-lives or four weeks prior to Day 1 of Cycle 1, whichever was longer.
   Radiotherapy, chemotherapy, hormonal therapy, or targeted small-molecule therapy within 2 weeks prior to Day 1 of Cycle 1;
Clinically significant toxicity (other than alopecia) from prior therapy that had not resolved to Grade ≤2 per National Cancer Institute (NCI) Common Terminology Criteria for Adverse Events (CTCAE) (Version 4.0) (available at the website: http://ctep[dot]cancer[dot]gov/protocolDevelopment/electronic_applications/ctc[dot]htm) prior to Day 1 of Cycle 1.
Treatment with systemic immunosuppressive medications, including, but not limited to, prednisone, azathioprine, methotrexate, thalidomide, and anti-tumor necrosis factor agents within 2 weeks prior to Day 1 of Cycle 1.
   Treatment with inhaled corticosteroids and mineralocorticoids was permitted. If corticosteroid treatment was urgently required for lymphoma symptom control prior to the start of study treatment, up to 100 mg/day of prednisone or equivalent were given for a maximum of 5 days, but all tumor assessments were completed prior to initiation of corticosteroid treatment.
History of severe allergic or anaphylactic reaction to humanized or murine monoclonal antibodies.
Known sensitivity or allergy to murine products or any component of obinutuzumab, polatuzumab vedotin, or lenalidomide formulations.
History of erythema multiforme, Grade ≥3 rash or desquamation (blistering) following prior treatment with immunomodulatory derivatives such as thalidomide and lenalidomide.
Active bacterial, viral, fungal, or other infection; caution was exercised when considering the use of obinutuzumab in patients with a history of recurring or chronic infections.
Positive for hepatitis B surface antigen (HBsAg), total hepatitis B core antibody (HbcAb), or hepatitis C virus antibody (HCV) at screening.
Known history of Human Immunodeficiency Virus (HIV) positive status. For patients with unknown HIV status, HIV testing was performed at screening if required by local regulations.
History of progressive multifocal leukoencephalopathy.
Vaccination with a live virus vaccine within 28 days prior to Day 1 of Cycle 1.
History of other malignancy that could have affected compliance with the protocol or interpretation of results, with the exception of the following: curatively treated carcinoma in situ of the cervix; good-prognosis ductal carcinoma in situ of the breast; basal- or squamous-cell skin cancer; Stage I melanoma; low-grade, early-stage localized prostate cancer; any previously treated malignancy that had been in remission without treatment for ≥2 years prior to enrollment.
Contraindication to treatment for thromboembolism (TE) prophylaxis.
Current grade >1 peripheral neuropathy.
Evidence of any significant, uncontrolled concomitant disease that could have affected compliance with the protocol or interpretation of results, including significant cardiovascular disease (such as New York Heart Association Class III or IV cardiac disease, myocardial infarction within the previous 6 months, unstable arrhythmia, or unstable angina) or significant pulmonary disease (such as obstructive pulmonary disease or history of bronchospasm).
Major surgical procedure other than for diagnosis within 28 days prior to Day 1 of Cycle 1 or an anticipated major surgical procedure during the course of the study.
Inadequate renal or liver function.
Inadequate hematologic function (unless due to underlying lymphoma), defined as follows: Hemoglobin <9 g/dL, Absolute Neutrophil Count (ANC)<1.5×10⁹/L, platelet count <75×10⁹/L.
Any of the following abnormal laboratory values (unless due to underlying lymphoma): calculated creatinine clearance <50 mL/min (using the Cockcroft-Gault formula), Aspartate Aminotransferase (AST) or Alanine Aminotransferase (ALT) >2.5× upper limit of normal (ULN), serum total bilirubin >1.5×ULN (or >3×ULN for patients with Gilbert syndrome), INR or PT >1.5×ULN in the absence of therapeutic anticoagulation, and PTT or aPTT >1.5×ULN in the absence of a lupus anticoagulant.
Pregnant or lactating, or intending to become pregnant during the study.
   Women of childbearing potential had two negative serum pregnancy test results (minimum sensitivity, 25 mIU/mL) prior to initiating therapy: at 10-14 days prior to Day 1 of Cycle 1 and within 24 hours prior to Day 1 of Cycle 1.
Life expectancy <3 months.

### Study Treatment

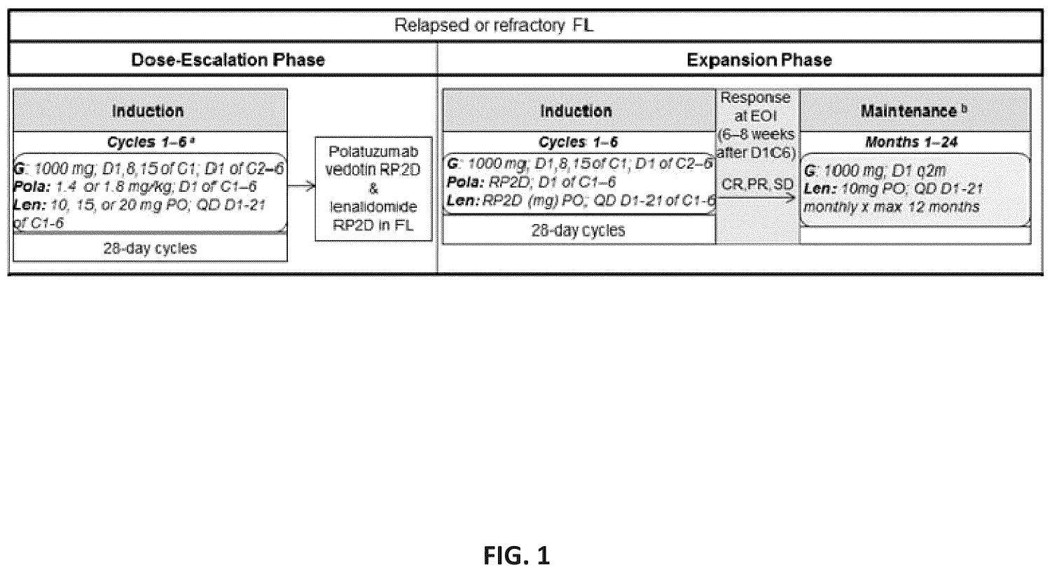

This study included an initial dose-escalation phase during which patients received obinutuzumab in combination with polatuzumab vedotin and lenalidomide. The dose-escalation phase was followed by an expansion phase, during which polatuzumab vedotin and lenalidomide were given at their RP2Ds in combination with obinutuzumab. Patients who achieved CR, PR or stable disease (SD) at EOI received maintenance treatment with lenalidomide and obinutuzumab. The dosing regiments for each phase are described below and provided in FIG. 1.

### Dose Escalation Phase

The purpose of the FL dose-escalation phase was to identify the RP2D for polatuzumab vedotin and the RP2D for lenalidomide when combined with a fixed dose of obinutuzumab as induction treatment.

Patients were closely monitored for adverse events during the dose limiting toxicity (DLT) assessment window, defined as the first treatment cycle (from Day 1 of Cycle 1 to Day 1 of Cycle 2). Patients experiencing a DLT during the DLT assessment period continued receiving study treatment once the event resolved if determined it was safe to continue treatment and there was potential for clinical benefit. Patients who discontinued from the study prior to completing the DLT assessment window for reasons other than a DLT were considered non-evaluable for dose-escalation decisions and RP2D assessments and were replaced by an additional patient at that same dose level. Patients who missed one or more doses of polatuzumab vedotin or obinutuzumab or five consecutive daily doses of lenalidomide during the DLT assessment window for reasons other than a DLT were also replaced and considered non-evaluable for dose-escalation decisions. Patients who received supportive care during the DLT assessment window that could confound the evaluation of DLTs were replaced at the discretion of the Medical Monitor. DLTs were defined as any one of the following events occurring during the first cycle of treatment and assessed by the investigator as related to study treatment and is not attributed to disease progression or another clearly identified cause:
Any adverse event of any grade that led to a delay of >14 days in the start of the next treatment cycle.
Any Grade 3 or 4 non-hematologic adverse event, except Grade 3 or 4 infusion related reactions (IRRs), Grade 3 diarrhea that responded to therapy within 72 hours.
Grade 3 nausea or vomiting that occurred in the absence of premedication and responded to adequate therapy within 72 hours, Grade 3 laboratory tumor lysis syndrome (TLS) without manifestations of clinical TLS (i.e., creatinine ≥1.5× upper limit of normal (ULN) and/or renal dysfunction, cardiac arrhythmias, seizures, or sudden death) that resolved within 7 days, Grade 3 fatigue that resolved to Grade ≤2 within 7 days, Grade 3 laboratory abnormality that was asymptomatic and deemed by the investigator not to be clinically significant, Grade 3 elevation in ALT or AST (provided that ALT or AST level was no greater than 8×ULN, ALT or AST elevation resolved to Grade <2 (<5 ULN) within 7 days, total and direct bilirubin and other laboratory parameters of liver synthetic function (e.g., prothrombin time) were normal, no clinical signs or symptoms of hepatic injury
Any increase in hepatic transaminase >3× baseline and an increase in direct bilirubin >2×ULN, without any findings of cholestasis or jaundice or signs of hepatic dysfunction and in the absence of other contributory factors (e.g., worsening of metastatic disease or concomitant exposure to known hepatotoxic agent or of a documented infectious etiology) is suggestive of potential drug-induced liver injury (according to Hy's Law) and was considered a DLT.
In patients with Grade 1 ALT or AST elevation at baseline as a result of liver metastases, only a Grade ≥3 elevation that is also ≥3× baseline lasting >7 days was considered a DLT.
Hematologic adverse event that met any of the following criteria: Grade 3 or 4 neutropenia in the presence of sustained fever of >38° C. (lasting >5 days) or a documented infection, Grade 4 neutropenia lasting >7 days, Grade 3 or 4 thrombocytopenia that resulted in significant bleeding per investigator judgment, Grade 4 thrombocytopenia lasting >7 days.
Other toxicities occurring during the first cycle that were considered to be clinically relevant and related to study treatment, as determined by the investigator and the Medical Monitor were considered DLTs.

### Induction Phase

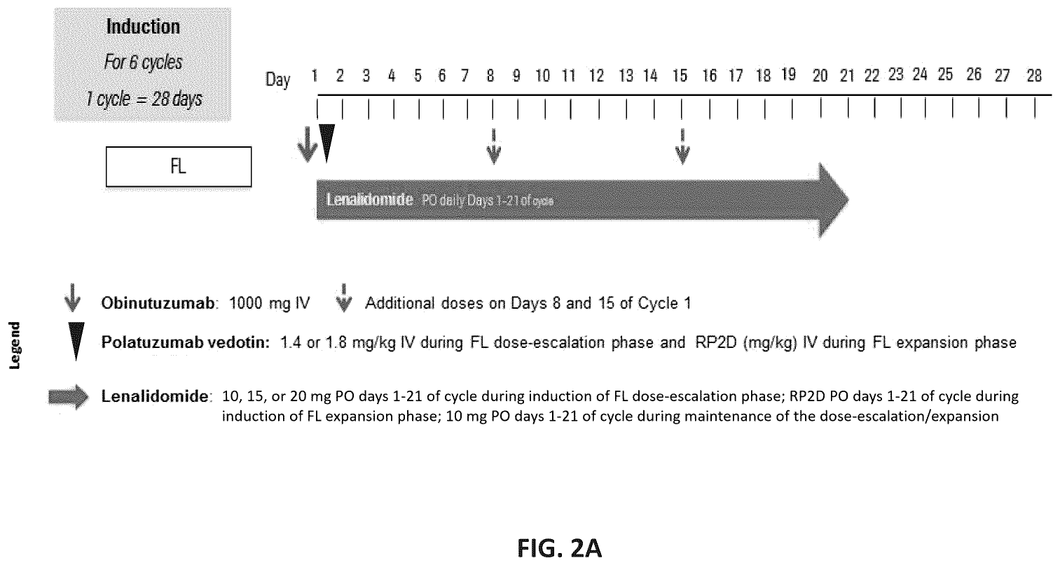

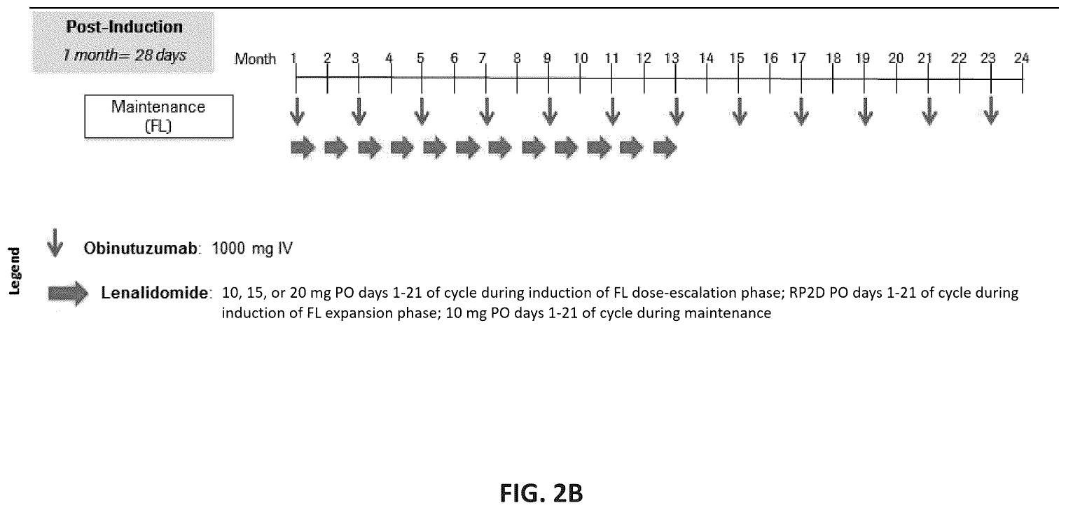

As shown in FIG. 2A, participants with R/R FL received 6 months of induction treatment with polatuzumab vedotin and lenalidomide at escalating doses to identify the recommended Phase 2 dose (RP2D) for polatuzumab vedotin and lenalidomide when combined with a fixed dose of obinutuzumab.

The induction treatment for the dose escalation phase is provided in Table 1. Patients received a fixed dose of 1000 mg obinutuzumab via intravenous (IV) infusion on Days 1, 8, and 15 of Cycle 1 and on Day 1 of each subsequent 28-day cycle for up to 6 cycles, polatuzumab vedotin doses of 1.4 mg/kg or 1.8 mg/kg via intravenous infusion on Day 1 of each 28-day cycle for up to 6 cycles, and lenalidomide doses of 10 mg, 15 mg, or 20 mg orally (PO) once daily on Days 1-21 of each 28-day cycle for up to 6 cycles. When study treatments were given on the same day, they were administered sequentially in the following order: lenalidomide, obinutuzumab, and polatuzumab vedotin.

TABLE 1
Induction treatment for the follicular lymphoma dose-escalation phase.

| | G + Pola + Len |
|---|---|
| Cycle | (28-Day Cycles) |
| Cycle 1 | Lenalidomide 10 mg, 15 mg, or 20 mg PO once daily on Days 1-21 |
| | Obinutuzumab 1000 mg IV on Days 1, 8, and 15 |
| | Polatuzumab vedotin 1.4 mg/kg or 1.8 mg/kg IV on Day 1 |
| Cycles 2-6 | Lenalidomide 10 mg, 15 mg, or 20 mg PO once daily on Days 1-21 |
| | Obinutuzumab 1000 mg IV on Day 1 |
| | Polatuzumab vedotin 1.4 mg/kg or 1.8 mg/kg IV on Day 1 |

G + Pola + Len = obinutuzumab in combination with polatuzumab vedotin and lenalidomide;
IV = intravenous; PO = by mouth.
Note:
Treatments were administered sequentially in the following order: lenalidomide, obinutuzumab, and polatuzumab vedotin.

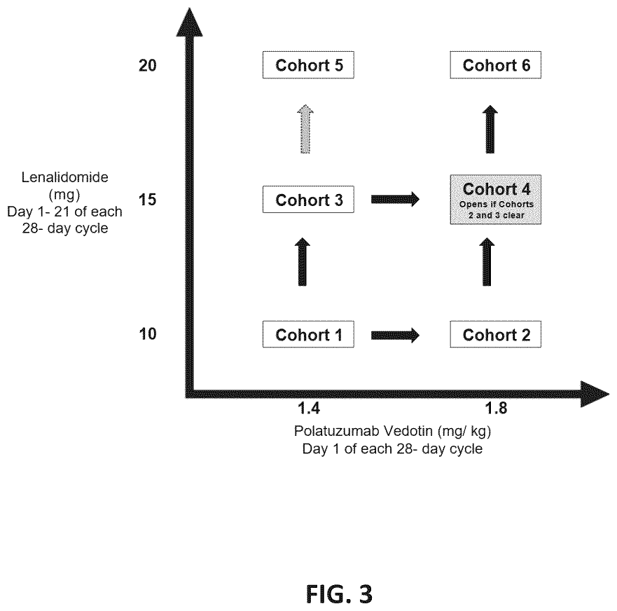

The FL dose-escalation plan is depicted in FIG. 3, and the doses for each cohort are summarized in Table 2. A standard 3+3 dose-escalation schema was used. The obinutuzumab dose remained fixed at 1000 mg during the dose-escalation phase. The starting doses in Cohort 1 were 1.4 mg/kg for polatuzumab vedotin and 10 mg for lenalidomide. In Cohorts 2-6, dose escalation of polatuzumab vedotin and lenalidomide proceeded in increments that paralleled the magnitude of dose increases tested in ongoing Phase Ib studies. For polatuzumab vedotin, there were two possible dose levels: 1.4 mg/kg or 1.8 mg/kg. For lenalidomide, there were three possible dose levels (10 mg, 15 mg, or 20 mg). Intrapatient dose escalation was not allowed.

TABLE 2
Follicular lymphoma dose-escalation cohorts.

| | | Polatuzumab^{b} | |
|---|---|---|---|
| Cohort | Obinutuzumab^{a} | Vedotin | Lenalidomide^{c} |
| 1 | 1000 mg | 1.4 mg/kg | 10 mg |
| 2 | 1000 mg | 1.8 mg/kg | 10 mg |
| 3 | 1000 mg | 1.4 mg/kg | 15 mg |
| 4 | 1000 mg | 1.8 mg/kg | 15 mg |
| 5 | 1000 mg | 1.4 mg/kg | 20 mg |
| 6 | 1000 mg | 1.8 mg/kg | 20 mg |

^{a}Obinutuzumab was administered intravenously at a fixed dose of 1000 mg. During Cycle 1, obinutuzumab was administered on Days 1, 8, and 15. During Cycles 2-6, obinutuzumab was administered on Day 1 only.
^{b}Polatuzumab vedotin was administered intravenously on Day 1 of each 28-day cycle.
^{c}Lenalidomide was administered orally on Days 1-21 of each 28-day cycle.

If Cohort 1 doses were deemed safe and tolerable, escalation continued with simultaneous enrollment of Cohort 2 (only the polatuzumab vedotin dose increased) and Cohort 3 (only the lenalidomide dose increased).

Escalation to Cohort 4 occurred only if Cohort 2 doses and Cohort 3 doses were deemed safe and tolerable.

If Cohort 4 doses were not tolerable, escalation continued with Cohort 5 (based on tolerated Cohort 3 dose combination, in which only the lenalidomide dose increased). If the Cohort 4 doses were safe and tolerable, further escalation occurred with enrollment of Cohort 6 (only the lenalidomide dose increased).

Dose escalation occurred in accordance with the rules listed below:
A minimum of three patients were initially enrolled in each cohort. The first three patients in each cohort were sequentially enrolled and dosed at least 48 hours apart.
If none of the first three DLT-evaluable patients experienced a DLT, the doses in that cohort were deemed safe and tolerable and escalation continued per the dose-escalation plan described above.
If one of the first three DLT-evaluable patients experienced a DLT, the cohort was expanded to six patients. If there were no further DLTs in the first six DLT-evaluable patients, the doses in that cohort were deemed safe and tolerable and escalation continued per the dose-escalation plan described above.
If a DLT was observed in ≥33% of patients (e.g., two or more of up to 6 DLT-evaluable patients), the dose combination at which this occurred was considered intolerable and the maximum tolerated dose (MTD) exceeded for polatuzumab vedotin and/or lenalidomide in the G+Pola+Len treatment combination. However, enrollment continued in alternative cohorts according to the dose-escalation plan described above.
If the MTD was exceeded in any cohort, the highest dose combination at which <33% of patients (e.g., 2 of 6 DLT-evaluable patients) experienced a DLT was declared the combination MTD (i.e., the MTDs for polatuzumab vedotin and lenalidomide in the G+Pola+Len treatment combination).
If the MTD was not exceeded at any dose level, the highest dose combination administered in this study was declared the maximum administered dose for polatuzumab vedotin and lenalidomide in the G+Pola+Len treatment combination.
If the MTD was exceeded in any cohort, de-escalation of the polatuzumab vedotin dose and/or the lenalidomide dose and adjustment of treatment schedules (e.g., lenalidomide treatment on Days 1-10) occurred.

### Expansion Phase

The expansion phase was designed to further assess the safety and efficacy of polatuzumab vedotin and lenalidomide at their respective RP2Ds when combined with a fixed dose of obinutuzumab in FL patients.

### Induction Phase

The induction treatment for the expansion phase is provided in Table 3. Patients received a fixed dose of 1000 mg obinutuzumab via intravenous infusion on Days 1, 8, and 15 of Cycle 1 and on Day 1 of each subsequent 28-day cycle for up to 6 cycles, polatuzumab vedotin RP2D doses (mg) IV on Day 1 of each 28-day cycle for up to 6 cycles, and lenalidomide RP2D doses orally once daily on Days 1-21 of each 28-day cycle for up to 6 cycles. When study treatments were given on the same day, they were administered sequentially in the following order: lenalidomide, obinutuzumab, and polatuzumab vedotin.

TABLE 3
Induction treatment for the FL expansion phase.

| | G + Pola + Len |
|---|---|
| Cycle | (28-Day Cycles) |
| Cycle 1 | Lenalidomide at the RP2D (mg) PO once daily on Days 1-21 |
| | Obinutuzumab 1000 mg IV on Days 1, 8, and 15 |
| | Polatuzumab vedotin at the RP2D (mg/kg) IV on Day 1 |
| Cycles 2-6 | Lenalidomide at the RP2D (mg) PO once daily on Days 1-21 |
| | Obinutuzumab 1000 mg IV on Day 1 |
| | Polatuzumab vedotin at the RP2D (mg/kg) IV on Day 1 |

G + Pola + Len = obinutuzumab in combination with polatuzumab vedotin and lenalidomide;
IV = intravenous; PO = by mouth; RP2D = recommended Phase II dose.
Note:
Treatments were administered sequentially in the following order: lenalidomide, obinutuzumab, and polatuzumab vedotin.

### Post-Induction Phase (Maintenance)

Patients who achieved CR, PR, or stable disease (SD) at the end of induction (EOI; 6-8 weeks after Day 1 of Cycle 6) received a 24-month maintenance regimen consisting of lenalidomide and obinutuzumab, which was initiated 8 weeks (+/−1 week) after Day 1 of Cycle 6 (induction cycle).

As shown in FIG. 2B, patients received a fixed dose of 1000 mg obinutuzumab intravenously on Day 1 of every other month for up to 24 months and lenalidomide doses of 10 mg orally once daily on Days 1-21 of each month for up to 12 months. Post-induction treatment continued for up to 24 months or until disease progression or unacceptable toxicity. No polatuzumab vedotin was administered post-induction.

### Assignment to Method of Treatment

During the dose-escalation phase, patients were assigned to cohorts with varying polatuzumab vedotin and lenalidomide dose combinations through use of an interactive voice or web-based response system (IxRS).

### Investigational Medicinal Products

### Obinutuzumab

Obinutuzumab was provided as a single-dose, sterile liquid formulation in a 50-mL glass vial containing 1000 mg/40 mL of obinutuzumab. In addition to the drug substance, the liquid was also composed of histidine, trehalose, and poloxamer 188.

### Polatuzumab Vedotin

Polatuzumab vedotin was supplied as a sterile, white to off-white, preservative-free lyophilisate in single-use vials.

### Lenalidomide

Lenalidomide was supplied as 5-, 10-, 15-, and 20-mg capsules.

### Administration of Study Treatment

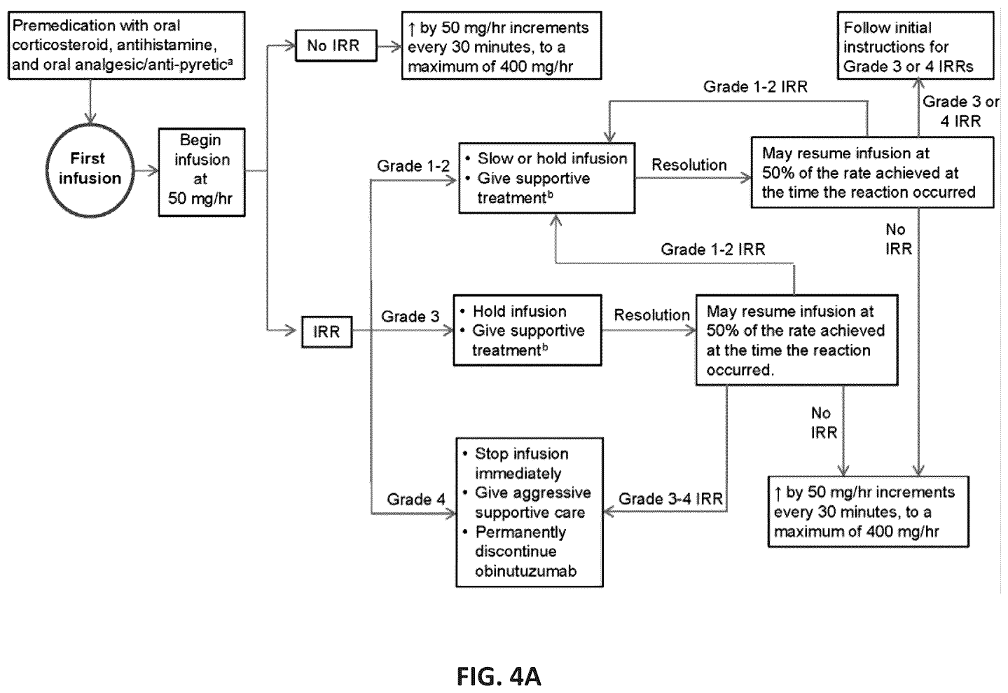

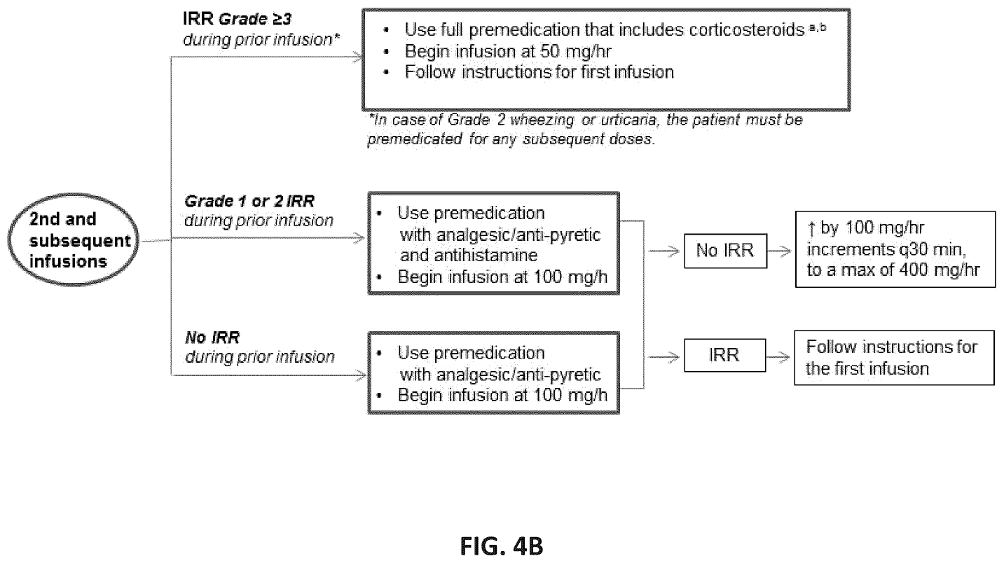

Obinutuzumab: Intravenous infusions at an absolute (flat) dose of 1000 mg were administered according to the instructions outlined in FIG. 4A for the first infusion and in FIG. 4B for the second and subsequent infusions. For patients with bulky lymphadenopathy, the infusion was given slowly over a longer period of time, or the dose was split and given over more than 1 day. No dose modifications for obinutuzumab were allowed.

Polatuzumab vedotin: The patient's weight obtained during screening (Days −28 to −1) was used for dose determination for all treatment cycles as described above. If the patient's weight within 96 hours prior to Day 1 of a given treatment cycle was >10% from the weight obtained during screening, the new weight was used to calculate the dose. After reconstitution with Sterile Water for Injection (SWFI) and dilution into IV bags containing isotonic sodium chloride solution (0.9% NaCl), polatuzumab vedotin was administered by IV infusion using dedicated standard administration sets with 0.2- or 0.22-μm in-line filters at a final polatuzumab vedotin concentration determined by the patient-specific dose. Compatibility of polatuzumab vedotin with IV bags, infusion lines, filters, and other infusion aids has been established with items made of specific materials of construction.

The initial dose was administered to patients who were well hydrated over 90 (+/−10) minutes. Premedications (e.g., 500-1000 mg of oral acetaminophen or paracetamol and 50-100 mg diphenhydramine as per institutional standard practice) were administered to an individual patient before administration of polatuzumab vedotin. Administration of corticosteroids was permitted at the discretion of the treating physician. If IRRs were observed with the first infusion in the absence of premedication, premedication was administered before subsequent doses.

The polatuzumab vedotin infusion was slowed or interrupted for patients experiencing infusion-associated symptoms. Following the initial dose, patients were observed for 90 minutes. If prior infusions were well tolerated, subsequent doses of polatuzumab vedotin were administered over 30 (+/−10) minutes, followed by a 30-minute observation period after the infusion.

The dose of polatuzumab vedotin was reduced due to neurotoxicity only according to the following dose reduction steps based on the starting dose as provided in Table 4.

TABLE 4
Polatuzumab vedotin dose-reduction steps.

| | |
|---|---|
| Starting | Dose Reduction |

| Dose | Step 1 | Step 2 |
|---|---|---|
| 1.8 mg/kg | 1.4 mg/kg | none |
| 1.4 mg/kg | none | none |

Lenalidomide: Lenalidomide was administered orally as described above. If a dose of lenalidomide was missed and it had been <12 hours since the time of the scheduled dose, the patient took the missed dose. If it had been >12 hours, the dose was skipped and the next dose was taken at the regularly scheduled time. Two doses were not taken at the same time. If a dose was vomited, the dose was not re-taken.

The dose of lenalidomide could be reduced in 5-mg increments one or two times during induction or post-induction, depending on the starting dose, as outlined in Table 5. No more than one dose reduction was allowed per treatment cycle. If the lenalidomide dose was reduced to 5 mg during induction, the maintenance dose was escalated to start 10 mg in post-induction if considered safe per the investigator judgement. In all other cases, if lenalidomide dose was reduced, re-escalation was not permitted.

If a lenalidomide-related toxicity occurred during lenalidomide treatment (i.e., before Day 21 of the cycle), lenalidomide was withheld until criteria for recovery were met (i.e., improved to Grade ≤2 or baseline values).

If recovery was observed prior or on Day 15 of the cycle, lenalidomide was resumed at the same dose for the remainder of the cycle (through Day 21; missed doses were not made up) at the discretion of the investigator. If the investigator considered that resuming lenalidomide at the same dose within the cycle represented an unacceptable risk for the patient, lenalidomide was resumed at reduced dose or withheld for the remainder of the cycle. For subsequent cycles, lenalidomide was resumed at reduced doses. If recovery was observed after Day 15 of the cycle, lenalidomide was not resumed for the current cycle. For subsequent cycles, lenalidomide was resumed at reduced doses.

TABLE 5
Lenalidomide dose-reduction steps.

| | |
|---|---|
| Starting | Dose Reduction |

| Dose | Step 1 | Step 2 |
|---|---|---|
| 20 mg | 15 mg | 10 mg |
| 15 mg | 10 mg | 5 mg |
| 10 mg | 5 mg | none |

Premedications and other required medications: Lenalidomide increases the risk of thromboembolism (TE). All patients were required to take daily aspirin (75-100 mg) for TE prophylaxis during lenalidomide treatment and until 28 days after the last dose of lenalidomide. Patients who were unable to tolerate aspirin, patients with a history of TE, and patients at high risk of TE received warfarin or low-molecular-weight heparin (LMWH). Patients received premedication as provided in Table 6.

TABLE 6
Outline of premedications.

| | Patients | | |
|---|---|---|---|
| | Requiring | | |
| Timepoint | Premedication | Premedication | Administration |
| Cycle 1, | All patients | Oral corticosteroid ^{a} | Complete ≥1 hour prior to |
| Day 1 | | | obinutuzumab infusion |
| | All patients | Antihistamine drug ^{b} | Administer ≥30 minutes prior |
| | | Oral | to obinutuzumab infusion |
| | | analgesic/antipyretic ^{c} | |
| | Patients at risk | Allopurinol or | Administer prior to |
| | for TLS (e.g., | suitable | obinutuzumab infusion |
| | because of | alternative, such | |
| | bulky disease | as rasburicase, | |
| | or renal | along with | |
| | impairment | adequate | |
| | [creatinine | hydration | |
| | clearance <70 | | |
| | mL/min]) | | |
| Cycle 1, | Patients | Oral analgesic/anti- | Administer at least 30 minutes |
| Days 8 | with no IRR | pyretic ^{c} | prior to obinutuzumab infusion. |
| and 15 | during the | | |
| Cycles | previous | | |
| 2 and | infusion | | |
| Beyond, | Patients | Antihistamine drug ^{b} | Administer ≥30 minutes |
| Day 1 | with | Oral | prior to obinutuzumab infusion |
| | Grade 1 or 2 IRR | analgesic/antipyretic ^{c} | |
| | during | | |
| | the | | |
| | previous | | |
| | infusion | | |
| | Patients with | Oral corticosteroid ^{a} | Complete ≥1 hour prior |
| | Grade 3 IRR, | | to obinutuzumab infusion |
| | wheezing, | Antihistamine drug ^{b} | Administer ≥30 minutes |
| | urticarial, or | Oral | prior to obinutuzumab infusion |
| | other | analgesic/antipyretic ^{c} | |
| | symptoms of | | |
| | anaphylaxis | | |
| | during the | | |
| | previous | | |
| | infusion | | |
| | Patients with | | |
| | bulky disease | | |
| | Patients still at | Allopurinol or | Administer prior to |
| | risk for TLS | suitable | obinutuzumab infusion |
| | | alternative, such | |
| | | as rasburicase, | |
| | | along with | |
| | | adequate | |
| | | hydration | |

IRR = infusion-related reaction; TLS = tumor lysis syndrome.
^{a} Treat with 100 mg of prednisone or prednisolone, 20 mg of dexamethasone, or 80 mg of methylprednisolone. Hydrocortisone was not used.
^{b} For example, 50 mg of diphenhydramine.
^{c} For example, 1000 mg of acetaminophen/paracetamol.

### Management of Toxicities and Adverse Events

Study treatment was delayed for toxicity for a maximum amount of time, as specified below (e.g., see Table 7 and Table 8). If study treatment was delayed for longer than the specified maximum amount of time, study treatment was permanently discontinued. When a treatment cycle was delayed because of toxicity resulting from any component of the regimen, all study treatment was held and resumed together to remain synchronized. If one drug was discontinued, treatment with the other two drugs was continued for patients experiencing clinical benefit as determined by the investigator after discussing with the Medical Monitor.

Treatment delays applied to all toxicities described below; dose modifications apply only to toxicities that were considered to be related to lenalidomide or polatuzumab vedotin (only for peripheral neuropathy). There were no dose reductions of obinutuzumab. For patients receiving obinutuzumab, if toxicity occurred before Cycle 1 Day 8 or Cycle 1 Day 15, these doses of obinutuzumab were not skipped but given after resolution of toxicity.

### Hematological Toxicities During Induction Treatment

Hematologic toxicity was defined as neutropenia, anemia, or thrombocytopenia. Lymphopenia was not considered a hematologic toxicity, but rather an expected outcome of therapy. Table 7 provides guidelines for management of hematologic toxicities that occurred during induction treatment, with the exception of Days 8 and 15 of Cycle 1 for patients receiving obinutuzumab.

TABLE 7
Guidelines for Management of Hematologic Toxicides That Occurred during Induction
Treatment (Except Days 8 and 15 of Cycle 1 for Patients Receiving Obinutuzumab).

| Event | Action Taken |
|---|---|
| Grade 3 or 4 | For patients on a lenalidomide dose ≥10 mg who have had one or no prior |
| hematologic | lenalidomide dose reductions: |
| toxicity^{a, b} | Withhold study treatment.^{a} |
| | Administer RBCs or platelets as required. |
| | If patient has not already initiated G-CSF, initiate prophylactic G-CSF for |
| | current and subsequent cycles. |
| | For patients who develop platelet count of <20,000/μL while receiving LMWH, |
| | reduce the dose of LMWH. For patients who develop platelet count of <20,000/μL |
| | while receiving platelet inhibitors, consider temporarily withholding platelet |
| | inhibitors. |
| | Permanently discontinue study treatment if any of the following events occur: |
| | Grade 3 or 4 thrombocytopenia that results in significant bleeding per |
| | investigator judgment |
| | Recurrent Grade 3 or 4 neutropenia associated with fever >38° C. lasting |
| | >5 days or documented infection despite use of G-CSF and after one |
| | lenalidomide dose reduction |
| | Recurrent Grade 4 neutropenia or thrombocytopenia lasting >7 days despite |
| | use of G-CSF (for neutropenia) and after one lenalidomide dose reduction |
| | If improvement to Grade ≤2 or baseline ≤14 days after the scheduled date for the |
| | next cycle, resume obinutuzumab and polatuzumab vedotin at full dose and resume |
| | lenalidomide at current dose. |
| | If improvement to Grade ≤2 or baseline 15-21 days after the scheduled date for the |
| | next cycle, resume obinutuzumab and polatuzumab vedotin at full dose and resume |
| | lenalidomide at a reduced dose for current and subsequent cycles. |
| | If study treatment is withheld for >21 days, permanently discontinue study |
| | treatment. |
| | For patients who have had two prior dose reductions: |
| | Permanently discontinue study treatment. |

G = obinutuzumab; G-CSF = granulocyte colony-stimulating factor; LMWH = low-molecular-weight heparin.
^{a}Treatment delays apply to all toxicities; dose modifications apply only to toxicities that are considered to be related to any of the study treatment components. Toxicities that occur during the cycle and subside prior to the next cycle should not trigger the suggested dose modifications.
^{b}If cytopenia is thought to be caused mainly by B-cell lymphoma infiltration of the bone marrow, the investigator may decide not to reduce the lenalidomide dose.

Table 8 provides guidelines for management of hematologic toxicities that occurred at Days 8 and 15 of Cycle 1, when patients received treatment with obinutuzumab only.

TABLE 8
Guidelines for Management of Hematologic Toxicides That Occurred
on Days 8 and 15 of Cycle 1 for Patients Receiving Obinutuzumab.

| Event | Action Taken |
|---|---|
| Febrile | Withhold obinutuzumab and lenalidomide until resolution of fever |
| neutropenia or | and infection (as applicable). |
| neutropenia with | If the event is ongoing at Day 1 of Cycle 2, follow instructions |
| documented | in Table 7. |
| infection | Note: |
| | Obinutuzumab and lenalidomide were not withheld for asymptomatic |
| | neutropenia. |
| Severe | Withhold obinutuzumab and lenalidomide until platelet |
| thrombocytopenia ^{a} | count is ≥50,000/pL and there is resolution of bleeding. |
| or bleeding | If receiving LMWH, reduce the dose. |
| | If receiving platelet inhibitors, consider temporarily withholding |
| | platelet inhibitors. |
| | If the event is ongoing at Day 1 of Cycle 2, follow instructions |
| | in Table 7. |

LMWH = low-molecular-weight heparin.
^{a} Severe thrombocytopenia is defined as a platelet count <10,000/μL for patients who are not receiving concomitant anticoagulants or platelet inhibitors and <20,000/μL for patients who are receiving concomitant anticoagulants or platelet inhibitors.

### Non-Hematological Toxicities During Induction Treatment

General guidance for treatment delays and discontinuation were:
If study treatment was withheld for >21 days because of a toxicity that was attributable to study treatment, permanently discontinue study treatment.
When a treatment cycle was delayed because of toxicity resulting from any component of the regimen, all study treatment was held and resumed together to remain synchronized.
If one drug was discontinued, treatment with the other two drugs was continued for patients experiencing clinical benefit as determined by the investigator after discussion with the Medical Monitor.

### Toxicities During Maintenance Treatment

Table 9 provides guidelines for management of toxicities that occurred during maintenance treatment.

TABLE 9
Guidelines for Management of Toxicities that
Occurred during Maintenance Treatment.

| Event | Action Taken |
|---|---|
| Hematologic toxicity: Grade 3 or 4 | Withhold obinutuzumab and |
| | lenalidomide. |
| | Administer G-CSF for neutropenia |
| | per institutional guidelines. |
| | Administer RBCs or platelets as |
| | required. |
| | If improvement to Grade ≤2, resume |
| | obinutuzumab and lenalidomide at same |
| | dose. Lenalidomide dose may be |
| | reduced by one dose level per |
| | investigator judgment following |
| | discussion with the medical |
| | monitor. |
| | If study treatment is withheld |
| | for >42 days, permanently |
| | discontinue study treatment. |
| Non-hematologic toxicity: Grade ≥2 | Withhold obinutuzumab and |
| | lenalidomide. |
| | If improvement to Grade ≤1 or baseline, |
| | administer study treatment at full dose. |
| | Lenalidomide dose may be reduced by |
| | one dose level per investigator judgment |
| | following discussion with the medical |
| | monitor. |
| | If study treatment is withheld |
| | for >42 days, permanently |
| | discontinue study treatment. |

G-CSF = granulocyte colony-stimulating factor.

### Study Treatment Discontinuation

Study treatment was permanently discontinued in patients who experienced any of the following:
Anaphylaxis, acute respiratory distress, or Grade 4 IRR.
If a Grade 3 IRR was recurrent during the second or subsequent cycles, study treatment was discontinued at the discretion of the investigator, following an individual benefit-risk assessment.
Any adverse event that met criteria for permanent discontinuation per guidelines provided above.
Pregnancy.
Disease progression.

### Safety and Efficacy Assessments

### Determination of Sample Size

It was anticipated that enrollment of 5 cohorts of 3-6 patients each, for a total of 18-30 patients, were required to establish the RP2D during the dose-escalation phase for patients with R/R FL. The primary efficacy analysis is the estimation of the true proportion of patients expected to obtain a PET-CT-defined CR at EOI. A sample size of 40 patients was deemed sufficient to provide adequate precision for the point estimate and for the lower bound of the two-sided 90% CI to rule out a clinically uninteresting probability of response of <55%, assuming an observed PET-CT-defined CR rate of 70%.

### Safety Assessments

Safety assessments consist of monitoring and recording adverse events, including serious adverse events and non-serious adverse events of special interest, performing protocol-specified safety laboratory assessments, measuring protocol-specified vital signs, and conducting other protocol-specified tests that were deemed critical to the safety evaluation of the study.

The safety analyses include all treated patients (i.e., patients who received any amount of study treatment). Safety is assessed through summaries of adverse events and changes from baseline in laboratory test results, shift-tables of ECGs findings, and vital signs. All adverse events occurring on or after first study treatment are summarized by mapped term, appropriate thesaurus levels, and NCI CTCAE, Version 4.0 grade. All serious adverse events, adverse events of special interest, and selected adverse events are summarized and listed. Deaths reported during the treatment period and during post-treatment follow-up are listed and summarized. Relevant laboratory results are displayed by time, with Grade 3 and 4 values identified as appropriate.

Adverse Events: NCI CTCAE, Version 4.0 is used for assessing adverse event severity. All adverse events are reported until 90 days after the last dose of study treatment. After this period, the investigator reports any serious adverse events that are believed to be related to prior study treatment and events of second malignancies for patients who received obinutuzumab. Grade 3 and 4 infections (both related and unrelated) are reported until up to 2 years after the last dose of obinutuzumab.

In general, adverse events that are secondary to other events (e.g., cascade events or clinical sequelae) are identified by their primary cause, with the exception of severe or serious secondary events. A medically significant secondary adverse event that is separated in time from the initiating event is recorded as an independent event.

Persistent adverse events (extend continuously, without resolution, between patient evaluation timepoints) are recorded once. Each recurrence of a recurrent adverse events (resolves between patient evaluation timepoints and subsequently recurs) is recorded as a separate event.

Infusion-Related Reactions: Adverse events that occur during or within 24 hours after the end of study treatment infusion and are judged to be related to infusion of any of the study treatment components are captured as a diagnosis (e.g., “infusion-related reaction”). If a patient experiences both a local and systemic reaction to the same dose of study treatment, each reaction is recorded separately, with signs and symptoms also recorded separately.

Abnormal Laboratory Values: Not every laboratory abnormality qualifies as an adverse event. A laboratory test result is reported as an adverse event if it meets any of the following criteria:
Accompanied by clinical symptoms.
Results in a change in study treatment (e.g., dosage modification, treatment interruption, or treatment discontinuation).
Results in a medical intervention (e.g., potassium supplementation for hypokalemia) or a change in concomitant therapy.
Clinically significant in the investigator's judgment.
For oncology trials, certain abnormal values may not qualify as adverse events.

Abnormal Vital Sign Values: Not every vital sign abnormality qualifies as an adverse event. A vital sign result is reported as an adverse event if it meets any of the following criteria:
Accompanied by clinical symptoms.
Results in a change in study treatment (e.g., dosage modification, treatment interruption, or treatment discontinuation)
Results in a medical intervention or a change in concomitant therapy.
Clinically significant in the investigator's judgment.

Abnormal Liver Function Tests: Treatment-emergent ALT or AST >3× baseline value in combination with total bilirubin >2×ULN (of which ≥35% is direct bilirubin) and Treatment-emergent ALT or AST >3× baseline value in combination with clinical jaundice are reported as adverse events.

Deaths: For this protocol, mortality is an efficacy endpoint. Deaths that occur during the protocol-specified adverse event reporting period that are attributed by the investigator solely to progression of lymphoma are recorded only on the Study Completion/Early Discontinuation electronic Case Report Form (eCRF). All other on-study deaths, regardless of relationship to study treatment, are recorded on the Adverse Event eCRF.

Pre-existing Medical Conditions: A preexisting medical condition is one that was present at the screening visit for this study. A preexisting medical condition is recorded as an adverse event only if the frequency, severity, or character of the condition worsened during the study.

Lack of Efficacy or Worsening of Lymphoma: Events that were clearly consistent with the expected pattern of progression of the underlying disease are not recorded as adverse events. These data are captured as efficacy assessment data only. In most cases, the expected pattern of progression is based on the Lugano 2014 criteria. In rare cases, the determination of clinical progression is based on symptomatic deterioration.

Hospitalization or Prolonged Hospitalization: Any adverse event that results in hospitalization (i.e., in-patient admission to a hospital) or prolonged hospitalization is documented and reported as a serious adverse event except as outlined below:
Hospitalization for respite care.
Planned hospitalization required by the protocol (e.g., for study treatment administration or insertion of access device for study treatment administration)
Hospitalization for a preexisting condition, provided that all of the following criteria are met:
   The hospitalization was planned prior to the study or was scheduled during the study when elective surgery became necessary because of the expected normal progression of the disease.
   The patient has not experienced an adverse event.
   Hospitalization due solely to progression of the underlying cancer.

An event that leads to hospitalization under the following circumstance is not considered to be a serious adverse event, but is reported as an adverse event instead: Hospitalization that was necessary because of patient requirement for outpatient care outside of normal outpatient clinic operating hours.

### Efficacy Assessments

The primary and secondary efficacy analyses include the primary efficacy population (patients who received at least one dose of any component of the combination) and the intent-to-treat population (all patients enrolled in the study) for patients enrolled in the expansion phase. In addition, patients with FL who receive polatuzumab vedotin and lenalidomide at the RP2D during the dose-escalation phases are pooled for analysis by histology with patients treated in the expansion phase at the same dose levels. Response is determined on the basis of PET-CT scans or CT scans alone, using the Revised/Modified Lugano 2014 criteria.

For the primary efficacy endpoint, secondary efficacy endpoints, and exploratory efficacy endpoints, point estimates are presented, along with the corresponding two-sided 90% Clopper-Pearson exact CIs. Patients without a post-baseline tumor assessment are considered non-responders.

PFS, EFS, DFS, and OS are summarized descriptively using the Kaplan-Meier method (Kaplan and Meier, 1958). For the PFS, EFS, and DFS analyses, data for patients without an event of interest is censored at the date of the last tumor assessment. For patients without post-baseline tumor assessments, data is censored at the date of initiation of study treatment plus 1. For the OS analysis, data for patients who have not died is censored at the date the patient was last known to be alive. Where medians are reached, the corresponding estimated median is provided, along with the 95% CI estimated using the method of Brookmeyer and Crowley (1982). In addition, landmark estimates of the proportion of patients who are event free at 6 months, 9 months, 1 year, and 2 years are provided, along with 95% asymptotic CIs using Greenwood's formula for standard errors.

In this study, minimal residual disease (MRD) is quantified by circulating lymphoma cells and circulating tumor DNA as an exploratory endpoint. The lymphoma clone is identified in DNA from the lymphoma tissue specimen. MRD levels are determined in blood samples collected prior to dosing and during treatment to explore a pharmacodynamic (PD) relationship. MRD assessments are performed at EOI to allow for an evaluation of the depth of response, and during and after post-induction treatment to allow for an evaluation of long-term response or possible disease recurrence.

### Pharmacokinetic Analyses

Plasma/serum concentrations of obinutuzumab, polatuzumab vedotin, and lenalidomide are tabulated, summarized, and plotted after appropriate grouping. As appropriate, PK parameters (e.g., area under the curve [AUC], time to maximum concentration [tₘₐₓ], maximum concentration [Cₘₐₓ], and half-life [t_{1/2}]) are also calculated, tabulated, and summarized after appropriate grouping. Additional PK and PK/PD analyses (e.g., population modelling including pooled analyses across studies) are also performed as appropriate. All analyses may be extended to include relevant biotransformation products of polatuzumab vedotin or lenalidomide.

### Immunogenicity Analyses

The numbers and proportions of post-treatment HAHA- and ATA-positive patients and HAHA- and ATA-negative patients at baseline and during both the treatment and follow-up periods are summarized by histologic subtype. Patients are considered to be ATA positive if they are ATA negative at baseline but develop an ATA response following study treatment administration (treatment-induced ATA response) or if they are ATA positive at baseline and the titer of one or more post-baseline samples is at least 4-fold greater (i.e., ≥0.60 titer units) than the titer of the baseline sample (treatment-enhanced ATA response). Patients are considered to be ATA negative if they are ATA negative at baseline and all post-baseline samples are negative or if they are ATA positive at baseline but do not have any post-baseline samples with a titer that is at least 4-fold greater than the titer of the baseline sample (treatment unaffected). The relationship between HAHA, and ATA status and safety, efficacy, PK, and biomarker endpoints are explored as appropriate.

### Biomarker Analyses

The association between candidate biomarkers and PET-CT-defined CR rate and objective response (CR+PR) rate, and potentially other measures of efficacy and safety, are explored to assess potential prognostic or predictive value.

Measurement of relevant protein, RNA, and DNA from tissue specimens is assessed for biomarkers associated with disease biology (immune gene expression profiles and disease subtype gene expression patterns and associated mutations, i.e., MYD88 and CD79b), mechanism of action of study drugs (i.e., including but not limited to regulated substrates of lenalidomide, i.e., CRBN, MYC, IRF4, or immune repertoire signatures), mechanisms of resistance, and improvement of diagnostic assays.

Exploratory biomarker research includes, but is not limited to: target expression BCL2 and CD79b, immune infiltrate, cereblon (and surrogates); Lymphoma-related genetic changes (DNA) and gene expression (mRNA) or protein expression (immunohistochemistry associated with response or potential resistance); Lymphoma index clone in MRD; Circulating lymphoma cells and/or cell-free circulating tumor DNA (detection of minimal residual disease); Lymphocyte immunophenotyping, including B-cell counts (CD19), T-cell counts (CD3, CD4, and CD8), and NK-cell counts (CD16 and CD56); Cytokines characteristic of T-cell activation and lenalidomide activity (e.g., IL-8 and IFNγ).

### Interim Analyses

One interim analysis was conducted during the expansion phase of the study, when at least 15 patients had been evaluated for PET-CT-defined CR at EOI. See results below.

### Post-Treatment and Survival Follow-Up

Patients who complete treatment or discontinue treatment for reasons other than disease progression undergo assessments every 3 months during the post-treatment follow-up period, which continues until disease progression, the start of new anti-lymphoma treatment, or the end of the study (as defined below), whichever occurs first. Patients who experience disease progression are evaluated for survival status and initiation of new anti-lymphoma treatment every 3 months until the end of the study.

### End of Study and Length of Study

The end of this study is defined as the time when all enrolled patients with FL have been followed for at least 90 days after they have completed or discontinued study treatment (including induction treatment and maintenance treatment as applicable). The total length of the study, from screening of the first patient to the end of the study, is approximately 5 years.

### Results

The results of a pre-planned interim analysis of the safety and efficacy of induction and maintenance with Pola-G-Len in patients with R/R FL in this study are reported herein.

### Patient Characteristics

### Evaluable Populations

The safety-evaluable population was 52 patients: 16 patients from the dose-escalation cohort (10 patients were not treated at the RP2D and 6 patients completed the RP2D induction) and 36 patients from the dose-expansion cohort (24 patients had ongoing induction treatment and 12 patients completed the RP2D induction). The median duration of follow-up was 6 months.

The efficacy-evaluable population included 18 patients: 6 patients from the dose-escalation cohort and 12 patients from the dose-expansion cohort that completed RP2D induction.

### Baseline Characteristics

Patient baseline characteristics are provided in Table 10. The median patient age was 62 years, with a range of 32-87 years. Patients were classified using the Follicular Lymphoma International Prognostic Index (FLIPI), showing that 58% of patients were classified as being in the High Risk Group, with 3-5 FLIPI Risk Factors. Seven patients (13%) were classified as being in the Low FLIPI Risk Group (0-1 Risk Factors), and 15 patients (29%) were classified as being in the Intermediate FLIPI Risk Group (2 Risk Factors). The percentage of patients that had ≥2 prior therapy lines was 79%, and the percentage of patients that were refractory to the last treatment was 50%

TABLE 10
Patient baseline characteristics.

| | SAFETY POPULATION | |
|---|---|---|
| CHARACTERISTIC | n = 52 | |
| Median age, years (range) | 62 | (32-87) |
| ECOG PS 0-1, n (%) | 51 | (98) |
| Ann Arbor Stage III/IV, n (%) | 34 | (65) |
| Bulky disease (≥7 cm), n (%) | 9 | (17) |
| FLIPI ≥3, n (%) | 30 | (58) |
| Number of prior lines of treatment, n (%) | | |
| 1 | 11 | (21) |
| 2 | 11 | (21) |
| ≥3 | 30 | (58) |
| Median prior lines of treatments (range) | 3 | (1-7) |
| Refractory to last prior therapy¹, n (%) | 26 | (50) |

### Safety

### Adverse Events

A summary of all adverse events (AEs) is provided in Table 11. Grade 3-4 adverse events were experienced by 75% of patients. One patient (2%) experienced a Grade 5 AE (septic shock after progressive disease in patient receiving new anti-lymphoma treatment (TAK-659, tyrosine kinase inhibitor)).

The majority of dose interruptions (29%) were due to neutropenia, followed by IRRs (12%).

AEs leading to lenalidomide dose reduction occurred in 31% of patients. AEs leading to lenalidomide dose interruptions occurred in 52% of patients.

TABLE 11
Summary of all adverse events.

| ALL ADVERSE EVENTS, n (%) | n = 52 | |
|---|---|---|
| Patients with at least one AE | 52 | (100) |
| Grade 5 AEs | 1 | (2) |
| Grade 3-4 AEs | 39 | (75) |
| Serious AEs | 21 | (40) |
| AEs leading to dose reduction | 16 | (31) |
| AEs leading to dose interruption | 31 | (60) |
| AEs leading to any drug discontinuation | 8 | (15) |

The most common AEs were infections (56%), neutropenia (52%), thrombocytopenia (37%), IRRs (35%), pyrexia (35%), anemia (33%), and diarrhea (29%). A summary of AEs occurring in ≥10% of patients is provided in Table 12.

TABLE 12
Summary of adverse events occurring in ≥10% of patients.

| ALL ADVERSE EVENTS n (%) | n = 52 | |
|---|---|---|
| Infections¹ | 29 | (56) |
| Neutropenia | 27 | (52) |
| Thrombocytopenia | 19 | (37) |
| Infusion-related reaction | 18 | (35) |
| Pyrexia | 18 | (35) |
| Anemia | 17 | (33) |
| Diarrhea | 15 | (29) |
| Rash | 11 | (21) |
| ALT increased | 10 | (19) |
| Fatigue | 10 | (19) |
| Peripheral neuropathy² | 9 | (17) |
| Asthenia | 8 | (15) |
| Cough | 8 | (15) |
| AST Increased | 7 | (14) |
| Blood creatinine increased | 7 | (14) |
| Constipation | 7 | (14) |
| Decreased appetite | 7 | (14) |
| Nausea | 7 | (14) |
| Hypokalemia | 6 | (12) |
| Nasopharyngitis | 6 | (12) |
| Pruritis | 6 | (12) |

¹Infections presented as Systems Organ Class terms; all other adverse events are reported by ‘preferred terms’.
²Peripheral neuropathy SMQ-w includes: peripheral motor neuropathy, peripheral sensory neuropathy, neuropathy peripheral, and paresthesia.

Of the 11 patients who experienced Rash, 9 patients (17.3%) experienced Rash, 1 patient (1.9%) experienced Rash Erythematous, and 1 patient (1.9%) experienced Rash Macular.

In addition, 4 patients (7.7%) experienced paraesthesia, 3 patients (5.8%) experienced neuropathy peripheral, 1 patient (1.9%) experienced peripheral motor neuropathy, and 1 patient (1.9%) experienced peripheral sensory neuropathy.

A summary of Grade 3-4 adverse events occurring in 2 patients is provided in Table 13. Grade 3-4 adverse events were experienced by 75% of patients. The most common hematologic Grade 3-4 AE was neutropenia (46%). The most common non-hematologic Grade 3-4 AE was Infections (12%).

TABLE 13
Summary of Grade 3-4 AEs occurring in in ≥2 patients.

| Grade 3-4 adverse events n (%) | n = 52 | |
|---|---|---|
| Total number of patients with Grade 3-4 AEs | 39 | (75) |

Hematologic

| | | |
|---|---|---|
| Neutropenia¹ | 24 | (46) |
| Thrombocytopenia | 9 | (17) |
| Anemia | 6 | (12) |
| Febrile neutropenia | 2 | (4) |

Non-hematologic

| | | |
|---|---|---|
| Infections² | 6 | (12)³ |
| ALT increased | 2 | (4) |
| Lipase increased | 2 | (4) |
| Hypokalemia | 2 | (4) |
| Tumor lysis syndrome | 2 | (4) |

ALT = alanine aminotransferase.
¹Granulocyte colony stimulating factor use reported in 24 (46%) patients.
²Infections presented as Systems Organ Class terms; all other adverse events are reported by ‘preferred terms’.
³Lower respiratory tract infection (n = 2), septic shock, epididymitis, cavernous sinus thrombosis, and urinary tract infection.

### Study Discontinuation

Of the 52 patients, nine discontinued study treatment: four patients died due to disease progression (PD) (all were in the dose escalation population, not at RP2D), and five patients being treated at the RP2D discontinued study treatment. Of the five patients treated at the RP2D who discontinued treatment, three patients discontinued study treatment due to adverse events, one patient withdrew from study treatment, one patient discontinued study treatment for other reasons (subsequent stem cell transplant), and none experienced death attributable to PD.

### Efficacy

The recommended Phase II doses (RP2D) for polatuzumab vedotin and lenalidomide when combined with a fixed dose of obinutuzumab were determined to be 1.4 mg/kg and 20 mg, respectively.

Preliminary efficacy data based on PET-CT show high activity of the combination of polatuzumab vedotin, lenalidomide, and obinutuzumab. As shown in Table 14, at the end of induction (EOI) treatment, the Best Overall Response percentage was 89%, irrespective of whether it was assessed by either the investigator or the IRC and regardless of whether the Modified Lugano 2014 or Lugano 2014 criteria were used. Complete responses were observed in at least 61% of patients (using the Modified Lugano 2014 criteria: 61% when assessed by the investigator and 67% when assessed by the IRC; using the Lugano 2014 criteria: 78% when assessed by the investigator and the IRC). Partial responses were observed in at least n % of patients (using the Modified Lugano 2014 criteria: 28% when assessed by the investigator and 22% when assessed by the IRC; using the Lugano 2014 criteria: 1% when assessed by the investigator and the IRC). One patient (6%) exhibited stable disease and no patients exhibited progressive disease.

TABLE 14
Responses at EOI (efficacy-evaluable population; RP2D; N = 18).

| End of induction Response | Modified Lugano 2014¹ | | | | Lugano 2014 | | | |
|---|---|---|---|---|---|---|---|---|
| n = 18, n (%) | INV | | IRC | | INV | | IRC | |
| Objective Response | 16 | (89) | 16 | (89) | 16 | (89) | 16 | (89) |
| Complete Response | 11 | (61)² | 12 | (67)² | 14 | (78) | 14 | (78) |
| Partial Response | 5 | (28) | 4 | (22) | 2 | (11) | 2 | (11) |
| Stable Disease | 1 | (6) | 1 | (6) | 1 | (6) | 1 | (6) |
| Progressive Disease | 0 | | 0 | | 0 | | 0 | |
| Missing/unevaluable | 1 | (6)³ | 1 | (6)³ | 1 | (6)³ | 1 | (6)³ |

¹Modified Lugano requires a negative bone marrow biopsy to confirm PET-CR; PET-PR must also meet CT-PR criteria.
²CR downgraded to PR due to missing bone marrow biopsy at EOI in 3 patients by INV and 2 patients by IRC.
³1 patient had partial response by CT (interim scan) but no PET at EOI performed before stem cell transplant.
CR = complete response; CT = computed tomography; EOI = end of induction; INV = investigator assessed; IRC = independent review committee assessed; PET = positron emission tomography; PR = partial response.
Reasons for missing bone marrow biopsies (BM): 1 patient declined BM, 1 investigator declined BM, 1 BM inadvertently missed.
No patients were downgraded due to persistent BM positivity.

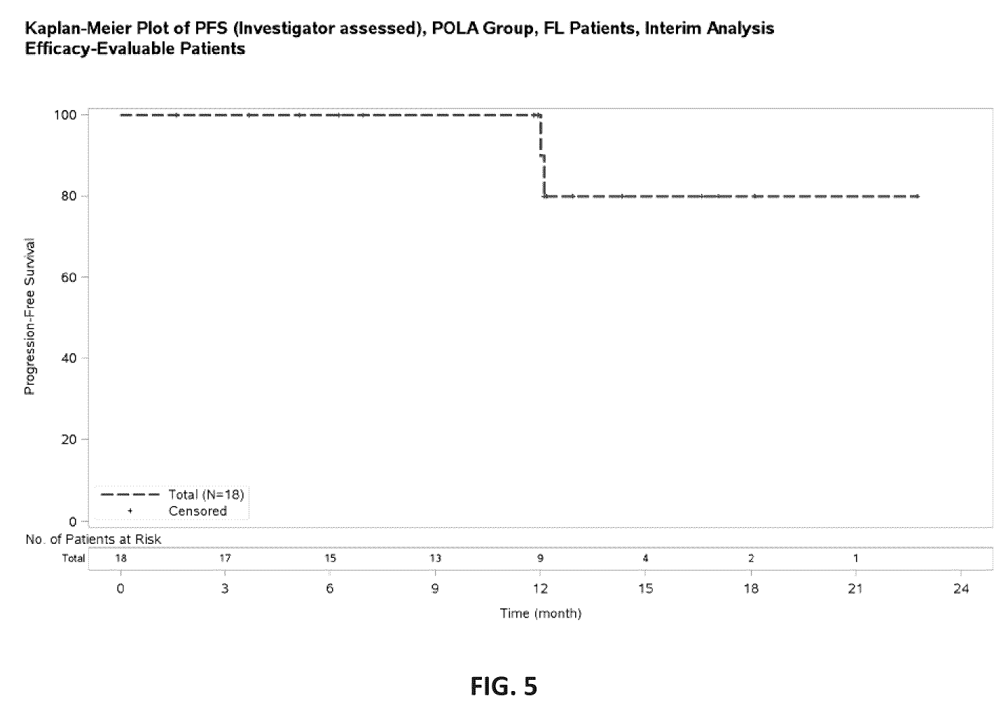

For the Efficacy-evaluable population (n=18), the median duration of follow up was 16.6 months (3.2-25.1 months). The median progression free survival was not reached. The 12-month progression-free survival (PFS) rate was 90% (FIG. 5). The 12-month PFS rate was measured starting from initiation of study treatment (Cycle 1, day 1 of the induction phase). Of 17 responders, two patients have experienced disease progression to date and the remaining patients have ongoing responses, with the longest response being >21 months (FIG. 5).

### SUMMARY

The safety data provided herein demonstrate that polatuzumab vedotin administered in combination with obinutuzumab and lenalidomide is tolerable. Further, the safety profile of the Pola-G-Len combination is consistent with known profiles of the individual drugs, and adverse events were manageable with supportive care.

Currently available data from other completed and ongoing studies with different treatments in similar disease settings indicate that the historical CR rate based on CT scans is 40% for R/R FL. For example, a study by Morschhauser et al., 2017 of the combination of obinutuzumab and lenalidomide in R/R FL showed a CR rate of 44% (using criteria from Cheson, 2007). Other studies of lenalidomide in combination with another anti-CD20 antibody (rituximab) in R/R FL showed CR rates of 34% (Leonard et al., Am Soc of Hematology, 2018; Cheson 2007 criteria, rituximab-sensitive patients), 49% (Rummel et al., Euro Hematology Assoc, 2018; IWG 1999 criteria, rituximab-sensitive patients), and 40% (Rummel et al., Euro Hematology Assoc, 2018; IWG 1999 criteria, rituximab-refractory patients).

In contrast, as shown in Table 14, response rates with the Pola-G-Len combination at the end of induction are promising, with high complete response rates. For example, the CR rate based on PET-CT in R/R FL patients administered the combination of polatuzumab vedotin, lenalidomide, and obinutuzumab was at least 61% when using the Modified Lugano 2014 Criteria and 78% when using the Lugano 2014 criteria.

Moreover, the 12-month progression free survival rate of 90% in the present study is superior to PFS rates observed in other completed and ongoing studies with different treatments in similar disease settings. For example, in Morschhauser et al., 2017 (obinutuzumab and lenalidomide in R/R FL patients) a 12 month PFS rate of 76% was observed (using criteria from Cheson, 2007). Other studies of lenalidomide in combination with another anti-CD20 antibody (rituximab) in R/R FL showed 12-month PFS rates of 75% (Rummel et al., Euro Hematology Assoc, 2018; IWG 1999 criteria, rituximab-sensitive patients), and 60% (Rummel et al., Euro Hematology Assoc, 2018; IWG 1999 criteria, rituximab-refractory patients). One study of lenalidomide in combination with rituximab in rituximab-sensitive R/R FL patients showed a PFS probability at 2 years of 58% when assessed by an IRC and 53% when assessed by the investigator (Leonard et al., (2019) J Clin Oncol, 37(14):1188-1199; Cheson 2007 criteria).

The high rate of CR and PFS observed in patients treated with the triple combination of polatuzumab vedotin, lenalidomide, and obinutuzumab is a significant improvement over treatments with double combinations of anti-CD20 antibodies (e.g., obinutuzumab or rituximab) with lenalidomide.

### CONCLUSIONS

The safety profile of Pola-G-Len is consistent with known profiles of the individual drugs. Response rates at EOI with Pola-G-Len are promising, with high CR compared with available R/R FL treatments. Furthermore, the PFS rate with Pola-G-Len is superior to PFS rates observed with available R/R FL treatments.

### Example 2: An Update to the Phase Ib/II Study of an Anti-CD79b Immunoconjugate (Polatuzumab Vedotin) in Combination with Anti-CD20 Antibody (Obinutuzumab) and Lenalidomide in Relapsed or Refractory Follicular Lymphoma (FL) Described in Example 1

In Example 1, an interim analysis of safety and efficacy results of a Phase Ib/II, open label, multicenter, non-randomized, dose-escalation study of polatuzumab vedotin in combination with obinutuzumab and lenalidomide in patients with relapsed or refractory Follicular Lymphoma (FL) was described. In the following Example, additional safety and efficacy results of the study described in Example 1 are provided.

### Results

### Dose Escalation and DLTs

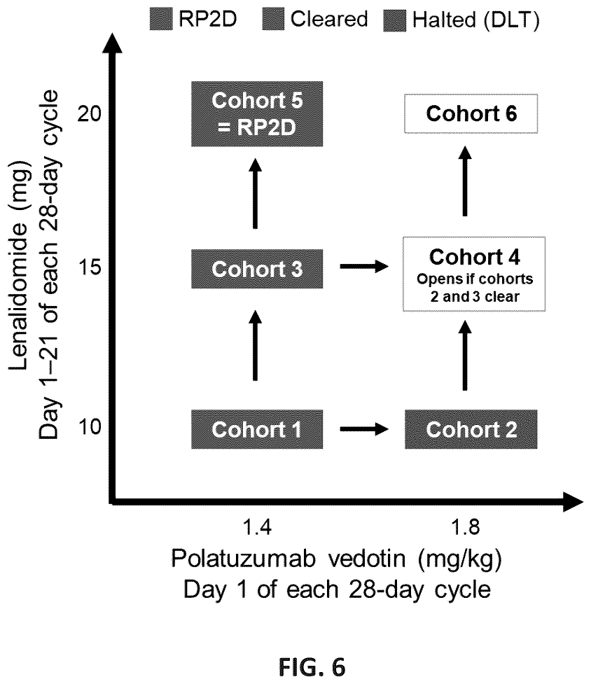

As shown in FIG. 6, during the Dose Escalation phase, dose-limiting toxicities (DLTs) led to the halt of treatment in Cohort 2. Consequently, Cohorts 4 and 6 were not opened. The DLTs that occurred in Cohort 2 were asymptomatic (no signs or symptoms of bleeding) Grade 3 thrombocytopenia and asymptomatic Grade 4 amylase/lipase elevation. Onset of the Grade 3 thrombocytopenia event occurred on Day 28 of Cycle 1 and led to a >14 day delay in the start of Cycle 2 (study treatment was held for 20 days). It was determined that the Grade 3 thrombocytopenia event was related to all three study drugs. Thrombocytopenia was an identified or potential risk for the study drugs. Onset of the Grade 4 amylase/lipase elevation event occurred on Day 25 of Cycle 1 and resolved with study treatment discontinuation and supportive care. CT scans did not show evidence of pancreatitis (pancreatitis was not an identified or potential risk for the study drugs). It was determined that the Grade 4 amylase/lipase elevation event was related to all three study drugs.

Safety data from Cohort 2 were further analyzed, showing that two patients experienced DLT events: one patient experienced Grade 4 amylase/lipase elevation and one patient had Grade 4 neutropenia and Grade 3 thrombocytopenia.

Cohorts 1 and 3 were cleared, and the dosing regimen for Cohort 5 of 1.4 mg/kg polatuzumab vedotin and 20 mg lenalidomide was determined to be the recommended Phase II doses (RP2D) when combined with a fixed dose of obinutuzumab (FIG. 6). No DLTs were observed in Cohort 3 or Cohort 5.

### Patient Characteristics

### Evaluable Populations

The safety-evaluable population was 56 patients: 16 patients from the dose-escalation cohort (10 patients were not treated at the RP2D and 6 patients completed the RP2D induction) and 40 patients from the dose-expansion cohort. The median duration of follow-up was 16.6 months (2.1-39.5).

The efficacy-evaluable population included 46 patients: 6 patients from the dose-escalation cohort and 40 patients from the dose-expansion cohort that completed RP2D induction. The median duration of follow-up was 15.1 months (2.1-29.5).

### Baseline Characteristics

Patient baseline characteristics for the safety-evaluable and efficacy-evaluable populations are provided in Table 15.

For the safety-evaluable population, the median patient age was 62 years, with a range of 32-87 years, 59% of patients were male, 98% had an ECOG performance status score of 0-1, 88% had disease with an Ann Arbor Stage III/IV, 16% had bulky disease (≥7 cm), 43% had bone marrow involvement, 55% were classified as being in the High Risk Group with 3-5 FLIPI Risk Factors, 23% had one prior line of treatment, 25% had two prior lines of treatment, 52% had ≥3 lines of treatment, 59% were refractory to the last prior therapy, 71% were refractory to any line of anti-CD20 therapy, and 25% had progression of disease within 24 months of initiation of the first anti-lymphoma treatment with chemoimmunotherapy (POD24 on first line treatment).

For the efficacy-evaluable population, the median patient age was 62 years, with a range of 32-87 years, 65% of patients were male, 98% had an ECOG performance status score of 0-1, 87% had disease with an Ann Arbor Stage III/IV, 15% had bulky disease (≥7 cm), 48% had bone marrow involvement, 57% were classified as being in the High Risk Group with 3-5 FLIPI Risk Factors, 24% had one prior line of treatment, 24% had two prior lines of treatment, 52% had ≥3 lines of prior treatment, 54% were refractory to the last prior therapy, 70% were refractory to any line of anti-CD20 therapy, and 24% had progression of disease within 24 months of initiation of the first anti-lymphoma treatment with chemoimmunotherapy (POD24 on first line treatment). All tested patients in the efficacy evaluable population (38) had moderate to strong expression of CD79b (IHC2+ and 3+).

TABLE 15
Patient baseline characteristics.

| | Safety Population | | Efficacy Population | |
|---|---|---|---|---|
| Characteristic | n = 56 | | N = 46 | |
| Median age, years (range) | 62 | (32-87) | 62 | (32-87) |
| Male, n (%) | 33 | (59) | 30 | (65) |
| ECOG PS 0-1, n (%) | 55 | (98) | 45 | (98) |
| Ann Arbor Stage III/IV, n (%) | 49 | (88) | 40 | (87) |
| Bulky disease (≥7 cm), n (%) | 9 | (16) | 7 | (15) |
| Bone marrow involvement, n (%) | 24 | (43) | 22 | (48) |
| FLIPI High ≥3, n (%) | 31 | (55) | 26 | (57) |
| Number of prior lines of treatment, n (%) | | | | |
| 1 | 13 | (23) | 11 | (24) |
| 2 | 14 | (25) | 11 | (24) |
| ≥3 | 29 | (52) | 24 | (52) |
| Median prior lines of treatment (range) | 3 | (1-7) | 3 | (1-5) |
| Refractory to last prior therapy¹, n (%) | 33 | (59) | 25 | (54) |
| Refractory to any line of anti-CD20 | 40 | (71) | 32 | (70) |
| therapy², n (%) | | | | |
| POD24 on first-line treatment³, n (%) | 14 | (25) | 11 | (24) |

¹Defined as no response, progression, or relapse within 6 months from the end date of the last anti-lymphoma therapy.
²Defined as no response, progression, or relapse within 6 months of therapy with an anti-CD20 agent during the last prior line of treatment.
³Defined as progression of disease within 24 months of initiation of the first anti-lymphoma treatment with chemoimmunotherapy.

### Safety

### Adverse Events

A summary of wall adverse events (AEs) is provided in Table 16. 84% of patients experienced a Grade 3-4 AE and 2% of patients (1 patient) experienced a Grade 5 AE (septic shock after progressive disease and new anti-lymphoma treatment (TAK-659, tyrosine kinase inhibitor)). 57% of patients experienced a serious AE. AEs leading to dose interruption occurred in 77% of patients, whereas AEs leading to dose reduction occurred in 34% of patients. 30% of patients experienced an AE leading to discontinuation of any drug. AEs leading to drug discontinuations included pneumonitis, lung neoplasm malignant, and thrombocytopenia. The majority of dose interruptions, reductions, and discontinuations occurred due to lenalidomide.

Seven patients required blood transfusions.

TABLE 16
Summary of all adverse events.

| Total number of patients | | |
|---|---|---|
| with at least one AE, n (%) | n = 56 | |
| Any grade AE | 56 | (100) |
| Grade 5 AEs | 1 | (2) |
| Grade 3-4 AEs | 47 | (84) |
| Serious AEs | 32 | (57) |
| AEs leading to dose interruption | 43 | (77) |
| AEs leading to dose reduction | 19 | (34) |
| AEs leading to any drug discontinuation | 17 | (30) |

The most common AEs were infections and infestations (75%), neutropenia (64%), thrombocytopenia (52%), diarrhea (41%), anemia (39%), pyrexia (39%), IRRs (34%), and peripheral neuropathy (29%). A summary of AEs occurring in ≥12.5% of patients is provided in Table 17.

TABLE 17
Summary of adverse events occurring in ≥12.5% of patients.

| All Adverse Events, n (%) | n = 56 | |
|---|---|---|
| Infections and infestations¹ | 42 | (75) |
| Neutropenia | 36 | (64) |
| Thrombocytopenia | 29 | (52) |
| Diarrhea | 23 | (41) |
| Anemia | 22 | (39) |
| Pyrexia | 22 | (39) |
| Infusion Related Reaction (IRR) | 19 | (34) |
| Peripheral neuropathy² | 17 | (30) |
| Cough | 15 | (27) |
| Fatigue | 14 | (25) |
| Rash³ | 14 | (25) |
| Nausea | 12 | (21) |
| ALT increased | 11 | (20) |
| Asthenia | 10 | (18) |
| Constipation | 10 | (18) |
| Decreased appetite | 10 | (18) |
| Arthralgias | 8 | (14) |
| Blood creatinine increased | 8 | (14) |
| Abdominal pain | 7 | (13) |
| AST increased | 7 | (13) |
| Back pain | 7 | (13) |
| Hypokalemia | 7 | (13) |

¹Infections are presented as Systems Organ Class terms; all other AEs are reported by ‘preferred terms’.
²Peripheral neuropathy standard MedDRA query included peripheral motor neuropathy, peripheral sensory neuropathy, neuropathy peripheral, paresthesia, hypoaesthesia, and neuralgia.
³Rash included maculo-popular rash and erythematous rash.

A summary of Grade 3-4 adverse events is provided in Table 18. The most common hematologic Grade 3-4 AE was neutropenia (55%). The most common non-hematologic Grade 3-4 AE was infections and infestations (20%).

TABLE 18
Summary of Grade 3-4 AEs.

| Total number of | | |
|---|---|---|
| patients, n (%) | n = 56 | |
| Total Grade 3-4 AEs (≥2 patients) | 47 | (84) |

Hematologic AEs

| | | |
|---|---|---|
| Neutropenia | 31 | (55) |
| Thrombocytopenia | 15 | (27) |
| Anemia | 8 | (14) |
| Febrile neutropenia | 6 | (11) |

Non-hematologic AEs

| | | |
|---|---|---|
| Infections and infestations¹ | 11 | (20) |
| Hypokalemia | 3 | (5) |
| Diarrhea | 2 | (4) |
| Lipase increased | 2 | (4) |
| Laboratory Tumor lysis syndrome | 2 | (4) |
| ALT increased | 2 | (4) |
| Syncope | 2 | (4) |

The Grade 3-4 infection and infestation AEs included 2 events of each of lower respiratory tract infection and neutropenic sepsis and one event of each of the following: bronchiolitis, cavernous sinus thrombosis, epididymitis, furuncle, lung infection, septic shock, sinusitis, and urinary tract infection. Filgrastim (granulocyte colony stimulating factor) was used by 31 patients (55%) during the Induction phase and by 20 patients (36%) during the maintenance phase. Platelet transfusions were given to 1 patient (2%) during the Induction phase and 1 patient (2%) during the maintenance phase.

A summary of adverse events of special interest (AESI) is provided in Table 19. 7% of patients experienced tumor flare, 2% experienced myelodysplastic syndrome (1 patient), and 2% experienced lung neoplasm malignant (1 patient).

TABLE 19
Summary of AEs of special interest.

| | |
|---|---|
| Total number of | |
| patients, n (%) | n = 56 |

Neoplasms, Benign, Malignant, and Unspecified

| | |
|---|---|
| Tumor Flare | 4 (7) |
| Myelodysplastic Syndrome | 1 (2) |
| Lung Neoplasm Malignant | 1 (2) |

A summary of additional selected AEs is provided in Table 20. Two events of Grade 3 laboratory tumor lysis syndrome (TLS) occurred. No clinical TLS was documented and the TLS events resolved with supportive care. A total of 5 events of peripheral neuropathy, 5 events of paraesthesia, 2 events of peripheral motor neuropathy, 2 events of peripheral sensory neuropathy, 1 event of hypoaesthesia, and 2 events of neuralgia occurred (See also, Table 17). In addition, a total of 4 events of tumor flare, 1 event of myelodysplastic syndrome, and 1 event of lung neoplasm malignant occurred (See also, Table 19). Myelodysplastic syndrome and lung neoplasm malignant were not classified as second malignancies according to standard MedDRA queries (SMQ-w).

TABLE 20
Summary of selected AEs.

| | | | | | |
|---|---|---|---|---|---|
| Total number of events, | | | | | |
| n (%) | Grade 1 | Grade 2 | Grade 3 | Grade 4 | Total |

TLS

| | | | | | |
|---|---|---|---|---|---|
| Laboratory TLS¹ | 0 | 0 | 2 (4) | 0 | 2 (4) |

Peripheral Neuropathy

| | | | | | |
|---|---|---|---|---|---|
| Peripheral neuropathy | 4 (7) | 1 (2) | 0 | 0 | 5 (9) |
| Paraesthesia | 5 (9) | 0 | 0 | 0 | 5 (9) |
| Peripheral motor neuropathy | 1 (2) | 1 (2) | 0 | 0 | 2 (4) |
| Peripheral sensory neuropathy | 2 (4) | 0 | 0 | 0 | 2 (4) |
| Hypoaesthesia | 1 (2) | 0 | 0 | 0 | 1 (2) |
| Neuralgia | 0 | 2 (4) | 0 | 0 | 2 (4) |

Neoplasms, Benign, Malignant, and Unspecified

| | | | | | |
|---|---|---|---|---|---|
| Tumor Flare | 2 (4) | 2 (4) | 0 | 0 | 4 (7) |
| Myelodysplastic Syndrome | 0 | 0 | 0 | 1 (2) | 1 (2) |
| Lung Neoplasm Malignant | 0 | 0 | 1 (2) | 0 | 1 (2) |

### Study Drug Discontinuations

Nineteen discontinuations of any study drug occurred. The most common hematologic AE leading to any study drug discontinuation was thrombocytopenia (4 events). One event of increased lipase occurred. The most common infection and infestation AE leading to any study drug discontinuation was lower respiratory tract infection (2 events). A summary of AEs leading to any study drug discontinuation is provided in Table 21.

TABLE 21
Summary of AE events leading to any study drug discontinuation.

| | |
|---|---|
| Total number of events | 19 |

Hematologic AEs

| | |
|---|---|
| Thrombocytopenia | 4 |
| Anemia | 1 |
| Neutropenia | 1 |

Infections and infestations

| | |
|---|---|
| Lower respiratory tract infection | 2 |
| Cavernous sinus thrombosis | 1 |
| Pneumonia | 1 |
| Sinusitis | 1 |

Other

| | |
|---|---|
| Acute coronary syndrome | 1 |
| Amylase/Lipase increased | 1 |
| Colitis | 1 |
| Eye hemorrhage | 1 |
| Interstitial lung disease | 1 |
| Lung Neoplasm Malignant | 1 |
| Myelodysplastic Syndrome | 1 |
| Pneumonitis | 1 |

Safety data were further analyzed, showing that 9 patients (16%) experienced an AE of rash, and Grade 3-4 adverse events of pyrexia, infusion-related reaction, and asthenia were experienced by one patient each. In addition, the most common serious AEs were febrile neutropenia (n=5, 9%) and pyrexia (n=4, 7%), and sixteen (29%) patients experienced peripheral neuropathy (all grade 1 or 2; no treatment modifications were required). This analysis also showed that lenalidomide dose reductions during induction were required in 18 (32%) patients due to AEs, most commonly due to neutropenia (n=5, 9%) and thrombocytopenia (n=5, 9%). Two patients required lenalidomide dose reductions during maintenance, one due to neutropenia and one due to peripheral neuropathy. There were no dose reductions of polatuzumab vedotin or obinutuzumab. In addition, of the 17 (30%) study treatment discontinuations, four were due to thrombocytopenia, two due to lower respiratory tract infections, and one due to each of acute coronary syndrome, amylase/lipase increased, anemia, cavernous sinus thrombosis, colitis, interstitial lung disease, malignant lung neoplasm, myelodysplastic syndrome, neutropenia, pneumonia, pneumonitis, and sinusitis. Overall, six patients died due to disease progression (PD).

### Efficacy

### Study Discontinuations

Of the 46 patients in the efficacy evaluable population, 39 patients completed the Induction phase. Five patients in the efficacy evaluable population were discontinued from the study due to death, one patient was discontinued from the study due to AE, and four patients withdrew from the study. Of the five deaths, three were due to disease progression and two were due to complications following a new anti-lymphoma therapy (stem-cell transplantation).

### Exposure

As shown in Table 22, during the Induction phase, the median number of doses administered of obinutuzumab, polatuzumab vedotin, and lenalidomide were 8, 6, and 124, respectively. The median duration of Induction treatment was 4.7 months for obinutuzumab and polatuzumab vedotin and 5.3 months for lenalidomide.

TABLE 22
Summary of treatment exposure during Induction treatment.

| | Obinutuzumab | | Polatuzumab vedotin | | Lenalidomide | |
|---|---|---|---|---|---|---|
| | (N = 46) | | (N = 46) | | (N = 46) | |
| Median number of doses received, n | 8 | (1-8) | 6 | (1-6) | 124 | (7-127) |
| Median dose intensity (range), % | 100 | (84-100) | 99.9 | (71-108) | 94 | (38-101) |
| Median Induction treatment duration | 4.7 | (0-6) | 4.7 | (0-6) | 5.3 | (0-7) |
| (months), n | | | | | | |

### Responses

Responses to treatment were assessed at the end of induction (EOI) treatment using the Modified Lugano 2014 criteria (required a negative bone marrow biopsy to confirm PET-CR and PET-PR, as well as meeting CT-PR criteria) and the Lugano 2014 criteria with PET results only (Table 23).

The objective response rate (ORR) using the Lugano 2014 criteria (PET only) or the Modified Lugano 2014 criteria was 83% when assessed by the investigator and 76% when assessed by the independent review committee (IRC).

Complete responses where observed in at least 61% of patients when using either the Modified Lugano 2014 criteria or the Lugano 2014 criteria with PET results only (using the Modified Lugano 2014 criteria: 61% when assessed by the investigator and 63% when assessed by the IRC; using the Lugano 2014 criteria with PET results only: 72% when assessed by the investigator or the IRC).

Complete responses assessed using the Modified Lugano 2014 criteria were downgraded to partial responses due to missing bone marrow biopsies in 6 patients by the investigator and 4 patients by the IRC. No patients were downgraded due to persistent BM positivity.

Partial responses assessed using the Modified Lugano 2014 criteria were observed in 22% of patients when determined by the investigator and 13% when determined by the IRC. Using the Lugano 2014 criteria with PET results only, partial responses were observed in 9% of patients when assessed by the investigator and 4% of patients when assessed by the IRC.

Up to 9% of patients exhibited stable disease (using the Modified Lugano 2014 criteria or the Lugano 2014 criteria with PET results only: 7% when assessed by the investigator and 9% when assessed by the IRC. Disease progression was observed in up to 7% of patients (using the Modified Lugano 2014 criteria or the Lugano 2014 criteria with PET results only: 7% when assessed by the investigator and 2% when assessed by the IRC).

Of the missing or unevaluable patients listed in Table 23 using either the Modified Lugano 2014 criteria or the Lugano 2014 criteria with PET results only, three patients were classified as missing due to early progressive disease and scans not being sent to the IRC. Two of the patients that experienced PD before reaching EOI died as a result of PD.

TABLE 23
Responses at EOI (efficacy-evaluable population; RP2D; n = 46).
Responses at End of Induction (N = 46)

| Best overall | | | | | | | | |
|---|---|---|---|---|---|---|---|---|
| response, n | Modified Lugano 2014¹ | | | | Lugano 2014 | | | |
| (%) | INV | | IRC | | INV | | IRC | |
| ORR | 38 | (83) | 35 | (76) | 38 | (83) | 35 | (76) |
| CR | 28 | (61)² | 29 | (63)² | 33 | (72) | 33 | (72) |
| PR | 10 | (22) | 6 | (13) | 4 | (9) | 2 | (4) |
| SD | 3 | (7) | 4 | (9) | 3 | (7) | 4 | (9) |
| PD | 3 | (7) | 1 | (2) | 3 | (7) | 1 | (2) |
| Missing/unevaluable | 2 | (4) | 6 | (13)³ | 2 | (4) | 6 | (13)³ |

¹Modified Lugano required a negative BMB to confirm PET-CR and PET-PR as well as meeting CT-PR criteria.
²CR downgraded to PR due to missing BMB in 6 patients by INV and 4 patients by IRC.
³Three patients experienced early PD, scans were not sent to IRC and therefore were classified as missing.
BMB = bone marrow biopsy; CR = complete response; CT = computed tomography; EOI = end of induction; INV = investigator assessment; IRC = independent review committee assessment; ORR = objective response rate; PD = progressive disease; PET = positron emission tomography; PR = partial response; SD = stable disease.

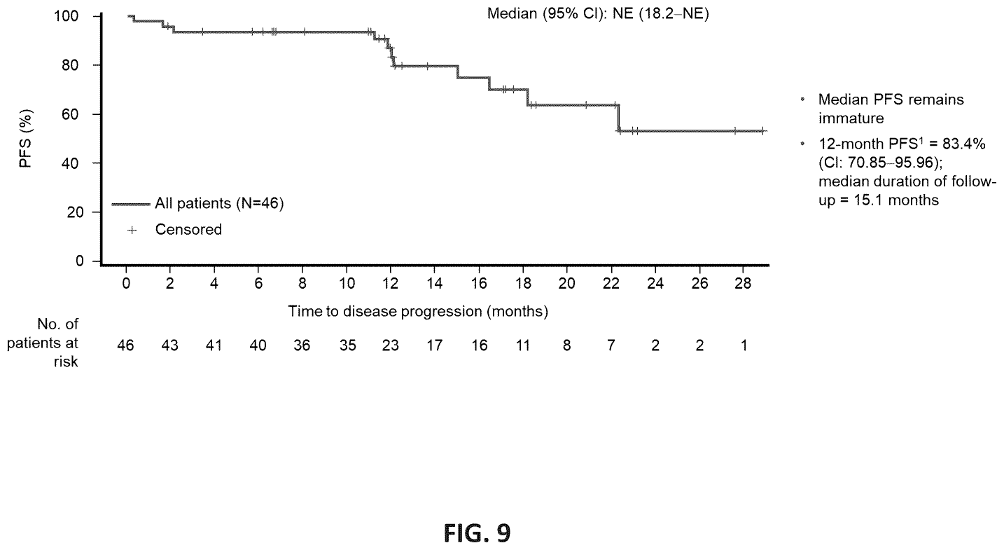

The median progression free survival (PFS) was not reached. As shown in the Kaplan-Meier plot provided in FIG. 9, the 12-month PFS rate as assessed by the investigator was 83.4% (Confidence interval: 70.85, 95.96), with a 15.1 month median duration of follow up. Of the 46 patients in the efficacy-evaluable population, 3 patients died due to progressive disease and 2 patients died due to complications following a new anti-lymphoma therapy (stem-cell transplantation).

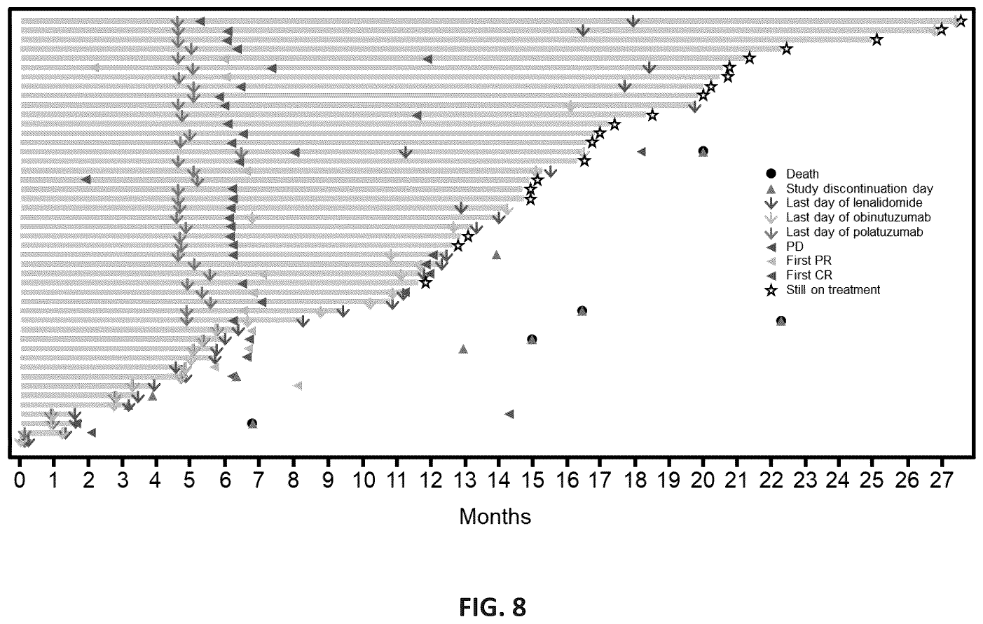

A summary of the follow-up period and response results for each patient in the efficacy-evaluable population is provided in FIG. 8.

Efficacy data were further analyzed, showing that 34 patients (74%) had a complete response (CR) as assessed by the investigator based on the Lugano 2014 criteria.

### Subgroup Analyses

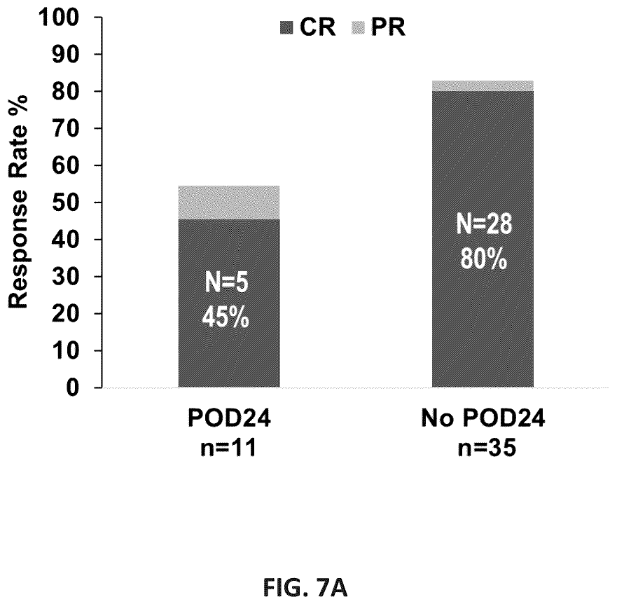

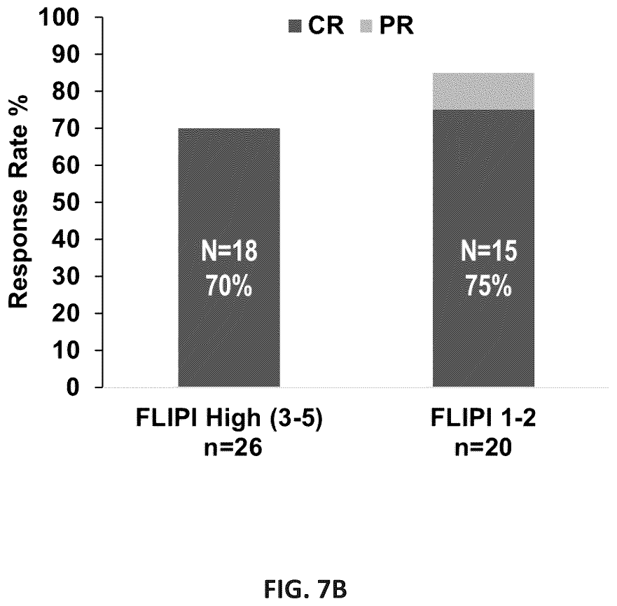

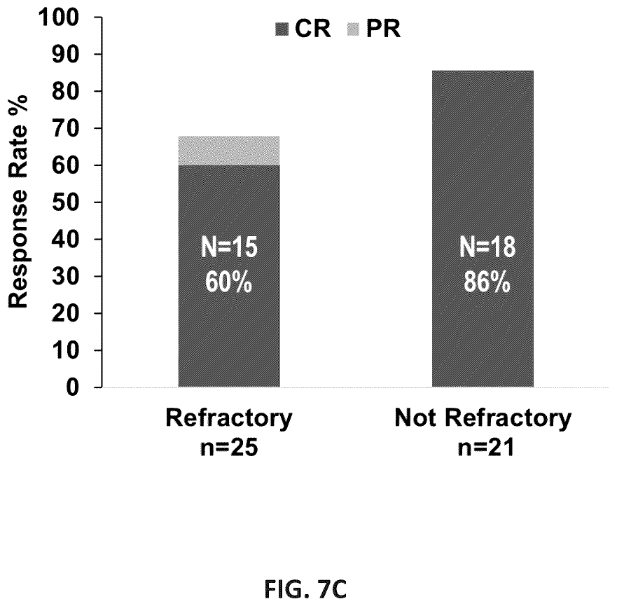

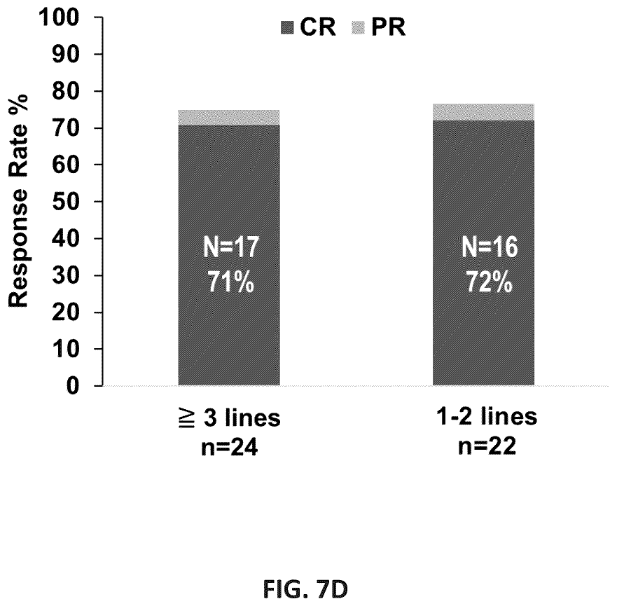

An analysis of patient subgroups with progression of disease within 24 months of initiation of the first anti-lymphoma treatment with chemoimmunotherapy (POD24 on first line treatment) or without POD24 on first line treatment showed that patients with POD24 on first line treatment had a 45% complete response rate, while patients without POD24 on first line treatment had an 80% complete response rate (FIG. 7A).

Comparison of patients classified as being in the High Risk Group, with 3-5 FLIPI Risk Factors (FLIPI High subgroup) to patients classified as having 1-2 FLIPI Risk Factors (FLIPI 1-2 subgroup) revealed that the FLIPI High subgroup had a 70% complete response rate while the FLIPI 1-2 subgroup had a 75% complete response rate (FIG. 7B).

The subgroup of patients that had disease refractory to the last line of treatment exhibited a 60% complete response rate, while patients with disease not refractory to the last line of treatment had an 86% complete response rate (FIG. 7C).

Patients that had ≥3 prior lines of treatment exhibited a 71% complete response rate, while patients that had 1-2 prior lines of treatment exhibited a 72% complete response rate (FIG. 7D).

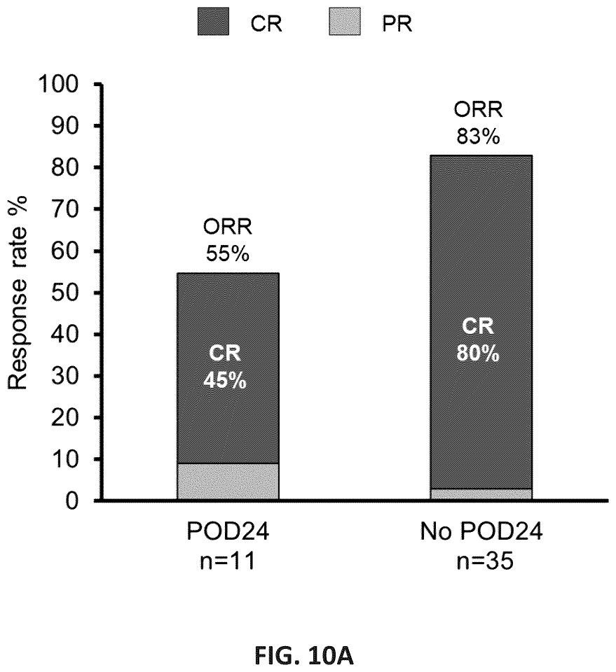

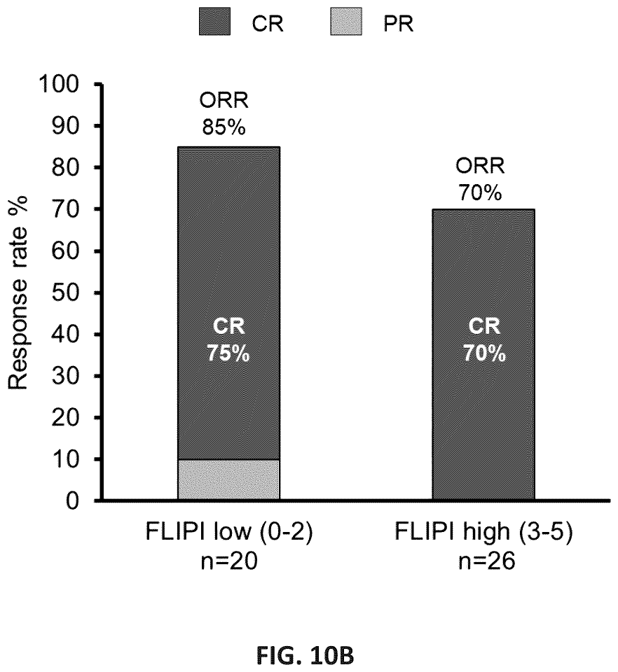

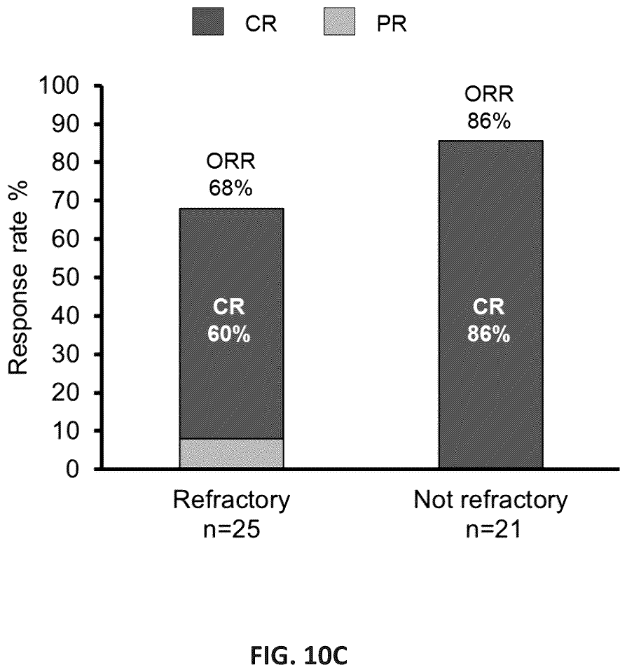

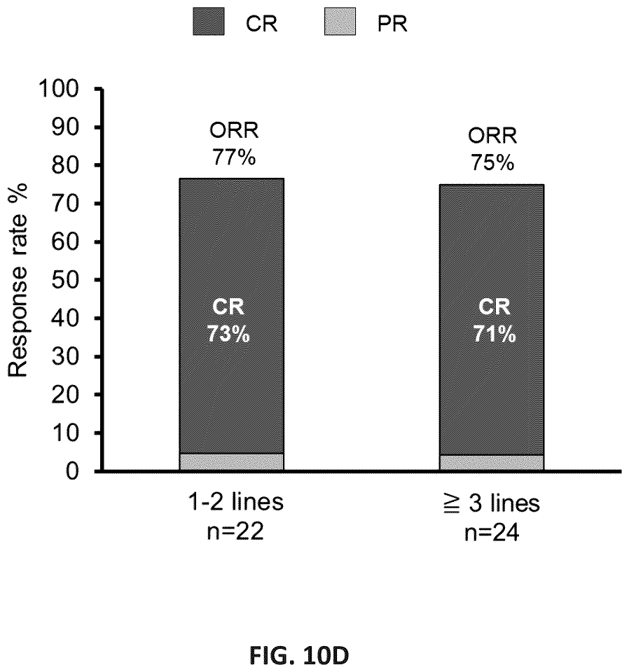

An additional analysis of patient subgroups is provided in FIGS. 10A-10D. As shown in FIG. 10A, patients with POD24 on first line treatment had a 55% overall response rate (ORR), while patients without POD24 on first line treatment had an 83% ORR. Patients in the FLIPI High subgroup had a 70% ORR, whereas patients in the FLIPI Low subgroup had an 85% ORR (FIG. 10B). Patients with refractory disease, defined as no response, progression, or relapse within 6 months of the last anti-lymphoma therapy end date, had a 68% ORR, while patients without refractory disease had an ORR of 86% (FIG. 10C). Finally, patients that had 1-2 prior lines of treatment had a 77% ORR, whereas patients that had ≥3 prior lines of treatment had an ORR of 75% (FIG. 10D).

### CONCLUSIONS

The results presented in this Example show that the novel triplet combination, Pola-G-Len, demonstrates a safety profile consistent with the known profiles of the individual drugs. In addition, the efficacy-evaluable population, which included patients that were heavily pre-treated and refractory to prior treatments, showed a 12-month PFS rate of about 83% and high CR rates at EOI.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method for treating follicular lymphoma (FL) in a human in need thereof comprising administering to the human an effective amount of:
(a) an immunoconjugate comprising the formula 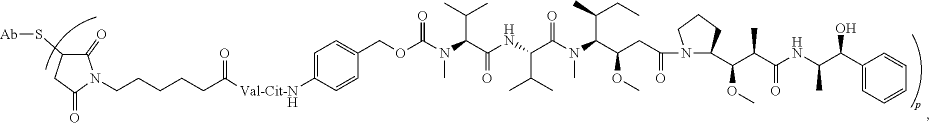
wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and
wherein p is between 1 and 8,
(b) an immunomodulatory agent, and
(c) an anti-CD20 antibody; and
wherein the human achieves at least a complete response (CR) following the treatment.

2. The method of claim 1, wherein, among a plurality of humans treated, at least 60%, at least 65%, at least 70%, or at least 75% of the humans achieve a complete response.

3. The method of claim 1 or claim 2, wherein the anti-CD79b antibody comprises (i) a heavy chain variable domain (VH) comprising the amino acid sequence of SEQ ID NO: 19 and (ii) a light chain variable domain (VL) comprising the amino acid sequence of SEQ ID NO: 20.

4. The method of any one of claims 1-3, wherein the anti-CD79b antibody comprises (i) a heavy chain comprising the amino acid sequence of SEQ ID NO: 36 and (ii) a light chain comprising the amino acid sequence of SEQ ID NO: 35.

5. The method of any one of claims 1-4, wherein the immunoconjugate is polatuzumab vedotin.

6. The method of any one of claims 1-5, wherein the immunomodulatory agent is lenalidomide.

7. The method of claim 6, wherein the anti-CD20 antibody is obinutuzumab.

8. The method of claim 7, wherein the immunoconjugate is administered at a dose between about 1.4 mg/kg and about 1.8 mg/kg, the lenalidomide is administered at a dose between about 10 mg and about 20 mg, and the obinutuzumab is administered at a dose of about 1000 mg.

9. The method of claim 8, wherein the immunoconjugate, the lenalidomide, and the obinutuzumab are administered during an induction phase for at least six 28-day cycles,
wherein the immunoconjugate is administered intravenously at a dose between about 1.4 mg/kg and about 1.8 mg/kg on Day 1, the lenalidomide is administered orally at a dose between about 10 mg and about 20 mg, on each of Days 1-21, and the obinutuzumab is administered intravenously at a dose of about 1000 mg on each of Days 1, 8, and 15 of the first 28-day cycle, and
wherein the immunoconjugate is administered intravenously at a dose between about 1.4 mg/kg and about 1.8 mg/kg on Day 1, the lenalidomide is administered orally at a dose between about 10 mg and about 20 mg on each of Days 1-21, and the obinutuzumab is administered intravenously at a dose of about 1000 mg on Day 1 of each of the second, third, fourth, fifth, and sixth 28-day cycles.

10. The method of claim 9, wherein the immunoconjugate, the immunomodulatory agent, and the anti-CD20 antibody are administered sequentially.

11. The method of claim 10,
wherein the lenalidomide is administered prior to the obinutuzumab, and wherein the obinutuzumab is administered prior to the immunoconjugate on Day 1 and wherein the lenalidomide is administered prior to the obinutuzumab on each of Days 8 and 15 of the first 28-day cycle, and
wherein the lenalidomide is administered prior to the obinutuzumab, and wherein the obinutuzumab is administered prior to the immunoconjugate on Day 1 of each of the second, third, fourth, fifth, and sixth 28-day cycles.

12. The method of any one of claims 9-11, wherein the lenalidomide and the obinutuzumab are further administered during a maintenance phase following the sixth 28-day cycle.

13. The method of claim 12, wherein the lenalidomide is administered orally at a dose of about 10 mg on each of Days 1-21 of each month during the maintenance phase following the sixth 28-day cycle, and wherein the obinutuzumab is administered intravenously at a dose of about 1000 mg on Day 1 of every other month during the maintenance phase following the sixth 28-day cycle.

14. The method of claim 13, wherein the lenalidomide is administered for a maximum of 12 months during the maintenance phase following the sixth 28-day cycle.

15. The method of claim 13 or 14, wherein the obinutuzumab is administered for a maximum of 24 months during the maintenance phase following the sixth 28-day cycle.

16. The method of any one of claims 12-15, wherein the lenalidomide and the obinutuzumab are administered sequentially during the maintenance phase following the sixth 28-day cycle.

17. The method of any one of claims 13-16, wherein the lenalidomide is administered prior to the obinutuzumab on Day 1 of each of the first, third, fifth, seventh, ninth, and eleventh months during the maintenance phase following the sixth 28-day cycle.

18. A method for treating follicular lymphoma (FL) in a human in need thereof comprising administering to the human an effective amount of:
(a) an immunoconjugate comprising the formula 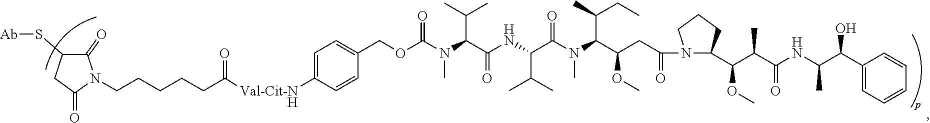
wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and
wherein p is between 1 and 8,
(b) an immunomodulatory agent, and
(c) an anti-CD20 antibody; and
wherein the human does not demonstrate disease progression within at least about 12 months.

19. The method of claim 18, wherein the human does not demonstrate disease progression within at least about 12 months after the start of treatment with the immunoconjugate, the immunomodulatory agent, and the anti-CD20 antibody.

20. The method of any one of claims 1-19, wherein, among a plurality of humans treated, at least 75%, at least 80%, at least 85%, or at least 90% of the humans do not demonstrate disease progression within at least about 12 months after the start of treatment with the immunoconjugate, the immunomodulatory agent, and the anti-CD20 antibody.

21. A method for treating follicular lymphoma (FL) in a human in need thereof comprising administering to the human an effective amount of:
(a) an immunoconjugate comprising the formula 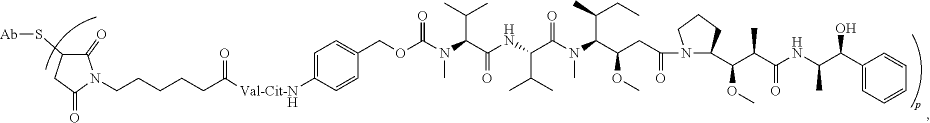
wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and
wherein p is between 1 and 8,
(b) an immunomodulatory agent, and
(c) an anti-CD20 antibody; and
wherein the human demonstrates 12-month progression-free survival.

22. The method of claim 21, wherein the human demonstrates 12-month progression-free survival, measured after the start of treatment with the immunoconjugate, the immunomodulatory agent, and the anti-CD20 antibody.

23. The method of any one of claims 1-22, wherein, among a plurality of humans treated, the 12-month progression-free survival rate is at least 75%, at least 80%, at least 85%, or at least 90%, measured after the start of treatment with the immunoconjugate, the immunomodulatory agent, and the anti-CD20 antibody.

24. A method of treating follicular lymphoma in a human in need thereof, comprising administering to the human an effective amount of:
(a) an immunoconjugate comprising the formula 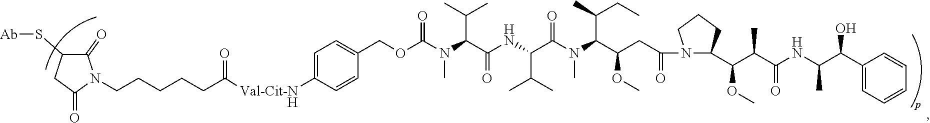
wherein Ab is an anti-CD79b antibody comprising (i) a heavy chain variable domain (VH) comprising the amino acid sequence of SEQ ID NO: 19 and (ii) a light chain variable domain (VL) comprising the amino acid sequence of SEQ ID NO: 20, and
wherein p is between 2 and 5,
(b) lenalidomide and
(c) obinutuzumab,
wherein the immunoconjugate is administered at a dose between about 1.4 mg/kg and about 1.8 mg/kg, the lenalidomide is administered at a dose between about 10 mg and about 20 mg, and the obinutuzumab is administered at a dose of about 1000 mg, and
wherein the human achieves at least complete response (CR) following the treatment.

25. The method of claim 24, wherein, among a plurality of humans treated, at least 60%, at least 65%, at least 70%, or at least 75% of the humans achieve a complete response.

26. The method of claim 24 or 25, wherein p is between 3 and 4.

27. The method of any one of claims 24-26, wherein the antibody comprises (i) a heavy chain comprising the amino acid sequence of SEQ ID NO: 36 and wherein (ii) a light chain comprising the amino acid sequence of SEQ ID NO: 35.

28. The method of any one of claims 24-27, wherein the immunoconjugate is polatuzumab vedotin.

29. The method of any one of claims 24-28, wherein the immunoconjugate, the lenalidomide, and the obinutuzumab are administered during an induction phase for at least six 28-day cycles,
wherein the immunoconjugate is administered intravenously at a dose between about 1.4 mg/kg and about 1.8 mg/kg on Day 1, the lenalidomide is administered orally at a dose between about 10 mg and about 20 mg on each of Days 1-21, and the obinutuzumab is administered intravenously at a dose of about 1000 mg on each of Days 1, 8, and 15 of the first 28 day cycle, and
wherein the immunoconjugate is administered intravenously at a dose between about 1.4 mg/kg and about 1.8 mg/kg on Day 1, the lenalidomide is administered orally at a dose between about 10 mg and about 20 mg on each of Days 1-21, and the obinutuzumab is administered intravenously at a dose of about 1000 mg on Day 1 of each of the second, third, fourth, fifth, and sixth 28-day cycles.

30. The method of claim 24-29, wherein the lenalidomide and the obinutuzumab are further administered during a maintenance phase following the sixth 28-day cycle.

31. The method of claim 30, wherein the lenalidomide is administered orally at a dose of about 10 mg on each of each of Days 1-21 of each month during the maintenance phase following the sixth 28-day cycle, and wherein the obinutuzumab is administered intravenously at a dose of about 1000 mg on Day 1 of every other month during the maintenance phase following the sixth 28-day cycle.

32. The method of claim 31, wherein the lenalidomide is administered for a maximum of 12 months during the maintenance phase following the sixth 28-day cycle.

33. The method of claim 31 or 32, wherein the obinutuzumab is administered for a maximum of 24 months during the maintenance phase following the sixth 28-day cycle.

34. The method of any one of claims 30-33, wherein the lenalidomide and the obinutuzumab are administered sequentially during the maintenance phase following the sixth 28-day cycle.

35. The method of any one of claims 31-34, wherein the lenalidomide is administered prior to the obinutuzumab on Day 1 of each of the first, third, fifth, seventh, ninth, and eleventh months during the maintenance phase following the sixth 28-day cycle.

36. The method of any one of claims 24-35, wherein, among a plurality of humans treated, at least 75%, at least 80%, at least 85%, or at least 90% of the humans do not demonstrate disease progression within at least about 12 months after the start of treatment with the immunoconjugate, the lenalidomide, and the obinutuzumab.

37. The method of any one of claims 24-36, wherein, among a plurality of humans treated, the 12-month progression-free survival rate is at least 75%, at least 80%, at least 85%, or at least 90%, measured after the start of treatment with the immunoconjugate, the lenalidomide, and the obinutuzumab.

38. The method of any one of claims 29-37, wherein, among a plurality of humans treated, at least 75%, at least 80%, at least 85%, or at least 90% of the humans do not demonstrate disease progression within at least about 12 months after Day 1 of the first 28 day cycle during the induction phase.

39. The method of any one of claims 29-38, wherein, among a plurality of humans treated, the 12-month progression-free survival rate is at least 75%, at least 80%, at least 85%, or at least 90%, measured after Day 1 of the first 28 day cycle during the induction phase.

40. A method of treating follicular lymphoma (FL) in a human in need thereof, comprising administering to the human, during an induction phase, an effective amount of:
(a) polatuzumab vedotin;
(b) lenalidomide; and
(c) obinutuzumab,
wherein, during the induction phase, the polatuzumab vedotin is administered at a dose of about 1.4 mg/kg, the lenalidomide is administered at a dose of about 20 mg, and the obinutuzumab is administered at a dose of about 1000 mg, and
wherein, the human achieves a complete response following the induction phase.

41. The method of claim 40, wherein the polatuzumab vedotin, the lenalidomide, and the obinutuzumab are administered during the induction phase for at least six 28-day cycles,
wherein the polatuzumab vedotin is administered intravenously at a dose of about 1.4 mg/kg on Day 1, the lenalidomide is administered orally at a dose of about 20 mg on each of Days 1-21, and the obinutuzumab is administered intravenously at a dose of about 1000 mg on each of Days 1, 8, and 15 of the first 28 day cycle, and
wherein the polatuzumab vedotin is administered intravenously at a dose of about 1.4 mg/kg on Day 1, the lenalidomide is administered orally at a dose between about 20 mg on each of Days 1-21, and the obinutuzumab is administered intravenously at a dose of about 1000 mg on Day 1 of each of the second, third, fourth, fifth, and sixth 28-day cycles.

42. The method of claim 40 or 41, wherein the induction phase is followed by a maintenance phase, wherein the lenalidomide is administered at a dose of about 10 mg and the obinutuzumab is administered at a dose of about 1000 mg during the maintenance phase.

43. The method of claim 42, wherein the lenalidomide is administered orally at a dose of about 10 mg on each of Days 1-21 of each month during the maintenance phase following the sixth 28-day cycle, and wherein the obinutuzumab is administered intravenously at a dose of about 1000 mg on Day 1 of every other month during the maintenance phase following the sixth 28-day cycle.

44. The method of any one of claims 40-43, wherein the human does not demonstrate disease progression within at least about 12 months after the start of the induction phase.

45. The method of any one of claims 40-44, wherein the human demonstrates 12-month progression-free survival, measured after the start of the induction phase.

46. A method of treating follicular lymphoma (FL) in a plurality of humans in need thereof, comprising administering to the humans, during an induction phase, an effective amount of:
(a) polatuzumab vedotin;
(b) lenalidomide; and
(c) obinutuzumab,
wherein, during the induction phase, the polatuzumab vedotin is administered at a dose of about 1.4 mg/kg, the lenalidomide is administered at a dose of about 20 mg, and the obinutuzumab is administered at a dose of about 1000 mg, and
wherein, at least 60% of the humans in the plurality achieve a complete response following the induction phase.

47. The method of claim 46, wherein the polatuzumab vedotin, the lenalidomide, and the obinutuzumab are administered during the induction phase for at least six 28-day cycles,
wherein the polatuzumab vedotin is administered intravenously at a dose of about 1.4 mg/kg on Day 1, the lenalidomide is administered orally at a dose of about 20 mg on each of Days 1-21, and the obinutuzumab is administered intravenously at a dose of about 1000 mg on each of Days 1, 8, and 15 of the first 28 day cycle, and
wherein the polatuzumab vedotin is administered intravenously at a dose of about 1.4 mg/kg on Day 1, the lenalidomide is administered orally at a dose between about 20 mg on each of Days 1-21, and the obinutuzumab is administered intravenously at a dose of about 1000 mg on Day 1 of each of the second, third, fourth, fifth, and sixth 28-day cycles.

48. The method of claim 46 or 47, wherein the induction phase is followed by a maintenance phase, wherein the lenalidomide is administered at a dose of about 10 mg and the obinutuzumab is administered at a dose of about 1000 mg during the maintenance phase.

49. The method of claim 48, wherein the lenalidomide is administered orally at a dose of about 10 mg on each of Days 1-21 of each month during the maintenance phase following the sixth 28-day cycle, and wherein the obinutuzumab is administered intravenously at a dose of about 1000 mg on Day 1 of every other month during the maintenance phase following the sixth 28-day cycle.

50. The method of any one of claims 1-49, wherein, among a plurality of humans treated, at least 75%, at least 80%, at least 85%, or at least 90% of the humans do not demonstrate disease progression within at least 12 months, measured after the start of treatment with the immunoconjugate or the polatuzumab vedotin, the immunomodulatory agent or the lenalidomide, and the anti-CD20 antibody or the obinutuzumab.

51. The method of any one of claims 1-50, wherein, among a plurality of humans treated, the 12-month progression-free survival rate is at least 75%, at least 80%, at least 85%, or at least 90%, measured after the start of treatment with the immunoconjugate or the polatuzumab vedotin, the immunomodulatory agent or the lenalidomide, and the anti-CD20 antibody or the obinutuzumab.

52. A method of treating follicular lymphoma (FL) in a human in need thereof, comprising administering to the human, during an induction phase, an effective amount of:
(a) polatuzumab vedotin;
(b) lenalidomide; and
(c) obinutuzumab,
wherein, during the induction phase, the polatuzumab vedotin is administered at a dose of about 1.8 mg/kg, the lenalidomide is administered at a dose of about 20 mg, and the obinutuzumab is administered at a dose of about 1000 mg, and
wherein, the human achieves a complete response following the induction phase.

53. The method of claim 52, wherein the polatuzumab vedotin, the lenalidomide, and the obinutuzumab are administered during the induction phase for at least six 28-day cycles,
wherein the polatuzumab vedotin is administered intravenously at a dose of about 1.8 mg/kg on Day 1, the lenalidomide is administered orally at a dose of about 20 mg on each of Days 1-21, and the obinutuzumab is administered intravenously at a dose of about 1000 mg on each of Days 1, 8, and 15 of the first 28 day cycle, and
wherein the polatuzumab vedotin is administered intravenously at a dose of about 1.8 mg/kg on Day 1, the lenalidomide is administered orally at a dose between about 20 mg on each of Days 1-21, and the obinutuzumab is administered intravenously at a dose of about 1000 mg on Day 1 of each of the second, third, fourth, fifth, and sixth 28-day cycles.

54. The method of claim 52 or 53, wherein the induction phase is followed by a maintenance phase, wherein the lenalidomide is administered at a dose of about 10 mg and the obinutuzumab is administered at a dose of about 1000 mg during the maintenance phase.

55. The method of claim 54, wherein the lenalidomide is administered orally at a dose of about 10 mg on each of Days 1-21 of each month during the maintenance phase following the sixth 28-day cycle, and wherein the obinutuzumab is administered intravenously at a dose of about 1000 mg on Day 1 of every other month during the maintenance phase following the sixth 28-day cycle.

56. The method of any one of claims 52-55, wherein the human does not demonstrate disease progression within at least about 12 months after the start of the induction phase.

57. The method of any one of claims 52-56, wherein the human demonstrates 12-month progression-free survival, measured after the start of the induction phase.

58. A method of treating follicular lymphoma (FL) in a plurality of humans in need thereof, comprising administering to the humans, during an induction phase, an effective amount of:
(a) polatuzumab vedotin;
(b) lenalidomide; and
(c) obinutuzumab,
wherein, during the induction phase, the polatuzumab vedotin is administered at a dose of about 1.8 mg/kg, the lenalidomide is administered at a dose of about 20 mg, and the obinutuzumab is administered at a dose of about 1000 mg, and
wherein, at least 60% of the humans in the plurality achieve a complete response following the induction phase.

59. The method of claim 58, wherein the polatuzumab vedotin, the lenalidomide, and the obinutuzumab are administered during the induction phase for at least six 28-day cycles,
wherein the polatuzumab vedotin is administered intravenously at a dose of about 1.8 mg/kg on Day 1, the lenalidomide is administered orally at a dose of about 20 mg on each of Days 1-21, and the obinutuzumab is administered intravenously at a dose of about 1000 mg on each of Days 1, 8, and 15 of the first 28 day cycle, and
wherein the polatuzumab vedotin is administered intravenously at a dose of about 1.8 mg/kg on Day 1, the lenalidomide is administered orally at a dose between about 20 mg on each of Days 1-21, and the obinutuzumab is administered intravenously at a dose of about 1000 mg on Day 1 of each of the second, third, fourth, fifth, and sixth 28-day cycles.

60. The method of claim 58 or 59, wherein the induction phase is followed by a maintenance phase, wherein the lenalidomide is administered at a dose of about 10 mg and the obinutuzumab is administered at a dose of about 1000 mg during the maintenance phase.

61. The method of claim 60, wherein the lenalidomide is administered orally at a dose of about 10 mg on each of Days 1-21 of each month during the maintenance phase following the sixth 28-day cycle, and wherein the obinutuzumab is administered intravenously at a dose of about 1000 mg on Day 1 of every other month during the maintenance phase following the sixth 28-day cycle.

62. The method of any one of claims 58-61, wherein, among a plurality of humans treated, at least 75%, at least 80%, at least 85%, or at least 90% of the humans do not demonstrate disease progression within at least 12 months after the start of the induction phase.

63. The method of any one of claims 58-62, wherein, among a plurality of humans treated, the 12-month progression-free survival rate is at least 75%, at least 80%, at least 85%, or at least 90%, measured after the start of the induction phase.

64. The method of any one of claims 1-63, wherein the human or a human in the plurality of humans has received at least one prior therapy for FL.

65. The method of claim 64, wherein the at least one prior therapy was a chemoimmunotherapy that included an anti-CD20 antibody.

66. The method of any one of claims 1-65, wherein the FL is CD20-positive FL.

67. The method of any one of claims 1-66, wherein the human or a human in the plurality of humans has received at least two prior therapies for FL.

68. The method of any one of claims 1-67, wherein the human or a human in the plurality of humans was refractory to their most recent therapy for FL.

69. The method of any one of claims 1-68, wherein the FL is relapsed/refractory FL.

70. The method of any one of claims 1-69, wherein the FL is a positron emission tomography (PET)-positive lymphoma.

71. The method of any one of claims 1-70, wherein the human or a human in the plurality of humans does not have central nervous system (CNS) lymphoma or leptomeningeal infiltration.

72. The method of any one of claims 1-71, wherein the human or a human in the plurality of humans has not received prior allogenic stem cell transplantation (SCT).

73. The method of any one of claims 1-72, wherein administration of the immunoconjugate or polatuzumab vedotin, the immunomodulatory agent or lenalidomide, and the anti-CD20 antibody or obinutuzumab does not result in peripheral neuropathy of grade 3 or greater in the human or in a human in the plurality of humans.

74. A kit comprising an immunoconjugate comprising the formula 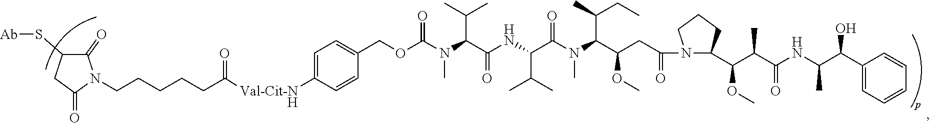
wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and
wherein p is between 1 and 8,
for use in combination with an immunomodulatory agent and an anti-CD20 antibody for treating a human in need thereof having follicular lymphoma (FL) according to a method of any one of claims 1-23 and 64-73.

75. A kit comprising an immunoconjugate comprising the formula 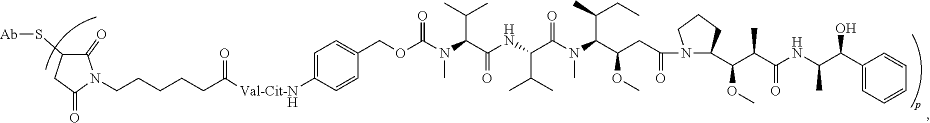
wherein Ab is an anti-CD79b antibody comprising (i) a heavy chain variable domain (VH) comprising the amino acid sequence of SEQ ID NO: 19 and (ii) a light chain variable domain (VL) comprising the amino acid sequence of SEQ ID NO: 20, and
wherein p is between 2 and 5,
for use in combination with lenalidomide and obinutuzumab for treating a human in need thereof having follicular lymphoma (FL) according to the method of any one of claims 24-39 and 64-73.

76. The kit of claim 74 or 75, wherein p is between 3 and 4.

77. The kit of any one of claims 74-76, wherein the antibody comprises (i) a heavy chain comprising the amino acid sequence of SEQ ID NO: 36 and (ii) a light chain comprising the amino acid sequence of SEQ ID NO: 35.

78. A kit comprising polatuzumab vedotin for use in combination with lenalidomide and obinutuzumab for treating a human in need thereof having follicular lymphoma (FL) according to the method of any one of claims 40-73.

79. The kit of any one of claims 74-78, wherein the FL is relapsed/refractory FL.

80. An immunoconjugate comprising the formula 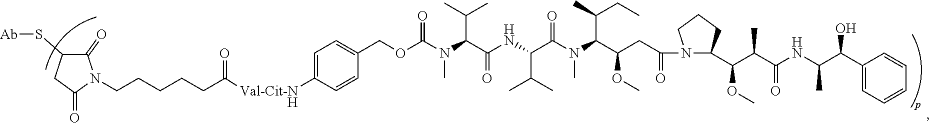
wherein is an anti-CD79b antibody comprising (i) an a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and
wherein p is between 1 and 8,
for use in a method of treating follicular lymphoma (FL) according to any one of claims 1-23 and 64-73.

81. The immunoconjugate of claim 80, wherein the anti-CD79b antibody comprises (i) a heavy chain variable domain (VH) that comprises the amino acid sequence of SEQ ID NO: 19 and (ii) a light chain variable domain (VL) that comprises the amino acid sequence of SEQ ID NO: 20.

82. An immunoconjugate comprising the formula 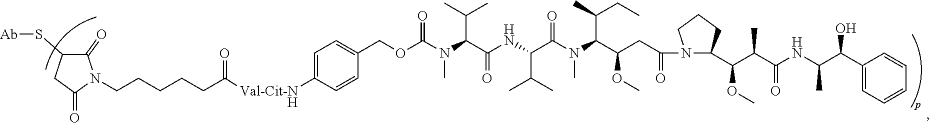
wherein Ab is an anti-CD79b antibody that comprises (i) a heavy chain variable domain (VH) comprising the amino acid sequence of SEQ ID NO: 19 and (ii) a light chain variable domain (VL) comprising the amino acid sequence of SEQ ID NO: 20, and
wherein p is between 2 and 5,
for use in a method of treating follicular lymphoma (FL) according to any one of claims 24-39 and 64-73.

83. The immunoconjugate of any one of claims 80-82, wherein p is between 3 and 4.

84. The immunoconjugate of any one of claims 80-83, wherein the anti-CD79b antibody comprises (i) a heavy chain comprising the amino acid sequence of SEQ ID NO: 36 and (ii) a light chain comprising the amino acid sequence of SEQ ID NO: 35.

85. Polatuzumab vedotin for use in a method of treating follicular lymphoma (FL) according to any one of claims 40-73.

86. The immunoconjugate for use according to any one of claims 80-84, or the polatuzumab vedotin for use according to claim 84 wherein the FL is relapsed/refractory FL.


SEQUENCE LISTING (56 sequences)

SEQ ID NO: 1 - protein, 179 aa, Homo sapiens
RFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 2 - protein, 201 aa, Homo sapiens
ARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 3 - protein, 112 aa, synthetic construct
  REGION 1..112: note = Synthetic Construct
GPELVKPGASVKISCKASGYAFSYSWMNWVKLRPGQGLEWIGRIFPGDGDTDYNGKFKGKATLTADKSSNTAYMQLTSLTSVDSAVYLCARNVFDGYWLVYWGQGTLVTVSA

SEQ ID NO: 4 - protein, 103 aa, synthetic construct
  REGION 1..103: note = Synthetic Construct
NPVTLGTSASISCRSSKSLLHSNGITYLYWYLQKPGQSPQLLIYQMSNLVSGVPDRFSSSGSGTDFTLRISRVEAEDVGVYYCAQNLELPYTFGGGTKLEIKR

SEQ ID NO: 5 - protein, 6 aa, synthetic construct
  REGION 1..6: note = Synthetic Construct
GYAFSY

SEQ ID NO: 6 - protein, 8 aa, synthetic construct
  REGION 1..8: note = Synthetic Construct
FPGDGDTD

SEQ ID NO: 7 - protein, 10 aa, synthetic construct
  REGION 1..10: note = Synthetic Construct
NVFDGYWLVY

SEQ ID NO: 8 - protein, 16 aa, synthetic construct
  REGION 1..16: note = Synthetic Construct
RSSKSLLHSNGITYLY

SEQ ID NO: 9 - protein, 7 aa, synthetic construct
  REGION 1..7: note = Synthetic Construct
QMSNLVS

SEQ ID NO: 10 - protein, 9 aa, synthetic construct
  REGION 1..9: note = Synthetic Construct
AQNLELPYT

SEQ ID NO: 11 - protein, 119 aa, synthetic construct
  REGION 1..119: note = Synthetic Construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 12 - protein, 115 aa, synthetic construct
  REGION 1..115: note = Synthetic Construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTV

SEQ ID NO: 13 - protein, 448 aa, synthetic construct
  REGION 1..448: note = Synthetic Construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 219 aa, synthetic construct
  REGION 1..219: note = Synthetic Construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 119 aa, synthetic construct
  REGION 1..119: note = Synthetic Construct
QVQLVQSGAEVKKPGSSVKVSCKASGYAFSYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 16 - protein, 119 aa, synthetic construct
  REGION 1..119: note = Synthetic Construct
QVQLVQSGAEVKKPGSSVKVSCKASGYAFSYSWMNWVRQAPGQGLEWMGRIFPGDGDTDYNGKFKGRVTITADKSTSTAYMELSSLRSEDTAVYLCARNVFDGYWLVYWGQGTLVTVSS

SEQ ID NO: 17 - protein, 448 aa, synthetic construct; identical to SEQ ID NO: 13
  REGION 1..448: note = Synthetic Construct

SEQ ID NO: 18 - protein, 219 aa, synthetic construct; identical to SEQ ID NO: 14
  REGION 1..219: note = Synthetic Construct

SEQ ID NO: 19 - protein, 117 aa, synthetic construct [sequence omitted]

SEQ ID NO: 20 - protein, 112 aa, synthetic construct
  REGION 1..112: note = Synthetic Construct
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKR

SEQ ID NO: 21 - protein, 10 aa, synthetic construct
  REGION 1..10: note = Synthetic Construct
GYTFSSYWIE

SEQ ID NO: 22 - protein, 18 aa, synthetic construct
  REGION 1..18: note = Synthetic Construct
GEILPGGGDTNYNEIFKG

SEQ ID NO: 23 - protein, 10 aa, synthetic construct
  REGION 1..10: note = Synthetic Construct
TRRVPIRLDY

SEQ ID NO: 24 - protein, 15 aa, synthetic construct
  REGION 1..15: note = Synthetic Construct
KASQSVDYEGDSFLN

SEQ ID NO: 25 - protein, 7 aa, synthetic construct
  REGION 1..7: note = Synthetic Construct
AASNLES

SEQ ID NO: 26 - protein, 9 aa, synthetic construct
  REGION 1..9: note = Synthetic Construct
QQSNEDPLT

SEQ ID NO: 27 - protein, 25 aa, synthetic construct [sequence omitted]

SEQ ID NO: 28 - protein, 13 aa, synthetic construct
  REGION 1..13: note = Synthetic Construct
WVRQAPGKGLEWI

SEQ ID NO: 29 - protein, 30 aa, synthetic construct
  REGION 1..30: note = Synthetic Construct
RATFSADTSKNTAYLQMNSLRAEDTAVYYC

SEQ ID NO: 30 - protein, 11 aa, synthetic construct
  REGION 1..11: note = Synthetic Construct
WGQGTLVTVSS

SEQ ID NO: 31 - protein, 23 aa, synthetic construct
  REGION 1..23: note = Synthetic Construct
DIQLTQSPSSLSASVGDRVTITC

SEQ ID NO: 32 - protein, 15 aa, synthetic construct
  REGION 1..15: note = Synthetic Construct
WYQQKPGKAPKLLIY

SEQ ID NO: 33 - protein, 32 aa, synthetic construct
  REGION 1..32: note = Synthetic Construct
GVPSRFSGSGSGTDFTLTISSLQPEDFATYYC

SEQ ID NO: 34 - protein, 11 aa, synthetic construct
  REGION 1..11: note = Synthetic Construct
FGQGTKVEIKR

SEQ ID NO: 35 - protein, 218 aa, synthetic construct
  REGION 1..218: note = Synthetic Construct
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 446 aa, synthetic construct
  REGION 1..446: note = Synthetic Construct
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 37 - protein, 446 aa, synthetic construct
  REGION 1..446: note = Synthetic Construct
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 38 - protein, 218 aa, synthetic construct
  REGION 1..218: note = Synthetic Construct
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

SEQ ID NO: 39 - protein, 447 aa, synthetic construct
  REGION 1..447: note = Synthetic Construct
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDCDGSFFLYSKLTVDKSRWQQGNVFSCSVMHEALHNHYTQKSLSLSPGK

SEQ ID NO: 40 - protein, 119 aa, synthetic construct
  REGION 1..119: note = Synthetic Construct
QVQLVQSGAEVKKPGASVKVSCKVSGYAFSYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 41 - protein, 119 aa, synthetic construct
  REGION 1..119: note = Synthetic Construct
QVQLVQSGAEVKKPGSSVKVSCKASGYAFSYSWMSWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 42 - protein, 119 aa, synthetic construct; identical to SEQ ID NO: 11
  REGION 1..119: note = Synthetic Construct

SEQ ID NO: 43 - protein, 119 aa, synthetic construct
  REGION 1..119: note = Synthetic Construct
QVQLVQSGAEVKKPGSSVKVSCKASGYAFSYSWISWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 44 - protein, 119 aa, synthetic construct
  REGION 1..119: note = Synthetic Construct
QVQLVQSGAEVKKPGASVKVSCKASGYTFTYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 45 - protein, 119 aa, synthetic construct
  REGION 1..119: note = Synthetic Construct
QVQLVQSGAEVKKPGASVKVSCKASGYTFSYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 46 - protein, 119 aa, synthetic construct
  REGION 1..119: note = Synthetic Construct
EVQLVESGGGLVKPGGSLRLSCAASGFTFSYSWMNWVRQAPGKGLEWVGRIFPGDGDTDYNGKFKGRVTITADKSTSTAYMELSSLRSEDTAVYYCARNVFDGYWLVYWGQGTLVTVSS

SEQ ID NO: 47 - protein, 119 aa, synthetic construct
  REGION 1..119: note = Synthetic Construct
EVQLVESGGGLVKPGGSLRLSCAASGFAFSYSWMNWVRQAPGKGLEWVGRIFPGDGDTDYNGKFKGRVTITADKSTSTAYMELSSLRSEDTAVYYCARNVFDGYWLVYWGQGTLVTVSS

SEQ ID NO: 48 - protein, 119 aa, synthetic construct
  REGION 1..119: note = Synthetic Construct
QVQLVESGGGLVKPGGSLRLSCAASGFTFSYSWMNWVRQAPGKGLEWVGRIFPGDGDTDYNGKFKGRVTITADKSTSTAYMELSSLRSEDTAVYYCARNVFDGYWLVYWGQGTLVTVSS

SEQ ID NO: 49 - protein, 119 aa, synthetic construct
  REGION 1..119: note = Synthetic Construct
EVQLVESGAGLVKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 50 - protein, 119 aa, synthetic construct
  REGION 1..119: note = Synthetic Construct
EVQLVESGGGVVKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 51 - protein, 119 aa, synthetic construct
  REGION 1..119: note = Synthetic Construct
EVQLVESGGGLKKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 52 - protein, 119 aa, synthetic construct
  REGION 1..119: note = Synthetic Construct
EVQLVESGGGLVKPGSSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 53 - protein, 119 aa, synthetic construct
  REGION 1..119: note = Synthetic Construct
EVQLVESGGGLVKPGGSLRVSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 54 - protein, 119 aa, synthetic construct
  REGION 1..119: note = Synthetic Construct
EVQLVESGGGLVKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 55 - protein, 115 aa, synthetic construct; identical to SEQ ID NO: 12
  REGION 1..115: note = Synthetic Construct

SEQ ID NO: 56 - protein, 447 aa, synthetic construct
  REGION 1..447: note = Synthetic Construct
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK